(19) United States
(12) Patent Application Publication
(10) Pub. No.: US 2018/0289692 A1
(43) Pub. Date: Oct. 11, 2018

(54) TLR MODULATORS AND METHODS OF USE

(71) Applicant: CELGENE CORPORATION, Summit, NJ (US)

(72) Inventor: Gerald Scott Barden HORAN, Winchester, MA (US)

(21) Appl. No.: 15/764,269

(22) PCT Filed: Sep. 30, 2016
(86) PCT No.: PCT/US16/54734

§ 371 Date: Mar. 28, 2018

Related U.S. Application Data
(60) Provisional application No. 62/235,475, filed on Sep. 30, 2015.
(60) Provisional application No. 62/360,256, filed on Jul. 8, 2016.

(51) Int. Cl. A61K 31/4706; A61P 37/00; C12N 15/113; A61K 31/7088; A61K 47/54

(52) U.S. Cl. CPC A61K 31/4706; A61P 37/00; C12N 15/1136; A61K 31/7088; A61K 47/549; C12N 2320/32; C12N 2310/351; C12N 2310/17; C12N 2310/315; C12N 2310/3341; C12N 2310/111

(57) ABSTRACT

Described herein are combinations comprising a SMAD7 oligonucleotide and a Toll-like receptor (TLR) modulator. Methods for using such combinations to treat disease conditions, including inflammatory disorders, such as inflammatory bowel disease (IBD), are also provided herein.

### 1. CROSS-REFERENCE TO RELATED APPLICATIONS

This application claims benefit of priority to U.S. Provisional Application Ser. No. 62/360,256, filed Jul. 8, 2016, and U.S. Provisional Application Ser. No. 62/235,475, filed Sep. 30, 2015, which are incorporated herein in their entireties.

### 2. INTRODUCTION

Described herein are combinations comprising a SMAD7 oligonucleotide (ODN) and a Toll-like receptor (TLR) modulator. Methods for using such combinations to treat disease conditions, including inflammatory disorders, such as inflammatory bowel disease (IBD), are also provided herein.

### 3. BACKGROUND

Recent studies have demonstrated an involvement of the tumor growth factor beta (TGF-β) signaling pathway in inflammatory diseases. Specifically, SMAD7, an intracellular protein binding to TGF-β receptor and inhibiting TGF-β receptor signaling, has emerged as a drug target candidate for inflammatory disease indications, such as IBD.

IBD is a chronic inflammatory disorder of the gastrointestinal tract. The two most common forms of IBD are Crohn's disease (CD) and ulcerative colitis (UC). Although CD primarily affects the terminal ileum and right colon CD can affect the entire gastrointestinal tract. UC primarily affects the colon and the rectum. Current treatments for both CD and UC include aminosalicylates, antibiotics, corticosteroid, immunosuppressants and tumor necrosis factor alpha (TNFα) antagonists. However, patient responses to these treatments can vary with disease severity and many current treatments are associated with undesirable side effects. Thus there is a need to identify new treatments for IBD, including CD and UC

A SMAD7 antisense oligonucleotide was shown to down-regulate, prevent and treat CD-like symptoms in mice and Phase I and Phase II clinical studies suggested clinical benefits in human CD patients resulting from the administration of a SMAD7 antisense oligonucleotide.

Toll-like receptors (TLRs) are a type of pattern recognition receptors that play a key role in the innate immune system. Expressed on sentinel cells, such as macrophages and dendritic cells, TLRs generally recognize molecules that are broadly shared by pathogens, but distinguishable from host molecules. TLRs have long been viewed as instrumental in fending off especially bacterial and viral infections of a host. However, more recently, TLRs have emerged as attractive targets for a number of disease indications, including inflammatory diseases, autoimmune diseases, cancer, and others.

### 4. SUMMARY

In one aspect, provided herein is a method of treating a disease in a patient in need thereof, comprising administering to the patient effective amounts of a SMAD7 antisense oligonucleotide (AON) comprising a nucleotide sequence complementary to region 108-128 of the human SMAD7 nucleotide sequence of SEQ ID NO: 1, and a compound capable of modulating a TLR, wherein the SMAD7 AON and the compound capable of modulating the TLR are different compounds.

In some embodiments, the SMAD7 AON targets nucleotides 403, 233, 294, 295, 296, 298, 299, or 533 of the nucleic acid sequence of SEQ ID NO: 1.

In some embodiments, the SMAD7 AON comprises COMPOUND (I).

In some embodiments, the TLR is TLR3, TLR7, TLR8, or TLR9. In some embodiments, the TLR is TLR9.

In some embodiments, a compound is capable of modulating the TLR if the compound can increase the expression of IP10, TNFα or IL-6 protein by a peripheral dendritic cell (pDC), when the compound is contacted with the pDC at a concentration of less than 1.0 μM, relative to a pDC control not contacted with the compound, as determined in an immunoassay.

In some embodiments, a compound is capable of modulating the TLR if the compound can increase the expression of TNFα, IFNγ, TGFβ, IL-6, IL-10, PD-L1, IDO, or ICOS-L protein and decrease the expression of IP10 protein by a pDC, when the compound is contacted with the pDC at a concentration of about 1.0 μM or more, relative to a pDC control not contacted with the compound, as determined in an immunoassay.

In some embodiments, the compound is capable of modulating the TLR if the compound increases the expression of ICOS-L proteins by a pDC by a factor of 5-fold or more, when contacted with the pDC at a concentration of 1.0 μM or more, in the presence of a quinoline or quinine relative to a pDC control not contacted with the compound, as determined in an immunoassay, wherein the quinoline or quinine is present at a concentration below the threshold concentration at which the quinoline or quinine alone can detectably increase ICOS-L expression.

In some embodiments, the quinoline is hydroxychloroquine.

In some embodiments, the compound is capable of modulating the TLR if the compound can reduce the PolyI:C-induced IFNα secretion of peripheral blood mononuclear cells (PBMCs), when the compound is contacted with the PBMCs at a concentration of 1.0 μM or less, relative to a PolyI:C-induced PBMC control not contacted with the compound.

In some embodiments, the compound is capable of modulating the TLR if the compound can reduce the imiquimod-induced IFNα secretion of peripheral blood mononuclear cells (PBMCs), when the compound is contacted with the PBMCs at a concentration of 0.1 μM or less, relative to an imiquimod-induced PBMC control not contacted with the compound.

In some embodiments, the compound is capable of modulating the TLR if the compound can induce IL-1β secretion from a PBMC and induce pDC differentiation, when the compound is contacted with the pDC or PBMC at a concentration of 1.0 μM or more.

In some embodiments, the compound is capable of modulating the TLR if the compound cannot detectably induce B-cell proliferation, or only weakly induces B-cell proliferation, when contacted with a B-cell at a concentration of 10.0 μM or less.

In some embodiments, the compound capable of modulating TLR is BL-7040 (ODN7040), CYT003, CYT003-QbG10, AZD1419, DIMS0150 (ODN150), E6446, CpG ODN2088, IMO-8400, IMO-3100, CL075, VTX-2337, ODN2006, or naltrexone.

In some embodiments, the compound capable of modulating the TLR is an antimalarial therapeutic selected from the group consisting of a quinine, a chloroquine, an amodiaquine, a mefloquine, a primaquine, or a derivative thereof.

In some embodiments, the antimalarial therapeutic is hydroxylchloroquine (Plaquenil).

In some embodiments, the disease is selected from the group consisting of an inflammatory disease, an autoimmune disorder, an airway disease, an allergic disorder, a metabolic disorder, cancer, central nervous system (CNS) disorder, and a skin disease.

In some embodiments, the disease is selected from the group consisting of inflammatory bowel disease (IBD), Crohn's disease (CD), ulcerative colitis (UC), Sjogren's Syndrome, systemic lupus erythematosus (SLE), dry eye, autoimmune encephalitis, rheumatoid arthritis, multiple sclerosis, systemic sclerosis, psoriasis, colitis, uveitis, asthma, chronic pulmonary disease (COPD), allergic rhinitis, atopic dermatitis, Malaria, Hashimoto's encephalopathy, amoebiasis, diabetes, hyperlipidemia, non-alcoholic fatty liver disease, lung cancer, pancreas cancer, leukemic cancer, lymphoid cancer, pancreas cancer, breast cancer, prostate cancer, ovarian cancer, testicular cancer, melanoma, myeloma, glioblastoma, neuroblastoma, colorectal cancer, stomach cancer, multiple sclerosis, basal cell carcinoma, actinic keratosis.

In another aspect, provided herein is a method of treating a disease in a patient in need thereof, comprising administering to the patient an effective amount of a chemically modified SMAD7 antisense oligonucleotide (AON) comprising a nucleotide sequence complementary to region 108-128 of the human SMAD7 nucleotide sequence of SEQ ID NO: 1, wherein the SMAD7 AON is capable of modulating a TLR.

In some embodiments, the TLR is TLR3, TLR7, TLR8, or TLR9. In some embodiments, the TLR is TLR9.

In some embodiments, the SMAD7 AON comprises COMPOUND (I).

In some embodiments, the disease is selected from the group consisting of an autoimmune disorder, an airway disease, an allergic disorder, and a skin disease.

In some embodiments, the disease is selected from the group consisting of Sjogren's Syndrome, systemic lupus erythematosus (SLE), dry eye, autoimmune encephalitis, rheumatoid arthritis, systemic sclerosis, psoriasis, colitis, uveitis, asthma, chronic pulmonary disease (COPD), allergic rhinitis, atopic dermatitis, Malaria, Hashimoto's encephalopathy, amoebiasis, hyperlipidemia, non-alcoholic fatty liver disease, lung cancer, pancreas cancer, leukemic cancer, lymphoid cancer, pancreas cancer, breast cancer, prostate cancer, ovarian cancer, testicular cancer, melanoma, myeloma, glioblastoma, neuroblastoma, stomach cancer, multiple sclerosis, basal cell carcinoma, and actinic keratosis.

In another aspect, provided herein is method of treating a disease in a patient in need thereof, comprising (a) administering to the patient an effective amount of a SMAD7 AON comprising a nucleotide sequence complementary to region 108-128 of the human SMAD7 nucleotide sequence of SEQ ID NO: 1; (b) determining the patient's response to the ODN, and (c) if the patient does not respond to the SMAD7 AON, then, administering to the patient an effective amount of the SMAD7 AON and an effective amount of a compound capable of modulating a TLR.

In some embodiments, determining the patient's response to the SMAD7 AON comprises (a) analyzing a first level of a biomarker before administering the SMAD7 AON to the patient, and (b) analyzing a second level of the biomarker after administering the SMAD7 AON to the patient, wherein the patient responds to the SMAD7 AON if the second biomarker level is lower than the first biomarker level.

In some embodiments, the SMAD7 AON comprises COMPOUND (I).

In some embodiments, the disease is IBD. In some embodiments, the disease is Crohn's Disease or Ulcerative Colitis.

In some embodiments, the SMAD7 AON is COMPOUND (I), and the compound capable of modulating a TLR is hydroxychloroquine.

In another aspect, provided herein is a method of screening for a compound capable of synergizing with a SMAD7 AON, comprising (a) contacting the SMAD7 AON with a cell of the immune system at a first concentration; (b) determining a first expression level of a TLR pathway component in the cell of the immune system; (c) contacting the cell of the immune system with the SMAD7 AON at the first concentration and with a test compound at a second concentration, and (d) determining a second expression level of the TLR pathway component in the cell of the immune system; wherein the test compound is capable of synergizing with the SMAD7 AON, if the second expression level of the TLR pathway component in the cell of the immune system is higher than the first expression level of the TLR pathway component in the cell of the immune system.

In some embodiments, the cell of the immune system is a PBMC or a pDC.

In some embodiments, the TLR pathway component is TNFα, IFNγ, IL-1β, IL-10, TGFβ, PD-L1, ICOS-L or IP-10 (CXCL10).

In some embodiments, the TLR pathway component is CCL2, CCL7, CD-69, IL1-β, IL-18, MCP-1, phospho-histone H3, phospho-p38 MAP kinase, or phospho-ZAP70

In some embodiments, the SMAD7 AON alone at the first concentration is capable of increasing the TLR pathway component level in the immune cell less than 2-fold, compared to a control sample in which the SMAD7 AON is absent, wherein the test compound alone at the second concentration does not detectably increase the expression level of the TLR pathway component in the immune cell compared to a control sample in which the SMAD7 AON is absent.

In some embodiments, the test compound is capable of synergizing with the SMAD7 AON, if the second expression level of the TLR pathway component in the cell of the immune system is at least 3-fold, at least 5-fold, at least 10-fold, at least 15-fold, or at least 20-fold higher than the first expression level of the TLR pathway component in the cell of the immune system.

In another aspect, provided herein is a pharmaceutical composition comprising a SMAD7 AON comprising a nucleotide sequence complementary to region 108-128 of the human SMAD7 nucleotide sequence of SEQ ID NO: 1, a compound capable of modulating a TLR, and an excipient.

In some embodiments, the SMAD7 AON is COMPOUND (I).

In some embodiments, the TLR is TLR3, TLR7, TLR8, or TLR9. In some embodiments, the TLR is TLR9.

In some embodiments, the compound capable of modulating the TLR is hydroxychloroquine.

In some embodiments, the SMAD7 AON and the compound capable of modulating TLR are covalently linked.

In another aspect, provided herein is a chemically modified SMAD7 AON comprising a nucleotide sequence complementary to region 108-128 of the human SMAD7 nucleotide sequence of SEQ ID NO: 1, wherein the SMAD7 AON is capable of modulating a TLR, for use in a method of treating a disease in a patient in need thereof, wherein the disease is selected from the group consisting of an autoimmune disorder, an airway disease, an allergic disorder, and a skin disease.

In another aspect, provided herein is a chemically modified SMAD7 AON comprising a nucleotide sequence complementary to region 108-128 of the human SMAD7 nucleotide sequence of SEQ ID NO: 1, wherein the SMAD7 AON is capable of modulating a TLR, for use in a method of treating a disease in a patient in need thereof, wherein the disease is selected from the group consisting of Sjogren's Syndrome, systemic lupus erythematosus (SLE), dry eye, autoimmune encephalitis, rheumatoid arthritis, systemic sclerosis, psoriasis, colitis, uveitis, asthma, chronic pulmonary disease (COPD), allergic rhinitis, atopic dermatitis, Malaria, Hashimoto's encephalopathy, amoebiasis, hyperlipidemia, non-alcoholic fatty liver disease, lung cancer, pancreas cancer, leukemic cancer, lymphoid cancer, pancreas cancer, breast cancer, prostate cancer, ovarian cancer, testicular cancer, melanoma, myeloma, glioblastoma, neuroblastoma, stomach cancer, multiple sclerosis, basal cell carcinoma, and actinic keratosis.

In some embodiments, the method further comprises administering a compound capable of modulating a TLR.

In some embodiments, the method further comprises (i) determining the patient's response to the ODN, and (ii) if the patient does not respond to the SMAD7 AON, then administering to the patient an effective amount of the SMAD7 AON and an effective amount of a compound capable of modulating a TLR.

### 5. BRIEF DESCRIPTION OF THE DRAWINGS

FIGS. 1A-C illustrate the effect of COMPOUND (I) on SMAD7 expression in human peripheral blood mononuclear cells (PBMCs) and in human plasmacytoid dendritic cells (pDCs). FIG. 1A shows bar graphs illustrating SMAD7 mRNA levels in human PBMCs following treatment with COMPOUND (I) or with the oligonucleotide ODN2006, at indicated concentrations, for 24 hrs, as determined by RT-PCR. FIG. 1B and FIG. 1C show bar graphs illustrating SMAD7 protein levels in human pDCs following treatment with COMPOUND (I) or with the oligonucleotides ODN7040, ODN150 or ODN2006, at indicated concentrations, for 24 hrs (FIG. 1B) or 48 h (FIG. 1C), as determined by FACS (quantification of CCR6+/CD123+ cells).

FIGS. 2A-B illustrate the effect of COMPOUND (I) on TGFβ or PD-L1 expression in human pDCs. FIGS. 2A-B show bar graphs illustrating TGFβ (FIG. 2A) or PD-L1 (FIG. 2B) protein expression levels in human pDCs following 24 hrs of treatments with COMPOUND (I) or with the oligonucleotides ODN301C, ODN150, ODN7040, ODN302 or ODN2006, at indicated concentrations, as determined by FACS. Average values and standard deviations for n=2 replicate experiments are shown.

FIGS. 3A-C illustrate the effects of COMPOUND (I) or of the oligonucleotides ODN2006 or ODN2216 on IDO or ICOS-L expression in human pDCs. FIGS. 3A-C show bar graphs illustrating IDO (FIG. 3A), ICOS-L (FIG. 3B), and IL-10 (FIG. 3C) protein expression levels in human pDCs following 48 hrs of treatments with COMPOUND (I) or with the oligonucleotides ODN2006 or ODN2216, at indicated concentrations, as determined by FACS. Average values and standard deviations for n=3 replicate experiments are shown.

FIGS. 4A-B show graphs illustrating the effects of COMPOUND (I) or of oligonucleotides ODN7040, ODN150 or ODN2006 on IDO protein expression in human pDCs by, as detected by FACS. FIG. 4A shows a graph illustrating the relative levels of IDO protein expression in human pDCs following 48 hrs of treatments with COMPOUND (I), ODN7040, or ODN150, at indicated concentrations, as determined by FACS. Average values and standard deviations for n=3 replicate experiments are shown. FIG. 4B shows a graph illustrating relative IDO protein expression levels in human pDCs following 48 hrs of treatments with COMPOUND (I) (solid line) or ODN2006 (dashed line), at indicated concentrations, as determined by FACS.

FIGS. 5A-B show graphs illustrating the effects of COMPOUND (I) or of the oligonucleotides ODN7040 and ODN150 on ICOS-L protein expression in human pDCs, following oligonucleotide treatments for 24 hrs (FIG. 5A) or 48h (FIG. 5B), at indicated concentrations, as determined by FACS.

FIGS. 6A-B show graphs illustrating the synergistic effect of COMPOUND (I) and hydroxychloroquine (HCQ; 10 μM) with respect to ICOS-L protein induction in pDCs. FIG. 6A and FIG. 6B show results of two independent experiments performed on pDC from three donors in each experiment. Controls: isotype control (Iso); vehicle control; ODN1826C. MFI=Mean Fluorescence Intensity.

FIGS. 7A-B show graphs illustrating the effects of COMPOUND (I) or of the oligonucleotides ODN2137, ODN2006, or ODN2216 on B-cell activation in human PBMCs, as determined by FACS measurements of CD86 protein expression. FIGS. 7A and 7B show graphs illustrating relative quantities of activated B-cells (CD86⁺ cells) in a B-cell population (CD19⁺ cells) following treatments with COMPOUND (I) or with the oligonucleotides ODN2137, ODN2006, or ODN2216, at indicated concentrations, for 24 hrs.

FIGS. 8A-C illustrate the effect of COMPOUND (I) on IFNα induction in human PBMCs through TLR3, TLR7 or TLR9 pathways, as determined by ELISA. FIG. 8A illustrates the effect of indicated concentrations of COMPOUND (I) on TLR3 (polyC)-induced IFNα. FIG. 8B illustrates the effect of indicated concentrations of COMPOUND (I) on TLR7 (imiquimod)-induced IFNα. FIG. 8C illustrates the effect of indicated concentrations of COMPOUND (I) on TLR3 (ODN2216)-induced IFNα.

FIGS. 9A-B show graphs illustrating the relative effects of indicated concentrations of COMPOUND (I) or of oligonucleotides ODN2006, ODN7040 or ODN150 on TLR7 (imiquimod)-induced IFNα (FIG. 9A) or TLR7 (imiquimod)-induced CXCL10 (FIG. 9B) in human PBMCs, as determined by ELISA.

FIG. 10 shows a graph illustrating the relative effects of indicated concentrations of COMPOUND (I) or of oligonucleotides ODN2006, ODN7040, ODN150, or ODN302 on the activation of a canonical NF-kB reporter construct in HEK293 cells.

FIG. 11 shows a graph illustrating the relative effects of indicated concentrations of COMPOUND (I) or of oligonucleotides ODN1826, ODN1826C, ODN7040, ODN7040C, ODN150, ODN150C, ODN302, or ODN301C on the activation of a mouse NF-kB reporter construct in mouse RAW 264.7 macrophage cells.

FIG. 12 shows a graph illustrating the relative effects of indicated concentrations of COMPOUND (I) or of oligonucleotides ODN1826, ODN1826C, ODN7040, ODN7040C, ODN150, ODN150C, ODN302, or ODN301C on the activation of a mouse TNFα reporter construct in mouse RAW 264.7 macrophage cells.

FIG. 13 shows a graph illustrating the relative effects of indicated concentrations of COMPOUND (I) or of oligonucleotides ODN1826, ODN1826C, ODN7040, ODN7040C, ODN150, ODN150C, ODN302, or ODN301C on the activation of a mouse IL10 reporter construct in mouse RAW 264.7 macrophage cells.

FIGS. 14A-C show graphs illustrating the relative effects of indicated concentrations of COMPOUND (I) or of oligonucleotide ODN2006 on IDO protein expression in pDCs derived from three human donors.

FIG. 15 illustrates the relative effects of COMPOUND (I) or of oligonucleotides ODN150, ODN2006, or ODN7040, at indicated concentrations, on IL-1β protein secretion from human PBMCs, as determined by ELISA.

FIGS. 16A-B show a graph illustrating the relative effects of COMPOUND (I) or of oligonucleotides ODN150, ODN2006, and ODN7040, at indicated concentrations, on the activation of a human NF-κB reporter construct in TLR9 expressing HEK293 cells. FIG. 16A shows a bar diagram illustrating the relative effects of COMPOUND (I) and hydroxychloroquine (HCQ) treatments of TLR9 expressing HEK293 reporter cells containing a human NF-κB reporter construct. FIG. 16B shows a graph with titration curves illustrating the concentration-dependent NF-kB induction by ODN150, ODN2006, or ODN7040, but not COMPOUND (I).

FIG. 17 shows a graph with titration curves, illustrating the effect of COMPOUND (I) or of oligonucleotides ODN150, ODN302, ODN2006, ODN2006C, or ODN7040 at indicated concentrations on B-cell proliferation following 96 hrs of treatments of B-cells with oligonucleotides, as determined by flow cytometry.

FIG. 18 shows a graph with bar diagrams, illustrating the effect of COMPOUND (I) or of oligonucleotides ODN150, ODN2006, or ODN7040 on IFNα production in pDCs, at a 3 μM concentration each, following 48 hrs of treatments of pDCs with oligonucleotides, as determined by ELISA.

FIG. 19 shows a graph with titration curves, illustrating the effect of COMPOUND (I) or of oligonucleotides ODN301C, ODN2006, ODN2006C, or ODN302, at indicated concentrations, on IL-1β secretion from NOD2 ligand L18-MDP (100 ng/ml) stimulated PBMCs, as determined by ELISA following 1 h PBMC pretreatment with control vehicle, COMPOUND(I) or other oligonucleotides, and following additional 24 hrs of L18-MDP stimulation.

FIGS. 20A-B show graphs with titration curves, illustrating the effect of COMPOUND (I) or of oligonucleotide ODN2006, at indicated concentrations, on IL-1β secretion from unstimulated PBMCs (FIG. 20A) and from LPS stimulated PBMCs (FIG. 20B), as determined by ELISA following 1 h PBMC pretreatment with control vehicle, COMPOUND(I) or ODN2006, and following additional 24 hrs of L18-MDP stimulation.

FIGS. 21A-D show bar diagrams, illustrating the effect of COMPOUND (I) or of oligonucleotides ODN2006 or ODN7040, at indicated concentrations, on pDC differentiation, as determined through the detection of pDC differentiation marker expression by flow cytometry. FIG. 21A illustrates the effect of COMPOUND (I), ODN2006, or ODN7040 on CD83 expression on pDCs. FIG. 21B illustrates the effect of COMPOUND (I), ODN2006, or ODN7040 on CD86 expression on pDCs. FIG. 21C illustrates the effect of COMPOUND (I), ODN2006, or ODN7040 on CCR6 expression on pDCs. FIG. 21D illustrates the effect of COMPOUND (I), ODN2006, or ODN7040 on CCR7 expression on pDCs.

FIG. 22 shows flow cytometry profiles illustrating results of flow cytometry experiments analyzing CD123 expression in human PBMCs following treatments with ODN2006, COMPOUND (I), hydroxychloroquine (HCQ), or a combination of COMPOUND (I) and hydroxychloroquine at 3 μM each for 24 hrs.

FIG. 23 shows scatter plots illustrating results of flow cytometry experiments analyzing CD123 and CCR6 differentiation marker expression in pDCs following treatment with COMPOUND (I) or the oligonucleotide ODN2006 at indicated concentrations for 24 hrs.

FIG. 24 shows a graph illustrating results of experiments analyzing TLR8 activation in a HEK Blue reporter cell line, following lipo-transfection with COMPOUND (I), ODN150, ODN2006, or ODN7040, at indicated concentrations, after 22 hrs incubation.

FIG. 25 shows a graph illustrating results of experiments analyzing the effect of treatments with COMPOUND(I), ODN2006, ODN150, ODN301C and ODN7040, at indicated concentrations, on IFNα secretion from PBMCs.

### 6. ABBREVIATIONS

Abbreviations

| | |
|---|---|
| AON | Antisense oligonucleotide |
| CCR6 | Chemokine (C-C motif) receptor 6 |
| CCR7 | Chemokine (C-C motif) receptor 7 |
| CCL2 | Chemokine (C-C motif) ligand 2 (MCP-1) |
| CCL7 | Chemokine (C-C motif) ligand 7 |
| CCL20 | Chemokine (C-C motif) ligand 20 |
| CD | Crohn's disease |
| CD4 | Cluster of differentiation 4 |
| CD8 | Cluster of differential on 8 |
| CD69 | Cluster of differentiation 69 |
| CD83 | Cluster of differentiation 83 |
| CD86 | Cluster of differentiation 86 |
| CD123 | Interleukin-3 receptor alpha chain |
| CRP | C-reactive protein |
| CXCL10 | Chemokine (C-X-C motif) ligand 10; IP-10; interferon |
| | gamma-induced protein 10 |
| DIMS | DNA immuno modulatory sequence |
| ELISA | Enzyme-linked immunosorbent assay |
| EOT-3 | Eotaxin-3 |
| FACS | Fluorescence associated cell sorting |
| FCP | Fecal calprotectin |
| HCQ | Hydroxychloroquine |
| HLA-DR | Human leukocyte antigen DR |
| IBD | Inflammatory bowel disease |
| IBD1 | Inflammatory bowel disease protein 1 |
| ICAM-1 | Intercellular adhesion molecule 1 |
| ICOS-L | Inducible T-cell co-stimulator ligand |
| IDO | Indoleamine 2,3-dioxygenase |
| IFNα | Interferon alpha |
| IFNγ | Interferon gamma |
| IgG | Immunoglobulin G |
| IL-1α | Interleukin-1 alpha |
| IL-1β | Interleukin-1 beta |
| IL-4 | Interleukin 4 |
| IL-6 | Interleukin 6 |
| IL-8 | Interleukin 8 |
| IL-10 | Interleukin 10 |
| IL-10Rα | Interleukin 10 receptor alpha chain |
| IL-12p40 | Interleukin 12, p40 |
| IL-17 | Interleukin 17 |
| IL-18 | Interleukin 18 |
| IL-18RAP | Interleukin receptor accessory protein |
| IL-23 | Interleukin 23 |
| IP-10 | Interferon gamma-induced protein 10; CXCL10; |
| | chemokine (C-X-C motif) ligand 10 |
| I-TAC | Interferon-inducible T-cell alpha chemoattractant |
| JAK | Janus kinase |
| LPS | Lipopolysaccharide |
| MAP kinase | Mitogen activated protein kinase |
| MCP-1 | Monocyte chemoattractant protein 1 (CCL2) |
| M-CSF | Macrophage colony-stimulating factor |
| MDP | Muramyl dipeptide |
| MFI | Mean fluorescence intensity |
| MIG | Monokine induced by gamma interferon |
| MIP-1 | Macrophage inflammatory protein 1 |
| MTD | Maximum tolerated dose |
| NLRP | Nod-like receptor protein |
| NALP3 | NACHT, LRR and PYD domains-containing protein 3 |
| NF-κB | Nuclear factor ‘kappa-light-chain-enhancer’ |
| | of activated B-cells |
| NOD2 | Nucleotide-binding oligomerization domain- |
| | containing protein 2 (NOD2) |
| ODN | Oligodeoxynucleotide |
| PBMC | Peripheral blood mononuclear cell |
| PO | Phosphate internucleoside linkage |
| PS | Phosphorothioate internucleoside linkage |
| pDC | Plasmacytoid dendritic cells |
| PD-L1 | Programmed death-ligand 1 |
| PYCARD | Apoptosis-associated speck-like protein containing |
| | a caspase-associated recruitment domain |
| RT-PCR | Reverse transcription polymerase chain reaction |
| SLAMF7 | Signaling lymphocyte activation molecule family |
| | member 7 |
| SMAD3 | SMAD family member 2 |
| SMAD7 | SMAD family member 7 |
| SPR | Surface plasmon resonance |
| TGFβ | Transforming growth factor beta |
| TLR3 | Toll-like receptor 3 |
| TLR4 | Toll-like receptor 4 |
| TLR7 | Toll-like receptor 7 |
| TLR8 | Toll-like receptor 8 |
| TLR9 | Toll-like receptor 9 |
| TNFα | Tumor necrosis factor alpha |
| Treg | Regulatory T-cell |
| Tyk2 | Tyrosine kinase 2 |
| UC | Ulcerative colitis |
| ZAP90 | Zeta chain (T-cell receptor) associated |
| | protein kinase |

### 7. DETAILED DESCRIPTION

Without wishing to be bound by theory, the present application provides data demonstrating that certain antisense oligonucleotides have dual activity, i.e., they can be designed so as to acquire activities beyond the inhibition of gene expression. More specifically, and surprisingly, in addition to its inhibition of SMAD7 expression, a SMAD7 antisense oligonucleotide can also induce certain cellular signaling events. These signaling events can involve Toll-Like Receptors (TLRs) and result in the expression of certain key mediators of immunosuppressive activity (such as, e.g., indoleamine dioxygenase (“IDO”)). Thus, downregulation of SMAD7 and induction of mediators of immunosuppressive activity can be combined to act in concert to treat certain inflammatory disorders (such as IBD) and other disease indications. Accordingly, provided herein are combinations of anti-SMAD7 therapeutics and modulators of TLRs. Compositions that combine anti-SMAD7 activity and modulation of TLRs in a single molecule are also provided herein. Furthermore, certain downstream targets of TLRs can serve as biomarkers for such treatments. The use of such compositions and combinations for the treatment and prevention of various diseases is also disclosed herein. In addition, methods for identifying such dual activity compounds are also disclosed.

The present application is further based on the surprising finding that certain TLR modulators (e.g., certain SMAD7 AONs) can have a unique biological activity profile with respect to TLR9-mediated immune cell activation that is similar to, and yet distinguishable from, the activity profile of canonical or otherwise known TLR9 modulators. For example, certain TLR modulators provided herein can induce pDC differentiation and/or PBMC inflammasome activation similar to canonical CpG-B class TLR9 agonists, without sharing the activity of the canonical CpG-B class TLR9 agonists with respect to the induction of B-cell proliferation or NF-κB pathway activation. Certain TLR modulators provided herein can also have TLR9-mediated activities on immune cells that are distinguishable from the activities of certain other known TLR9 modulators such as Kappaproct® (ODN150) or Monarsen® (ODN7040). For example, certain TLR9 modulators provided herein can strongly induce inflammasome activity in PBMCs and/or strongly induce pDC differentiation, both of which activities are either lacking or much weaker in Kappaproct® and Monarsen®. The unique TLR9-mediated activity profiles of certain TLR modulators provided herein can be beneficial in the treatment of the diseases described herein, such as inflammatory bowel disease (IBD). Accordingly, methods for using the TLR modulators with the unique TLR9-mediated immunce cell activity profiles provided herein in the treatment of the diseases described herein are also provided, as are methods for identifying TLR modulators with the described unique activity profiles.

Provided herein are combinations comprising a SMAD7 oligonucleotide (SMAD7 ODN) and a Toll-Like Receptor (TLR) modulator, pharmaceutical compositions comprising such combinations, and uses of such combinations for the treatment of diseases. See Sections 7.1., 7.6. and 7.7. Also provided herein are SMAD7 ODNs capable of modulating a TLR and methods of screening for such SMAD7 ODNs. See Sections 7.8. and 7.9. In some embodiments, the SMAD7 ODN is an anti-SMAD7 ODN (e.g., SMAD7 AON, SMAD7 RNAi, SMAD7 miRNA).

As used herein, “anti-SMAD7 ODN” refers to an oligonucleotide (ODN) comprising a nucleic acid sequence that is complementary to a nucleic acid sequence in the coding region of SMAD7. In some embodiments, the anti-SMAD7 ODN comprises a SMAD7 AON, SMAD7 RNAi, or SMAD7 miRNA. See, e.g., Sections 7.4(a) and (b). For example, a SMAD7 AON is an ODN comprising a nucleic acid sequence that is complementary to the nucleic acid sequence of a SMAD7 mRNA, SMAD7 cDNA, or the coding strand of a SMAD7 DNA. In some embodiments, the anti-SMAD7 ODN (e.g., SMAD7 AON) can reduce the expression of a gene that comprises a complementary nucleic acid sequence when the anti-SMAD7 ODN is introduced into a cell (e.g., an immune cell, such as PBMC, pDC, or B-cell). In some embodiments, the anti-SMAD7 ODN (e.g., SMAD7 AON) can reduce expression of an mRNA transcribed from the gene. In some embodiments, the anti-SMAD7 ODN (e.g., SMAD7 AON) can reduce expression of a protein encoded by the gene. In some embodiments, the anti-SMAD7 ODN can reduce secretion of a protein encoded by the gene from the cell into which the anti-SMAD7 ODN was introduced.

As used herein, “oligonucleotide (ODN)” refers to nucleic acids comprising a nucleic acid sequence corresponding to a nucleic acid sequence in the coding region of a gene or of an mRNA transcribed from the gene, and to nucleic acids comprising a nucleic acid sequence that is complementary to a nucleic acid sequence in the coding region of a gene or in an mRNA transcribed from the gene (anti-ODN). For example, “SMAD7 ODN” refers to nucleic acids comprising a nucleic acid sequence corresponding to a nucleic acid sequence in the coding region of SMAD7 or in a SMAD7 mRNA, and to anti-SMAD7 ODNs.

As used herein, “chemically modified” refers to any chemical modification of a compound, such as an oligonucleotide (ODN). In some embodiments, the chemically modified ODN is a chemically modified AON. In some embodiments, the chemically modified ODN can be derived, e.g., from a naturally occurring ODN by modifying in a chemical reaction, e.g., one or more bases, sugar residues or internucleoside linkages. In some embodiments, the chemically modified ODN comprises one or more phosphorothioate linkages and/or one or more methylated bases (e.g., 5-methyl-cytosine, 6-O-methyl-guanine, or 7-methyl-guanine). Additional exemplary chemical modifications of an ODN are described, e.g., in Section 7.10. COMPOUND (I) is one example of a chemically modified ODN.

As used herein, “inflammasome” refers to a multiprotein oligomer typically expressed in myeloid cells (e.g., monocytes, macrophages, neutrophiles, basophiles, eosinophiles, erythrocytes, dendritic cells, and megakaryocytes or platelets), which forms a component of the innate immune system. The inflammasome can comprise, e.g., proteins such as caspase 1, caspase 5, IL-18, IL-18RAP, PYCARD, NALP, NLRP3, NLRP6, and NLRP12. Inflammasome activation can promote maturation and secretion of inflammatory cytokines, such as IL-1β and IL-18.

As used herein, “therapeutically effective amount” refers to the amount of a therapeutic agent (e.g., a SMAD7 AON or TLR modulator described herein) that is sufficient to reduce and/or ameliorate the severity and/or duration of a given disease and/or a symptom related thereto. A therapeutically effective amount of a therapeutic agent can be an amount necessary for the reduction or amelioration of the advancement or progression of a given disease, reduction or amelioration of the recurrence, development or onset of a given disease, and/or to improve or enhance the prophylactic or therapeutic effect of another therapy (e.g., a therapy other than the SMAD7 AON or TLR modulator described herein).

As used herein, “SMAD7” (also known as CRCS3, FLJ16482, MADH7, MADH8, MAD (mothers against decapentaplegic, Drosophila) homolog 7, MAD homolog 8, SMAD, mothers against DPP homolog 7, mothers against DPP homolog 8) means the human protein or any of the mRNA transcripts encoded by the gene identified by Entrez GeneID No. 4092 and allelic variants thereof.

As used herein, “TLR3” (also known as Toll-Like Receptor 3; IIAE2; CD283 Antigen; CD283) means the human protein or any of the mRNA transcripts encoded by the gene identified by Entrez GeneID No. 7098 and allelic variants thereof.

As used herein, “TLR4” (also known as Toll-Like Receptor 4; ARMD10; CD284) means the human protein or any of the mRNA transcripts encoded by the gene identified by Entrez GeneID No. 7099 and allelic variants thereof.

As used herein, “TLR7” (also known as Toll-Like Receptor 7; CD287 Antigen; CD287) means the human protein or any of the mRNA transcripts encoded by the gene identified by Entrez GeneID No. 51284 and allelic variants thereof.

As used herein, “TLR8” (also known as Toll-Like Receptor 8; CD288 Antigen; CD288) means the human protein or any of the mRNA transcripts encoded by the gene identified by Entrez GeneID No. 51311 and allelic variants thereof.

As used herein, “TLR9” (also known as Toll-Like Receptor 9; CD289 Antigen; CD289) means the human protein or any of the mRNA transcripts encoded by the gene identified by Entrez GeneID No. 54106 and allelic variants thereof.

As used herein, “TLR modulator” refers to a compound capable of modulating a TLR. The TLR modulator can be capable of activating or inhibiting a TLR. In some embodiments, the TLR modulator is capable of activating or inhibiting two or more TLRs. In some embodiments, the TLR modulator is capable of activating one or more TLRs and capable of inhibiting one or more TLRs. TLR modulation can be detected in a biological assay, including a biological assay analyzing the expression and/or secretion of cytokines or other proteins from cells of the immune system, e.g., in the format of an ELISA, a radioimmunoassay, a FACS assay, a TR-FRET assay, a Western Blot, an RT-PCR, or a SPR assay. In some embodiments, the TLR modulators described herein are capable of increasing or inhibiting the expression and/or secretion of certain cytokines or other proteins by peripheral blood mononuclear cells (PBMCs) and/or plasmacytoid dendritic cells (pDCs). See, e.g., Section 7.2.(a). In some embodiments, the TLR modulators described herein are capable of increasing the expression and/or secretion of IP10, TNFα, IFNγ, TGFβ, IL-6, IL-10, PD-L1, IDO, or ICOS-L by PBMCs and/or pDCs. In some embodiments, the TLR modulators described herein are capable of decreasing the expression and/or secretion of IP10 by PBMCs and/or pDCs. A TLR synergist is a TLR modulator capable of increasing the activity of another TLR modulator (e.g., a TLR agonist) under conditions (e.g., at low TLR synergist concentrations) where the TLR synergist alone does not detectably modulate a TLR (e.g., as determined by analyzing cytokine secretion by a cell of the immune system).

As used herein, “TLR agonist” refers to a compound capable of activating a TLR. In some embodiments, the TLR agonist is capable of activating one or more TLRs. TLR activation can be detected in a biological assay, including a biological assay analyzing the expression and/or secretion of cytokines or other proteins from cells of the immune system, e.g., in the format of an ELISA, a radioimmunoassay, a FACS assay, a TR-FRET assay, a Western Blot, an RT-PCR, or a SPR assay. In some embodiments, the TLR agonists described herein are capable of increasing or inhibiting the expression and/or secretion of certain cytokines or other proteins by peripheral blood mononuclear cells (PBMCs) and/or plasmacytoid dendritic cells (pDCs). See, e.g., Section 7.2.(a). In some embodiments, the TLR agonist described herein is capable of increasing the expression and/or secretion of IP10, TNFα, IFNγ, TGFβ, IL-6, IL-10, PD-L1, IDO, or ICOS-L by PBMCs and/or pDCs. In some embodiments, the TLR agonist described herein is capable of decreasing the expression and/or secretion of IP10 by PBMCs and/or pDCs.

As used herein, “TLR antagonist” refers to a compound capable of inhibiting a TLR. Some TLR modulators can be capable of inhibiting one or more TLRs. TLR inhibition can be detected in a biological assay, including a biological assay analyzing the expression and/or secretion of cytokines or other proteins from cells of the immune system, e.g., in the format of an ELISA, a radioimmunoassay, a FACS assay, a TR-FRET assay, a Western Blot, an RT-PCR, or a SPR assay. In some embodiments, the TLR antagonist described herein is capable of inhibiting TLR activator-induced expression and/or secretion of certain cytokines or other proteins by peripheral blood mononuclear cells (PBMCs) and/or plasmacytoid dendritic cells (pDCs). See, e.g., Section 7.2.(a). In some embodiments, the TLR antagonist is capable of inhibiting the PolyI:C-induced, imiquimod-induced, or ODN2216-induced expression and/or secretion of IFNα by PBMCs and/or pDCs.

Provided herein are combinations comprising a SMAD7 ODN and a Toll-Like Receptor (TLR) modulator. In some embodiments, the SMAD7 ODN is an anti-SMAD7 therapeutic (e.g., SMAD7 AON, SMAD7 RNAi, SMAD7 miRNA). In some embodiments, the SMAD7 ODN (e.g., anti-SMAD7 therapeutic) is capable of modulating a TLR. Methods for using these combinations for the treatment of a disease in a patient in need thereof are also provided herein.

In one aspect, provided herein is a combination comprising a SMAD7 ODN and a TLR modulator. In some embodiments, the combination is a pharmaceutical composition. In some embodiments, the pharmaceutical composition comprises a SMAD7 ODN, a TLR modulator, and a pharmaceutically acceptable excipient.

In another aspect, provided herein is a combination comprising an anti-SMAD7 therapeutic and a TLR modulator. In some embodiments, the combination is a pharmaceutical composition. In some embodiments, the pharmaceutical composition comprises an anti-SMAD7 therapeutic, a TLR modulator, and a pharmaceutically acceptable excipient.

In some embodiments, the anti-SMAD7 therapeutic is a SMAD7 antisense oligonucleotide (AON). In some embodiments, the SMAD7 AON is COMPOUND (I).

In some embodiments, the TLR modulator is a TLR agonist. In some embodiments, the TLR modulator is a TLR antagonist. In some embodiments, the TLR modulator is a TLR3, TLR4, TLR7, TLR8, or TLR9 modulator, such as a TLR3, TLR4, TLR7, TLR8, or TLR9 agonist or a TLR3, TLR4, TLR7, TLR8, or TLR9 antagonist. In some embodiments, the TLR modulator is an antimalarial therapeutic, such as a quinine (e.g., quinacrine or quinidine). In some embodiments, the TLR modulator is hydroxychloroquine.

In another aspect, provided herein is a method of treating a disease in a patient in need thereof, comprising administering to the patient effective amounts of a SMAD7 ODN (e.g., a SMAD7 AON) and of a TLR modulator.

In another aspect, provided herein is a method of treating a disease in a patient in need thereof, comprising administering to the patient an effective amount of a SMAD7 ODN (e.g., a SMAD7 AON) capable of modulating a TLR.

In another aspect, provided herein is a method of treating a disease in a patient in need thereof, comprising (a) administering to the patient an effective amount of a SMAD7 AON; (b) determining the patient's response to the SMAD7 AON, and (c) if the patient does not respond to the SMAD7 AON, then, administering to the patient an effective amount of the SMAD7 AON and an effective amount of a TLR modulator.

In some embodiments, determining the patient's response to the AON comprises (a) analyzing a first level of a biomarker before administering the SMAD7 AON to the patient, and (b) analyzing a second level of the biomarker after administering the SMAD7 AON to the patient, wherein the patient responds to the SMAD7 AON if the second level of the biomarker is lower than the first level of the biomarker.

In some embodiments, the disease is an inflammatory disease (e.g., inflammatory bowel syndrome (IBD), and the like). In some embodiments, the disease is an autoimmune disease (e.g., Sjogren's Syndrome, systemic lupus erythematosus, and the like). In some embodiments, the disease is an airway disease (e.g., asthma, chronic pulmonary disease (COPD), and the like). In some embodiments, the disease is an allergic disorder (e.g., atopic dermatitis, and the like). In some embodiments, the disease is an infectious disease (e.g., Malaria, and the like). In some embodiments, the disease is a metabolic disease (e.g., diabetes, hyperlipidemia, non-alcoholic fatty liver disease, and the like). In some embodiments, the disease is cancer (e.g., colorectal cancer, and the like). In some embodiments, the disease is a central nervous system (CNS) disease (e.g., multiple sclerosis, and the like). In some embodiments, the disease is a skin disease (e.g., basal cell carcinoma, actinic keratosis).

7.1 Anti-SMAD7 Therapeutic Combinations

In one aspect, provided herein is a combination comprising an anti-SMAD7 therapeutic described herein and a TLR modulator described herein that can be used in the methods of treatment provided herein. See, e.g., Section 7.2, 7.4 and 7.7. In some embodiments, the anti-SMAD7 therapeutic is capable of modulating a TLR. See also, Section 7.8.(a). In some embodiments, the anti-SMAD7 therapeutic is an anti-SMAD7 ODN. In some embodiments, the anti-SMAD7 therapeutic is a SMAD7 antisense oligonucleotide (AON). In some embodiments, the SMAD7 AON is COMPOUND (I).

In some embodiments, the SMAD7 AON and the TLR modulator are formulated separately, e.g., in separate unit dosage forms.

In some embodiments, the SMAD7 AON and the TLR modulator are components of a pharmaceutical composition.

In some embodiments, the SMAD7 AON is covalently linked to the TLR modulator.

In some embodiments, the SMAD7 AON is capable of modulating a TLR.

In another aspect, provided herein is a combination comprising an anti-SMAD7 therapeutic described herein and an additional agent, other than a TLR modulator, that can be used in the methods of treatment provided herein. See, e.g., Section 7.3, 7.4 and 7.7. In some embodiments, the anti-SMAD7 therapeutic is an anti-SMAD7 ODN. In some embodiments, the anti-SMAD7 therapeutic is a SMAD7 antisense oligonucleotide (AON). In some embodiments, the SMAD7 AON is COMPOUND (I). In some embodiments, the SMAD7 AON and the additional agent other than a TLR modulator are formulated separately, e.g., in separate unit dosage forms. In some embodiments, the SMAD7 AON and the additional agent other than a TLR modulator are components of a pharmaceutical composition.

7.2 TLR Modulators

In some embodiments, the TLR modulator is a TLR3 modulator. In some embodiments, the TLR modulator is a TLR4 modulator. In some embodiments, the TLR modulator is a TLR7 modulator. In some embodiments, the TLR modulator is a TLR8 modulator. In some embodiments, the TLR modulator is a TLR9 modulator.

In some embodiments, the TLR modulator is a TLR3 agonist. In some embodiments, the TLR modulator is a TLR4 agonist. In some embodiments, the TLR modulator is a TLR7 agonist. In some embodiments, the TLR modulator is a TLR8 agonist. In some embodiments, the TLR modulator is a TLR9 agonist.

In some embodiments, the TLR modulator is a TLR3 antagonist. In some embodiments, the TLR modulator is a TLR4 antagonist. In some embodiments, the TLR modulator is a TLR7 antagonist. In some embodiments, the TLR modulator is a TLR8 antagonist. In some embodiments, the TLR modulator is a TLR9 antagonist.

In some embodiments, the TLR modulator described herein can modulate two or more TLRs. In some embodiments, the TLR modulator can modulate TLR3 and TLR7. In some embodiments, the TLR modulator can modulate TLR7 and TLR9. In some embodiments, the TLR modulator can modulate TLR3, TLR7 and TLR8. In some embodiments, the TLR modulator can modulate TLR3, TLR7 and TLR9.

In some embodiments, the TLR modulator has a stronger effect on one TLR than on another TLR. In some embodiments, the TLR modulator has a stronger effect on TLR 9 and/or TLR7 than on TLR3.

In some embodiments, the TLR modulator can inhibit TLR9. In some embodiments, the TLR modulator can inhibit TLR7 and TLR9. In some embodiments, the TLR modulator can inhibit TLR7, TLR8 and TLR9. In some embodiments, the TLR modulator can inhibit TLR 3, TLR7, TLR8 and TLR9. In some embodiments, the TLR modulator can inhibit TLR4.

In some embodiments, the TLR modulator can activate one or more TLRs and inhibit one or more TLR. In some embodiments, the TLR modulator can activate TLR9 and inhibit TLR7 and/or TLR3. In some embodiments, the TLR modulator can activate the TLR under certain conditions (e.g., TLR modulator concentration, nature of target cell, presence of additional signaling molecules in cell medium, and the like) and inhibit the TLR under certain other conditions. In some embodiments, the TLR modulator can activate TLR9 under certain conditions and inhibit TLR9 under certain other conditions.

In some embodiments, the TLR modulator (e.g., TLR9 agonist) can induce an inflammasome in a cell of the immune system, such as a PBMC. In some embodiments, inducing the inflammasome comprises inducing the expression or secretion of an inflammasome component (e.g., caspase 1, capase-5, PYCARD, NALP, NLRP3, NLRP6 or NLRP12). In some embodiments, inducing the inflammasome comprises inducing a cell of the immune system (e.g., PBMC) to express or secrete IL-1β and/or IL-18, e.g., as determined by RT-PCR or ELISA.

In some embodiments, the TLR modulator (e.g., TLR9 agonist) can induce differentiation of a cell of the immunce system, such as a pDC, e.g., as determined by detection of a pDC differentiation marker by FACS (e.g., CD80, CD83, CD86, CCR6, CCR7, CD123, SLAMF7, and the like).

In some embodiments, the TLR modulator (e.g., TLR9 agonist) can induce expression or secretion of TNFα or IFNγ from a cell of the immune system, such as a PBMC, e.g., as determined by RT-PCR or ELISA.

In some embodiments, the TLR modulator (e.g., TLR9 agonist) can induce a tolerogenic pDC. In some embodiments, the TLR modulator can induce a Treg. In some embodiments, the TLR modulator can suppress a pathogenic T-effector cell in a patient.

In some embodiments, the TLR modulator (e.g., TLR9 agonist) can induce an inflammasome in a cell of the immune system, such as a PBMC, and the TLR modulator can induce differentiation of a cell of the immunce system, such as a pDC.

In some embodiments, the TLR modulator (e.g., TLR9 agonist) can induce an inflammasome in a cell of the immune system, such as a PBMC, and the TLR modulator can induce differentiation of a cell of the immunce system, such as a pDC, and the TLR modulator can induce a tolerogenic pDC. In some embodiments, the TLR modulator can induce expression or secretion of TNFα or IFNγ from a cell of the immune system, such as a PBMC,

In some embodiments, the TLR modulator (e.g., TLR9 agonist) can induce epithelial restitution or mucosal healing, e.g., in an animal model of IBD, or in a human IBD patient, e.g., as determined by endoscopy.

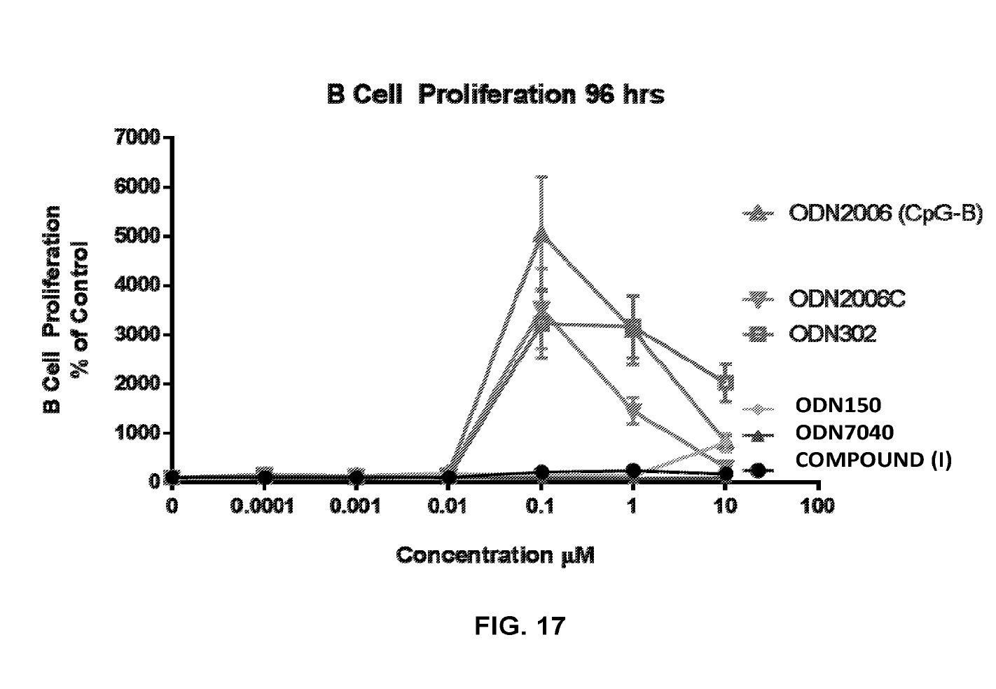

In some embodiments, the TLR modulator (e.g., TLR9 agonist) cannot induce detectable levels of proliferation or only induces low levels of proliferation (e.g., less than 10-fold, less than 9-fold, less than 8-fold, less than 7-fold, less than 6-fold, less than 5-fold, less than 4-fold, less than 3-fold, or less than 2-fold increase in proliferation relative to baseline proliferation level observed in the absence or the TLR modulator) of a cell of the immune system, such as a B-cell, e.g., as determined by a H³-thymidine incorporation assay (see, e.g., FIG. 17).

In some embodiments, the TLR modulator (e.g., TLR9 agonist) cannot detectably activate the NF-κB pathway, e.g., in a cell of the immune system (e.g., B-cell) or in a recombinant cell comprising a NF-κB reporter reporter gene construct (e.g., a HEK293 cell comprising a construct comprising a NF-κB promoter coupled to a reporter gene, such as luciferase) or the TLR modulator can activate only low levels of the NF-κB pathway (e.g., less than 10-fold, less than 9-fold, less than 8-fold, less than 7-fold, less than 6-fold, less than 5-fold, less than 4-fold, less than 3-fold, or less than 2-fold activation above the baseline level of NF-κB pathway activation observed in the absence or the TLR modulator).

In some embodiments, the TLR modulator (e.g., TLR9 agonist) cannot induce IFN-α or can only weakly induce IFN-α (e.g., less than 10-fold, less than 9-fold, less than 8-fold, less than 7-fold, less than 6-fold, less than 5-fold, less than 4-fold, less than 3-fold, or less than 2-fold the baseline level of IFN-α observed in the absence or the TLR modulator) in a cell of the immune system (e.g., a purified or mature pDC).

In some embodiments, the TLR modulator (e.g., TLR9 agonist) cannot induce detectable levels of proliferation or induces only low levels of proliferation of a cell of the immune system, such as a B-cell, and the TLR modulator cannot detectably activate the NF-κB pathway, e.g., in a cell of the immune system (e.g., B-cell) or in a recombinant cell comprising a NF-κB reporter reporter gene construct (e.g., a HEK293 cell comprising a construct comprising a NF-κB promoter coupled to a reporter gene, such as luciferase) or the TLR modulator can activate only low levels of the NF-κB pathway.

In some embodiments, the TLR modulator (e.g., TLR9 agonist) cannot induce detectable levels of proliferation or only induces low levels of proliferation of a cell of the immune system, such as a B-cell, and the TLR modulator cannot detectably activate the NF-κB pathway, e.g., in a cell of the immune system (e.g., B-cell) or in a recombinant cell comprising a NF-κB reporter reporter gene construct (e.g., a HEK293 cell comprising a construct comprising a NF-κB promoter coupled to a reporter gene, such as luciferase) or the TLR modulator can activate only low levels of the NF-κB pathway, and the TLR modulator cannot induce IFN-α or can only weakly induce IFN-α in a cell of the immune system (e.g., a purified or mature pDC).

In some embodiments, the TLR modulator (e.g., TLR9 agonist) can induce an inflammasome in a cell of the immune system, such as a PBMC, and the TLR modulator can induce differentiation of a cell of the immune system, such as a pDC, and the TLR modulator can induce a tolerogenic pDC, and the TLR modulator cannot induce detectable levels of proliferation or only induces low levels of proliferation of a cell of the immune system, such as a B-cell. In some embodiments, the TLR modulator cannot detectably activate the NF-κB pathway, e.g., in a cell of the immune system (e.g., B-cell) or in a recombinant cell comprising a NF-κB reporter reporter gene construct (e.g., a HEK293 cell comprising a construct comprising a NF-κB promoter coupled to a reporter gene, such as luciferase) or the TLR modulator can activate only low levels of the NF-κB pathway. In some embodiments, the TLR modulator cannot induce IFN-α or can only weakly induce IFN-α in a cell of the immune system (e.g., a purified or mature pDC). In some embodiments, the TLR modulator can induce expression or secretion of TNFα or IFNγ from a cell of the immune system, such as a PBMC.

In some embodiments, the TLR modulator (e.g., TLR9 agonist) can induce pDC differentiation to higher levels than a CpG-A ODN, Kappaproct® (ODN150), or Monarsen® (ODN7040) (e.g., levels of pDC differentiation observed with the TLR modulator are at least 2-fold, 4-fold, 6-fold, 8-fold, or 10-fold higher than levels observed with CpG-A ODN, Kappaproct®, or Monarsen® under otherwise comparable or identical experimental conditions).

In some embodiments, the TLR modulator (e.g., TLR9 agonist) can induce IFN-α in a cell of the immune system at lower levels than a CpG-A ODN, a CpG-B ODN (ODN2006), Kappaproct® (ODN150), or Monarsen® (ODN7040) (e.g., IFN-α levels observed with the TLR modulator are either undetectable, or at least 2-fold, 4-fold, 6-fold, 8-fold, or 10-fold lower than IFN-α levels observed with the CpG-A ODN, CpG-B ODN, Kappaproct®, or Monarsen® under otherwise comparable or identical experimental conditions).

In some embodiments, the TLR modulator (e.g., TLR9 agonist) induces proliferation in a cell of the immune system (e.g., B-cell) at lower levels than a CpG-B ODN (e.g., B-cell proliferation with the TLR modulator is either undetectable, or at least 2-fold, 4-fold, 6-fold, 8-fold, or 10-fold lower than B-cell proliferation observed with the CpG-B ODN under otherwise comparable or identical experimental conditions).

In some embodiments, the TLR modulator (e.g., TLR9 agonist) does not activate the NF-kB pathway or induces activation of the NF-kB pathway at lower levels than a CpG-B ODN (e.g., ODN2006) (e.g., NF-kB pathway activation with the compound is either undetectable, or at least 2-fold, 4-fold, 6-fold, 8-fold, or 10-fold lower than NF-kB pathway activation observed with the CpG-B ODN under otherwise comparable or identical experimental conditions).

In some embodiments, the TLR modulator (e.g., TLR9 agonist) can induce inflammasome activation (e.g., secretion of IL-1β, IL-18 or both) more strongly than a CpG-A ODN, a CpG-B ODN, Kappaproct®, or Monarsen® and can induce pDC differentiation more strongly than Kappaproct®, or Monarsen®. In some embodiments, the TLR modulator can induce pDC differentiation to similar levels as a CpG-B ODN (e.g., ODN2006). See, e.g., Examples 8 and 9, Tables 5 and 6.

In some embodiments, the TLR modulator (e.g., TLR9 agonist) can induce inflammasome activation (e.g., secretion of IL-1β, IL-18, or both) more strongly than a CpG-A ODN, CpG-B ODN, Kappaproct®, or Monarsen® and can induce pDC differentiation more strongly than Kappaproct®, or Monarsen®, and induces B-cell proliferation at lower levels than a CpG-B ODN (e.g., ODN2006). In some embodiments, the TLR modulator can induce pDC differentiation to similar levels as a CpG-B ODN (e.g., ODN2006). In some embodiments, B-cell proliferation with the compound is only weak or is not detectible. See, e.g., Examples 8 and 9, Tables 5 and 6.

In some embodiments, the TLR modulator (e.g., TLR9 agonist) can induce inflammasome activation (e.g., secretion of IL-1β, IL-18, or both) more strongly than a CpG-A ODN, a CpG-B ODN, Kappaproct®, or Monarsen® and can induce pDC differentiation more strongly than Kappaproct®, or Monarsen®, and induces B-cell proliferation and NF-kB pathway activation at lower levels than a CpG-B ODN (e.g., ODN2006). In some embodiments, the TLR modulator can induce pDC differentiation to similar levels as a CpG-B ODN (e.g., ODN2006). In some embodiments, B-cell proliferation with the TLR modulator is only weak or is not detectible. See, e.g., Examples 8 and 9, Tables 5 and 6.

In some embodiments, the TLR modulator (e.g., TLR9 agonist) can induce inflammasome activation (e.g., secretion of IL-1β, IL-18, or both) more strongly than a CpG-A ODN, CpG-B ODN, Kappaproct®, or Monarsen® and can induce pDC differentiation more strongly than Kappaproct®, or Monarsen®, and induces B-cell proliferation, IFN-α secretion and NF-kB pathway activation at lower levels than a CpG-B ODN (e.g., ODN2006). In some embodiments, the TLR modulator can induce pDC differentiation to similar levels as a CpG-B ODN (e.g., ODN2006). In some embodiments, B-cell proliferation with the TLR modulator is only weak or is not detectible. See, e.g., Examples 8 and 9, Tables 5 and 6.

In some embodiments, the TLR modulator can have any one or more of the activities of COMPOUND (I) in any one of Tables 3-7. In some embodiments, the activity of the TLR modulator can have a similar strength as the activity of COMPOUND (I) relative to a TLR modulator listed in any one of Tables 3-7 (e.g., ODN150, ODN7040, CpG-A, CpG-A).

In some embodiments, the TLR modulator (e.g., TLR9 agonist) is a SMAD7 ODN. See, e.g., Section 7.8(a). In some embodiments, the SMAD7 ODN does not comprise a nucleotide sequence complementary to region 108-128 of the human SMAD7 nucleotide sequence of SEQ ID NO: 1, or the corresponding RNA sequence, or fragments thereof (e.g., fragments having 11 or more, 12 or more, 13 or more, 14 or more, 15, or more, 16 or more, 17 or more, 18 or more, or 19 or more nucleotides). In some embodiments, the SMAD7 ODN does not comprise a nucleotide sequence complementary to region 1-30, 16-45, 31-60, 46-75, 61-90, 76-105, 91-120, 106-135, 121-150, 136-165, 151-180, 166-195, 181-210, 196-225, 211-240, 226-255, 241-270, 256-285, 271-300, 286-315, 301-330, 316-345, 331-360, 346-375, 361-390, 376-405, 391-420, 406-435, 421-450, 436-465, 451-180, 466-495, 481-510, 496-525, 511-540, 526-555, 541-570, 556-585, 571-600, 586-615, 601-630, 616-645, 631-660, 646-675, 661-690, 676-705, 691-720, 706-735, 721-750, 736-765, 751-780, 766-195, 781-810, 796-825, 811-840, 826-855, 841-870, 856-885, 871-900, 896-915, 901-930, 916-45, 931-960, 946-975, 961-990, 976-1005, 991-1120, 1106-1135, 1121-1150, 1136-1165, 1151-1180, 1166-1195, 1181-1210, 1196-1225, 1211-1240, 1226-1255, 1241-1270, or 1256-281 of the human SMAD7 nucleotide sequence of SEQ ID NO: 1, or the corresponding RNA sequence, or fragments thereof (e.g., fragments having 11 or more, 12 or more, 13 or more, 14 or more, 15, or more, 16 or more, 17 or more, 18 or more, or 19 or more nucleotides), or combinations thereof (e.g., region 1-45, 16-60, 1-60, 30-90, and the like). In some embodiments, the SMAD7 ODN does not comprise a nucleotide sequence complementary to nucleotides 403, 233, 294, 295, 296, 298, 299 or 533 of the human SMAD7 nucleotide sequence of SEQ ID NO: 1, or the corresponding RNA sequence. In some embodiments, the SMAD7 ODN does not comprise the nucleotide sequence of SEQ ID NO: 3 (5′-GTCGCCCCTTCTCCCCGCAGC-3′). In some embodiments, the SMAD7 ODN does not comprise a sequence of 10 or more nucleotides of the nucleotide sequence of SEQ ID NO: 3 (e.g., 11 or more, 12 or more, 13 or more, 14 or more, 15, or more, 16 or more, 17 or more, 18 or more, 19 or more, or 20 or more nucleotides). In some embodiments, the SMAD7 ODN does not comprise COMPOUND (I). In some embodiments, the SMAD7 ODN does not comprise a nucleotide sequence of SEQ ID NOs:2-7, SEQ ID NOs: 11-87, and/or SEQ ID NOs:91-144, and/or the nucleotide sequence of any oligonucleotides listed in Table 1, and/or the nucleotide sequence of the oligonucleotides listed in Table 2. In some embodiments, the SMAD7 ODN does not comprise a sequence of 10 or more nucleotides of the nucleotide sequence of SEQ ID NOs:2-6, SEQ ID NOs: 11-87, or SEQ ID NOs:91-144 (e.g., 11 or more, 12 or more, 13 or more, 14 or more, 15, or more, 16 or more, 17 or more, 18 or more, 19 or more, or 20 or more nucleotides).

In some embodiments, the TLR modulator is a SMAD7 AON.

In some embodiments, the TLR modulator is an ODN other than a SMAD7 ODN.

In some embodiments, the cell of the immune system (e.g., B-cell, pDC, PBMC) is a human cell.

In some embodiments, the TLR modulator is an oligonucleotide (ODN). In some embodiments, the TLR modulator is a SMAD7 ODN. In some embodiments, the TLR modulator is an anti-SMAD7 ODN (e.g., SMAD7 AON, SMAD7 RNAi or SMAD7 miRNA), such as COMPOUND (I). See, e.g., Section 7.8.(a).

In some embodiments, the TLR modulator is an oligonucleotide comprising one or more “CG (or GC) dinucleotides” or “CG (or GC) islands.” The CG or GC dinucleotide can be methylated or unmethylated. The CG or GC dinucleotide can include a phosphate linkage, or a modified linkage, such as a phosphorothioate linkage. In some embodiments, the TLR modulator comprises one or more methylated CG or GC dinucleotides comprising a phosphorothioate linkage. A TLR modulator comprising at least one unmethylated CG or GC dinucleotide is an oligonucleotide molecule which comprises an unmethylated cytosine-guanine dinucleotide sequence (an unmethylated 5′ cytidine followed by 3′ guanosine and linked by a phosphate bond) and which modulates a TLR activity. CG or GC oligonucleotides have been described, e.g., in U.S. Pat. Nos. 6,194,388; 6,207,646; 6,214,806; 6,218,371; 6,239,116; and 6,339,068, the contents of which are incorporated herein by reference.

In some embodiments, the TLR modulator is a single-stranded RNA. In some embodiments, the TLR modulator is a double-stranded RNA. In some embodiments, the TLR modulator is a DNA ODN comprising a CG or GC dinucleotide sequence. In some embodiments, the CG or GC dinucleotide sequence is a plurality of CG or GC dinucleotide sequences (e.g., 2, 3, 4, 5, 6, or more CG or GC dinucleotide sequences). In some embodiments, the plurality of CG or GC dinucleotide sequences comprises one or more CG dinucleotide sequence and one or more GC dinucleotide sequence. In some embodiments, the plurality of CG or GC dinucleotide sequences comprises only CG dinucleotide sequences. In some embodiments, the plurality of CG or GC dinucleotide sequences comprises only GC dinucleotide sequences.

In some embodiments, the TLR modulator is a chemically modified SMAD7 ODN (e.g., SMAD7 AON). In some embodiments, the TLR modulator is covalently linked to a SMAD7 ODN (e.g., a chemically modified SMAD7 ODN or an unmodified SMAD7 ODN). In some embodiments, the TLR modulator is covalently linked to COMPOUND(I). In some embodiments, the TLR modulator is an ODN other than a SMAD7 ODN (e.g., BL-7040 (ODN7040), CYT003, CYT003-QbG10, AZD1419, DIMS0150 (ODN150), E6446, CpG ODN2088, ODN2006, IMO-8400, IMO-3100, CL075, VTX-2337, or naltrexone).

In some embodiments, the TLR modulator is a stereo-defined ODN. Stereo-defined ODNs are described, e.g., in U.S. 2015/021106. In some embodiments, the stereo-defined ODNs can exhibit greater affinity for a TLR, as compared to stereo-random counterparts. In some embodiments, stereo-defined ODNs can elicit one or more immune responses, when administered to subjects that meet certain clinical criteria, with less degree of variables amongst the population, as compared to stereo-random counterparts. In some embodiments, stereo-defined ODNs can cause a lesser degree of toxic side effects or fewer side effects, when administered to subjects that meet certain clinical criteria, as compared to stereo-random counterparts.

In some embodiments, the TLR modulator is free of CG dinucleotide motifs. ODNs that are free of CG dinucleotides are referred to as non-CG ODNs, and can comprise non-CG immunostimulatory motifs. In some embodiments, the non-CG ODNs are T-rich immunostimulatory ODNs, such as ODNs having at least 80% T.

In some embodiments, the TLR modulator is an “A class” immunomodulator ODN. A class ODNs are generally potent inducers of IFN-α and NK cell activation and relatively weak at stimulating B-cells. The A class ODNs typically have stabilized poly-G sequences at 5′ and 3′ ends and a palindromic phosphodiester CG dinucleotide-containing sequence of at least 6 nucleotides. See, e.g., International Patent Application No. PCT/US00/26527 (published as WO 01/22990)

In some embodiments, the TLR modulator is a B-class immunomodulatory ODN. B class ODNs are generally potent at activating B-cells and relatively weak inducers of IFN-α and NK cell activation. The B class ODNs are typically fully stabilized and include an unmethylated CG dinucleotide within certain preferred base contexts. See, e.g., U.S. Pat. Nos. 6,194,388; 6,207,646; 6,214,806; 6,218,371; 6,239,116, and 6,339,068.

In some embodiments, the TLR modulator is a “C class” immunomodulatory ODN. C class ODNs can generally activate B-cells and NK cells and induce IFN-α. C class immunostimulatory ODNs typically contain at least two distinct motifs and can stimulate cells of the immune system. Some of these ODN have both a traditional “stimulatory” CG sequence and a “GC-rich” or “B-cell neutralizing” motif. These combination motif oligonucleotides can have immune stimulating effects that fall somewhere between those effects associated with traditional class B ODN, which are strong inducers of B-cell activation and dendritic cell (DC) activation, and those effects associated with a more recently described class of immune stimulatory ODNs (class A ODN) which are strong inducers of IFN-α and natural killer (NK) cell activation, and relatively poor inducers of B-cell and DC activation. See, e.g., Krieg A M et al. (1995) Nature 374:546-9; Ballas Z K et al. (1996) J Immunol 157:1840-5; Yamamoto S et al. (1992) J Immunol 48:4072-6. While preferred class B ODN often have phosphorothioate backbones and preferred class A ODN have mixed or chimeric backbones, the C class of combination motif immune stimulatory oligonucleotides can have either stabilized, e.g., phosphorothioate, chimeric, or phosphodiester backbones, and in some preferred embodiments, they have semi-soft backbones. This class has been described, e.g., in U.S. Pat. No. 8,834,900, the entire contents of which is incorporated herein by reference.

In some embodiments, the TLR modulator is an “E class” immunostimulatory ODN. E class ODNs typically have an enhanced ability to induce secretion of IFN-α. Structurally, E class ODNs generally have a lipophilic substituted nucleotide analog 5′ and/or 3′ of a YGZ motif. The compound of the E class formula can be, e.g., any of the following lipophilic substituted nucleotide analogs: a substituted pyrimidine, a substituted uracil, a hydrophobic T analog, a substituted toluene, a substituted imidazole or pyrazole, a substituted triazole, 5-chloro-uracil, 5-bromo-uracil, 5-iodo-uracil, 5-ethyl-uracil, 5-propyl-uracil, 5-propinyl-uracil, (E)-5-(2-bromovinyl)-uracil, or 2.4-difluoro-toluene.

In some embodiments, the TLR modulator is a “T class” immunostimulatory ODN. T class ODNs typically induce secretion of lower levels of IFN-α and IFN-related cytokines and chemokines than B class or C class ODNs, while retaining the ability to induce levels of IL-10 similar to B class ODNs. T class ODNs are described, e.g., in U.S. Patent Publication No. 2006/0019916, the entire contents of which are hereby incorporated by reference.

In some embodiments, the TLR modulator is a “P class” immunostimulatory ODN The P class immunostimulatory ODNs typically have several domains, including a 5′ TLR activation domain, 2 duplex forming regions and an optional spacer and 3′ tail. This class of ODNs has the ability, in some instances, to induce much higher levels of IFN-α secretion than C-Class ODNs. P-Class ODNs often have the ability to spontaneously self-assemble into concatamers, in vitro or in vivo. P class ODNs are described, e.g., in U.S. Patent Publication No. 2008/0045473.

In some embodiments, the TLR modulator is an “S class” immunosuppressive ODN oligonucleotides. S class ODNs can inhibit immunestimulation, which can be useful, e.g., in the treatment or prevention of septic shock, inflammation, allergy, asthma, graft rejection, graft-versus host disease (GvHD), autoimmune diseases, Th1- or Th2-mediated diseases, bacterial infections, parasitic infections, spontaneous abortions, and tumors. S class ODNs can be used generally to inhibit activation of all cells expressing the relevant TLRs, and more specifically to inhibit activation of antigen-presenting cells, B-cells, plasmacytoid dendritic cells (pDCs), monocytes, monocyte-derived cells, eosinophils, and neutrophils. S class ODN are described, e.g., in U.S. Patent Publication No. US 2005/0239733.

In some embodiments, the TLR modulator is a small molecule (e.g., <1,000 Da) other than an ODN. In some embodiments the small molecule TLR modulator has a molecular weight of 1,000 Da or less. In some embodiments, the small molecule TLR modulator has a molecular weight of 500 Da or less.

In some embodiments, the small molecule TLR modulator is covalently linked to an anti-SMAD7 ODN (e.g., a modified or unmodified SMAD7 AON, SMAD7 RNAi, or SMAD7 miRNA). In some embodiments, the small molecule TLR modulator is covalently linked to COMPOUND (I).

In some embodiments, the TLR modulator is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, a atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or a derivative thereof.

In some embodiments, the TLR modulator is a quinoline, or a derivative thereof. In some embodiments, the quinoline includes, e.g., chloroquine (Aralen), hydroxychloroquine (Plaquenil), a 4-aminoquinoline (e.g., amodiaquine (Camoquin, Flavoquine)), a mefloquine (Lariam, Mephaquin or Mefliam), a 8-aminoquinoline (e.g., primaquine or primaquine phosphate), or atovaquenone/proguanil (Malarone). In some embodiments, the TLR modulator is a quinine (Qualaquin, Quinate, Quinbisul), or derivative thereof. In some embodiments, the quinine includes, e.g., quinacrine (Mepacrine, Atebrine) or quinidine (Quinaglute, Quinidex). In some embodiments, the TLR modulator is hydroxychloroquine.

Table 1 lists exemplary TLR modulators that can be used in connection with the compounds, pharmaceutical compositions and methods described herein. The TLR modulators of Table 1 can be useful in the treatment of certain diseases, some of which are exemplified in Table 1.

TABLE 1
Exemplary TLR Modulators (Agonists & Antagonists)
and Exemplary Indicated Diseases

| | TLR | INDICATIONS |
|---|---|---|
| TLR AGONISTS | | |
| BL-7040 (ODN7040) | TLR9 | Sjogren's Syndrome |
| CYT003 | TLR9 | Asthma |
| CYT003-QbG10 | TLR9 | Allergic asthma |
| AZD1419 | TLR9 | Asthma |
| DIMS0150 (ODN150) | TLR9 | Ulcerative colitis |
| Imiquimod | TLR7, TLR8 | Basal cell carcinoma, actinic keratosis, genital |
| | | warts |
| Resiquimod | TLR7, TLR8 | Basal cell carcinoma, actinic keratosis, genital |
| | | warts |
| E6446 | TLR9 | Malaria |
| Hydroxychloroquine | TLR7 and TLR9 | Malaria, lyme-disease, lupus erythematosus; |
| | | rheumatoid arthritis, post-Lyme arthritis, |
| | | Sjogren's Syndrome, porphyria; metabolic |
| | | syndrome; dry eye; |
| Chloroquine | TLR3, TLR7, | Malaria, rheumatoid arthritis |
| | TLR8 and TLR9 | |
| CpG ODN2088 | TLR9 | Reduces pain hypersensitivity and the |
| | | inflammatory response in spinal cord injury; |
| | | improves bladder function and white matter |
| | | sparing in spinal cord injury |
| IMO-8400 | TLR7, TLR8 and | Lupus erythematosus and other autoimmune |
| | TLR9 | diseases; Waldenstrom's macroglobulinemia; |
| | | dermatomyositis |
| IMO-3100 | TLR7 and TLR9 | Autoimmune and inflammatory diseases, such |
| | | as lupus, rheumatoid arthritis, multiple |
| | | sclerosis, psoriasis, and colitis |
| COV08-0064 | TLR9 | Reduces sterile inflammation-induced organ |
| | | damage |
| CL075 | TLR7 | Cancer (e.g., squamous cell carcinoma) |
| VTX-2337 | TLR8 | Cancer (e.g., ovarian cancer) |
| ODN2006 | TLR9 | Vaccine development, cancer (adjuvant) |
| TLR ANTAGONISTS | | |
| Naltrexone | TLR4, TLR9 | Reverses neuropathic pain, cancer (supportive |
| | | care) |

(a) TLR Modulator and TLR Synergist Assays

TLR modulators and TLR synergists can be identified, e.g., by analyzing the effect of a test compound on the expression or secretion of certain TLR pathway components by a cell of the immune system.

In some embodiments, the TLR pathway components can include TNFα, IFNγ, TGFβ, IL-6, IL-10, IP10 (CXCL10), PD-L1, IDO, or ICOS-L.

In some embodiments, the TLR pathway components can include bFGF, CCR6, CCR7, CD80, CD83, CD86, CD-69, CD123 (IL-3Rα), EGFR, Eot3, GARP, ICAM-1, IgG, IL-1α, IL1-β, IL-2, IL-4, IL-10Rα, IL-18, IL-23p19, ILT7, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1.

In some embodiments, the TLR pathway component can include the inflammasome, or an inflammasome component, e.g., in a cell of the immune system. In some embodiments, the TLR pathway component comprises IL1-β or IL-18, or both. In some embodiments, the TLR pathway component comprises IL1-β.

In some embodiments, a cell of the immune system can include a peripheral blood mononuclear cell (PBMCs) or plasmacytoid dendritic cell (pDCs). In some embodiments, the cell of the immune system can include a B-cell.

TLR pathway component expression or secretion by a cell of the immune system can be tested at the mRNA or protein level using detection methods well known in the art. In some embodiments, TLR pathway component expression can be detected by ELISA, radioimmunoassay (RIA), FACS, SPR, TR-FRET, western blot, RT-PCR, immunocytochemistry, or fluorescence microscopy.

A TLR modulator (e.g., a TLR agonist or TLR antagonist) can upregulate or downregulate the expression or secretion of a TLR pathway component by a cell of the immune system. In some embodiments, a TLR modulator can upregulate the expression or secretion of certain TLR pathway components and downregulate the expression or secretion of certain other TLR pathway components by a cell of the immune system. In some embodiments, a TLR modulator can upregulate the expression or secretion of a TLR pathway component at one concentration and downregulate the expression or secretion at another concentration. In some embodiments, a TLR modulator (e.g., a TLR antagonist) can modulate the expression or secretion of a TLR pathway component by the cell of the immune system that is induced by another TLR modulator (e.g., a TLR agonist).

In some embodiments, a compound capable of modulating a TLR (e.g., a TLR9 agonist, such as a SMAD7 ODN) can increase the expression or secretion of IP10, TNFα or IL-6 proteins by a pDC, when contacted with the pDC at a concentration of less than 1.0 μM (e.g., about 0.05 μM, about 0.1 μM, about 0.2 μM, about 0.3 μM, about 0.4 μM, about 0.5 μM, about 0.6 μM, about 0.7 μM, about 0.8 μM, or about 0.9 μM), relative to a pDC control not contacted with the compound capable of modulating the TLR, as determined in an immunoassay (e.g., FACS, ELISA, and the like). In some embodiments, the compound capable of modulating a TLR can increase the expression or secretion of IP10, TNFα or IL-6 protein by the pDC. In some embodiments, the compound capable of modulating a TLR can increase the expression of IP10, TNFα or IL-6 protein by a factor of at least 2-fold (e.g., at least 3-fold, at least 4-fold, at least 5-fold, at least 6-fold, at least 7-fold, at least 8-fold, at least 9-fold, or at least 10-fold). See, e.g., Example 4, Table 3.

In some embodiments, a compound capable of modulating a TLR (e.g., a TLR9 agonist, such as a SMAD7 ODN) can increase the expression or secretion of TNFα, IFNγ, TGFβ, IL-6, IL-10, PD-L1, IDO, or ICOS-L protein or decrease the expression or secretion of IP10 by a pDC, when contacted with the pDC at a concentration of more than 1.0 μM (e.g., about 2.0 μM, about 4.0 μM, about 6.0 μM, about 8.0 μM, about 10 μM, about 15.0 μM, about 20.0 μM, about 25.0 μM, about 30.0 μM, or more), relative to a pDC control not contacted with the compound capable of modulating the TLR, as determined in an immunoassay (e.g., FACS, ELISA, and the like). In some embodiments, the compound capable of modulating the TLR can increase the secretion or expression of TNFα, IFNγ, TGFβ, IL-6, IL-10, PD-L1, IDO, and ICOS-L and decrease the expression or secretion of IP-10. In some embodiments, the compound capable of modulating the TLR can increase the expression or secretion of TNFα, IFNγ, TGFβ, IL-6, IL-10, PD-L1, IDO, or ICOS-L by a factor of at least 2-fold (e.g., at least 3-fold, at least 4-fold, at least 5-fold, at least 6-fold, at least 7-fold, at least 8-fold, at least 9-fold, or at least 10-fold). In some embodiments, the compound capable of modulating the TLR can increase the expression or secretion of TNFα, IL-6, and ICOS-L by a factor of at least 2-fold (e.g., at least 3-fold, at least 4-fold, at least 5-fold, at least 6-fold, at least 7-fold, at least 8-fold, at least 9-fold, or at least 10-fold). In some embodiments, the compound capable of modulating the TLR can decrease the expression or secretion of IP-10 by a factor of at least 10-fold (e.g., at least 12-fold, at least 14-fold, at least 16-fold, at least 18-fold, or at least 20-fold). In some embodiments, the compound capable of modulating the TLR can increase the expression or secretion of TNFα, IFNγ, TGFβ, IL-6, IL-10, PD-L1, IDO, or ICOS-L by a factor of at least 2-fold (e.g., at least 3-fold, at least 4-fold, at least 5-fold, at least 6-fold, at least 7-fold, at least 8-fold, at least 9-fold, or at least 10-fold). In some embodiments, the compound capable of modulating the TLR can increase the expression or secretion of TNFα, IL-6, and ICOS-L by a factor of at least 2-fold (e.g., at least 3-fold, at least 4-fold, at least 5-fold, at least 6-fold, at least 7-fold, at least 8-fold, at least 9-fold, or at least 10-fold) and decrease the expression of IP-10 by a factor of at least 10-fold (e.g., at least 12-fold, at least 14-fold, at least 16-fold, at least 18-fold, or at least 20-fold). See, e.g., Example 4, Table 4.

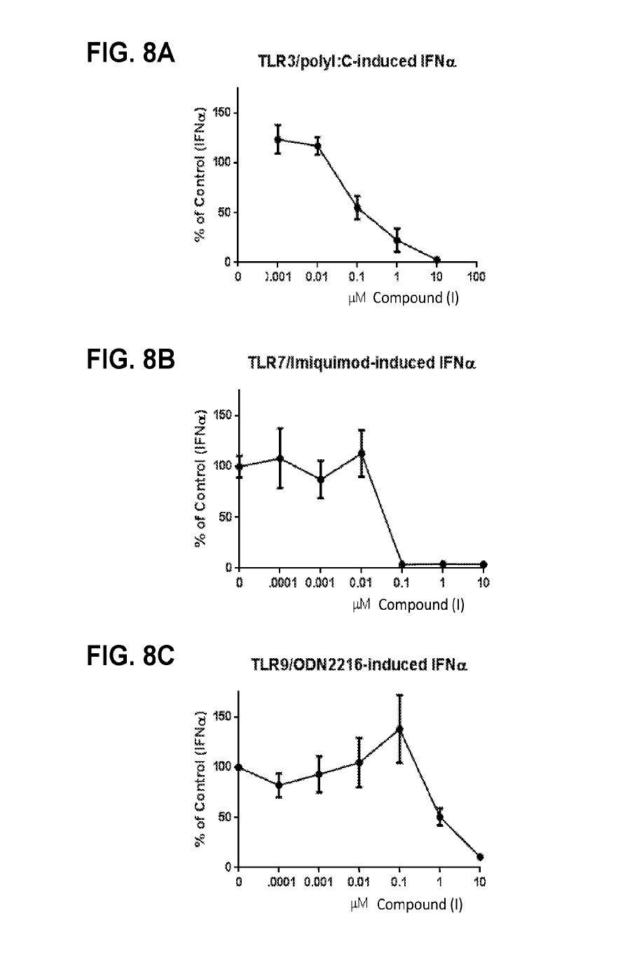

In some embodiments, the compound capable of modulating a TLR (e.g., a TLR3 antagonist, such as a SMAD7 ODN) can reduce the PolyI:C-induced IFNα expression or secretion by PBMCs, when the compound capable of modulating the TLR is contacted with the PBMCs at a concentration of 1.0 μM or less (e.g., about 0.05 μM, about 0.1 μM, about 0.2 μM, about 0.3 μM, about 0.4 μM, about 0.5 μM, about 0.6 μM, about 0.7 μM, about 0.8 μM, or about 0.9 μM), relative to a PolyI:C-induced PBMC control not contacted with the compound capable of modulating the TLR, as determined in an immunoassay (e.g., FACS, ELISA, and the like). In some embodiments, the compound capable of modulating the TLR can reduce the PolyI:C-induced IFNα expression or secretion by the PBMCs by 50% or more (e.g., 60% or more, 70% or more, 80% or more, or 90% or more). See, e.g., Example 6, FIG. 8A.

In some embodiments, the compound capable of modulating a TLR (e.g., a TLR7 antagonist, such as a SMAD7 ODN) can reduce the imiquimod-induced IFNα expression or secretion by PBMCs, when the compound capable of modulating the TLR is contacted with the PBMCs at a concentration of 1.0 μM or less (e.g., about 0.05 μM, about 0.1 μM, about 0.2 μM, about 0.3 μM, about 0.4 μM, about 0.5 μM, about 0.6 μM, about 0.7 μM, about 0.8 μM, or about 0.9 μM), relative to an imiquimod-induced PBMC control not contacted with the compound capable of modulating the TLR, as determined in an immunoassay (e.g., FACS, ELISA, and the like). In some embodiments, the compound capable of modulating the TLR can reduce the imiquimod-induced IFNα expression or secretion by the PBMCs by 50% or more (e.g., 60% or more, 70% or more, 80% or more, or 90% or more). See, e.g., Example 6, FIG. 8B.

In some embodiments, the compound capable of modulating a TLR (e.g., a TLR9 antagonist, such as a SMAD7 ODN) can reduce the ODN2216-induced IFNα expression or secretion by PBMCs, when the compound capable of modulating the TLR is contacted with the PBMCs at a concentration of 1.0 μM or less (e.g., about 0.05 μM, about 0.1 μM, about 0.2 μM, about 0.3 μM, about 0.4 μM, about 0.5 μM, about 0.6 μM, about 0.7 μM, about 0.8 μM, or about 0.9 μM), relative to an ODN2216-induced PBMC control not contacted with the compound capable of modulating the TLR, as determined in an immunoassay (e.g., FACS, ELISA, and the like). In some embodiments, the compound capable of modulating the TLR can reduce the ODN2216-induced IFNα expression or secretion of the PBMCs by 50% or more (e.g., 60% or more, 70% or more, 80% or more, or 90% or more). See, e.g., Example 6, FIG. 8C.

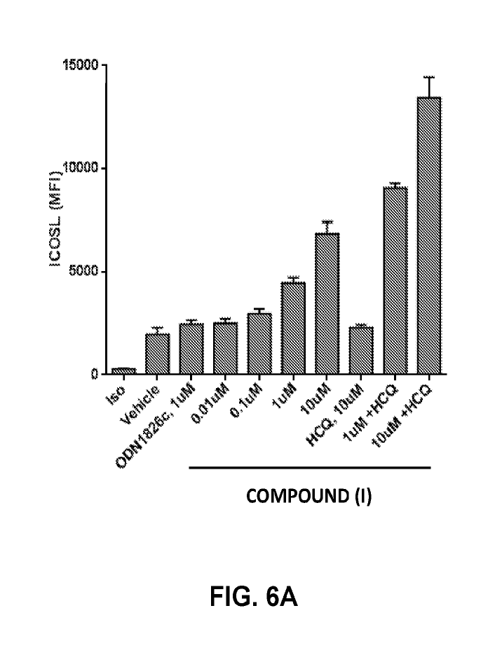

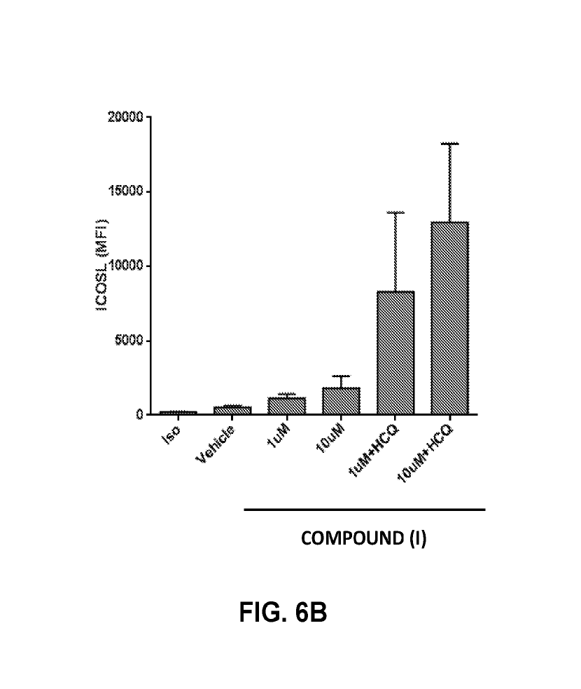

In some embodiments, the compound capable of modulating the TLR (e.g., a TLR synergist, such as a SMAD7 ODN) can increase the expression of ICOS-L proteins by a pDC by a factor of 5-fold or more, when contacted with the pDC at a concentration of 0.5 μM or more, in the presence of a quinoline or quinine relative to a pDC control not contacted with the compound capable of modulating the TLR, as determined in an immunoassay (e.g., FACS, ELISA, and the like), wherein the quinoline or quinine is present at a concentration below the threshold concentration at which the quinoline or quinine alone detectably increases ICOS-L expression. See, e.g., Example 3, FIGS. 6A-B. In some embodiments, the compound capable of modulating the TLR is contacted with the pDC at a concentration of 1.0 μM or more, 2.0 μM or more, 3.0 μM or more, 4.0 μM or more, 5.0 μM or more, 6.0 μM or more, 7.0 μM or more, 8.0 μM or more, 9.0 μM or more, 10.0 μM or more, or 15.0 μM or more. In some embodiments, the compound capable of modulating the TLR is contacted with the pDC at a concentration of about 1.0 μM. In some embodiments, the compound capable of modulating the TLR is contacted with the pDC at a concentration of about 10.0 μM. In some embodiments, the compound capable of modulating the TLR is hydroxychloroquine. In some embodiments, ICOS-L expression is increased by at least 7-fold, at least 10-fold, at least 15-fold, or at least 20-fold.

In some embodiments, a compound capable of modulating a TLR (e.g., a TLR9 agonist, such as a SMAD7 ODN) can increase expression or secretion of IL1-β, IL-18, CD-69, CCL2, CCL7, phospho-histone H3, phospho-p38 MAP kinase, phospho-ZAP70, or MCP-1 in a cell of the immune system, e.g., by at least 2-fold, at least 3-fold, at least 4-fold, at least 5-fold, at least 6-fold, at least 7-fold, at least 8-fold, at least 9-fold, or at least 10-fold relative to a baseline level observed in the absence of the TLR modulator, when the TLR modulator is contacted with the cell at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). In some embodiments, the compound capable of modulating a TLR can increase expression or secretion of 2, 3, 4, 5, 6, 7, 8, or 9 markers selected from IL1-β, IL-18, CD-69, CCL2, CCL7, phospho-histone H3, phospho-p38 MAP kinase, phospho-ZAP70, and MCP-1.

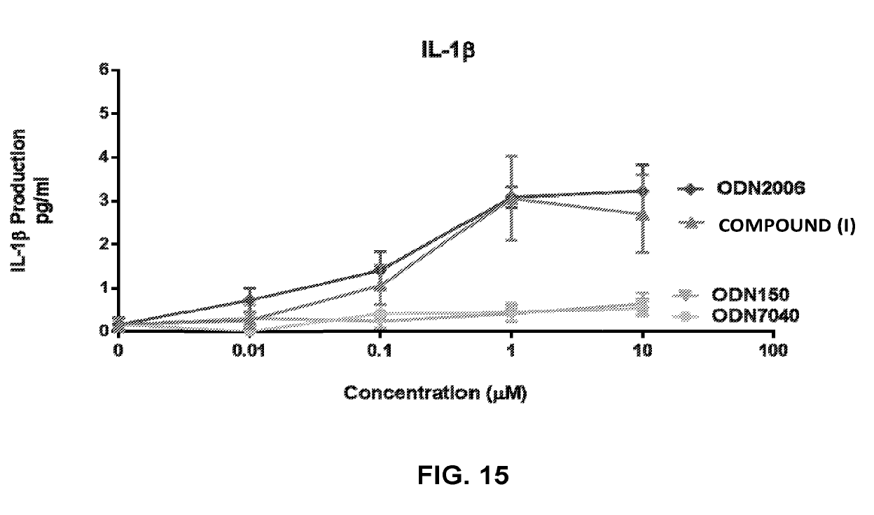

In some embodiments, a compound capable of modulating a TLR (e.g., a TLR9 agonist, such as a SMAD7 ODN) can increase secretion of a component of the inflammasome, such as IL1-β, IL-18, or both, from a PBMC by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold above a baseline level observed in the absence of the TLR modulator when the PBMC is contacted with the TLR modulator at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 15. In some embodiments, the compound capable of modulating a TLR can increase PBMC secretion of IL1-β, IL-18, or both.

In some embodiments, a compound capable of modulating a TLR (e.g., a TLR9 agonist, such as a SMAD7 ODN) can increase secretion of a component of the inflammasome, such as IL-1β, IL-18, or both, from a PBMC to a level of 5.0 pg/ml or more, 4.5 pg/ml or more, 4.0 pg/ml or more, 3.5 pg/ml or more, 3.0 pg/ml or more, 2.5 pg/ml or more, 2.0 pg/ml or more, 1.0 pg/ml or more, or 0.5 pg/ml or more (e.g., in a PBMC cell culture) when the TLR modulator is contacted with the PBMC at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 15. In some embodiments, the compound capable of modulating a TLR can increase PBMC secretion of IL1-β, IL-18, or both.

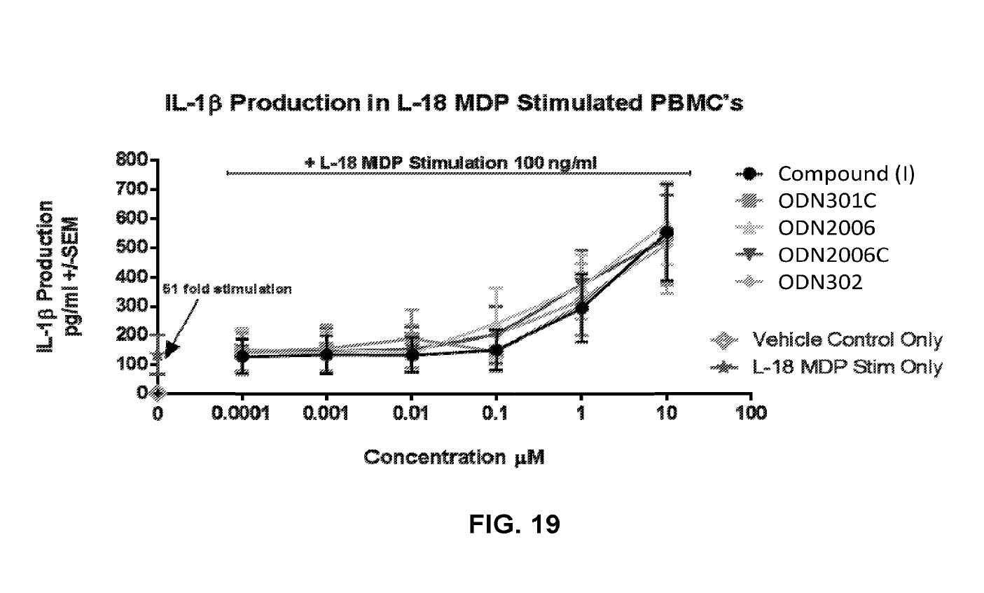

In some embodiments, a compound capable of modulating a TLR (e.g., a TLR9 agonist, such as a SMAD7 ODN) can increase secretion of a component of the inflammasome, such as IL1-β or IL-18 from a NOD2-ligand stimulated cell of the immune system (e.g., a PBMC contacted with L-18 MDP at 100 ng/ml) by at least 2-fold, at least 3-fold, at least 4-fold, at least 5-fold, at least 6-fold, at least 7-fold, at least 8-fold, at least 9-fold, or at least 10-fold above a baseline level observed for the NOD2-ligand stimulated cell in the absence of the TLR modulator when the TLR modulator is contacted with the NOD2-ligand stimulated cell at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 19. In some embodiments, the compound capable of modulating a TLR can increase PBMC secretion of IL1-β, IL-18, or both.

In some embodiments, a compound capable of modulating a TLR (e.g., a TLR9 agonist, such as a SMAD7 ODN) can activate the inflammasome (e.g., as determine by secretion of IL-1β, IL-18, or both) in a cell of the immune system to a level comparable to the level of activation observed with a CpG-B ODN (e.g., levels of inflammasome induction observed with compound or CpG-B ODN differ by less than 10-fold, less than 8-fold, less than 6-fold, less than 4-fold, or less than 2-fold under otherwise comparable or identical experimental conditions). In some embodiments, the compound capable of modulating the TLR can increase secretion of the inflammasome component to higher levels than a CpG-A ODN, Kappaproct® (ODN150), or Monarsen® (ODN7040) (e.g., secretion levels the inflammasome component observed with compound are at least 2-fold, 4-fold, 6-fold, 8-fold, or 10-fold higher than levels observed with CpG-A ODN, Kappaproct®, or Monarsen® under otherwise comparable or identical experimental conditions).

In some embodiments, a compound capable of modulating a TLR (e.g., a TLR9 agonist, such as a SMAD7 ODN) can decrease the expression or secretion of IL-1α, CCR6, CD123 (IL-3Rα), Eot3, ICAM-1, IgG, ITAC, M-CSF, MIG, or MIP-1α, in a cell of the immune system (e.g., to less than 90%, less than 80%, less than 70%, less than 60%, less than 50%, less than 40%, less than 30%, less than 20%, or less than 10% of a baseline level observed in the absence of the TLR modulator) when the compound is contacted with the cell at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). In some embodiments, the compound capable of modulating a TLR can decrease the expression or secretion of 1, 2, 3, 4, 5, 6, 7, 8, 9, 10, 11, or 12 markers selected from IL-1α, CCR6, CD123 (IL-3Rα), Eot3, ICAM-1, IgG, ITAC, M-CSF, MIG, or MIP-1α, in a cell of the immune system.

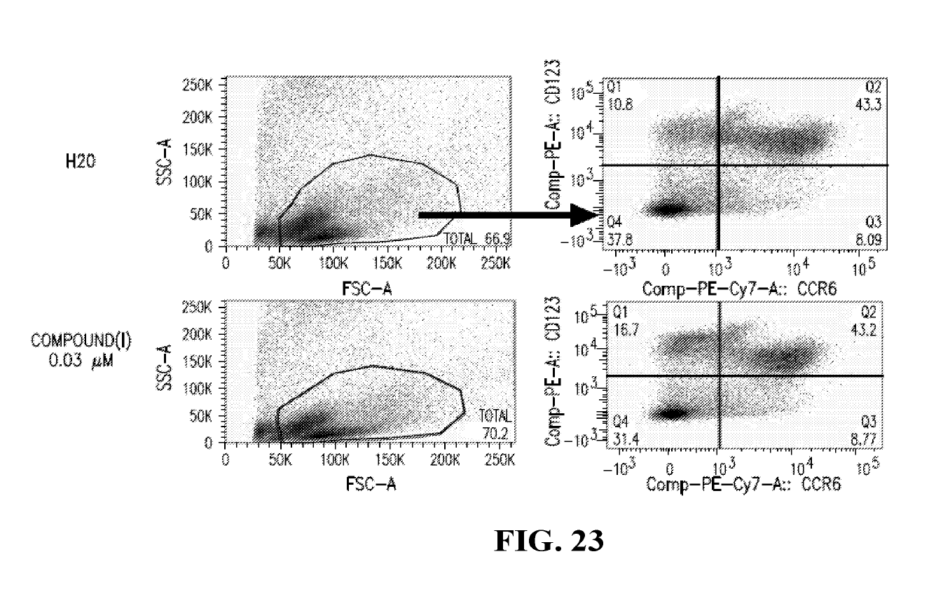

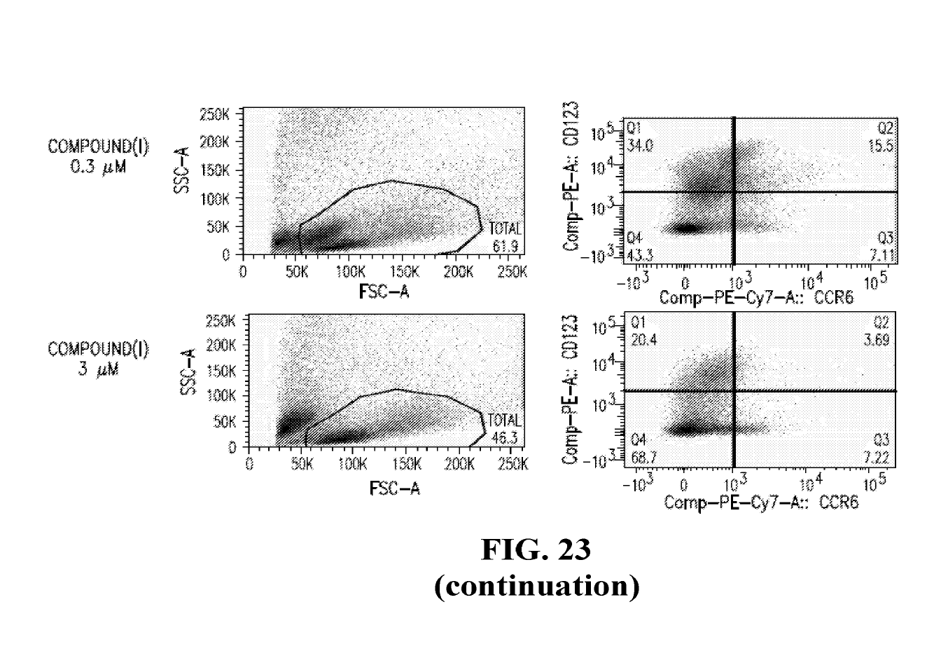

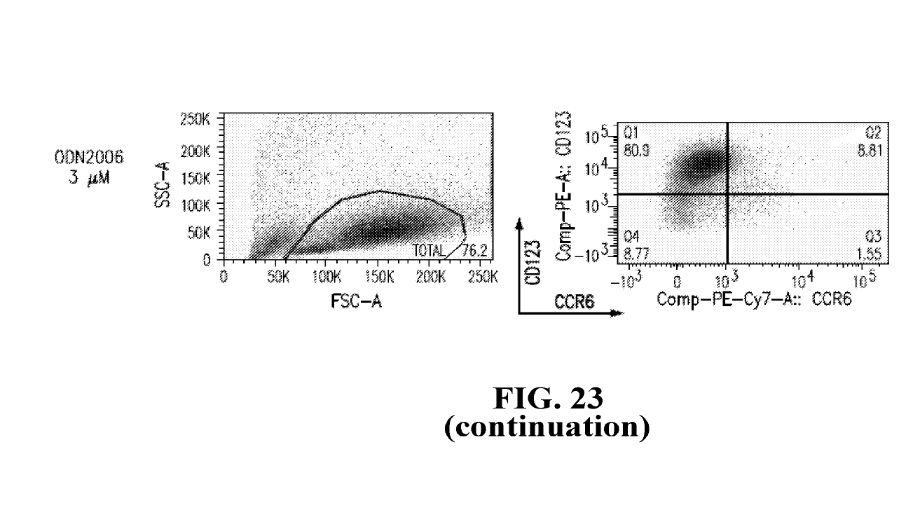

In some embodiments, a compound capable of modulating a TLR (e.g., a SMAD7 ODN) can induce pDC differentiation, e.g., as determined by detection of a pDC differentiation marker by FACS (e.g., CD80, CD83, CD86, CCR6, CCR7, CD123, SLAMF7, and the like) by at least 2-fold, at least 3-fold, at least 4-fold, at least 5-fold, at least 6-fold, at least 7-fold, at least 8-fold, at least 9-fold, or at least 10-fold above a baseline level observed for the pDC in the absence of the TLR modulator when the TLR modulator is contacted with the pDC at a concentration of 10.0 μM or less (e.g., about 10.0 μM, about 8.0 μM, about 6.0 μM, about 4.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 23.

In some embodiments, a compound capable of modulating a TLR (e.g., a TLR9 agonist, such as a SMAD7 ODN) can induce pDC differentiation, e.g., as determined by detection of a pDC differentiation marker by FACS (e.g., CD80, CD83, CD86, CCR6, CCR7, CD123, SLAMF7, and the like) to a level comparable to the level of activation observed with a CpG-B ODN (e.g., levels of pDC differentiation observed with compound or CpG-B ODN differ by less than 10-fold, less than 8-fold, less than 6-fold, less than 4-fold, or less than 2-fold under otherwise comparable or identical experimental conditions). In some embodiments, the compound capable of modulating the TLR can induce pDC differentiation to higher levels than a CpG-A ODN, Kappaproct® (ODN150), or Monarsen® (ODN7040) (e.g., levels of pDC differentiation observed with compound are at least 2-fold, 4-fold, 6-fold, 8-fold, or 10-fold higher than levels observed with CpG-A ODN, Kappaproct®, or Monarsen® under otherwise comparable or identical experimental conditions).

In some embodiments, a compound capable of modulating a TLR can induce epithelial restitution or mucosal healing, e.g., in an animal model of IBD, or in a human IBD patient, e.g., as determined by endoscopy.

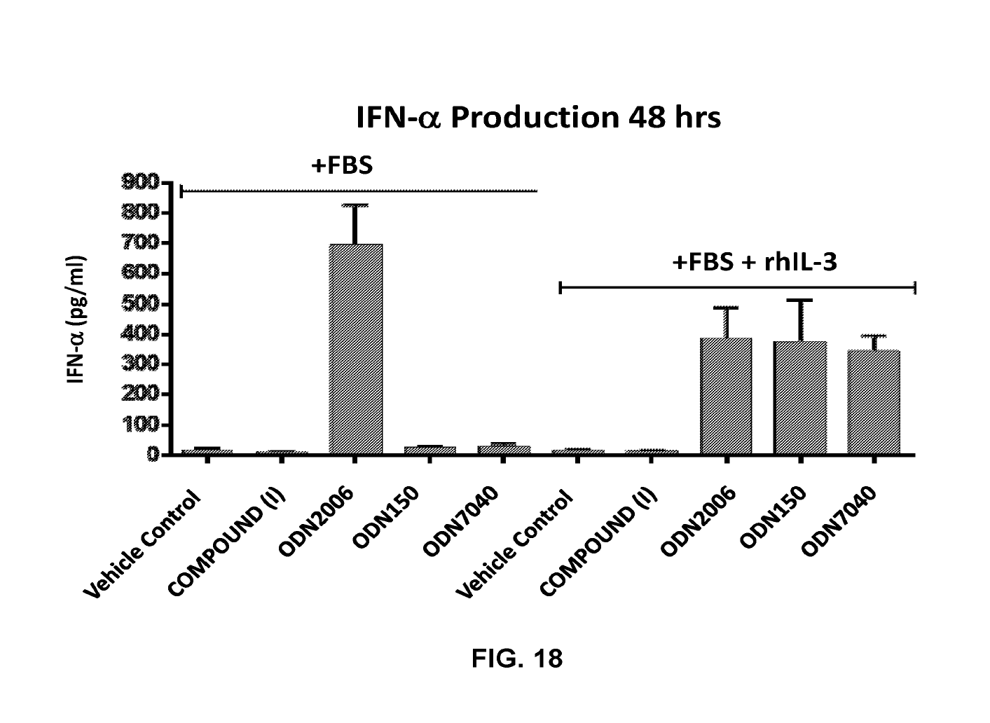

In some embodiments, a compound capable of modulating a TLR (e.g., a TLR9 agonist, such as a SMAD7 ODN) cannot detectably induce IFN-α or only weakly induces IFN-α (e.g., less than 100 pg/ml, less than 80 pg/ml, less than 60 pg/ml, less than 40 pg/ml, less than 20 pg/ml, or less than 10 pg/ml, e.g., in a pDC culture) in a cell of the immune system (e.g., a purified or mature pDC) when the TLR modulator is contacted with the cell at a concentration of 10.0 μM or less (e.g., about 10.0 μM, about 8.0 μM, about 6.0 μM, about 4.0 μM, about 3.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 18.

In some embodiments, the compound capable of modulating the TLR (e.g., a SMAD7 ODN) induces IFN-α in a cell of the immune system at lower levels than a CpG-A ODN, a CpG-B ODN (ODN2006), Kappaproct® (ODN150), or Monarsen® (ODN7040) (e.g., IFN-α levels observed with compound are either undetectable, or at least 2-fold, 4-fold, 6-fold, 8-fold, or 10-fold lower than IFN-α levels observed with the CpG-A ODN, CpG-B ODN, Kappaproct®, or Monarsen® under otherwise comparable or identical experimental conditions).

In some embodiments, a compound capable of modulating a TLR (e.g., a TLR9 agonist, such as a SMAD7 ODN) cannot detectably induce proliferation of a cell of the immune system (e.g., B-cell) or only weakly induces proliferation (e.g., less than 4-fold, less than 3-fold, or less than 2-fold induction of cell proliferation relative to cell proliferation observed in the absence of the TLR modulator) in the cell of the immune system (e.g., B-cell) when the TLR modulator is contacted with the cell at a concentration of 10.0 μM or less (e.g., about 10.0 μM, about 8.0 μM, about 6.0 μM, about 4.0 μM, about 3.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM), e.g., in a thymidine incorporation assay. See, e.g., FIG. 17.

In some embodiments, the compound capable of modulating the TLR (e.g., a SMAD7 ODN) induces proliferation in the cell of the immune system (e.g., B-cell) at lower levels than a CpG-B ODN (e.g., B-cell proliferation with compound is either undetectable, or at least 2-fold, 4-fold, 6-fold, 8-fold, or 10-fold lower than B-cell proliferation observed with the CpG-B ODN under otherwise comparable or identical experimental conditions).

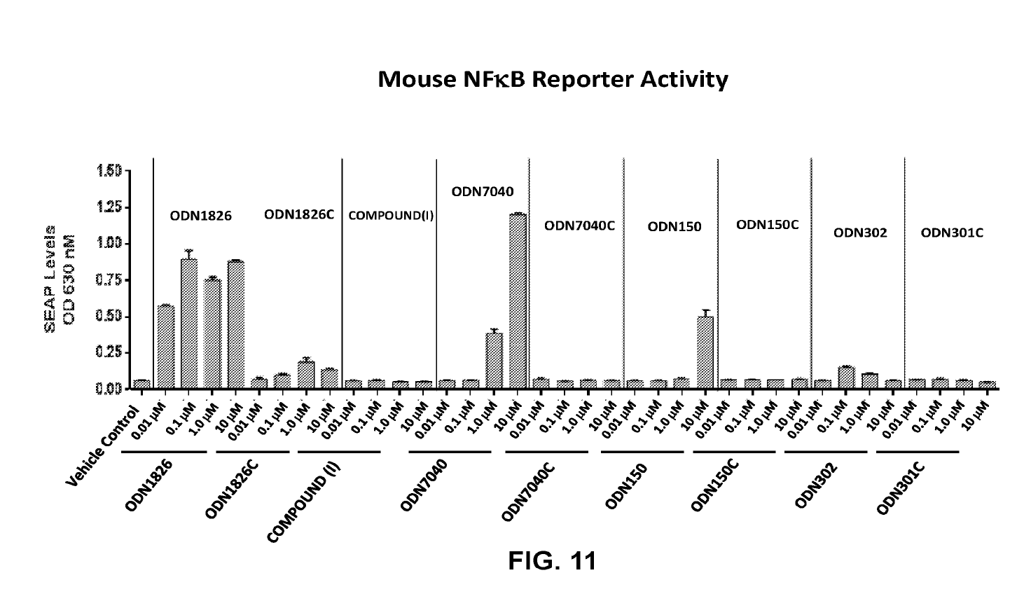

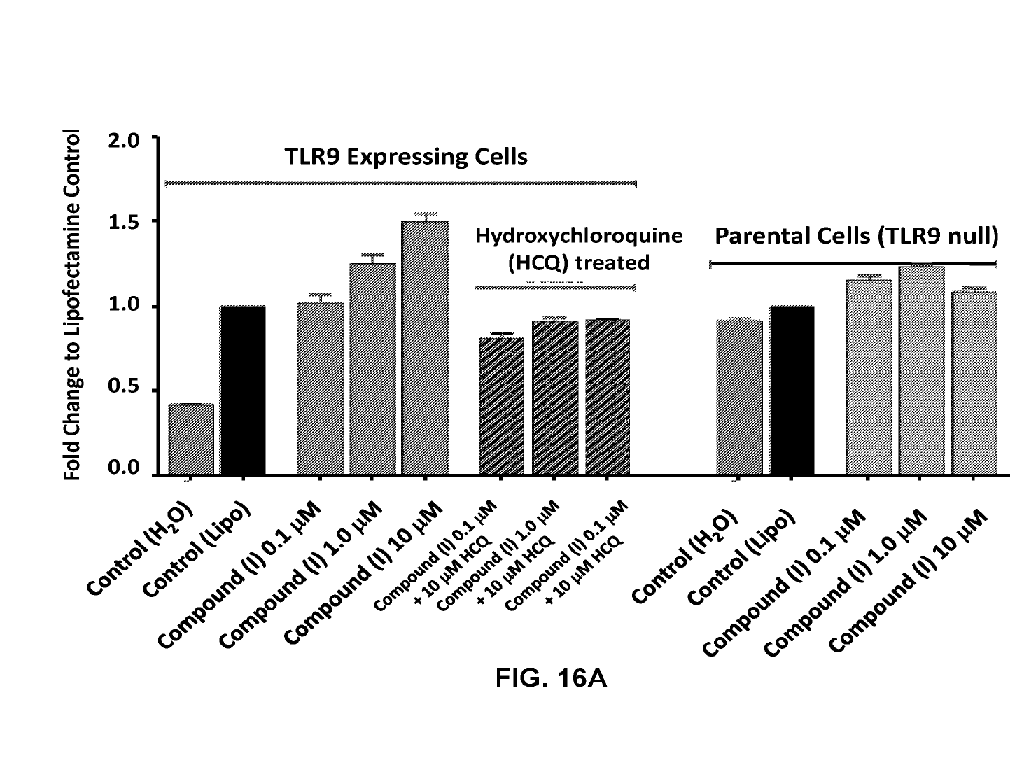

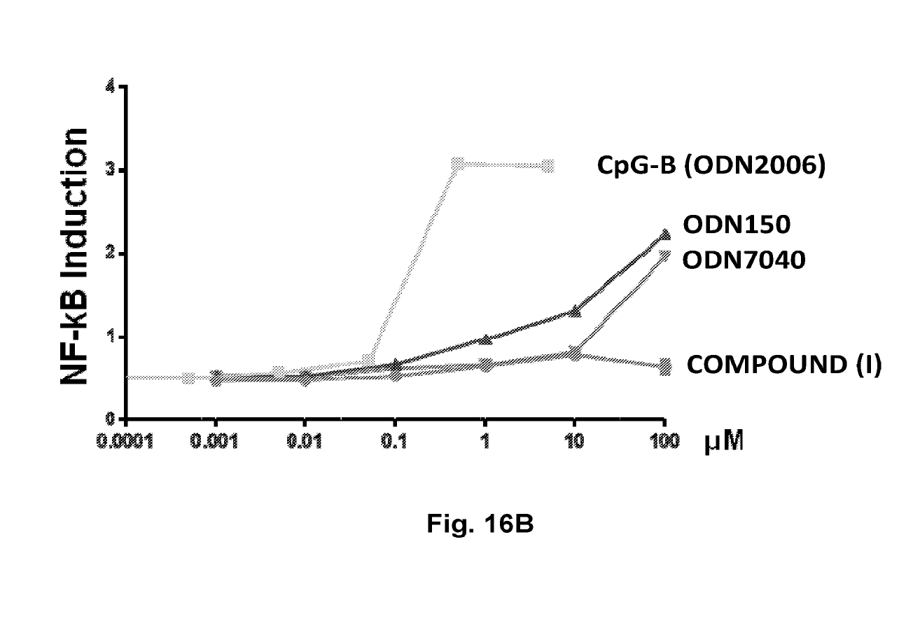

In some embodiments, a compound capable of modulating a TLR (e.g., a TLR9 agonist, such as a SMAD7 ODN) cannot detectably or only weakly induces activation of the NF-kB pathway (e.g., less than 4-fold, less than 3-fold, or less than 2-fold induction of the NF-kB pathway relative to the NF-kB pathway activity observed in the absence of the TLR modulator), e.g., in a cell of the immune system (e.g., a pDC), or in a reporter cell, when the TLR modulator is contacted with the cell at a concentration of 10.0 μM or less (e.g., about 10.0 μM, about 8.0 μM, about 6.0 μM, about 4.0 μM, about 3.0 μM, about 2.0 μM, about 1.0 μM, about 0.9 μM, about 0.8 μM, about 0.7 μM, about 0.6 μM, about 0.5 μM, about 0.4 μM, about 0.3 μM, about 0.2 μM, about 0.1 μM, or about 0.05 μM). See, e.g., FIG. 11, FIGS. 16A-B.

In some embodiments, the compound capable of modulating the TLR (e.g., a SMAD7 ODN) does not activate the NF-kB pathway or induces activation of the NF-kB pathway at lower levels than a CpG-B (e.g., ODN2006) (e.g., NF-kB pathway activation with the compound is either undetectable, or at least 2-fold, 4-fold, 6-fold, 8-fold, or 10-fold lower than NF-kB pathway activation observed with the CpG-B ODN under otherwise comparable or identical experimental conditions).

In some embodiments, a compound capable of modulating a TLR (e.g., a SMAD7 ODN) can induce inflammasome activation in a cell of the immune system (e.g., PBMC) and can induce pDC differentiation.

In some embodiments, a compound capable of modulating a TLR (e.g., a SMAD7 ODN) can induce inflammasome activation in a cell of the immune system (e.g., PBMC), can induce pDC differentiation, and cannot induce detectable levels of B-cell proliferation or only induces low levels of B-cell proliferation.

In some embodiments, a compound capable of modulating a TLR (e.g., a SMAD7 ODN) can induce inflammasome activation in a cell of the immune system (e.g., PBMC), can induce pDC differentiation, cannot induce detectable levels of B-cell proliferation or only induces low levels of B-cell proliferation, and cannot induce, or only weakly induce the NF-kB pathway in a cell of the immune system, or in a reporter cell. See, e.g., Examples 8 and 9, Tables 5 and 6.

In some embodiments, a compound capable of modulating a TLR (e.g., a SMAD7 ODN) can induce inflammasome activation (e.g., IL-1β or IL-18) more strongly than a CpG-A ODN, CpG-B ODN, Kappaproct®, or Monarsen® and can induce pDC differentiation more strongly than Kappaproct®, or Monarsen®. In some embodiments, the compound can induce pDC differentiation to similar levels as a CpG-B ODN (e.g., ODN2006). See, e.g., Examples 8 and 9, Tables 5 and 6.

In some embodiments, a compound capable of modulating a TLR (e.g., a SMAD7 ODN) can induce inflammasome activation (e.g., IL-1β or IL-18) more strongly than a CpG-A ODN, CpG-B ODN, Kappaproct®, or Monarsen® and can induce pDC differentiation more strongly than Kappaproct®, or Monarsen®, and induces B-cell proliferation at lower levels than a CpG-B ODN (e.g., ODN2006). In some embodiments, the compound can induce pDC differentiation to similar levels as a CpG-B ODN (e.g., ODN2006). In some embodiments, B-cell proliferation with the compound is only weak or not detectible. See, e.g., Examples 8 and 9, Tables 5 and 6.

In some embodiments, a compound capable of modulating a TLR (e.g., a SMAD7 ODN) can induce inflammasome activation (e.g., IL-1β or IL-18) more strongly than a CpG-A ODN, CpG-B ODN, Kappaproct®, or Monarsen® and can induce pDC differentiation more strongly than Kappaproct®, or Monarsen®, and induces B-cell proliferation and NF-kB pathway activation at lower levels than a CpG-B ODN (e.g., ODN2006). In some embodiments, the compound can induce pDC differentiation to similar levels as a CpG-B ODN (e.g., ODN2006). In some embodiments, B-cell proliferation with the compound is only weak or is not detectible. See, e.g., Examples 8 and 9, Tables 5 and 6.

In some embodiments, a compound capable of modulating a TLR (e.g., a SMAD7 ODN) can induce inflammasome activation (e.g., IL-1β or IL-18) more strongly than a CpG-A ODN, CpG-B ODN, Kappaproct®, or Monarsen® and can induce pDC differentiation more strongly than Kappaproct®, or Monarsen®, and induces B-cell proliferation, IFN-α secretion and NF-kB pathway activation at lower levels than a CpG-B ODN (e.g., ODN2006). In some embodiments, the compound can induce pDC differentiation to similar levels as a CpG-B ODN (e.g., ODN2006). In some embodiments, B-cell proliferation with the compound is only weak or is not detectible. See, e.g., Examples 8 and 9, Tables 5 and 6.

In another aspect, provided herein is a method of identifying or analyzing the activity of a TLR modulator, comprising a) analyzing a baseline level of a biomarker in a cell culture; b) contacting a test compound with the cell culture for a period of time, and c) analyzing a second level of the biomarker following the contacting, wherein, the test compound is a TLR modulator if the second level of the biomarker is increased or decreased relative to the baseline level of the biomarker.

In another aspect, provided herein is a method of identifying or analyzing the activity of a TLR modulator, comprising a) analyzing a level of a biomarker in a first cell culture in the absence of a test compound; b) contacting a test compound with a second cell culture for a period of time, and c) analyzing a level of the biomarker in the second cell culture following the contacting, wherein the test compound is a TLR modulator if the level of the biomarker in the second cell culture is increased or decreased relative to the level of the biomarker in the first cell culture.

In some embodiments, the TLR modulator can increase or decrease the level of a biomarker by at least 2-fold, at least 3-fold, at least 4-fold, at least 5-fold, at least 6-fold, at least 7-fold at least 8-fold, at least 9-fold, at least 10-fold, at least 20-fold, at least 30-fold, at least 40-fold, or at least 50-fold. In some embodiments, the TLR modulator can increase the level of a biomarker from undetectable levels to detectable levels. In some embodiments, the TLR modulator can decrease the level of a biomarker from detectable levels to undetectable levels.

In some embodiments, the cell culture is a primary cell culture. In some embodiments, the cell culture is a B-cell, PBMC or pDC cell culture. In some embodiments, the cultured pDCs are purified pDCs (e.g., cultured in the absence of IL-3). In some embodiments, the cultured pDCs are mature pDCs (e.g., cultured in the presence of IL-3). In some embodiments, the B-cell, PBMC or pDC is a human, monkey, ape, mouse, rat, rabbit, hamster, dog, cat, cow, or goat B-cell, PBMC or pDC.

In some embodiments, the cell culture comprises a reporter cell, e.g., a cell line comprising a recombinant reporter construct. In some embodiments, the reporter cell comprises a reporter gene driven by a cytokine promoter, such as an IP-10, TNFα, IFNγ, or IL-1β promoter. In some embodiments, the reporter cell comprises a reporter gene driven by an NF-κB promoter (e.g., a canonical NF-κB promoter). In some embodiments, the reporter gene is a luciferase, peroxidase, or phosphatase gene. In some embodiments, the reporter cell is derived from a cell line, e.g., a human, hamster, or mouse cell line (e.g., HEK, CHO or RAW 264.7 cells).

In some embodiments, the cell culture comprises a cell comprising an inflammasome. In some embodiments, inflammasome activity can be analyzed by analyzing the expression or secretion of an inflammasome component, such as IL-1β or IL-18 (e.g., by RT-PCT or ELISA).

In some embodiments, the biomarker level is analyzed at the transcriptional level, using, e.g., a reporter gene assay or a quantitative RT-PCR assay, or the like. In some embodiments, the biomarker is secreted into the medium of a cell culture. In some embodiments, the biomarker level is analyzed by analyzing the biomarker level in cell culture sample, e.g., using ELISA, SPR, TR-FRET, or the like. In some embodiments, the biomarker remains attached to the cell. In some embodiments, the biomarker level is analyzed, e.g., by FACS, immunohistochemistry, or microscopic imaging (e.g., fluorescence microscopy), or the like.

In some embodiments, the biomarker is a cytokine. In some embodiments, the cytokine is TNFα, TGFβ, IFNγ, IL-1β, IL6, IL10, or IP-10 (CXCL10). In some embodiments, the biomarker is PD-L1, IDO, or ICOS-L

In some embodiments, the biomarker is bFGF, CCR6, CCR7, CD80, CD83, CD86, CD-69, CD123 (IL-3Rα), EGFR, Eot3, GARP, ICAM-1, IgG, IL-1α, IL1-β, IL-2, IL-4, IL-10Rα, IL-18, IL-23p19, ILT7, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1

In some embodiments, the TLR modulator is a TLR3, TLR4, TLR7, TLR8, or TRL9 modulator.

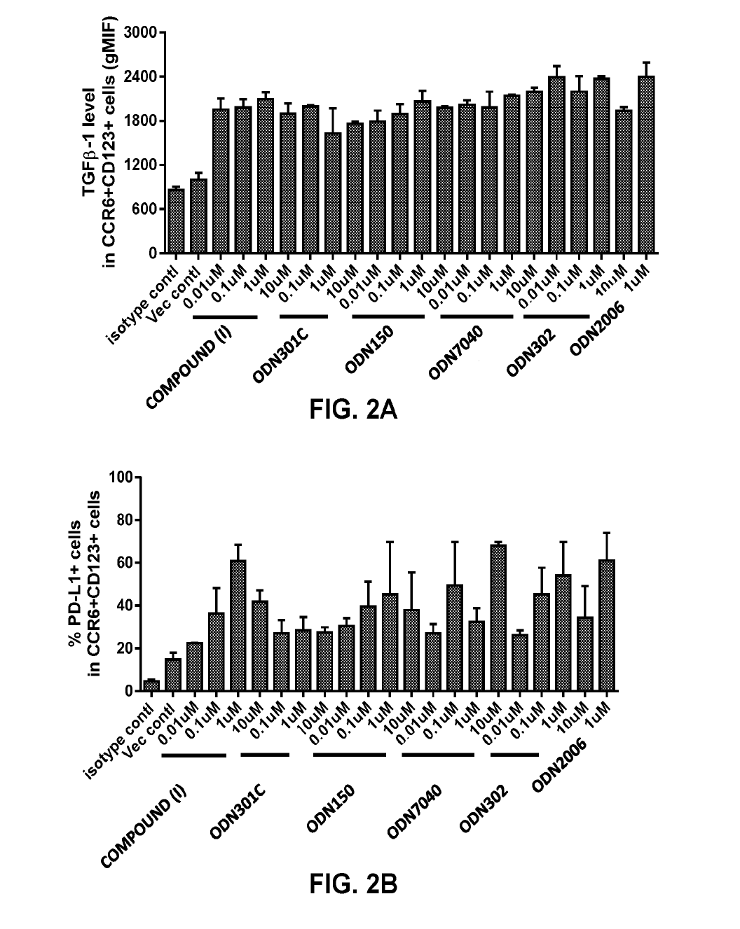

In some embodiments, the TLR modulator is a TLR9 agonist if the second level of IP-10 is lower than the first level of IP-10. In some embodiments, the TLR modulator is a TLR9 agonist, if the second level of TNFα, IFNγ, IL-1β, IL-10, TGFβ, PD-L1, or ICOS-L is increased relative to the first level of TNFα, IFNγ, IL-1β, IL-10, TGFβ, PD-L1, or ICOS-L. See, e.g., Examples 2 and 4, Table 4, FIGS. 2A-5B.

7.3 Additional Agents

In some embodiments, the anti-SMAD7 therapeutic described herein can be combined with an additional agent other than a TLR modulator. In some embodiments the combinations of the anti-SMAD7 therapeutic and the additional agent can be used in the methods of treatment provided herein. See, e.g., Section 7.3, 7.4 and 7.7.

In some embodiments, the additional agent is an additional therapeutic agent. In some embodiments, the additional agent can comprise a kinase inhibitor, a dihydrofolate reductase inhibitor, or a NOD2 ligand.

In some embodiments, the kinase inhibitor can comprise a Jak (e.g., Jak1, Jak2, Jak3, or Tyk2) inhibitor or a Src-family kinase (e.g., Src, Yes, Fyn, Fgr, Lck, Hck, Blk, Lyn, or Frk kinase) inhibitor. In some embodiments, the Jak inhibitor can comprise tofacitinib, filgotinib, or baricitinib. In some embodiments, the Src kinase inhibitor can include SU6656.

In some embodiments, the dihydrofolate reductase inhibitor can comprise methotrexate, trimethoprim, or pyrimethamine.

In some embodiments, the NOD2 ligand can comprise muramyl dipeptide (MDP) or a modified MDP comprising a 6-O-acyl derivative with a stearoyl fatty acid (L18-MDP).

In some embodiments, the additional agent combined with the anti-SMAD7 therapeutic is administered to a patient at a low dose, e.g., less than 0.3-fold, less than 0.1-fold, less than 0.03-fold, less than 0.01-fold the MTD of the additional agent (e.g., MTD in a human patient).

7.4 Anti-SMAD7 Therapeutics

In some embodiments, the anti-SMAD7 therapeutics described herein comprise an anti-SMAD7 ODN. In some embodiments, the anti-SMAD7 ODN is capable of reducing the expression level of a SMAD7 mRNA or of a SMAD7 protein when introduced into a cell (e.g., a cell of a cell culture, or a cell of a tissue sample obtained from a patient). In some embodiments, the cell is a cell of the immune system, such as a PBMC or a pDC. In some embodiments, the cell is a cell of a tissue sample obtained from a patient, such as a human IBD patient (e.g., an intestinal biopsy sample) or in a sample obtained from the subject (e.g., a tissue biopsy sample). In some embodiments, the anti-SMAD7 ODN is introduced into the cell by transfection (e.g., using lipid transfection reagents, or viral vectors) or by electroporation. In some embodiments, the anti-SMAD7 ODN is a SMAD7 antisense oligonucleotide (SMAD7 AON). In some embodiments, the anti-SMAD7 ODN is a SMAD7 inhibitory RNA (SMAD7 RNAi). In some embodiments, the anti-SMAD7 ODN is a SMAD7 microRNA (SMAD7 miRNA).

In some embodiments, the anti-SMAD7 ODN comprises deoxyribonucleic acid (DNA) or ribonucleic acid (RNA) oligonucleotides, or hybrids thereof.

In some embodiments, the anti-SMAD7 ODN is capable of modulating a TLR (e.g., inhibit or activate a TLR). In some embodiments, the TLR is TLR3, TLR4, TLR7, TLR8 or TLR9. See, e.g., Sections 7.2 and 7.8.(a).

In some embodiments, the anti-SMAD7 ODN is a chemically modified anti-SMAD7 ODN. In some embodiments, the chemically modified anti-SMAD7 ODN comprises, e.g., a non-naturally occurring internucleoside linkage, a non-naturally occurring sugar residue, a non-naturally occurring base, a label (e.g., a fluorescence label or isotope label, such as a deuterium or tritium label), or another modification.

In some embodiments, the anti-SMAD7 ODN comprises a CG dinucleotide sequence. In some embodiments, the anti-SMAD7 ODN comprises a GC dinucleotide sequence. In some embodiments, the CG or the GC dinucleotide sequence is a plurality of CG dinucleotide sequences. In some embodiments, the plurality of CG or GC dinucleotide sequences is 2 or more, 3 or more, 4 or more, 5 or more, or 6 or more CG or GC dinucleotide sequences. In some embodiments, the plurality of CG or GC dinucleotide sequences comprises one or more CG dinucleotide sequences and one or more GC dinucleotide sequences. In some embodiments, the plurality of CG or GC dinucleotide sequences comprises only CG dinucleotide sequences or only GC dinucleotide sequences.

In some embodiments the anti-SMAD7 ODN comprises at least one CG or GC dinucleotide sequence comprising a methylated base (e.g., 5-methyl-cytosine, 6-O-methyl-guanine; 7-methyl-guanine). In some embodiments, the cytosine in a CG or GC dinucleotide sequence is methylated (e.g., 5-methyl-cytosine). In some embodiments, the guanine in the CG or GC dinucleotide sequence is methylated (e.g., 6-O-methyl-guanine; 7-methyl-guanine). In some embodiments, the cytosine and the guanine in the CG or GC dinucleotide sequence is methylated. In some embodiments, the anti-SMAD7 ODN comprises a plurality of CG or GC dinucleotide sequences comprising a methylated base (e.g., 5-methyl-cytosine, 6-O-methyl-guanine; 7-methyl-guanine). In some embodiments, the plurality of CG or GC dinucleotide sequences comprising a methylated base (e.g., 5-methyl-cytosine, 6-O-methyl-guanine; 7-methyl-guanine) is 2 or more, 3 or more, 4 or more, 5 or more, or 6 or more CG or GC dinucleotide sequences.

In some embodiments, the CG or GC dinucleotide sequence in the anti-SMAD7 ODN is a CG or GC phosphate dinucleotide sequence. In some embodiments one or more CG or GC dinucleotide sequences in the anti-SMAD7 ODN comprise a non-natural internucleoside linkage (e.g., a phosphorothioate linkage). In some embodiments, the CG or GC dinucleotide is a CG or GC phosphorothioate dinucleotide sequence. In some embodiments, a two or more CG or GC dinucleotide sequences in the anti-SMAD7 ODN are phosphorothioate dinucleotide sequences. In some embodiments, all CG or GC dinucleotide sequences in the anti-SMAD7 ODN are phosphorothioate dinucleotide sequences. In some embodiments, one or more of the CG or GC phosphorothioate dinucleotide sequences in the anti-SMAD7 ODN comprise one or two methylated bases (e.g., 5-methyl-cytosine, 6-O-methyl-guanine; 7-methyl-guanine). In some embodiments, one or more CG or GC dinucleotide sequences in the anti-SMAD7 ODN comprising a methylated base are phosphorothioate dinucleotide sequences. In some embodiments, all CG or GC dinucleotide sequences in the anti-SMAD7 ODN comprising a methylated base are phosphorothioate dinucleotide sequences.

The anti-SMAD7 ODN described herein can comprise a SMAD7 nucleotide sequence from any mammalian organism, for example, and without limitation, a primate (e.g., human, monkey, chimpanzee, orangutan, or gorilla), a cat, a dog, a rabbit, a farm animal (e.g., cow, horse, goat, sheep, pig), or a rodent (e.g., mouse, rat, hamster, or guinea pig).

In some embodiments, the anti-SMAD7 ODN comprises a nucleotide sequence complementary to a region in human SMAD7. In some embodiments, the anti-SMAD7 ODN comprises a nucleotide sequence complementary to a region of 8 or more, 10 or more, 12 or more, 14 or more, 16 or more, 18 or more, or 20 or more nucleotides of human SMAD7. In some embodiments, the anti-SMAD7 ODN comprises a nucleotide sequence complementary to a human SMAD7 sequence comprising the nucleotide sequence of SEQ ID NO: 1, or the corresponding RNA sequence.

SEQ ID NO: 1 (Coding Sequence: CDS (288-1568) of NM_005904.3; Homo sapiens SMAD family member 7 (SMAD7), transcript variant 1, mRNA) (region 108-128 underlined):

ATG TTCAGGACCA AACGATCTGC GCTCGTCCGG
CGTCTCTGGA GGAGCCGTGC GCCCGGCGGC GAGGACGAGG
AGGAGGGCGC AGGGGGAGGT GGAGGAGGAG
GCGA [symbol] GGACA GCCGAGCGCA
TGGGGCCGGT GGCGGCGGCC CGGGCAGGGC TGGATGCTGC
CTGGGCAAGG CGGTGCGAGG TGCCAAAGGT CACCACCATC
CCCACCCGCC AGCCGCGGGC GCCGGCGCGG CCGGGGGCGC
CGAGGCGGAT CTGAAGGCGC TCACGCACTC GGTGCTCAAG
AAACTGAAGG AGCGGCAGCT GGAGCTGCTG CTCCAGGCCG
TGGAGTCCCG CGGCGGGACG CGCACCGCGT GCCTCCTGCT
GCCCGGCCGC CTGGACTGCA GGCTGGGCCC GGGGGCGCCC
GCCGGCGCGC AGCCTGCGCA GCCGCCCTCG TCCTACTCGC
TCCCCCTCCT GCTGTGCAAA GTGTTCAGGT GGCCGGATCT
CAGGCATTCC TCGGAAGTCA AGAGGCTGTG TTGCTGTGAA
TCTTACGGGA AGATCAACCC CGAGCTGGTG TGCTGCAACC
CCCATCACCT TAGCCGACTC TGCGAACTAG AGTCTCCCCC
CCCTCCTTAC TCCAGATACC CGATGGATTT TCTCAAACCA
ACTGCAGACT GTCCAGATGC TGTGCCTTCC TCCGCTGAAA
CAGGGGGAAC GAATTATCTG GCCCCTGGGG GGCTTTCAGA
TTCCCAACTT CTTCTGGAGC CTGGGGATCG GTCACACTGG
TGCGTGGTGG CATACTGGGA GGAGAAGACG AGAGTGGGGA
GGCTCTACTG TGTCCAGGAG CCCTCTCTGG ATATCTTCTA
TGATCTACCT CAGGGGAATG GCTTTTGCCT CGGACAGCTC
AATTCGGACA ACAAGAGTCA GCTGGTGCAG AAGGTGCGGA
GCAAAATCGG CTGCGGCATC CAGCTGACGC GGGAGGTGGA
TGGTGTGTGG GTGTACAACC GCAGCAGTTA CCCCATCTTC
ATCAAGTCCG CCACACTGGA CAACCCGGAC TCCAGGACGC
TGTTGGTACA CAAGGTGTTC CCCGGTTTCT CCATCAAGGC
TTTCGACTAC GAGAAGGCGT ACAGCCTGCA GCGGCCCAAT
GACCACGAGT TTATGCAGCA GCCGTGGACG GGCTTTACCG
TGCAGATCAG CTTTGTGAAG GGCTGGGGCC AGTGCTACAC
CCGCCAGTTC ATCAGCAGCT GCCCGTGCTG GCTAGAGGTC
ATCTTCAACA GCCGGTAG

In some embodiments, the anti-SMAD7 ODN comprises a nucleotide sequence complementary to a human SMAD7 sequence comprising the nucleotide sequence of SEQ ID NOS: 8-10 or 88-90, or the corresponding RNA sequence.

In some embodiments, the anti-SMAD7 ODN comprises a nucleotide sequence complementary to region 108-128 of the human SMAD7 nucleotide sequence of SEQ ID NO: 1, or the corresponding RNA sequence.

In some embodiments, the anti-SMAD7 ODN comprises a nucleotide sequence complementary to nucleotides 403, 233, 294, 295, 296, 298, 299 or 533 of the human SMAD7 nucleotide sequence of SEQ ID NO: 1, or the corresponding RNA sequence.

In some embodiments, the anti-SMAD7 ODN comprises the nucleotide sequence of SEQ ID NO: 2 (5′-GTCGCCCCTTCTCCCCGCAG-3′).

In some embodiments, the anti-SMAD7 ODN comprises the nucleotide sequence of SEQ ID NO: 3 (5′-GTCGCCCCTTCTCCCCGCAGC-3′). In some embodiments, the anti-SMAD7 ODN comprises a sequence of 10 or more nucleotides of the nucleotide sequence of SEQ ID NO: 3 (e.g., 11 or more, 12 or more, 13 or more, 14 or more, 15, or more, 16 or more, 17 or more, 18 or more, 19 or more, or 20 or more nucleotides).

In some embodiments, the anti-SMAD7 ODN comprises COMPOUND (I). The following structure of COMPOUND (I) is drawn over four pages:

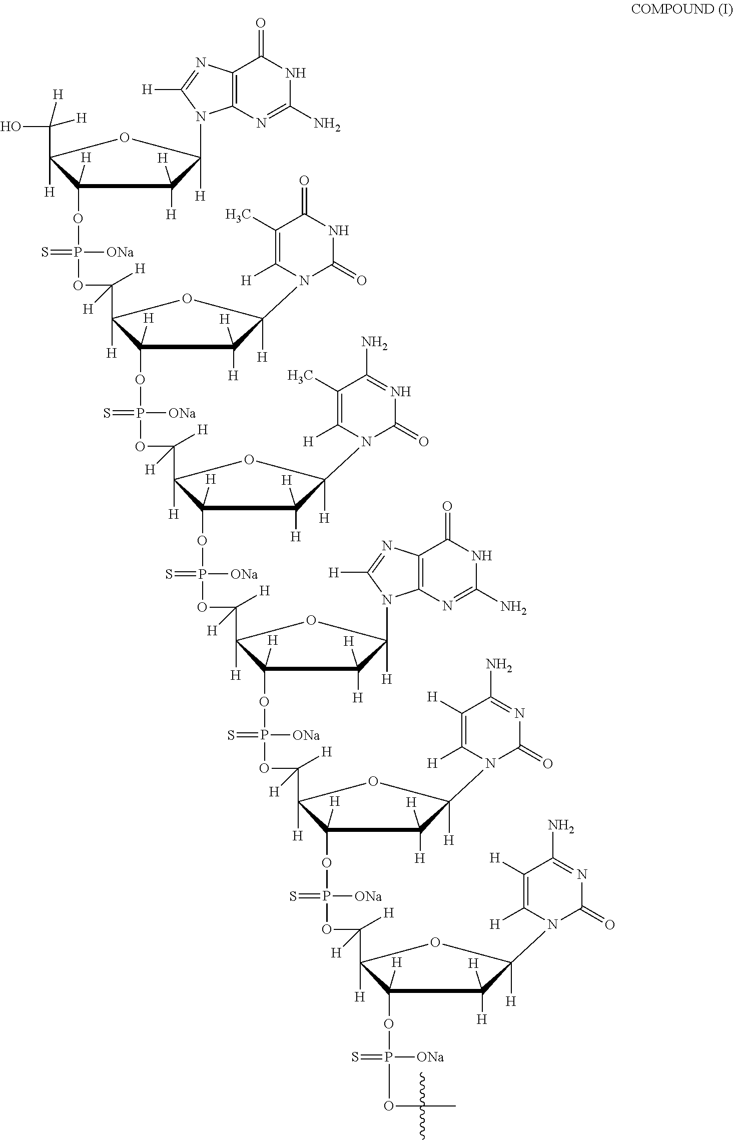

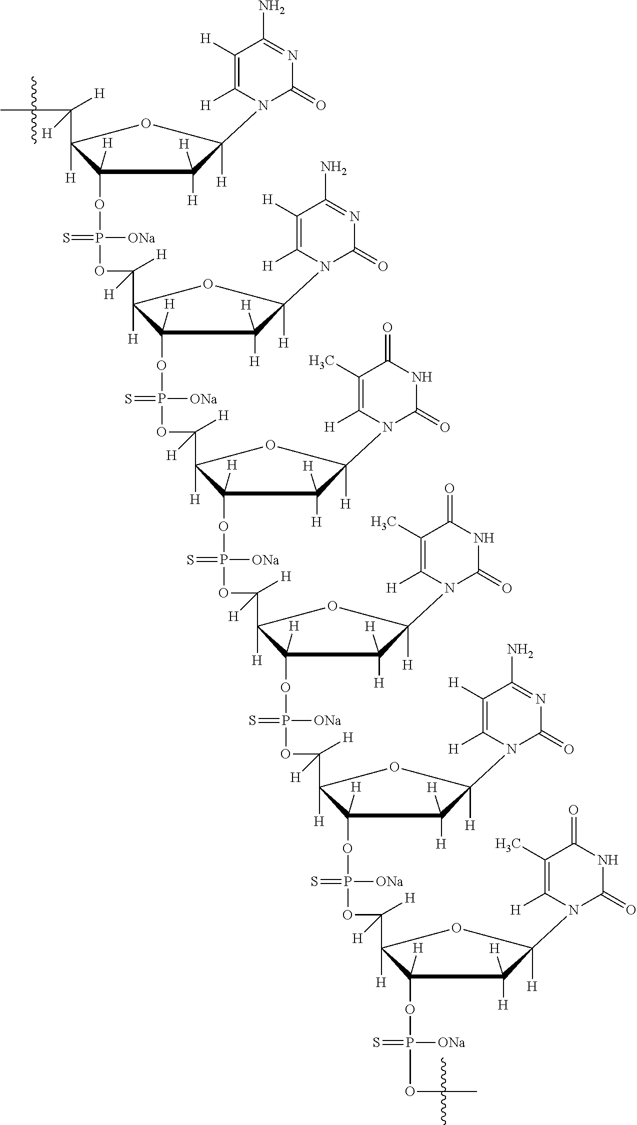

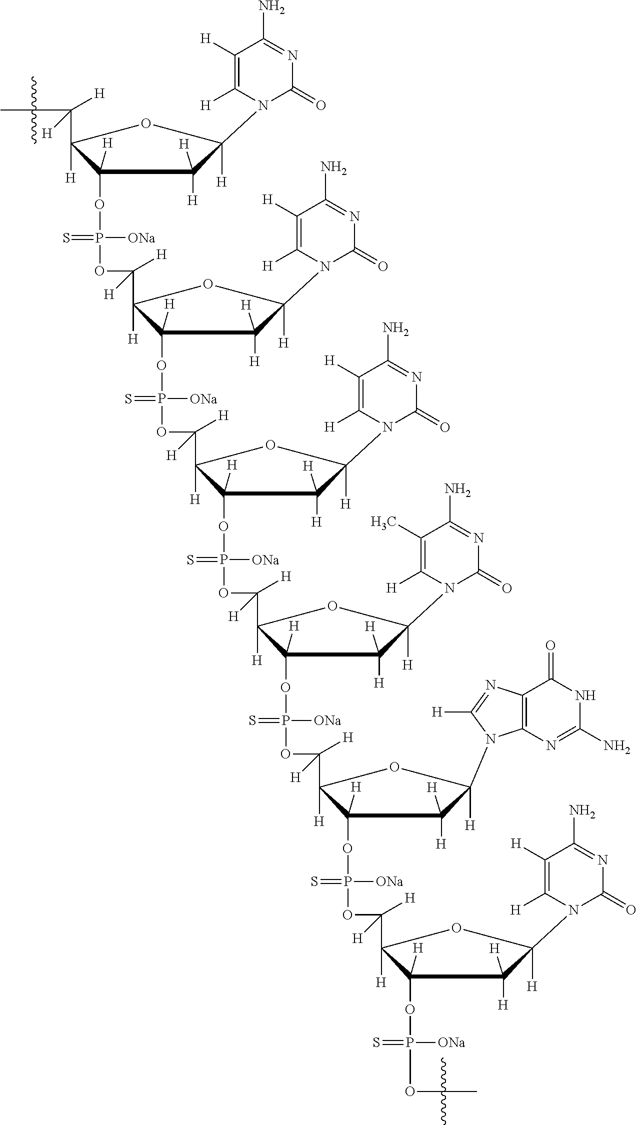

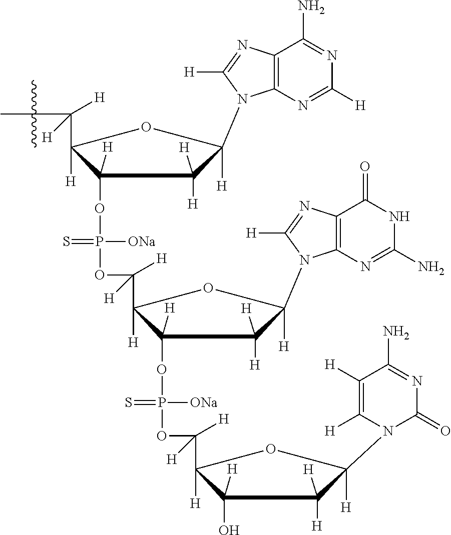

The structure of COMPOUND (I) is presented herein to show the sodium counterion (“Na”). A skilled artisan will understand that COMPOUND (I) may also refer to the anionic form without counterion. A skilled artisan will further understand that an anionic form of COMPOUND (I) can be protonated to form an acidic form of COMPOUND (I). In some embodiments, the phosphorothioate backbone of COMPOUND (I) can be fully or partially protonated to form an acidic form of COMPOUND (I).

The sequence of heterocyclic bases of COMPOUND (I) is depicted by SEQ ID NO: 5.

(a) SMAD7 Antisense Oligonucleotides

In some embodiments, the anti-SMAD7 ODNs described herein are SMAD7 antisense oligonucleotides (SMAD7 AONs). In some embodiments, the SMAD7 AON is a chemically modified SMAD7 AON.

Antisense oligonucleotides are short synthetic oligonucleotide sequences complementary to the messenger RNA (mRNA), which encodes for the target protein (e.g., SMAD7). Antisense oligonucleotide sequences hybridize to the mRNA producing a double-strand hybrid that can lead to the activation of ubiquitous catalytic enzymes, such as RNase H, which degrades DNA/RNA hybrid strands, thus preventing protein translation. Without being bound by theory, an antisense oligonucleotide provided herein can hybridize to its target sequence as RNA or DNA. Thus, even if a DNA sequence is provided as target, the corresponding RNA sequence (including uracil instead of thymine) is included.

The SMAD7 AONs described herein, when introduced into a cell (e.g., by transfection or electroporation), can reduce SMAD7 expression in the cell by reducing the level of a SMAD7 mRNA in the cell or by reducing the level of a SMAD7 protein in the cell. The SMAD7 AONs described herein can reduce SMAD7 expression in vitro, e.g., in a cultured cell, or in vivo, e.g., in a subject (such as a human patient or in an animal model organism).

Methods of determining SMAD7 mRNA or SMAD7 protein levels are well known in the art and can include, e.g., RT-PCR, Southern Blot, Western Blot, ELISA, or immunocytochemistry. SMAD7 expression in vivo can be analyzed, e.g., in a sample taken from a subject, such as a solid or liquid biopsy sample.

In connection with the compositions and methods provided herein, a reduction of SMAD7 mRNA or protein levels in a cell can be determined, e.g., by comparing the SMAD7 mRNA or protein levels in a test sample treated with a SMAD7 AON with the SMAD7 mRNA or proteins levels in a control sample. Control samples can include, e.g., untreated samples, samples treated with transfection reagents alone (“vehicle control”), or samples treated with a control ODN. Control ODNs can include, e.g., ODNs having a nucleotide sequence complementary to the nucleotide sequence of the SMAD7 AON. In some embodiments, the control ODN has a nucleotide sequence unrelated to the nucleotide sequence of the modified SMAD7 AON, such as a randomized nucleic acid sequence. In some embodiments, the control ODN is an AON for a gene other than SMAD7. In some embodiments, the control sample resembles the sample treated with a modified SMAD7 AON at a timepoint prior to SMAD7 AON administration (e.g., a sample taken at T=0 min in a timecourse experiment). In some embodiments, the test sample was obtained from a subject having received a SMAD7 AON treatment (e.g., a tissue sample). In some embodiments, the control sample was obtained from a treatment naive subject, which had never received a SMAD7 AON treatment.

In some embodiments, the SMAD7 AON can reduce the level of a SMAD7 mRNA in a cell by 10% or more, 20% or more, 30% or more, 40% or more, 50% or more, 60% or more, 70% or more, 80% or more, 90% or more, 95% or more, or 99% or more.

In some embodiments, the SMAD7 AON can reduce the level of a SMAD7 protein in a cell by 10% or more, 20% or more, 30% or more, 40% or more, 50% or more, 60% or more, 70% or more, 80% or more, 90% or more, 95% or more, or 99% or more.

In some embodiments, the SMAD7 AON can reduce the level of a SMAD7 mRNA or a SMAD7 protein within 1 hr, 2 hrs, 3 hrs, 6 hrs, 9 hrs, 12, hrs, 15 hrs, 18 hrs, 21 hrs, 24 hrs, 30 hrs, 36 hrs, 42 hrs, or 48 hrs of contacting the SMAD7 AON with a test sample (e.g., a cell in a cell culture) or of administering the SMAD7 AON to a subject.

In some embodiments, the SMAD7 AON is capable of modulating a TLR (e.g., inhibit or activate a TLR). In some embodiments, the TLR is TLR3, TLR4, TLR7, TLR8 or TLR9. See, e.g., Sections 7.2 and 7.8.(a).

In some embodiments, the SMAD7 AON is chemically modified.

(b) SMAD7 RNAi or miRNA

In some embodiments, the anti-SMAD7 ODN is a SMAD7 inhibitory RNA (RNAi or siRNA) or a SMAD7 micro RNA (SMAD7 miRNA). In some embodiments, the SMAD7 RNAi or SMAD7 miRNA is a chemically modified SMAD7 RNAi or a chemically modified SMAD7 miRNA.

RNAis are small RNA molecules that can be introduced into a cell or generated in a cell. Without wishing to be bound by theory, the RNAi pathway is initiated in a cell by the enzyme Dicer, which cleaves long double-stranded RNA (dsRNA) molecules into short double stranded fragments of ˜20 nucleotide siRNAs. Each siRNA is unwound into single-stranded RNAs (ssRNAs), the passenger strand and the guide strand. The passenger strand is degraded and the guide strand is incorporated into the RNA-induced silencing complex (RISC). Typically gene silencing ensues, which occurs when the guide strand pairs with a complementary sequence in a messenger RNA molecule and induces cleavage by Argonaute, the catalytic component of the RISC complex.

miRNAs are genomically encoded non-coding RNAs that help regulate gene expression, particularly during development. Mature miRNAs are structurally similar to siRNAs produced from exogenous dsRNA, but before reaching maturity, miRNAs must first undergo extensive post-translational modification. A miRNA is expressed from a much longer RNA-coding gene as a primary transcript known as pre-miRNA, which is processed in the cell nucleus to a 70-nucleotide stem-loop structure called a pre-miRNA by the microprocessor complex. This complex consists of an RNaseIII enzyme called Drosha and a dsRNA-binding protein DGCR8. The dsRNA portion of this pre-miRNA is bound and cleaved by Dicer to produce the mature miRNA molecule that can be integrated into the RISC complex. Thus miRNA and siRNA share the same downstream cellular machinery.

siRNAs derived from long dsRNA precursors differ from miRNAs in that miRNAs, typically have incomplete base pairing to a target an inhibit the translation of many different mRNAs with similar sequences. In contrast, siRNAs typically base-pair perfectly and induce mRNA cleavage only in a single, specific target.

The SMAD7 RNAis or SMAD7 microRNAs described herein, when introduced into a cell (e.g., by transfection or electroporation), can reduce SMAD7 expression in the cell by reducing the level of a SMAD7 mRNA or by reducing the level of a SMAD7 protein. The SMAD7 RNAis or SMAD7 microRNAs provided herein can reduce SMAD7 expression in vitro, e.g., in a cultured cell, or in vivo, e.g., in a subject (such as in a human patient or in an animal model organism).

In some embodiments, the SMAD7 RNAi or SMAD7 miRNA can reduce the level of a SMAD7 mRNA in a cell by 10% or more, 20% or more, 30% or more, 40% or more, 50% or more, 60% or more, 70% or more, 80% or more, 90% or more, 95% or more, or 99% or more.

In some embodiments, the SMAD7 RNAi or SMAD7 miRNA can reduce the level of a SMAD7 protein in a cell by 10% or more, 20% or more, 30% or more, 40% or more, 50% or more, 60% or more, 70% or more, 80% or more, 90% or more, 95% or more, or 99% or more.

In some embodiments, the SMAD7 RNAi or SMAD7 miRNA can reduce the level of a SMAD7 mRNA or a SMAD7 protein within 1 hr, 2 hrs, 3 hrs, 6 hrs, 9 hrs, 12, hrs, 15 hrs, 18 hrs, 21 hrs, 24 hrs, 30 hrs, 36 hrs, 42 hrs, or 48 hrs of contacting the SMAD7 RNAi or SMAD7 miRNA with a test sample (e.g., a cell in a cell culture) or of administering the SMAD7 AON to a subject.

The SMAD7 miRNAs described herein can include, e.g., miR-17-5, miR-21, miR-25, miR-181, miR-195 or miR-216a/217, or naturally occurring variations or synthetic derivatives thereof.

In some embodiments, the SMAD7 RNAi or SMAD7 miRNA is capable of modulating a TLR (e.g., inhibit or activate a TLR). In some embodiments, the TLR is TLR3, TLR4, TLR7, TLR8 or TLR9. See, e.g., Sections 7.2 and 7.8.(a).

In some embodiments, the SMAD7 RNAi or SMAD7 miRNA is capable of activating a TLR. In some embodiments, the TLR is TLR3, TLR4, TLR7, TLR8 or TLR9.

In some embodiments, the SMAD7 RNAi or SMAD7 miRNA is capable of inhibiting a TLR. In some embodiments, the TLR is TLR3, TLR4, TLR7, TLR8 or TLR9.

7.5 Illustrative Combinations

In some embodiments, the combinations described herein comprising an anti-SMAD7 therapeutic and a TLR modulator comprise a SMAD7 AON comprising a nucleotide sequence complementary to region 108-128 of the human SMAD7 nucleotide sequence of SEQ ID NO: 1, or the corresponding RNA sequence and a compound capable of modulating TLR3, TLR7, TLR8 and/or TLR9. In some embodiments, the compound is capable of activating TLR9. In some embodiments, the compound is capable of inhibiting one or more of TLR3, TLR7, TLR8, and/or TLR9. In some embodiments, the compound is capable of activating TLR9 and of inhibiting TLR3 and TLR7. In some embodiments, the compound capable of modulating TLR3, TLR7, TLR8 and/or TLR9 is a TLR synergist. In some embodiments, the compound capable of modulating TLR3, TLR7, TLR8 and/or TLR9 is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof. In some embodiments, the compound capable of modulating TLR3, TLR7, TLR8 and/or TLR9 is selected from chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), atovaquenone/proguanil (Malarone) quinacrine (Mepacrine, Atebrine), or quinidine (Quinaglute, Quinidex). In some embodiments, the compound capable of modulating TLR3, TLR7, TLR8 and/or TLR9 is hydroxychloroquine. In some embodiments, the compound capable of modulating TLR3, TLR7, TLR8 and/or TLR9 is BL-7040, CYT003, CYT003-QbG10, AZD1419, DIMS0150, E6446, CpG ODN2088, IMO-8400, IMO-3100, ODN2006, CL075, VTX-2337, or naltrexone.

In some embodiments, the combinations comprise a SMAD7 AON targeting nucleotides 403, 233, 294, 295, 296, 298, 299, or 533 of the nucleic acid sequence of SEQ ID NO: 1 and a compound capable of modulating TLR3, TLR7, TLR8 and/or TLR9. In some embodiments, the compound is capable of activating TLR9. In some embodiments, the compound is capable of inhibiting one or more of TLR3, TLR7, TLR8, and/or TLR9. In some embodiments, the compound capable of modulating TLR3, TLR7, TLR8 and/or TLR9 is a TLR synergist. In some embodiments, the compound capable of modulating TLR3, TLR7, TLR8 and/or TLR9 is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof. In some embodiments, the compound capable of modulating TLR3, TLR7, TLR8 and/or TLR9 is a compound selected from chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), atovaquenone/proguanil (Malarone), quinacrine (Mepacrine, Atebrine) or quinidine (Quinaglute, Quinidex). In some embodiments, the compound capable of modulating TLR3, TLR7, TLR8 and/or TLR9 is hydroxychloroquine. In some embodiments, the compound capable of modulating TLR3, TLR7, TLR8 and/or TLR9 is BL-7040, CYT003, CYT003-QbG10, AZD1419, DIMS0150, E6446, CpG ODN2088, IMO-8400, IMO-3100, ODN2006, CL075, VTX-2337, or naltrexone.

In some embodiments, the combinations comprise a SMAD7 AON comprising SEQ ID NO: 2 and a compound capable of modulating TLR3, TLR7, TLR8 and/or TLR9. In some embodiments, the compound is capable of activating TLR9. In some embodiments, the compound is capable of inhibiting one or more of TLR3, TLR7, TLR8, and/or TLR9. In some embodiments, the compound is capable of inhibiting one or more of TLR3, TLR7, TLR8, and/or TLR9 comprises the nucleotide sequence of SEQ ID NO: 2. In some embodiments, the compound capable of modulating TLR3, TLR7, TLR8 and/or TLR9 is a TLR synergist. In some embodiments, the compound capable of modulating TLR3, TLR7, TLR8 and/or TLR9 is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof. In some embodiments, the compound capable of modulating TLR3, TLR7, TLR8 and/or TLR9 is a compound selected from chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), atovaquenone/proguanil (Malarone), quinacrine (Mepacrine, Atebrine) or quinidine (Quinaglute, Quinidex). In some embodiments, the compound capable of modulating TLR3, TLR7, TLR8 and/or TLR9 is hydroxychloroquine. In some embodiments, the compound capable of modulating TLR3, TLR7, TLR8 and/or TLR9 is BL-7040, CYT003, CYT003-QbG10, AZD1419, DIMS0150, E6446, CpG ODN2088, IMO-8400, IMO-3100, ODN2006, VTX-2337, CL075, or naltrexone.

In some embodiments, the combinations described herein comprise a SMAD7 AON comprising a nucleotide sequence of SEQ ID NO: 2, SEQ ID NO: 3, SEQ ID NO: 4, SEQ ID NO: 5, SEQ ID NO: 6, or SEQ ID NO: 7, or fragments thereof (e.g., fragments of 10 or more nucleotides) and a compound capable of modulating TLR3, TLR7, TLR8 and/or TLR9. In some embodiments, the compound is capable of activating TLR9. In some embodiments, the compound capable of modulating TLR3, TLR7, TLR8 and/or TLR9 is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof. In some embodiments, the compound is capable of inhibiting one or more of TLR3, TLR7, TLR8, and/or TLR9. In some embodiments, the compound capable of modulating TLR3, TLR7, TLR8 and/or TLR9 is a TLR synergist. In some embodiments, the compound capable of modulating TLR3, TLR7, TLR8 and/or TLR9 is a compound selected from chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), atovaquenone/proguanil (Malarone), quinacrine (Mepacrine, Atebrine) or quinidine (Quinaglute, Quinidex). In some embodiments, the compound capable of modulating TLR3, TLR7, TLR8 and/or TLR9 is hydroxychloroquine. In some embodiments, the compound capable of modulating TLR3, TLR7, TLR8 and/or TLR9 is BL-7040, CYT003, CYT003-QbG10, AZD1419, DIMS0150, E6446, CpG ODN2088, IMO-8400, IMO-3100, ODN2006, CL075, VTX-2337, or naltrexone.

In some embodiments, the combinations comprise COMPOUND (I) and a compound capable of modulating TLR3, TLR7, TLR8 and/or TLR9. In some embodiments, COMPOUND (I) is the compound capable of modulating TLR3, TLR7, TLR8 and/or TLR9. In some embodiments, the compound is capable of activating TLR9. In some embodiments, the compound is capable of inhibiting one or more of TLR3, TLR7, TLR8, and/or TLR9. In some embodiments, the compound capable of modulating TLR3, TLR7, TLR8 and/or TLR9 is a TLR synergist. In some embodiments, the compound capable of modulating TLR3, TLR7, TLR8 and/or TLR9 is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof. In some embodiments, the compound is capable of modulating TLR3, TLR7, TLR8 and/or TLR9 is a compound selected from chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), atovaquenone/proguanil (Malarone), quinacrine (Mepacrine, Atebrine) or quinidine (Quinaglute, Quinidex). In some embodiments, the compound capable of modulating TLR3, TLR7, TLR8 and/or TLR9 is hydroxychloroquine. In some embodiments, the compound capable of modulating TLR3, TLR7, TLR8 and/or TLR9 is BL-7040, CYT003, CYT003-QbG10, AZD1419, DIMS0150, E6446, CpG ODN2088, IMO-8400, IMO-3100, ODN2006, CL075, VTX-2337, or naltrexone.

In some embodiments, the combinations comprise COMPOUND (I) and a compound capable of modulating TLR3. In some embodiments, the compound capable of modulating TLR3 is a TLR3 antagonist.

In some embodiments, the combinations comprise COMPOUND (I) and a compound capable of modulating TLR4. In some embodiments, the compound capable of modulating TLR4 is a TLR4 antagonist.

In some embodiments, the combinations comprise COMPOUND (I) and a compound capable of modulating TLR7. In some embodiments, the compound capable of modulating TLR7 is a TLR7 antagonist.

In some embodiments, the combinations comprise COMPOUND (I) and a compound capable of modulating TLR8. In some embodiments, the compound capable of modulating TLR8 is a TLR8 antagonist.

In some embodiments, the combinations comprise COMPOUND (I) and a compound capable of modulating TLR9. In some embodiments, the compound capable of inhibiting TLR9 is a TLR9 antagonist. In some embodiments, the compound capable of inhibiting TLR9 is a TLR9 agonist.

In some embodiments, the combinations comprise COMPOUND (I) and hydroxychloroquine.

In some embodiments, the combinations comprise COMPOUND (I) and a compound selected from BL-7040, CYT003, CYT003-QbG10, AZD1419, DIMS0150, E6446, CpG ODN2088, IMO-8400, IMO-3100, ODN2006, CL075, VTX-2337, or naltrexone.

7.6 Pharmaceutical Compositions

In some embodiments, an ODN, SMAD7 ODN, or anti-SMAD ODN or combinations comprising an anti-SMAD7 ODN (e.g., an SMAD7 AON) and a TLR modulator described herein can be formulated as pharmaceutical compositions. In some embodiments, the anti-SMAD7 ODN and the TLR modulator of a combination are formulated together in the same unit-dosage form (“Two-in-One,” e.g., a pill, tablet, capsule, powder, and the like).

In one aspect, provided herein is a pharmaceutical composition comprising a SMAD7 ODN described herein, a TLR modulator described herein, and a pharmaceutically acceptable excipient. See, e.g., Sections 7.2 and 7.4.(c).

In some embodiments, the pharmaceutical composition comprises the SMAD7 ODN and the TLR modulator in equimolar ratios (e.g., a 1:1 ratio).

In some embodiments, the pharmaceutical composition comprises a molar excess of SMAD7 ODN over the TLR modulator. In some embodiments, the excess of SMAD7 ODN over the TLR modulator is at least 2-fold, at least 3-fold, at least 4-fold, at least 5-fold, at least 6-fold, at least 8-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1,000-fold.

In some embodiments, the pharmaceutical composition comprises a molar excess of TLR modulator over the SMAD7 ODN. In some embodiments, the excess of TLR modulator over the SMAD7 ODN is at least 2-fold, at least 3-fold, at least 4-fold, at least 5-fold, at least 6-fold, at least 8-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1,000-fold.

In some embodiments, the pharmaceutical composition comprises two or more different SMAD7 ODNs, such as 2 or more, 3 or more, 4 or more, 5 or more, 6 or more, 8 or more, or 10 or more SMAD7 ODNs.

In some embodiments, the pharmaceutical composition comprises a plurality of different TLR modulators, such as 2 or more, 3 or more, 4 or more, 5 or more, 6 or more, 8 or more, or 10 or more TLR modulators. In some embodiments, the pharmaceutical composition comprises two or more TLR modulators capable of modulating the same TLR (e.g., TLR7). In some embodiments, the pharmaceutical composition comprises two or more TLR modulators capable of modulating different TLRs (e.g., TLR7 and TLR9). In some embodiments, the pharmaceutical composition comprises one or more compounds capable of activating a TLR and one or more compounds inhibiting a TLR. In some embodiments, the pharmaceutical composition comprises two or more TLR agonists (e.g., 2 or more, 3 or more, 4 or more, 5 or more, 6 or more, 8 or more, or 10 or more). In some embodiments, the pharmaceutical composition comprises two or more TLR antagonists (e.g., 2 or more, 3 or more, 4 or more, 5 or more, 6 or more, 8 or more, or 10 or more).

In some embodiments, the pharmaceutical composition comprises a TLR9 agonist and one or more additional TLR modulators. In some embodiments, the pharmaceutical composition comprises a TLR7 antagonist and a TLR9 antagonist. In some embodiments the pharmaceutical compositions comprises a TLR3 antagonist, a TLR7 antagonist, and a TLR9 antagonist. In some embodiments, the pharmaceutical composition comprises a TLR3 antagonist, a TLR 7 antagonist, a TLR8 antagonist and a TLR9 antagonist. In some embodiments, the pharmaceutical composition comprises a TLR4 antagonist.

In some embodiments, the pharmaceutical composition comprises hydroxychloroquine or chloroquine and one or more additional TLR modulators. The one or additional TLR modulators can include one or more TLR agonists and/or one or more TLR antagonists.

In some embodiments, the pharmaceutical compositions described herein (e.g., comprising a SMAD7 ODN and a TLR modulator) are formulated with a pharmaceutically acceptable excipient or carrier for use with the methods described herein.

The pharmaceutical compositions described herein (e.g., comprising a SMAD7 ODN or a SMAD7 ODN and a TLR modulator) can be administered using different formulations and routes of administration, depending on whether the pharmaceutical composition is targeted for topical or systemic delivery. Administration can include, e.g., topical, pulmonary, oral, or parenteral administration. Topical administration can include ophthalmic administration or administration to mucous membranes, such as vaginal or rectal delivery. Pulmonary administration can include, e.g,. inhalation or insufflations of powders or aerosols, including by nebulizer, intratracheal, intranasal, epidermal and transdermal. Parenteral administration can include, e.g., intravenous, intraarterial, subcutaneous, intraperitoneal or intramuscular injection or infusion.

In some embodiment, the pharmaceutical compositions described herein (e.g., comprising a SMAD7 ODN and a SMAD7 ODN and a TLR modulator) are formulated with a pharmaceutically acceptable carrier for use with the methods described herein. For example, a SMAD7 ODN and a TLR modulator can be administered alone or as a component of a pharmaceutical formulation (therapeutic composition). The subject compounds may be formulated for administration in any convenient way for use in human or veterinary medicine.

In some embodiments, the therapeutic methods described herein include administering the composition systemically, or locally as an implant or device. When administered, the therapeutic compositions used herein can be in a pyrogen-free, physiologically acceptable form. Therapeutically useful agents other than the SMAD7 ODN and the TLR modulator, which may also optionally be included in the composition as described above, may be administered simultaneously or sequentially with the subject compounds.

In some embodiments, compositions comprising the SMAD7 ODN or the SMAD7 ODN and the TLR modulator will be administered parenterally. Pharmaceutical compositions suitable for parenteral administration may comprise one or more SMAD7 ODNs or one or more SMAD7 ODNs and one or more TLR modulator in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions used in the methods described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Further, the composition may be encapsulated or injected in a form for delivery to a target tissue site (e.g., bone). In certain embodiments, compositions used in the methods described herein may include a matrix capable of delivering one or more therapeutic compositions (e.g., comprising a SMAD7 ODN or a SMAD7 ODN and a TLR modulator) to a target tissue site (e.g., bone), providing a structure for the developing tissue and optimally capable of being resorbed into the body. For example, the matrix may provide slow release of the SMAD7 ODN or the SMAD7 ODN and/or TLR modulator. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalciumphosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

In certain embodiments, the compositions used in the methods described herein can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds used in the methods described herein may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In some embodiments, the pharmaceutical composition is comprised in a unit dosage form, e.g., a tablet, capsule, or powder.

In some embodiments, the unit dosage form comprises between about 5 mg and about 80 mg, between about 10 mg and about 70 mg, between about 20 mg and about 60 mg, or between about 30 mg and about 50 mg of SMAD7 ODN.

In some embodiments, the unit dosage form comprises between about 10 mg and about 150 mg, between about 30 mg and about 130 mg, between about 50 mg and about 110 mg, or between about 70 mg and about 90 mg of SMAD7 ODN.

In some embodiments, the unit dosage form comprises between about 10 mg and about 330 mg, between about 50 mg and about 280 mg, between about 90 mg and about 240 mg, or between about 130 mg and about 200 mg of SMAD7 ODN.

In some embodiments, the unit dosage form comprises between about 20 mg and about 600 mg, between about 60 mg and about 560 mg, between about 100 mg and about 520 mg, between about 140 mg and about 480 mg, between about 180 mg and about 440 mg, between about 220 mg and about 400 mg, between about 260 mg and about 360 mg, or between about 280 mg and about 340 mg SMAD7 ODN.

In some embodiments, the unit dosage form comprises about 40 mg, about 80 mg, about 160 mg, or about 320 mg of SMAD7 ODN.

In some embodiments, the pharmaceutical composition is an oral pharmaceutical composition. In some embodiments, the pharmaceutical composition includes an enteric coating to topically deliver the SMAD7 ODN and/or the TLR modulator to the terminal ileum and/or right colon of a patient, such as an IBD patient. In some embodiments, the enteric coating comprises an ethylacrylate-methacrylic acid copolymer.

Disclosed therapies can, when administered orally to a patient, e.g., an IBD patient, deliver an effective amount of a SMAD7 ODN and/or a TLR modulator the intestinal system of a patient, e.g., deliver an effective amount of a SMAD7 ODN and/or a TLR modulator to the terminal ileum and/or right colon of a patient.

In some embodiments of the invention, the SMAD7 ODN and TLR modulator can be suitable for oral delivery of a the SMAD7 ODN and TLR modulator, e.g., tablets, that include an enteric coating, e.g., a gastro-resistant coating, such that the compositions can deliver the SMAD7 ODN and TLR modulator to, e.g., the terminal ileum and right colon of a patient. For example, such administration can result in a topical effect, substantially topically applying the SMAD7 ODN and TLR modulator directly to an affected portion of the intestine of a patient. Such administration, can, in some embodiments, substantially avoid unwanted systemic absorption of at least the SMAD7 ODN.

For example, a tablet for oral administration can comprise granules (e.g., is at least partially formed from granules) that include a described composition comprising a SMAD7 ODN, a TLR modulator and pharmaceutically acceptable excipients. Such a tablet can be coated with an enteric coating. Contemplated tablets can include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a SMAD7 ODN and/or a TLR modulator and/or a pharmaceutically acceptable salt and a pharmaceutically acceptable filler. For example, COMPOUND (I) and/or a TLR modulator and a filler can be blended together, with optionally other excipients, and formed into granules. In some embodiments, the intragranular phase can be formed using wet granulation, e.g., a liquid (e.g., water) is added to the blended antisense compound and filler, and then the combination is dried, milled and/or sieved to produce granules. One of skill in the art would understand that other processes can be used to achieve an intragranular phase.

In some embodiments, contemplated formulations include an extra-granular phase, which can include one or more pharmaceutically acceptable excipients, and which can be blended with the intragranular phase to form a disclosed formulation.

A SMAD7 ODN and/or TLR modulator formulation can include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, a SMAD7 ODN and/or TLR modulator formulation can include an intragranular phase and/or an extragranular phase that includes a binder, which can generally function to hold the ingredients of the pharmaceutical formulation together. Exemplary binders include, for example, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others, including combinations thereof.

Contemplated SMAD7 ODN and/or/TLR modulator formulations, e.g., that include an intragranular phase and/or an extragranular phase, can include a disintegrant, such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase can include a disintegrant.

In some embodiments, a contemplated SMAD7 ODN and/or TLR modulator formulation includes an intra-granular phase comprising a disclosed antisense compound and excipients chosen from: mannitol, microcrystalline cellulose, hydroxypropylmethyl cellulose, and sodium starch glycolate, or combinations thereof, and an extra-granular phase comprising one or more of: microcrystalline cellulose, sodium starch glycolate, and magnesium stearate, or mixtures thereof.

In some embodiments, a contemplated SMAD7 ODN and/or TLR modulator formulation can include a lubricant, e.g., an extra-granular phase can contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings can include a polymer that disintegrates a different rates according to pH. Enteric coatings can include, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C (e.g., U.S. Pharmacopeia, National Formulary; European Pharmacopeia), polyvinyl acetate-phthalate, and cellulose acetate phthalate.

In some embodiments, the enteric coating includes an anionic, cationic, or neutral copolymer based on methacrylic acid, methacrylic/acrylic esters or their derivatives. In some embodiments, the enteric coating includes an ethylacrylate-methacrylic acid copolymer. Commercially available enteric coatings include Opadry® AMB, Acryl-EZE®, Eudragit® grades. In some embodiments, the enteric coating makes up about 5% to about 10%, about 5% to about 20%, about 8 to about 15%, about 8% to about 18%, about 10% to about 12%, or about 12% to about 16% of a contemplated tablet by weight.

For example, a SMAD7 ODN and/or TLR modulator composition in the form of a tablet is provided that comprises or consists essentially of about 0.5% to about 70%, e.g., about 0.5% to about 10%, or about 1% to about 20% by weight of a combination of a SMAD AON, a TLR modulator, or pharmaceutically acceptable salts thereof. Such a tablet can include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose.

Exemplary SMAD7 ODN and/or TLR modulator formulations include dosage forms that include or consist essentially of about 10 mg to about 500 mg of a combination of COMPOUND (I) and a TLR modulator, for example, tablets that include about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 150 mg, about 200 mg, or about 250 mg of COMPOUND (I) are contemplated herein. In one embodiment, a COMPOUND (I)/TLR modulator composition can be formulated as a tablet for oral use comprising: about 0.5% to about 10% by weight of COMPOUND (I); about 0.5% to about 10% by weight of TLR modulator; about 30% to about 50% by weight mannitol; and about 10% to about 30% by weight microcrystalline cellulose.

Contemplated tablets can also include an enteric coating, e.g., a disclosed tablet can include about 13%, about 14%, about 15%, about 16%, about 17% by weight of an enteric coating, e.g., ethylacrylate-methacrylic acid copolymers (e.g., AcyrlEZE®).

Contemplated formulations, e.g., tablets, in some embodiments, when orally administered to the patient can result in minimal plasma concentration of the oligonucleotide in the patient. In another embodiment, contemplated formulations, when orally administered to a patient, topically deliver to the terminal ileum and/or right colon of a patient, e.g., to an affected or diseased intestinal site of a patient.

7.7 Treatment Methods

In another aspect, provided herein is a method for treating or managing a disease in a patient in need thereof, comprising administering to the patient a combination of a therapeutically effective amount of an anti-SMAD7 therapeutic (e.g., an anti-SMAD7 ODN, such as a SMAD7 AON) and a therapeutically effective amount of a TLR modulator described herein (see, e.g., Section 7.2). In some embodiments, the anti-SMAD7 therapeutic is COMPOUND (I). In some embodiments, the anti-SMAD7 therapeutic is COMPOUND (I) and the TLR modulator is hydroxychloroquine.

In another aspect, provided herein is a method for inducing or promoting epithelial restitution or mucosal healing in a patient in need thereof (e.g., a human IBD patient), comprising administering to the patient a combination of a therapeutically effective amount of an anti-SMAD7 therapeutic (e.g., an anti-SMAD7 ODN) and a therapeutically effective amount of a TLR modulator described herein (see, e.g., Section 7.2). In some embodiments, the anti-SMAD7 therapeutic is COMPOUND (I). In some embodiments, the anti-SMAD7 therapeutic is COMPOUND (I) and the TLR modulator is hydroxychloroquine.

In some embodiments, the anti-SMAD7 therapeutic (e.g., a SMAD7 ODN) and the TLR modulator are formulated together as components of a pharmaceutical composition. See, e.g., Section 7.6.

In some embodiments, the anti-SMAD7 therapeutic (e.g., a SMAD7 ODN) is covalently linked to a compound capable of modulating a TLR (e.g., TLR3, TLR4, TLR7, TLR8, or TLR9). See Section 7.8.(b).

In some embodiments, the anti-SMAD7 therapeutic (e.g., a SMAD7 ODN) is capable of modulating a TLR (e.g., TLR3, TLR4, TLR7, TLR8, or TLR9). See Section 7.8.(a).

In another aspect, provided herein is a method of treating or managing a disease in a patient in need thereof, comprising (a) administering to the patient an effective amount of an anti-SMAD7 therapeutic (e.g., a SMAD7 ODN); (b) determining the patient's response to the anti-SMAD7 therapeutic, and (c) if the patient does not respond to the anti-SMAD7 therapeutic, then, administering to the patient an effective amount of the anti-SMAD7 therapeutic (e.g., a SMAD7 ODN) and an effective amount of a compound capable of modulating a TLR.

In some embodiments, determining the patient's response to the anti-SMAD7 therapeutic (e.g., a SMAD7 ODN) comprises (a) analyzing a first level of a biomarker before administering the anti-SMAD7 therapeutic to the patient, and (b) analyzing a second level of the biomarker after administering the anti-SMAD7 therapeutic to the patient, wherein the patient responds to the anti-SMAD7 therapeutic if the second biomarker level is lower than the first biomarker level.

In some embodiments, the patient responds to the anti-SMAD7 therapeutic (e.g., a SMAD7 ODN), if the second biomarker level is at least 10%, at least 20%, at least 30%, at least 40%, at least 50%, at least 60%, at least 70%, at least 80%, or at least 90% lower than the first biomarker level.

In some embodiments, determining the patient's response to the anti-SMAD7 therapeutic (e.g., a SMAD7 ODN) comprises (a) analyzing a first level of a biomarker before administering the anti-SMAD7 therapeutic to the patient, and (b) analyzing a second level of the biomarker after administering the anti-SMAD7 therapeutic to the patient, wherein the patient responds to the anti-SMAD7 therapeutic if the second biomarker level is higher than the first biomarker level.

In some embodiments, the patient responds to the anti-SMAD7 therapeutic (e.g., a SMAD7 ODN), if the second biomarker level is at least 2-fold, at least 3-fold, at least 4-fold, at least 5-fold, at least 6-fold, at least 7-fold, at least 8-fold, at least 9-fold, or at least 10-fold modified relative to the first biomarker level.

In some embodiments, the biomarker can include, without limitation, CRP, TNFα, TGFβ, IL4, IL6, IL8, IL10, IL12, IL17, IL23, CCL20, CD4, CD5, CD8, FCP, HLA-DR, phospho-SMAD2, phospho-SMAD3, SMAD7 mRNA or SMAD7 protein.

In some embodiments, the biomarker indicative of responsiveness to a treatment provided herein is increase in expression of TNFα, IFNγ, TGFβ, IL-6, IL-10, PD-L1, IDO, or ICOS-L protein or decrease in expression or secretion of IP10 by a pDC.

In some embodiments, the biomarker indicative of responsiveness to a treatment provided herein is increase in expression or secretion of CCR7, CD80, CD83, CD86, CD-69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR, or decrease in expression or secretion of bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1 in a cell of the immune system, such as a PBMC, pDC, or B-cell

In the methods provided herein, the biomarkers bFGF, CCR6, CCR7, CD80, CD83, CD86, CD-69, CD123 (IL-3Rα), EGFR, GARP, ICAM-1, IgG, IL-1α, IL1-β, IL-2, IL-4, IL-10Rα, IL-18, IL-23p19, ILT7, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1 (collectively “recited biomarkers”) can be used to monitor the activities of a combination treatment comprising a therapeutically effective amount of an anti-SMAD7 therapeutic (e.g., a SMAD7 ODN) and a therapeutically effective amount of a TLR modulator described herein (see, e.g., Section 7.2) (e.g., to analyze if a patient shows a clinical response to an anti-SMAD7 therapeutic/TLR modulator combination or if a patient experiences an improvement in a disease condition or symptom, such as remission of IBD). In some embodiments, recited biomarker levels in a patient sample can inform a decision regarding whether a patient (e.g., an IBD patient) is transitioning from a first to a second treatment phase (e.g., if the patient shows a clinical response to an anti-SMAD7 therapeutic/TLR modulator combination or if the patient shows remission as indicated by recited biomarker levels). In other methods provided herein, the recited biomarkers can be used as biomarkers for patient selection.

In some embodiments, the patient's response to the anti-SMAD7 therapeutic (e.g., a SMAD7 ODN) is determined by a person of skill using a disease-specific clinical parameter (e.g., CDAI score).

(a) Disease Indications

In some embodiments, the disease is an inflammatory disease. In some embodiments, the inflammatory disease is inflammatory bowel disease (IBD). In some embodiments, the IBD is Crohn's disease (CD), incl. gastroduodenal Crohn's disease, Crohn's (granulomatous) colitis, ulcerative colitis (UC), collagenous colitis, lymphocytic colitis, ischaemic colitis, diversion colitis, Behcet's disease, microscopic colitis, ulcerative proctitis, proctosigmoiditis, jejunoilitis, left-sided colitis, pancolitis, ileocolitis, ileitis, and indeterminate colitis.

In some embodiments, the disease is an autoimmune disorder. In some embodiments, the autoimmune disorder is Sjogren's Syndrome, systemic lupus erythematosus (SLE), dry eye, autoimmune encephalitis, rheumatoid arthritis, multiple sclerosis, systemic sclerosis, psoriasis, colitis, or uveitis.

In some embodiments, the disease is an airway disease. In some embodiments, the airway disease is asthma or chronic pulmonary disease (COPD).

In some embodiments, the disease is an allergic disorder. In some embodiments, the allergic disorder is asthma, allergic rhinitis, or atopic dermatitis.

In some embodiments, the disease is an infectious disease. In some embodiments, the infectious disease is Malaria, Hashimoto's encephalopathy or amoebiasis.

In some embodiments, the disease is a metabolic disorder. In some embodiments, the metabolic disorder is diabetes, hyperlipidemia, or non-alcoholic fatty liver disease.

In some embodiments, the disease is cancer. In some embodiments, cancer is a lung cancer, a pancreatic cancer, a leukemic cancer, a lymphoid cancer, a breast cancer, a prostate cancer, an ovarian cancer, a testicular cancer, a melanoma, a myeloma, a glioblastoma, a neuroblastoma, a colorectal cancer, or a stomach cancer.

In some embodiments, the disease is a central nervous system (CNS) disease. In some embodiments, the disease is multiple sclerosis.

In some embodiments, the disease is a skin disease. In some embodiments, the skin disease is basal cell carcinoma or actinic keratosis.

(b) Monotherapies

In another aspect, provided herein is a method for treating or managing a disease in a patient in need thereof, comprising administering to the patient a therapeutically effective amount of an anti-SMAD7 therapeutic (e.g., a SMAD7 ODN) described herein (see, e.g., Sections 7.4 or 7.8), wherein the anti-SMAD7 therapeutic (e.g., a SMAD7 ODN) is capable of modulating a TLR. In some embodiments, the anti-SMAD7 therapeutic is COMPOUND (I).

In another aspect, provided herein is a method of treating or managing a disease in a patient in need thereof, comprising administering to the patient a therapeutically effective amount of a TLR modulator described herein (see, e.g., Sections 7.2 or 7.8).

In some embodiments, the disease is an inflammatory disease. In some embodiments, the inflammatory disease is inflammatory bowel disease (IBD). In some embodiments, the IBD is Crohn's disease (CD), incl. gastroduodenal Crohn's disease, Crohn's (granulomatous) colitis, ulcerative colitis (UC), collagenous colitis, lymphocytic colitis, ischaemic colitis, diversion colitis, Behcet's disease, microscopic colitis, ulcerative proctitis, proctosigmoiditis, jejunoilitis, left-sided colitis, pancolitis, ileocolitis, ileitis, and indeterminate colitis.

In some embodiments, the disease is an autoimmune disorder. In some embodiments, the autoimmune disorder is Sjogren's Syndrome, systemic lupus erythematosus (SLE), dry eye, autoimmune encephalitis, rheumatoid arthritis, multiple sclerosis, systemic sclerosis, psoriasis, colitis, or uveitis.

In some embodiments, the disease is an airway disease. In some embodiments, the airway disease is asthma or chronic pulmonary disease (COPD).

In some embodiments, the disease is an allergic disorder. In some embodiments, the allergic disorder is asthma, allergic rhinitis, or atopic dermatitis.

In some embodiments, the disease is an infectious disease. In some embodiments, the infectious disease is Malaria, Hashimoto's encephalopathy or amoebiasis.

In some embodiments, the disease is a metabolic disorder. In some embodiments, the metabolic disorder is diabetes, hyperlipidemia, or non-alcoholic fatty liver disease.

In some embodiments, the disease is cancer. In some embodiments, cancer is a lung cancer, a pancreatic cancer, a leukemic cancer, a lymphoid cancer, a breast cancer, a prostate cancer, an ovarian cancer, a testicular cancer, a melanoma, a myeloma, a glioblastoma, a neuroblastoma, a colorectal cancer, or a stomach cancer.

In some embodiments, the disease is a central nervous system (CNS) disease. In some embodiments, the disease is multiple sclerosis.

In some embodiments, the disease is a skin disease. In some embodiments, the skin disease is basal cell carcinoma or actinic keratosis.

(c) Administration Regimens

In the methods provided herein, the anti-SMAD7 therapeutic and the TLR modulator can be administered to the patient at about the same time (e.g., within 1 min, within 5 min, within 10 min, within 15 min, within 30 min, within 45 min, or within 1 hour of each other), or at different times.

In some embodiments, the anti-SMAD7 therapeutic is administered first and the TLR modulator is administered second (e.g., during the same day, same week, or same month depending on administration frequencies).

In some embodiments, the TLR modulator is administered first and the anti-SMAD7 therapeutic is administered second (e.g., during the same day, same week, or same month depending on administration frequencies).

In some embodiments, the patient received a anti-SMAD7 therapeutic for a period of time prior to administration of the TLR modulator. In some embodiments, the period of time is for at least 1 day, at least 3 days, at least 1 week, at least 2 weeks, at least 1 month, at least 2 months, at least 3 months, at least 6 months, at least 1 year, at least 2 years, at least 3 years, at least 4 years, at least 5 years, at least 6 years, at least 7 years, at least 8 years, at least 9 years, or at least 10 years).

In some embodiments the anti-SMAD7 therapeutic and the TLR modulator are administered using the same administration schedules. In some embodiments, the anti-SMAD7 therapeutic and the TLR are administered using different administration schedules. In some embodiments, the anti-SMAD7 therapeutic and/or the TLR modulator are administered using a continuous dosing schedule (e.g., once a day, twice as day, and the like, for a continuous period of, e.g., 1 week, 2 weeks, 3 weeks, one month, two months, and longer). In some embodiments, the anti-SMAD7 therapeutic and/or the TLR modulator are administered using an alternating dosing schedule (e.g., four weeks of treatment followed by four weeks of no-treatment). In some embodiments, the anti-SMAD7 therapeutic and the TLR modulator are administered following a continuous dosing schedule during a first time period and following an alternating dosing schedule during a second time period.

The anti-SMAD7 therapeutic and the TLR modulator can be administered at the same frequency or at different frequencies. In some embodiments, the anti-SMAD7 therapeutic is administered more frequently than the TLR modulator. In some embodiments, the TLR modulator is administered more frequently than the anti-SMAD7 therapeutic.

In some embodiments, the anti-SMAD7 therapeutic and the TLR modulator are administered in combination in a unit dosage form. In some embodiments, the anti-SMAD7 therapeutic and the TLR modulator are administered in separate unit dosage forms.

In some embodiments, the anti-SMAD7 therapeutic and the TLR modulator are administered using the same routes of administration, e.g., oral, nasal, or i.v. In some embodiments, the anti-SMAD7 therapeutic and the TLR modulator are administered using different routes of administration, e.g., the anti-SMAD7 therapeutic is administered nasally as an aerosol, and the TLR modulator is administered per i.v.

In another embodiment, provided herein is a method for treating or managing a disease described herein (see, e.g., Section 7.7.(a) or (b)) in a patient in need thereof, wherein the method comprises (a) analyzing a first level of IL-1β, IP10, PD-L1, IDO, ICOS-L in the patient; (b) administering to the patient an initial dose of an anti-SMAD7 therapeutic (e.g., a SMAD7 ODN); (c) analyzing a second level of IL-1β, IP10, PD-L1, IDO, or ICOS-L in the patient after the administering step; and wherein: if the second level of IL-1β, IP10, PD-L1, IDO, or ICOS-L is the same or higher than the first level of L-1β, IP10, PD-L1, IDO, or ICOS-L, then: administering to the patient a subsequent dose that is equal to or higher than the initial dose, and/or administering to the patient a subsequent dose at an equal or higher frequency than the initial dose; or if the second level of IL-1β, IP10, PD-L1, IDO, or ICOS-L is lower than the first level of IL-1β, IP10, PD-L1, IDO, or ICOS-L, then administering to the patient a subsequent dose that is equal to or smaller than the initial dose, and/or administering to the patient a subsequent dose at an equal or lower frequency than the initial dose.

In another embodiment, provided herein is a method for treating or managing a disease described herein (see, e.g., Section 7.7.(a) or (b)) in a patient in need thereof, wherein the method comprises (a) analyzing a first level of IL-1β, IP10, PD-L1, IDO, or ICOS-L in the patient; (b) administering to the patient an initial dose of an anti-SMAD7 therapeutic (e.g., a SMAD7 ODN); (c) analyzing a second level of IL-1β, IP10, PD-L1, IDO, or ICOS-L in the patient after the administering step; and wherein: if the second level of IL-1β, IP10, PD-L1, IDO, or ICOS-L is the higher than the first level of IL-1β, IP10, PD-L1, IDO, or ICOS-L, then: administering to the patient a subsequent dose that is equal to or lower than the initial dose, and/or administering to the patient a subsequent dose at an equal or lower frequency than the initial dose; or if the second level of IL-1β, IP10, PD-L1, IDO, or ICOS-L is equal to or lower than the first level of IL-1β, IP10, PD-L1, IDO, or ICOS-L, then administering to the patient a subsequent dose that is higher than the initial dose, and/or administering to the patient a subsequent dose at a higher frequency than the initial dose.

In another aspect, provided herein is a method for treating or managing a disease described herein (see, e.g., Section 7.7.(a) or (b)) in a patient in need thereof, wherein the method comprises (a) administering to the patient an initial dose of an anti-SMAD7 therapeutic (e.g., a SMAD7 ODN); (b) analyzing the level of IL-1β, IP10, PD-L1, IDO, or ICOS-L in the patient after the administering step; and wherein, if the level of IL-1β, IP10, PD-L1, IDO, or ICOS-L is above a control level (e.g., a level observed in a healthy or normal control subject or control group) of IL-1β, IP10, PD-L1, IDO, or ICOS-L, then administering to the patient a subsequent dose that is greater than or equal to the initial dose, and/or administering to the patient a subsequent dose at an equal or higher frequency than the initial dose; or, if the level of IL-1β, IP10, PD-L1, IDO, or ICOS-L is below the control level of IL-1β, IP10, PD-L1, IDO, or ICOS-L, then administering to the patient a subsequent dose that is equal to or smaller than the initial dose and/or administering to the patient a subsequent dose at an equal or lower frequency than the initial dose.

In another aspect, provided herein is a method for treating or managing inflammatory a disease described herein (see, e.g., Section 7.7.(a) or (b)) in a patient in need thereof, wherein the method comprises (a) analyzing a control level (e.g., a base level observed prior to administration of a treatment) of IL-1β, IP10, PD-L1, IDO, or ICOS-L in the patient; and (b) if the base level of IL-1β, IP10, PD-L1, IDO, or ICOS-L is above a control level (e.g., above a level in a healthy or normal control subject) of IL-1β, IP10, PD-L1, IDO, or ICOS-L, then administering to the patient an initial dose of an anti-SMAD7 therapeutic.

In some embodiments, the method further comprises: (c) analyzing the level of IL-1β, IP10, PD-L1, IDO, or ICOS-L in the patient after said administering step; and wherein if the level of IL-1β, IP10, PD-L1, IDO, or ICOS-L after said administering step is above a healthy or normal control level of IL-1β, IP10, PD-L1, IDO, or ICOS-L, or above or equal to the patient's base level, then administering to the patient a subsequent dose that is greater than or equal to the initial dose and/or administering to the patient a subsequent dose at an equal or higher frequency than the initial dose, or, if the level of IL-1β, IP10, PD-L1, IDO, or ICOS-L after said administering step is below the patient's base level or below a healthy or normal control level of IL-1β, IP10, PD-L1, IDO, or ICOS-L, then administering to the patient a subsequent dose that is equal to or lower than the initial dose and/or administering to the patient a subsequent dose at an equal or lower frequency than the initial dose.

In some embodiments, if the subsequent dose is equal to or greater than the maximum tolerated dose (MTD), then the treatment is terminated.

In some embodiments, the method further comprises: (c) analyzing the level of IL-1β, IP10, PD-L1, IDO, or ICOS-L in the patient after said administering step; and wherein if the level of IL-1β, IP10, PD-L1, IDO, or ICOS-L after said administering step is above a healthy or normal control level of IL-1β, IP10, PD-L1, IDO, or ICOS-L, or above or equal to the patient's base level, then administering to the patient a subsequent dose that is lower than or equal to the initial dose and/or administering to the patient a subsequent dose at an equal or lower frequency than the initial dose, or, if the level of IL-1β, IP10, PD-L1, IDO, or ICOS-L after said administering step is below the patient's base level or below a healthy or normal control level of IL-1β, IP10, PD-L1, IDO, or ICOS-L, then administering to the patient a subsequent dose that is equal to or higher than the initial dose and/or administering to the patient a subsequent dose at an equal or higher frequency than the initial dose.

In some embodiments, treatment adjustments to higher doses or higher administration frequencies can occur, if the IL-1β, IP10, PD-L1, IDO, or ICOS-L level after administration of the anti-SMAD7 therapeutic remains unchanged of if it is at least 10%, at least 20%, at least 30%, at least 40% or at least 50% higher than the IL-1β, IP10, PD-L1, IDO, or ICOS-L level at the patient's baseline or relative to control levels (e.g., levels observed in a healthy or normal control subject) of IL-1β, IP10, PD-L1, IDO, or ICOS-L (e.g., levels observed in a healthy or normal control subject).

In some embodiments, treatment adjustments to lower doses or lower administration frequencies can occur, if the IL-1β, IP10, PD-L1, IDO, or ICOS-L level after administration of the anti-SMAD7 therapeutic remains unchanged of if it is at least 10%, at least 20%, at least 30%, at least 40%, at least 50%, at least 60%, at least 70%, at least 80%, or at least 90% lower than the IL-1β, IP10, PD-L1, IDO, or ICOS-L level at the patient's baseline or relative to control levels (e.g., levels observed in a healthy or normal control subject) IL-1β, IP10, PD-L1, IDO, or ICOS-L.

In some embodiments, treatment adjustments to higher doses or higher administration frequencies can occur, if the IL-1β, IP10, PD-L1, IDO, or ICOS-L level after administration of the anti-SMAD7 therapeutic remains unchanged of if it is at least 10%, at least 20%, at least 30%, at least 40% or at least 50% lower than the IL-1β, IP10, PD-L1, IDO, or ICOS-L level at the patient's baseline or relative to control levels (e.g., levels observed in a healthy or normal control subject) of IL-1β, IP10, PD-L1, IDO, or ICOS-L.

In some embodiments, treatment adjustments to lower doses or lower administration frequencies can occur, if the IL-1β, IP10, PD-L1, IDO, or ICOS-L level after administration of the anti-SMAD7 therapeutic remains is at least at least 1.5-fold, at least 2.0-fold, at least 3.0-fold, at least 4.0-fold, at least 5.0-fold, at least 6.0-fold, at least 7.0-fold, at least 8.0-fold, at least 9.0-fold, or at least 10.0-fold higher than the IL-1β, IP10, PD-L1, IDO, or ICOS-L level at the patient's baseline or relative to control levels (e.g., levels observed in a healthy or normal control subject) of IL-1β, IP10, PD-L1, IDO, or ICOS-L.

In some embodiments, the initial dose of the anti-SMAD7 therapeutic (e.g., a SMAD7 ODN) is 40 mg/day or 160 mg/day or 320 mg/day, and wherein the subsequent dose is 40 mg/day or 160 mg/day or 320 mg/day.

In some embodiments, administering at a lower frequency comprises administering at an alternating schedule (e.g., 4 weeks of anti-SMAD7 therapeutic administration alternating with 4 weeks of no treatment or placebo).

In some embodiments, if the patient is in clinical remission and the level of IL-1β, IP10, PD-L1, IDO, or ICOS-L is at a control level (e.g., levels observed in a healthy or normal control subject), then terminating the treatment.

In another aspect, provided herein is a method for treating or managing a disease described herein (see, e.g., Section 7.7(a)) in a patient having the disease (e.g., IBD). In one embodiment, the method comprises the following steps: (a) administering to the patient an initial dose of an anti-SMAD7 therapeutic (e.g., a SMAD7 ODN); (b) analyzing the level of a biomarker selected from the group consisting of bFGF, CCR6, CD123 (IL-3Rα), Eot3, ICAM-1, IgG, IL-1α, IL-4, ILT7, IP-10, ITAC, MCP-1, M-CSF, MIG, MIP-1α, PAI-1, uPA, or VCAM-1 in the patient; and (c) if the level of the biomarker is above control levels, then administering to the patient a subsequent dose of the anti-SMAD7 therapeutic that is greater than or equal to the initial dose. Alternatively, if in step (c), the level of the biomarker is below control levels as determined in step (b), then step (c) comprises administering to the patient a subsequent dose of the anti-SMAD7 therapeutic that is equal to or smaller than the initial dose.

In another aspect, provided herein is a method for treating or managing a disease described herein (see, e.g., Section 7.7(a)) in a patient having the disease (e.g., IBD). In one embodiment, the method comprises the following steps: (a) administering to the patient an initial dose of an anti-SMAD7 therapeutic; (b) analyzing the level of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR in the patient; and (c) if the level of the biomarker is not detectable or at or below a control level, then administering to the patient a subsequent dose of the anti-SMAD7 therapeutic that is greater than or equal to the initial dose. Alternatively, if in step (c), the level of the biomarker is above the control level, then step (c) comprises administering to the patient a subsequent dose of the anti-SMAD7 therapeutic that is equal to or smaller than the initial dose.

In another aspect, provided herein is a method for treating or managing a disease described herein (see, e.g., Section 7.7(a)) in a patient having the disease (e.g., IBD), wherein the method comprises (a) establishing a control level of a biomarker selected from the group consisting of bFGF, CCR6, CD123 (IL-3Rα), Eot3, ICAM-1, IgG, IL-1α, IL-4, ILT7, IP-10, ITAC, MCP-1, M-CSF, MIG, MIP-1α, PAI-1, uPA, or VCAM-1 for the patient; (b) administering to the patient an initial dose of an anti-SMAD7 therapeutic; c) analyzing the level of the biomarker in the patient; and (d) if the level of the biomarker is lower than the control level, then administering to the patient a subsequent dose of the anti-SMAD7 therapeutic that is the same as the initial dose or smaller than the initial dose, or, if the level of the biomarker is unchanged or increased compared to the control level, then administering to the patient a subsequent dose of the anti-SMAD7 therapeutic that is the same as the initial dose or greater than the initial dose or terminating the treatment.

In another aspect, provided herein is a method for treating or managing a disease described herein (see, e.g., Section 7.7(a)) in a patient having the disease (e.g., IBD), wherein the method comprises (a) establishing a control level of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR for the patient; (b) administering to the patient an initial dose of an anti-SMAD7 therapeutic; c) analyzing the level of the biomarker in the patient; and (d) if the level of the biomarker is higher than the control level (e.g., the biomarker is undetectable at the control level and detectable following administration of the anti-SMAD7 therapeutic), then administering to the patient a subsequent dose that is the same as the initial dose or smaller than the initial dose, or, if the level of the biomarker is unchanged (e.g., the biomarker remains undetectable) or decreased compared to the control level, then administering to the patient a subsequent dose that is the same as the initial dose or greater than the initial dose or terminating the treatment.

In some embodiments, the control level for the patient (e.g., IBD patient) is the biomarker level in a sample obtained from the patient prior to administration of the first anti-SMAD7 therapeutic combination treatment, e.g., during a chronic disease period, e.g., when the patient was in remission. In some embodiments, the control level for the patient is the biomarker level in a sample obtained from the patient prior to administration of the first anti-SMAD7 therapeutic combination treatment during an acute disease period (e.g., CD patient: CDAI>150; CDAI≥250 and ≤450; UC patient: MMS≥4 and ≤9, and ES≥2). In some embodiments, the control level for the patient is the biomarker level in a sample obtained from the patient during a period when the patient is administered with an anti-SMAD7 therapeutic combination treatment, or at the beginning of a treatment period (e.g., during week 0, baseline level). In some embodiments, the control level of the patient is the biomarker level in a sample obtained from the patient at an earlier timepoint during an anti-SMAD7 therapeutic combination treatment period.

In some embodiments, the control level for the patient (e.g., IBD patient) is the biomarker level (e.g., median or average biomarker level) in a control group of subjects. In some embodiments, the subjects are healthy subjects. In some embodiments, the subjects are patients. Control groups can be defined based on various criteria related to genetic background, habits, and physical attributes matched to the same set of criteria in the patient. For instance, in some embodiments, the healthy control group and the patient receiving anti-SMAD7 therapeutic combination treatment are matched with respect to age, gender, ethnic origin, smoking habits, dietary habits, body-mass index (BMI), and/or exercise habits. In some embodiments, the control level for the patient is a known reference level for the respective biomarker obtained, e.g., from a scientific or medical publication.

In another aspect, provided herein is a method for treating or managing a disease described herein in a patient having the disease with respect to administration of an initial dose of an anti-SMAD7 therapeutic combination treatment. In one embodiment, provided herein is a method for treating or managing a disease described herein in a patient having the disease, wherein the method comprises the following steps: (a) analyzing the level of a biomarker selected from the group consisting of bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1, in the patient; and (b) if the level of the biomarker is above a control of the biomarker, then administering to the patient an initial dose of an anti-SMAD7 therapeutic. Additionally, the method can further comprise the steps of: (c) analyzing the level of the biomarker in the patient after said administering step, i.e., step (b); and (d) if the level of the biomarker is above the control level of the biomarker, then administering to the patient a subsequent dose of the anti-SMAD7 therapeutic that is greater than or equal to the initial dose. Alternatively, if in step (d), the level of the biomarker is below the control level of the biomarker, as determined in step (c), then step (d) comprises administering to the patient a subsequent dose of the anti-SMAD7 therapeutic that is equal to or smaller than the initial dose.

In another aspect, provided herein is a method for treating or managing a disease described herein in a patient having the disease with respect to administration of an initial dose of an anti-SMAD7 therapeutic. In one embodiment, provided herein is a method for treating or managing a disease described herein in a patient having the disease, wherein the method comprises the following steps: (a) analyzing the level of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR, in the patient; and (b) if the level of the biomarker is undetectable, or at or below a control level of the biomarker (e.g., median biomarker level in a control group of healthy subjects), then administering to the patient an initial dose of an anti-SMAD7 therapeutic. Additionally, the method can further comprise the steps of: (c) analyzing the level of the biomarker in the patient after said administering step, i.e., step (b); and (d) if the level of the biomarker is increased relative to the biomarker levels prior to said administration step or above the control level of the biomarker, then administering to the patient a subsequent dose of the anti-SMAD7 therapeutic that is smaller than or equal to the initial dose. Alternatively, if in step (d) the level of the biomarker is unchanged relative to the biomarker levels prior to said administration step, as determined in step (c), or at or below the control levels of the biomarker, then step (d) comprises administering to the patient a subsequent dose of the anti-SMAD7 therapeutic that is equal to or larger than the initial dose. In some instances, if the subsequent dose administered in step (d) is equal to or greater than the maximum tolerated dose (MTD), then the method comprises the step of terminating the treatment

The level of bFGF, CCR6, CCR7, CD80, CD83, CD86, CD69, CD123 (IL-3Rα), EGFR, GARP, ICAM-1, IgG, IL-1α, IL1-β, IL-2, IL-4, IL-10Rα, IL-18, IL-23p19, ILT7, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1 (collectively “recited biomarkers”) can be analyzed at any timepoint during an administration schedule in a method for treating a disease provided herein. For example, the recited biomarker level can be analyzed before or after administering an anti-SMAD7 therapeutic (e.g., at least 1 day, at least 3 days, at least 5 days, at least 1 week, at least 2 weeks, at least 3 weeks, at least 1 month, at least 2 months, at least 4 months, or at least 6 months), or concurrently with administering the an anti-SMAD7 therapeutic.

The level of a recited biomarker can be analyzed at varying time points following an administering step (b). For instance, in some embodiments, following an administering step (b), the level of the recited biomarker is analyzed at least 1 day, at least 3 days, at least 5 days, at least 1 week, at least 2 weeks, at least 3 weeks, at least 1 month, at least 2 months, at least 4 months, or at least 6 months after said administration step. In some embodiments, the level of the recited biomarker is analyzed immediately after said administration step. In yet other embodiments, the level of the recorded biomarker is analyzed about 7 days, about 10 days, about 15 days, about 20 days, about 25 days, or about 28 days after said administration step.

Control levels of a recited biomarker can be determined based on numerical reference values or with respect to levels of the recited biomarker in a healthy control group. For instance, in some embodiments, control levels of IL1-13 in a blood, serum or plasma sample of a healthy individual can range from 0.0 pg/ml (undetectable level) to 2 pg/ml. In some embodiments, control levels of IL-18 in a blood, serum or plasma sample of a healthy individual can range from 0 pg/ml (e.g., undetectable levels) to 300 pg/ml.

In various embodiments of the invention, the initial dose of an anti-SMAD7 therapeutic administered to a patient having a disease described herein (e.g., IBD) can vary. For instance, in some embodiments, the initial dose of an anti-SMAD7 therapeutic administered to a patient is less than 500 mg/day, less than 400 mg/day, less than 300 mg/day, less than 200 mg/day, less than 100 mg/day, less than 90 mg/day, less than 80 mg/day, less than 70 mg/day, less than 60 mg/day, less than 50 mg/day, less than 40 mg/day, less than 30 mg/day, less than 20 mg/day, or less than 10 mg/day. Alternatively, in other embodiments, the initial dose is at least 1 mg/day, at least 5 mg/day, at least 10 mg/day, at least 20 mg/day, at least 30 mg/day, at least 40 mg/day, at least 50 mg/day, at least 60 mg/day, at least 70 mg/day, at least 80 mg/day, at least 90 mg/day, at least 100 mg/day, at least 200 mg/day, at least 300 mg/day, at least 400 mg/day, or at least 500 mg/day. In yet other embodiments, the initial dose is about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 200 mg/day, about 300 mg/day, about 400 mg/day, or about 500 mg/day. In some embodiments, the initial dose is 5 mg/day, 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day

In some embodiments of a method for treating or managing a disease provided in this section, after analyzing the level of a recited biomarker in the patient in a step (b) or (c), if the level of a biomarker selected from the group consisting of bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 is above control levels of the biomarker, then the method can comprise the step of administering to the patient a subsequent dose of an anti-SMAD7 therapeutic that is greater than the initial dose. In some embodiments, after analyzing the level of the biomarker in the patient in a step (b) or (c), if the level of the biomarker is below a control level of the biomarker, then the method can comprise the step of administering to the patient a subsequent dose of an anti-SMAD7 therapeutic that is smaller than the initial dose.

In some embodiments of a method for treating or managing a disease provided in this section, after analyzing the level of a recited bioma in the patient in a step (b) or (c), if the level of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR is undetectable or at or below a control level of the biomarker, then the method can comprise the step of administering to the patient a subsequent dose of an anti-SMAD7 therapeutic that is greater than the initial dose. In some embodiments, after analyzing the level of the biomarker in the patient in a step (b) or (c), if the level of the biomarker is detectable or above the control level of the biomarker, then the method can comprise the step of administering to the patient a subsequent dose of an anti-SMAD7 therapeutic that is smaller than the initial dose.

In another aspect, provided herein is a method for determining the level of a subsequent dose of an anti-SMAD7 therapeutic with respect to an initial dose of the anti-SMAD7 therapeutic based on levels of a biomarker selected from the group consisting of bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1 in a patient having a disease described herein (e.g., IBD). For instance, in embodiments of the invention described herein, if the biomarker levels in the patient are above control levels following an initial administration step (a) or (b), the subsequent dose of the anti-SMAD7 therapeutic administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day, at least about 170 mg/day, at least about 180 mg/day, at least about 190 mg/day, or at least about 200 mg/day greater than the initial dose. Alternatively, in some embodiments, if the recited biomarker levels in the patient are below a control level following an initial administration step (a) or (b), the subsequent dose of the anti-SMAD7 therapeutic administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day lower than the initial dose. Furthermore, in some embodiments, the initial dose administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

In another aspect, provided herein is a method for determining the level of a subsequent dose of an anti-SMAD7 therapeutic with respect to an initial dose of an anti-SMAD7 therapeutic based on levels of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR in a patient having a disease described herein (e.g., IBD). For instance, in embodiments of the invention described herein, if the biomarker levels in the patient are undetectable or at or below a control level following an initial administration step (a) or (b), the subsequent dose of the anti-SMAD7 therapeutic administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, at least about 100 mg/day, at least about 110 mg/day, at least about 120 mg/day, at least about 130 mg/day, at least about 140 mg/day, at least about 150 mg/day, or at least about 160 mg/day, at least about 170 mg/day, at least about 180 mg/day, at least about 190 mg/day, or at least about 200 mg/day greater than the initial dose. Alternatively, in some embodiments, if the recited biomarker levels in the patient are above the control level following an initial administration step (a) or (b), the subsequent dose of the anti-SMAD7 therapeutic administered in a step (c) or (d) is at least about 5 mg/day, at least about 10 mg/day, at least about 20 mg/day, at least about 30 mg/day, at least about 40 mg/day, at least about 50 mg/day, at least about 60 mg/day, at least about 70 mg/day, at least about 80 mg/day, at least about 90 mg/day, or at least about 100 mg/day lower than the initial dose. Furthermore, in some embodiments, the initial dose of the anti-SMAD7 therapeutic administered in an initial administration step (a) or (b) is between about 10 mg/day and 100 mg/day, about 5 mg/day and 200 mg/day, about 10 mg/day and 50 mg/day, about 50 mg/day and 100 mg/day, and about 100 mg/day and about 200 mg/day, and the subsequent dose of the anti-SMAD7 therapeutic administered in a step (c) or (d) is between about 30 mg/day and 200 mg/day, about 5 mg/day and 30 mg/day, about 20 mg/day and 50 mg/day, about 50 mg/day and 100 mg/day, or about 100 mg/day and 200 mg/day.

In another aspect, provided herein is a method for modulating treatment with an anti-SMAD7 therapeutic in a patient (e.g., an IBD patient) based on a comparison of relative levels of a biomarker selected from the group consisting of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR in a patient before and after an initial administering step. The method comprises the following steps: (a) analyzing the level of the biomarker in the patient; and (b) if the level of the biomarker is undetectable, at or below the control level of the biomarker, then administering to the patient an initial dose of the anti-SMAD7 therapeutic; (c) analyzing the level of the biomarker in the patient after said administering step; and (d) if the level of the biomarker is unchanged or decreased after said administration step relative to the level of biomarker before said administration step, then administering to the patient a subsequent dose of the anti-SMAD7 therapeutic that is the same as the initial dose or higher than the initial dose, or terminating the treatment. Alternatively, in step (d) if the level of the biomarker is unchanged or increased after said administration step (i.e., step (b)) compared to the level of the biomarker before said administration step, then step (d) comprises administering to the patient a subsequent dose of the anti-SMAD7 therapeutic that is lower than the initial dose. Alternatively, in step (d) if the patient is in clinical remission and the level of the biomarker is unchanged or decreased after said administration step (i.e., step (b)) compared to the level of the biomarker before said administration step, then step (d) comprises terminating the treatment.

In another aspect, provided herein is a method for modulating treatment with an anti-SMAD7 therapeutic in a patient (e.g., an IBD patient) based on a comparison of relative levels of a biomarker selected from the group consisting of bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1 in a patient before and after an initial administering step. The method comprises the following steps: (a) analyzing the level of the biomarker in the patient; and (b) if the level of the biomarker is above a control level of the biomarker, then administering to the patient an initial dose of the anti-SMAD7 therapeutic; (c) analyzing the level of the biomarker in the patient after said administering step; and (d) if the level of the biomarker is lower after said administration step than the level of biomarker before said administration step, then administering to the patient a subsequent dose of the anti-SMAD7 therapeutic that is the same as the initial dose or smaller than the initial dose. Alternatively, in step (d), if the level of the biomarker is unchanged or increased after said administration step (i.e., step (b)) compared to the level of the biomarker before said administration step, then step (d) comprises administering to the patient a subsequent dose of the anti-SMAD7 therapeutic that is greater than the initial dose or terminating the treatment. Alternatively, in step (d) if the patient is in clinical remission and the level of the biomarker is unchanged or increased after said administration step (i.e., step (b)) compared to the level of the biomarker before said administration step, then step (d) comprises terminating the treatment.

In some embodiments of a method provided in this section, a change in a recited biomarker level observed after an initial administration step (of SMAD7 ODN) compared to the recited biomarker level prior to the administration step can be analyzed, for example, as a change in percent of recited biomarker levels, to determine the amount of a subsequent dose of the anti-SMAD7 therapeutic to be administered to a patient (e.g., IBD patient). For example, in some embodiments, if the level of a recited biomarker is at least 10%, at least 20%, at least 30%, at least 40%, at least 50%, at least 60%, at least 70%, at least 80%, at least 90%, or at least 95% decreased after said administration step (e.g., an administration step (b)) compared to the level of the recited biomarker before said administration step, then the method comprises a step (e.g., an administration step (d)) of administering to the patient a subsequent dose that is the same as the initial dose or smaller than the initial dose.

In another aspect, provided herein is a method for determining the probability that a patient (e.g., IBD patient) will experience clinical remission following treatment with an anti-SMAD7 therapeutic based on a comparison of a recited biomarker level, for example, based on a comparison of percent change in a recited biomarker level before and after treatment with the anti-SMAD7 therapeutic. For example, in some embodiments, the methods described herein further comprise the step of determining that the patient has a greater than 20%, greater than 30%, greater than 40%, greater than 50%, greater than 60%, greater than 70%, greater than 80%, greater than 90% or greater than 100% chance of experiencing clinical remission of the IBD for a time period of at least 1 week, at least 2 weeks, at least 3 weeks, at least 4 weeks, at least 6 weeks or at least 8 weeks, if the level of the recited biomarker after an administering step (e.g., an administering step (b)) is at least 5%, at least 10%, at least 20%, at least 30%, at least 40%, at least 50%, at least 60%, at least 70%, at least 80%, at least 90%, or at least 95% decreased compared to the recited biomarker level before the administration step.

Clinical remission, as described herein, can be determined by comparison to a reference value, for example, a Crohn's Disease Activity Index (CDAI) or a Modified Mayo Score (MMS). In some embodiments of the invention, clinical remission in a patient having IBD is indicated by a CDAI score of less than 150 (CDAI<150), or a MMS≤2.

In some embodiments of a method provided in this section, a clinical response or clinical remission can be observed at a given time point or within a given time frame with respect to administration of the anti-SMAD7 therapeutic (e.g., using CDAI score or MMS). For example, in some embodiments, clinical remission is observed about one day, about 3 days, about one week, about two weeks, about three weeks, about four weeks, about six weeks, about eight weeks, or about ten weeks after an administration step (for example, an administration step (b)) and maintained for a period of at least 3 days, at least 1 week, at least 2 weeks, at least 3 weeks, at least 4 weeks, at least 6 weeks at least 8 weeks, or at least 10 weeks. Similarly, some embodiments of the invention comprise a method of determining that a patient having IBD has a chance of experiencing clinical remission of IBD, where the patient having IBD had a CDAI of between about 220 and about 400, between about 150 and about 200, between about 200 and about 250, between about 250 and about 300, between about 300 and about 350, between about 350 and about 400, between about 400 and about 450, or greater than about 450 one week prior to an anti-SMAD7 therapy administration step (for example, an administration step (b)). Some embodiments of the invention comprise a method of determining that the patient having IBD has a chance of experiencing clinical remission of IBD, where the patient having IBD had a MMS of between about 4 and about 9, between about 2 and about 4, between about 4 and about 6, between about 6 and about 8, or greater than about 8 one week prior to an anti-SMAD7 therapeutic administration step (for example, an administration step (b)).

In some embodiments, a method of treating or managing a disease described herein in a patient with biomarker levels above a control level (e.g., bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1), comprises administering to the patient a dose of an anti-SMAD7 therapeutic. Furthermore, in some embodiments, the methods for treating or managing a disease described herein in a patient who has above control levels of a recited biomarker following administration of a dose of an anti-SMAD7 therapeutic, comprises administering a further dose of the anti-SMAD7 therapeutic that is greater than or equal to the prior dose. Similarly, in some embodiments of the method of treating or managing a disease described herein, the patient having the disease has below control levels of a recited biomarker following administration of a dose of an anti-SMAD7 therapeutic. In the latter case, the method will comprise administering to the patient a further dose of the anti-SMAD7 therapeutic that is less than or equal to the prior dose. In some embodiments, administration of the anti-SMAD7 therapeutic to the patient is repeated until the patient shows a clinical response or remission, e.g., until the levels of a biomarker, e.g., SMAD7, SMAD3-phosphorylation, bFGF, CCR6, CD123 (IL-3Rα), Eot3, ICAM-1, IgG, IL-1α, IL-4, ILT7, IP-10, ITAC, MCP-1, M-CSF, MIG, MIP-1α, PAI-1, uPA, or VCAM-1, reach control levels, or based on any other clinical parameter known to a skilled artisan, such as a clinician.

In some embodiments, a method of treating or managing a disease described herein in a patient with undetectable or control (e.g., pre-treatment control) levels of a biomarker (e.g., CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR), comprises administering to the patient a dose of an anti-SMAD7 therapeutic. Furthermore, in some embodiments, a methods for treating or managing a disease described herein in a patient who has undetectable or control levels of a recited biomarker following administration of a dose of an anti-SMAD7 therapeutic, comprises administered a further dose of the anti-SMAD7 therapeutic that is greater than or equal to the prior dose. Similarly, in some embodiments of a method of treating or managing a disease described herein, the patient having the disease has above control levels of a recited biomarker following administration of a dose of an anti-SMAD7 therapeutic. In the latter case, the method will comprise administering to the patient a further dose of the anti-SMAD7 therapeutic that is less than or equal to the prior dose. In some embodiments, administration of the anti-SMAD7 therapeutic to the patient is repeated until the patient shows a clinical response or remission, e.g., until the levels of a biomarker, e.g., bFGF, CCR6, CD123 (IL-3Rα), Eot3, ICAM-1, IgG, IL-1α, IL-4, ILT7, IP-10, ITAC, MCP-1, M-CSF, MIG, MIP-1α, PAI-1, uPA, or VCAM-1, reach normal levels, or based on any other clinical parameter known to a skilled artisan, such as a clinician.

In some embodiments of a method of treating or managing a disease described herein in a patient having above control (e.g., healthy control) levels of a biomarker (e.g., bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1), the amount of a anti-SMAD7 therapeutic administered to the patient is increased until the biomarker levels in the patient decrease. In such embodiments, levels of the anti-SMAD7 therapeutic administered to the patient can be increased until the level of a biomarker (e.g., bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1) in the patient decreases to about a normal level of the biomarker or a below normal level of the biomarker.

In some embodiments of a method of treating or managing a disease described herein in a patient having undetectable or control (e.g., pre-treatment control) levels of a biomarker (e.g., CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR), the amount of a anti-SMAD7 therapeutic administered to the patient is increased until the biomarker levels in the patient increase. In such embodiments, levels of the anti-SMAD7 therapeutic administered to the patient can be increased until the level of another biomarker (e.g., bFGF, CCR6, CD123 (IL-3Rα), Eot3, ICAM-1, IgG, IL-1α, IL-4, ILT7, IP-10, ITAC, MCP-1, M-CSF, MIG, MIP-1α, PAI-1, uPA, or VCAM-1) in the patient decreases to about a control level of the biomarker or a below control level of the biomarker.

In some embodiments, the method of treating or managing a disease described herein comprises analyzing a biomarker level (e.g., bFGF, CCR6, CCR7, CD80, CD83, CD86, CD69, CD123 (IL-3Rα), EGFR, GARP, ICAM-1, IgG, IL-1α, IL1-β, IL-2, IL-4, IL-10Rα, IL-18, IL-23p19, ILT7, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1 level) in the patient following each administration of an anti-SMAD7 therapeutic. In some embodiments, utilizing these methods, the absence of a decrease in biomarker levels (e.g., bFGF, CCR6, CD123 (IL-3Rα), ICAM-1, IgG, IL-1α, IL-4, ILT7, ITAC, MCP-1, M-CSF, MIG, MIP-1α, PAI-1, uPA, or VCAM-1) indicates that the treatment or management is not effective. In some embodiments, utilizing these methods, the absence of an increase in biomarker levels (e.g., CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR) indicates that the treatment or management is not effective. In such embodiments, the biomarker levels can be analyzed one time or multiple times, for instance, two times, three times, four times, about five times, about 10 times, about 15 times, about 20 times, or about 30 times, after each administration of the anti-SMAD7 therapeutic. Furthermore, the timing of the measurement of the biomarker levels can vary with respect to the time of the anti-SMAD7 therapeutic administration such that the biomarker levels can be analyzed immediately after, about 1 hour after, about 3 hours after, about 6 hours after, about 12 hours after, about 1 day after, about 3 days after, about 1 week after, about 2 weeks after, and/or about 1 month after the anti-SMAD7 therapeutic administration.

In order to determine levels of a recited biomarker or analyte (e.g., bFGF, CCR6, CCR7, CD80, CD83, CD86, CD69, CD123 (IL-3Rα), EGFR, Eot3, GARP, ICAM-1, IgG, IL-1α, IL1-β, IL-2, IL-4, IL-10Rα, IL-18, IL-23p19, ILT7, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1), for example, IL-1β, in a patient having a disease described herein (e.g., IBD) and using the methods described herein, a sample can be obtained from the patient. Therefore, in some embodiments of a method of treating or managing the disease provided in this section, the level of IL-1β in the patient is determined in a sample obtained from the patient, such as a blood, serum, or plasma sample. Analytes other than or in addition to a recited biomarker, for example, but not limited to Interleukin-6 (IL6), Interleukin-8 (IL8), Interleukin-12 (IL12), Interleukin-17A (IL17A), Interferon gamma (IFN-γ,) Tumor Necrosis Factor alpha (TNFα), Cluster of Differentiation 4 (CD4), Cluster of Differentiation 8 (CD8), Human Leukocyte Antigen-DR (HLA-DR), Chemokine (C—C motif) ligand 20 (CCL20) and C-Reactive Protein (CRP), can also be determined in methods of the invention. Thus, in some embodiments, the method comprises determining a level, or multiple levels, of one or more additional analytes in the patient.

Samples containing a recited biomarker of interest, obtained from the patient, can comprise blood, serum, or plasma samples. Samples can also comprise tissue samples such as, but not limited to, tissue, gastrointestinal, mucosal, submucosal, intestinal, esophageal, ileal, rectal, or lymphatic samples. Levels of analytes of interest in a sample from a patient can be determined using various assays. For example, in methods of the invention, the level of IL-1β and/or another analyte can be determined by immunochemistry, for example, by an enzyme-linked immunosorbent assay (ELISA), or by nucleotide analysis.

In some embodiments, treatment adjustments to higher doses or higher administration frequencies can occur, if the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level after administration of the anti-SMAD7 therapeutic remains unchanged of if it is at least 10%, at least 20%, at least 30%, at least 40% or at least 50% higher than the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level at the patient's baseline or relative to control levels (e.g., levels observed in a healthy or normal control subject) of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR (e.g., levels observed in a healthy or normal control subject).

In some embodiments, treatment adjustments to lower doses or lower administration frequencies can occur, if the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level after administration of the anti-SMAD7 therapeutic remains unchanged of if it is at least 10%, at least 20%, at least 30%, at least 40%, at least 50%, at least 60%, at least 70%, at least 80%, or at least 90% lower than the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR at the patient's baseline or relative to control levels (e.g., levels observed in a healthy or normal control subject) CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR.

In some embodiments, treatment adjustments to higher doses or higher administration frequencies can occur, if the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level after administration of the anti-SMAD7 therapeutic remains unchanged of if it is at least 10%, at least 20%, at least 30%, at least 40% or at least 50% lower than the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level at the patient's baseline or relative to control levels (e.g., levels observed in a healthy or normal control subject) of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR.

In some embodiments, treatment adjustments to lower doses or lower administration frequencies can occur, if the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level after administration of the anti-SMAD7 therapeutic remains is at least at least 1.5-fold, at least 2.0-fold, at least 3.0-fold, at least 4.0-fold, at least 5.0-fold, at least 6.0-fold, at least 7.0-fold, at least 8.0-fold, at least 9.0-fold, or at least 10.0-fold higher than the CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR level at the patient's baseline or relative to control levels (e.g., levels observed in a healthy or normal control subject) of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR.

In some embodiments, the initial dose of the anti-SMAD7 therapeutic (e.g., a SMAD7 ODN) is 40 mg/day or 160 mg/day or 320 mg/day, and wherein the subsequent dose is 40 mg/day or 160 mg/day or 320 mg/day.

In some embodiments, administering at a lower frequency comprises administering at an alternating schedule (e.g., 4 weeks of anti-SMAD7 therapeutic administration alternating with 4 weeks of no treatment or placebo).

In some embodiments, if the patient is in clinical remission and the level of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR is at a control level (e.g., levels observed in a healthy or normal control subject), then terminating the treatment.

In some embodiments, treatment adjustments to higher doses or higher administration frequencies can occur, if the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level after administration of the anti-SMAD7 therapeutic remains unchanged of if it is at least 10%, at least 20%, at least 30%, at least 40% or at least 50% higher than the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level at the patient's baseline or relative to control levels (e.g., levels observed in a healthy or normal control subject) of bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 (e.g., levels observed in a healthy or normal control subject).

In some embodiments, treatment adjustments to lower doses or lower administration frequencies can occur, if the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level after administration of the anti-SMAD7 therapeutic remains unchanged of if it is at least 10%, at least 20%, at least 30%, at least 40%, at least 50%, at least 60%, at least 70%, at least 80%, or at least 90% lower than the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 at the patient's baseline or relative to control levels (e.g., levels observed in a healthy or normal control subject) bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1.

In some embodiments, treatment adjustments to higher doses or higher administration frequencies can occur, if the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level after administration of the anti-SMAD7 therapeutic remains unchanged of if it is at least 10%, at least 20%, at least 30%, at least 40% or at least 50% lower than the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level at the patient's baseline or relative to control levels (e.g., levels observed in a healthy or normal control subject) of bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1.

In some embodiments, treatment adjustments to lower doses or lower administration frequencies can occur, if the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level after administration of the anti-SMAD7 therapeutic remains is at least at least 1.5-fold, at least 2.0-fold, at least 3.0-fold, at least 4.0-fold, at least 5.0-fold, at least 6.0-fold, at least 7.0-fold, at least 8.0-fold, at least 9.0-fold, or at least 10.0-fold higher than the bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 level at the patient's baseline or relative to control levels (e.g., levels observed in a healthy or normal control subject) of bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1.

In some embodiments, the initial dose of the anti-SMAD7 therapeutic (e.g., a SMAD7 ODN) is 40 mg/day or 160 mg/day or 320 mg/day, and wherein the subsequent dose is 40 mg/day or 160 mg/day or 320 mg/day.

In some embodiments, administering at a lower frequency comprises administering at an alternating schedule (e.g., 4 weeks of anti-SMAD7 therapeutic administration alternating with 4 weeks of no treatment or placebo).

In some embodiments, if the patient is in clinical remission and the level of bFGF, CCR6, CD123 (IL-3Rα), ICAM-1, IgG, IL-1α, IL-4, ILT77, ITAC, MCP-1, M-CSF, MIG, MIP-1α, PAI-1, uPA, or VCAM-1 is at a control level (e.g., levels observed in a healthy or normal control subject), then terminating the treatment.

7.8 Additional SMAD7 Therapeutics

(a) SMAD7 ODN as TLR Modulator

In n another aspect, provided herein is a SMAD7 ODN, wherein the SMAD7 ODN is capable of modulating a Toll-Like Receptor (TLR). See, e.g., Section 7.2. In some embodiments, the SMAD7 ODN capable of modulating a TLR comprises a sequence of 10 or more nucleotides, wherein the sequence is complementary to a SMAD7 mRNA. In some embodiments, the SMAD7 ODN is a chemically modified SMAD7 ODN. See, e.g., Section 7.10.

In some embodiments, the SMAD7 ODN is an anti-SMAD7 therapeutic. In some embodiments, the SMAD7 ODN is an anti-SMAD7 ODN (e.g., a SMAD7 ODN, a SMAD7 RNAi, or a SMAD7 miRNA). In some embodiments, the anti-SMAD7 ODN is capable of reducing the expression level of a SMAD7 mRNA or of a SMAD7 protein when introduced into a cell (e.g., a cell of a cell culture, or a cell of a tissue sample obtained from a patient). See, e.g., Section 7.4.

In some embodiments, the SMAD7 ODN is not an anti-SMAD7 therapeutic (e.g., the SMAD7 ODN is not a SMAD7 AON, SMAD7 RNAi, or SMAD7 miRNA). In some embodiments, the SMAD7 ODN does not detectably reduce the SMAD7 expression when introduced into a cell, e.g., by lipotransfection or electroporation. In some embodiments, the SMAD7 ODN does not reduce the expression of a SMAD7 mRNA or of a SMAD7 protein by more than 5%, more than 10%, more than 15%, or more than 20%.

In some embodiments, the SMAD7 ODN does not comprise a nucleotide sequence complementary to region 108-128 of the human SMAD7 nucleotide sequence of SEQ ID NO: 1, or the corresponding RNA sequence, or fragments thereof (e.g., fragments having 11 or more, 12 or more, 13 or more, 14 or more, 15, or more, 16 or more, 17 or more, 18 or more, or 19 or more nucleotides).

In some embodiments, the SMAD7 ODN does not comprise a nucleotide sequence complementary to region 1-30, 16-45, 31-60, 46-75, 61-90, 76-105, 91-120, 106-135, 121-150, 136-165, 151-180, 166-195, 181-210, 196-225, 211-240, 226-255, 241-270, 256-285, 271-300, 286-315, 301-330, 316-345, 331-360, 346-375, 361-390, 376-405, 391-420, 406-435, 421-450, 436-465, 451-180, 466-495, 481-510, 496-525, 511-540, 526-555, 541-570, 556-585, 571-600, 586-615, 601-630, 616-645, 631-660, 646-675, 661-690, 676-705, 691-720, 706-735, 721-750, 736-765, 751-780, 766-195, 781-810, 796-825, 811-840, 826-855, 841-870, 856-885, 871-900, 896-915, 901-930, 916-45, 931-960, 946-975, 961-990, 976-1005, 991-1120, 1106-1135, 1121-1150, 1136-1165, 1151-1180, 1166-1195, 1181-1210, 1196-1225, 1211-1240, 1226-1255, 1241-1270, or 1256-281 of the human SMAD7 nucleotide sequence of SEQ ID NO: 1, or the corresponding RNA sequence, or fragments thereof (e.g., fragments having 11 or more, 12 or more, 13 or more, 14 or more, 15, or more, 16 or more, 17 or more, 18 or more, or 19 or more nucleotides), or combinations thereof (e.g., region 1-45, 16-60, 1-60, 30-90, and the like).

In some embodiments, the SMAD7 ODN does not comprise a nucleotide sequence complementary to nucleotides 403, 233, 294, 295, 296, 298, 299 or 533 of the human SMAD7 nucleotide sequence of SEQ ID NO: 1, or the corresponding RNA sequence.

In some embodiments, the SMAD7 ODN does not comprise the nucleotide sequence of SEQ ID NO: 3 (5′-GTCGCCCCTTCTCCCCGCAGC-3′). In some embodiments, the SMAD7 ODN does not comprise a sequence of 10 or more nucleotides of the nucleotide sequence of SEQ ID NO: 3 (e.g., 11 or more, 12 or more, 13 or more, 14 or more, 15, or more, 16 or more, 17 or more, 18 or more, 19 or more, or 20 or more nucleotides).

In some embodiments, the SMAD7 ODN does not comprise COMPOUND (I).

In some embodiments, the SMAD7 ODN does not comprise a nucleotide sequence of SEQ ID NOs:2-7, SEQ ID NOs: 11-87, and/or SEQ ID NOs:91-144, and/or the nucleotide sequence of any oligonucleotides listed in Table 1, and/or the nucleotide sequence of the oligonucleotides listed in Table 2.

In some embodiments, the SMAD7 ODN does not comprise a sequence of 10 or more nucleotides of the nucleotide sequence of SEQ ID NOs:2-6, SEQ ID NOs: 11-87, or SEQ ID NOs:91-144 (e.g., 11 or more, 12 or more, 13 or more, 14 or more, 15, or more, 16 or more, 17 or more, 18 or more, 19 or more, or 20 or more nucleotides).

In some embodiments, the SMAD7 ODN is capable of modulating a TLR. In some embodiments, the SMAD7 ODN comprises an oligonucleotide modification as described, e.g., in Section 7.13. In some embodiments, the oligonucleotide modification includes non-naturally occurring internucleoside linkages (e.g., phosphorothioate linkages), or methylated bases (e.g., 5-methyl-cytosine, 6-O-methyl-guanine; 7-methyl-guanine). In some embodiments, the methylated bases can occur in GC or CG dinucleotide sequences in the SMAD7 ODN. In some embodiments, the SMAD7 ODN is capable of activating a TLR. In some embodiments, the SMAD7 ODN is capable of inhibiting a TLR. Methods for analyzing if a SMAD7 ODN is capable of modulating a TLR are described, e.g., in Section 7.2.(a).

In some embodiments, the SMAD7 ODN can modulate a TLR. In some embodiments, the SMAD7 ODN can modulate TLR3, TLR4, TLR7, TLR8 or TLR9.

In some embodiments, the SMAD7 ODN can activate a TLR. In some embodiments, the SMAD7 ODN can activate TLR3, TLR4, TLR7, TLR8 or TLR9.

In some embodiments, the SMAD7 ODN can inhibit a TLR. In some embodiments, the SMAD7 ODN can inhibit TLR3, TLR4, TLR7, TLR8 or TLR9.

In some embodiments, the SMAD7 ODN comprises a double-stranded RNA. In some embodiments, the SMAD7 ODN comprises a single-stranded RNA.

In some embodiments, the SMAD7 ODN comprises a CG dinucleotide sequence. In some embodiments, the SMAD7 ODN comprises a GC dinucleotide sequence. In some embodiments, the CG or the GC dinucleotide sequence is a plurality of CG dinucleotide sequences and/or a plurality of GC dinucleotide sequences. In some embodiments, the plurality of CG or GC dinucleotide sequences is 2 or more, 3 or more, 4 or more, 5 or more, or 6 or more CG or GC dinucleotide sequences. In some embodiments, the plurality of CG or GC dinucleotide sequences comprises one or more CG dinucleotide sequences and one or more GC dinucleotide sequences. In some embodiments, the plurality of CG or GC dinucleotide sequences comprises only CG dinucleotide sequences or only GC dinucleotide sequences.

In some embodiments the SMAD7 ODN comprises at least one CG or GC dinucleotide sequence comprising a methylated base (e.g., 5-methyl-cytosine, 6-O-methyl-guanine; 7-methyl-guanine). In some embodiments, the cytosine in a CG or GC dinucleotide sequence is methylated (e.g., 5-methyl-cytosine). In some embodiments, the guanine in the CG or GC dinucleotide sequence is methylated (e.g., 6-O-methyl-guanine; 7-methyl-guanine). In some embodiments, the cytosine and the guanine in the CG or GC dinucleotide sequence is methylated. In some embodiments, the SMAD7 ODN comprises a plurality of CG or GC dinucleotide sequences comprising a methylated base (e.g., 5-methyl-cytosine, 6-O-methyl-guanine; 7-methyl-guanine). In some embodiments, the plurality of CG or GC dinucleotide sequences comprising a methylated base (e.g., 5-methyl-cytosine, 6-O-methyl-guanine; 7-methyl-guanine) is 2 or more, 3 or more, 4 or more, 5 or more, or 6 or more CG or GC dinucleotide sequences.

In some embodiments, the CG or GC dinucleotide sequence in the SMAD7 ODN is a CG or GC phosphate dinucleotide sequence. In some embodiments one or more CG or GC dinucleotide sequences in the SMAD7 ODN comprise a non-natural internucleoside linkage (e.g., a phosphorothioate linkage). In some embodiments, the CG or GC dinucleotide is a CG or GC phosphorothioate dinucleotide sequence. In some embodiments, a two or more CG or GC dinucleotide sequences in the SMAD7 ODN are phosphorothioate dinucleotide sequences. In some embodiments, all CG or GC dinucleotide sequences in the SMAD7 ODN are phosphorothioate dinucleotide sequences. In some embodiments, one or more of the CG or GC phosphorothioate dinucleotide sequences in the SMAD7 ODN comprise one or two methylated bases (e.g., 5-methyl-cytosine, 6-O-methyl-guanine; 7-methyl-guanine). In some embodiments, one or more CG or GC dinucleotide sequences in the SMAD7 ODN comprising a methylated base are phosphorothioate dinucleotide sequences. In some embodiments, all CG or GC dinucleotide sequences in the SMAD7 ODN comprising a methylated base are phosphorothioate dinucleotide sequences.

In some embodiments, the SMAD7 ODN comprises a polypyrimidine tract. In some embodiments, the polypyrimidine tract is between about 10 and about 30 nucleotides or between about 15 and about 25 nucleotides. In some embodiments, the polypyrimidine tract comprises at least 10%, at least 20%, at least 30%, at least 40%, at least 50%, at least 60%, at least 70%, at least 80%, or at least 90% of the SMAD7 ODN sequence. In some embodiments, at least 30%, at least 40%, at least 50%, at least 60%, at least 70%, at least 80% or at least 90% of nucleotides in the SMAD7 ODN are pyrimidines (e.g., cytosine, thymine, uracil, or a non-naturally occurring base). In some embodiments, the SMAD7 ODN is rich in uracil or thymine residues, e.g., at least 30%, at least 40%, at least 50%, at least 60%, at least 70%, at least 80% or at least 90% of residues in the SMAD7 ODN are uracil or thymine. In some embodiments, the SMAD7 ODN is rich in cytosine, e.g., at least 30%, at least 40%, at least 50%, at least 60%, at least 70%, at least 80% or at least 90% of nucleotides in the polypyrimidine tract are cytosine.

In some embodiments, the SMAD7 ODN is formulated in a pharmaceutical composition.

(b) SMAD7 ODN-TLR Modulator Fusion Constructs

In some embodiments, a chemically modified SMAD7 ODN (e.g., SMAD7 AON, SMAD7 RNAi, SMAD7 miRNA) comprises a SMAD7 ODN (SMAD7 ODN portion of the chemically modified SMAD7 ODN) covalently linked to a TLR modulator.

In some embodiments, the chemically modified SMAD7 ODN (e.g., SMAD7 AON) is a SMAD7 ODN (chemically modified or unmodified) that is covalently linked to a compound capable of modulating a TLR. In some embodiments, the compound capable of modulating a TLR is an ODN. In some embodiments, the chemically modified SMAD7 ODN comprises a chemically unmodified SMAD7 ODN that is covalently linked to a non-SMAD7 nucleotide sequence (chemically modified or unmodified) to form a chemically modified SMAD7 ODN with a non-naturally occurring sequence. In some embodiments, the compound capable of modulating the TLR is a small molecule (<1,000 Da), other than an ODN. In some embodiments, the SMAD7 ODN covalently linked to the compound capable of modulating the TLR is a chemically modified SMAD7 ODN. In some embodiments, the SMAD7 ODN is covalently linked to a compound of Table 1. See Section 7.2.

In some embodiments, the SMAD7 ODN (e.g., SMAD7 AON) is covalently linked to a compound capable of activating a TLR. In some embodiments, the TLR is TLR3, TLR4, TLR7, TLR8 or TLR9.

In some embodiments, the SMAD7 ODN (e.g., SMAD7 AON) is covalently linked to a compound capable of inhibiting a TLR. In some embodiments, the TLR is TLR3, TLR4, TLR7, TLR8 or TLR9.

In some embodiments, the chemically modified SMAD7 ODN (e.g., SMAD7 AON) comprises a SMAD7 ODN that is covalently linked to a compound capable of modulating a TLR (e.g., a TLR3, TLR4, TLR7, TLR8 or TLR9 modulator). In some embodiments, the SMAD7 ODN sequence in the chemically modified SMAD7 ODN comprises a further modification, such as a non-naturally occurring internucleoside linkage, a non-naturally occurring sugar residue, or a non-naturally occurring base (e.g., a methylated base). In some embodiments, the SMAD7 ODN in the chemically modified SMAD7 ODN only includes naturally occurring nucleotides. In some embodiments, the chemically modified SMAD7 ODN includes a SMAD7 ODN (modified or unmodified) that is covalently linked to an ODN capable of activating a TLR (e.g., a TLR3, TLR4, TLR7, or TLR9 agonist). In some embodiments, the chemically modified SMAD7 ODN includes a SMAD7 ODN (chemically modified or unmodified) that is covalently linked to an ODN capable of inhibiting a TLR (e.g., a TLR3, TLR4, TLR7, or TLR9 agonist). In some embodiments the chemically modified SMAD7 ODN comprises a compound of Table 1. See Section 7.2. In some embodiments the chemically modified SMAD7 ODN comprises BL-7040, CYT003, CYT003-QbG10, AZD1419, or DIMS0150.

In some embodiments, the SMAD7 ODN (e.g., SMAD7 AON) portion of the chemically modified SMAD7 ODN comprises a sequence of 10 or more nucleotides, wherein the sequence is complementary to a SMAD7 mRNA. In some embodiments, the SMAD7 mRNA comprises the nucleotide sequence of SEQ ID NO: 1.

In some embodiments, the SMAD7 ODN (e.g., SMAD7 AON) portion of the chemically modified SMAD7 ODN comprises a nucleotide sequence complementary to region 108-128 of the human SMAD7 nucleotide sequence of SEQ ID NO: 1, or the corresponding RNA sequence.

In some embodiments, the SMAD7 ODN (e.g., SMAD7 AON) portion of the chemically modified SMAD7 ODN comprises a nucleotide sequence complementary to nucleotides 403, 233, 294, 295, 296, 298, 299 or 533 of the human SMAD7 nucleotide sequence of SEQ ID NO: 1, or the corresponding RNA sequence.

In some embodiments, the SMAD7 ODN portion (e.g., SMAD7 AON) of the chemically modified SMAD7 ODN comprises the nucleic acid sequence of SEQ ID NO: 3. In some embodiments, the SMAD7 ODN in the chemically modified SMAD7 ODN comprises a sequence of 10 or more nucleotides of the nucleotide sequence of SEQ ID NO: 3 (e.g., 11 or more, 12 or more, 13 or more, 14 or more, 15, or more, 16 or more, 17 or more, 18 or more, 19 or more, or 20 or more nucleotides). In some embodiments, the chemically modified SMAD7 ODN comprises COMPOUND (I).

In some embodiments, the SMAD7 ODN (e.g., SMAD7 AON) portion of the chemically modified SMAD7 ODN comprises a nucleotide sequence of SEQ ID NOs:2-7, SEQ ID NOs: 11-87, or SEQ ID NOs:91-144.

In some embodiments, the SMAD7 ODN (e.g., SMAD7 AON) portion of the chemically modified SMAD7 ODN comprises a nucleotide sequence of 10 or more nucleotides of the nucleotide sequence SEQ ID NOs:2-7, SEQ ID NOs: 11-87, or SEQ ID NOs:91-144 (e.g., 11 or more, 12 or more, 13 or more, 14 or more, 15, or more, 16 or more, 17 or more, 18 or more, 19 or more, or 20 or more nucleic acids).

In some embodiments, the SMAD7 ODN (e.g., SMAD7 AON) portion of the chemically modified SMAD7 ODN comprises a nucleotide sequence of SEQ ID NO: 111, SEQ ID NO: 113, SEQ ID NO: 115, SEQ ID NO: 118, SEQ ID NO: 123, SEQ ID NO: 124, SEQ ID NO: 129, SEQ ID NO: 132, SEQ ID NO: 134, SEQ ID NO: 137, or SEQ ID NO: 139.

In some embodiments, the SMAD7 ODN (e.g., SMAD7 AON) portion of the chemically modified SMAD7 ODN comprises a nucleotide sequence of 10 or more nucleotides of the nucleotide sequence SEQ ID NO: 111, SEQ ID NO: 113, SEQ ID NO: 115, SEQ ID NO: 118, SEQ ID NO: 123, SEQ ID NO: 124, SEQ ID NO: 129, SEQ ID NO: 132, SEQ ID NO: 134, SEQ ID NO: 137, or SEQ ID NO: 139 (e.g., 11 or more, 12 or more, 13 or more, 14 or more, 15, or more, 16 or more, 17 or more, 18 or more, 19 or more, or 20 or more nucleic acids).

In some embodiments, the chemically modified SMAD7 ODN (e.g., SMAD7 AON) comprises a TLR modulator, which is an antimalarial therapeutic. In some embodiments, the antimalarial therapeutic is a quinine (e.g., quinacrine or quinidine), a chloriquine, an amodiaquine, a pyrimethamine, a proguanil, a sulfonamide, a mefloquine, an atovaquone, a primaquine, an artemisinin, a haflofantrine, a doxycycline, a clindamycin, or a derivative thereof. In some embodiments, the antimalarial therapeutic is a quinine, a chloroquine, an amodiaquine, a mefloquine, a primaquine, or derivative thereof.

In some embodiments, the chemically modified SMAD7 ODN (e.g., SMAD7 AON) comprises a TLR modulator, which is a quinoline, or derivative thereof. In some embodiments, the quinoline includes, e.g., chloroquine (Aralen), hydroxylchloroquine (Plaquenil), a 4-aminoquinoline (e.g., amodiaquine (Camoquin, Flavoquine)), mefloquine (Lariam, Mephaquin or Mefliam), an 8-aminoquinoline (e.g., primaquine or primaquine phosphate), or atovaquenone/proguanil (Malarone). In some embodiments, the TLR modulator is a quinine (Qualaquin, Quinate, Quinbisul), or derivative thereof. In some embodiments, the quinine includes, e.g., quinacrine (Mepacrine, Atebrine) or quinidine (Quinaglute, Quinidex). In some embodiments, the TLR modulator is hydroxychloroquine.

In some embodiments, the chemically modified SMAD7 ODN (e.g., SMAD7 AON) comprises hydroxychloroquine, or a chemical derivative thereof (e.g., chloroquine).

In some embodiments, the SMAD7 ODN (e.g., SMAD7 AON) and the TLR modulator are linked directly to one another in the chemically modified SMAD7 ODN. In some embodiments the SMAD7 ODN and the TLR modulator are linked via a chemical linker component. In some embodiments, the chemical linker is a cleavable linker. In some embodiments, the SMAD7 ODN and the TLR modulator are separately attached to a scaffold structure, e.g., a nanoparticle. In some embodiments, the attachment of the SMAD7 ODN and the TLR modulator to the scaffold structure is reversible.

In some embodiments, the SMAD7 ODN (e.g., SMAD7 AON) and the TLR modulator are covalently linked to one another in equimolar ratios (e.g., a 1:1 ratio) in the fusion compound. In some embodiments, an excess number of SMAD7 ODN compounds are covalently linked with each TLR modulator compound (e.g., a 2-fold, 3-fold, 4-fold, 5-fold, 6-fold, 8-fold or 10-fold excess of SMAD7 ODN compounds). In some embodiments, an excess number of TLR modulators are covalently linked with each SMAD7 ODN compound (e.g., a 2-fold, 3-fold, 4-fold, 5-fold, 6-fold, 8-fold or 10-fold excess of TLR modulator compounds).

In some embodiments, the chemically modified SMAD7 ODN (e.g., SMAD7 AON) comprises a plurality of different SMAD7 ODNs, such as 2 or more, 3 or more, 4 or more, 5 or more, 6 or more, 8 or more, or 10 or more SMAD7 ODNs. In some embodiments, the chemically modified SMAD7 ODN comprises a SMAD 7 ODN comprising SEQ ID NO: 1, or a fragment thereof, and one or more additional SMAD7 ODNs. In some embodiments, the chemically modified SMAD7 ODN comprises COMPOUND (I), or a fragment thereof, and one or more additional TLR modulators.

In some embodiments, the chemically modified SMAD7 ODN (e.g., SMAD7 AON) comprises a plurality of different TLR modulators, such as 2 or more, 3 or more, 4 or more, 5 or more, 6 or more, 8 or more, or 10 or more TLR modulators. In some embodiments, the chemically modified SMAD7 ODN comprises a TLR9 agonist and one or more additional TLR modulators. In some embodiments, the compound comprises a TLR7 antagonist and a TLR9 antagonist. In some embodiments, the chemically modified SMAD7 ODN comprises hydroxychloroquine and one or more additional TLR modulator.

(i) Illustrative SMAD7 ODN-TLR Modulator Fusion Constructs

In some embodiments, the chemically modified SMAD7 ODN (e.g., SMAD7 AON) comprises a SMAD7 ODN comprising a nucleic acid sequence of SEQ ID NOs:2-7, SEQ ID NOs: 11-87, or SEQ ID NOs:91-144, or a fragment thereof (e.g., a fragment of 10 or more nucleotides), and a TLR7 antagonist and/or a TLR9 antagonist. In some embodiments, the fusion compound comprises a SMAD7 ODN comprising a nucleotide sequence of SEQ ID NO: 111, SEQ ID NO: 113, SEQ ID NO: 115, SEQ ID NO: 118, SEQ ID NO: 123, SEQ ID NO: 124, SEQ ID NO: 129, SEQ ID NO: 132, SEQ ID NO: 134, SEQ ID NO: 137, or SEQ ID NO: 139, or a fragment thereof (e.g., a fragment of 10 or more nucleotides), and a TLR7 agonist and/or a TLR9 antagonist.

In some embodiments the chemically modified SMAD7 ODN (e.g., SMAD7 AON) comprises hydroxychloroquine, CpG ODN2088, IMO-8400, IMO-3100, COV08-0064, or a derivative thereof.

In some embodiments, the chemically modified SMAD7 ODN (e.g., SMAD7 AON) comprises a SMAD7 ODN comprising a nucleotide sequence of SEQ ID NO: 1, or a fragment thereof (e.g., a fragment of 10 or more nucleic acids) and hydroxychloroquine.

In some embodiments, the chemically modified SMAD7 ODN (e.g., SMAD7 AON) comprises a SMAD7 ODN comprising COMPOUND (I), or a fragment thereof (e.g., a fragment of 10 or more nucleotides), and hydroxychloroquine.

In some embodiments, the chemically modified SMAD7 ODN (e.g., SMAD7 AON) comprises a SMAD7 ODN comprising COMPOUND (I), or a fragment thereof (e.g., a fragment of 10 or more nucleotides), and a compound of Table 1.

7.9 Screening Methods

In some embodiments, the SMAD7 ODNs (e.g., anti-SMAD7 ODN, such as SMAD7 AON, SMAD7 RNAi, or SMAD7 miRNA) described herein can act as TLR modulators (e.g., TLR agonists or TLR antagonists).

In some embodiments, the chemically modified SMAD7 ODNs described herein can act as TLR modulators (e.g., TLR agonists or TLR antagonists). Chemically modified SMAD7 ODNs that are capable of modulating a TLR can be identified, e.g., by analyzing the effect of a candidate ODN on TLR pathway components in a cell.

In another aspect, provided herein is a method of screening for a chemically modified SMAD7 ODN capable of modulating a TLR, comprising a) analyzing a baseline level of a TLR pathway component in a cell; b) contacting the chemically modified SMAD7 ODN with the cell for a period of time, and c) analyzing a second level of the biomarker following the contacting, wherein, the chemically modified SMAD7 ODN can modulate a TLR if the second level of the TLR pathway component is increased or decreased relative to the baseline level of the biomarker. In some embodiments, the chemically modified SMAD7 ODN is contacted with the cell in the absence of a transfection agent.

In some embodiments, the baseline level of the TLR pathways component is the level of the TLR pathway component in a control cell.

In some embodiments, the period of time is up to 3 h, up to 6 h, up to 9 h, up to 12 h, up to 15 h, up to 18 h, up to 24 h, up to 30 h, up to 36 h, up to 42 h, or up to 48 h.

In some embodiments, the cell is a primary cell. In some embodiments, the cell is a PBMC or pDC. In some embodiments, the cell is a human, monkey, ape, mouse, rat, rabbit, hamster, dog, cat, cow, or goat PBMC or pDC.

In some embodiments, the cell is a reporter cell. In some embodiments, the reporter cell comprises a reporter gene driven by a cytokine promoter, such as an IP-10, TNFα, IFNγ, or IL-1β promoter. In some embodiments, the reporter cell comprises a reporter gene driven by an NF-κB promoter (e.g., a canonical NF-κB promoter).

In some embodiments, the reporter cell comprises a reporter gene driven by a promoter selected from a bFGF, CCR6, CCR7, CD80, CD83, CD86, CD-69, CD123 (IL-3Rα), EGFR, Eot3 (CCL20), GARP, ICAM-1, IgG, IL-1α, IL1-β, IL-2, IL-4, IL-10Rα, IL-18, IL-23p19, ILT77, IP-10, ITAC, MCP-1, M-CSF, MIG, MIP-1α, PAI-1, RANKL, SLAMF7, tPA, uPA, uPAR, or VCAM-1 promoter.

In some embodiments, the reporter gene is a luciferase, peroxidase, or phosphatase gene. In some embodiments, the reporter cell is derived from a cell line, e.g., a human, hamster, or mouse cell line (e.g., HEK, CHO or RAW 264.7 cells).

In some embodiments, the TLR pathway component level is analyzed at the transcriptional level, using, e.g., a reporter gene assay or a quantitative RT-PCR assay, or the like. In some embodiments, the a TLR pathway components is secreted into the medium of a cell culture. In some embodiments, the TLR pathway component level is analyzed by analyzing the TLR pathway component level in cell culture sample, e.g., by ELISA, a TR-FRET assay, or the like. In some embodiments, the TLR pathway component remains attached to the cell. In some embodiments, the TLR pathway component is exposed at the cell surface. In some embodiments, the TLR pathway component level is analyzed, e.g., by FACS, immunohistochemistry, or microscopic imaging. In some embodiments, the TLR pathway component level (e.g., NIK phosphorylation) is analyzed by western blotting.

In some embodiments, the TLR pathway component comprises a cytokine. In some embodiments, the cytokine comprises TNFα, TGFβ, IFNγ, IL-1β, IL10 or IP-10 (CXCL10). In some embodiments, the TLR pathway component comprises PD-L1, IDO, or ICOS-L. In some embodiments, the TLR pathway component comprises MyD88, TRIF, NIK, IKK (e.g., IKKα or IKKβ), IkB, NFkB, or RelB.

In some embodiments, the TLR pathway component comprises an imflammasome component. In some embodiments, the inflammasome component comprises IL-1β, IL-18, IL18RAP, NOD2, or NLRP3.

In some embodiments, the TLR pathway component comprises bFGF, CCR6, CCR7, CD80, CD83, CD86, CD-69, CD123 (IL-3Rα), EGFR, Eot3, GARP, ICAM-1, IgG, IL-1α, IL1-β, IL-2, IL-4, IL-10Rα, IL-18, IL-23p19, ILT77, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1.

In some embodiments, the TLR modulator is a TLR9 agonist. In some embodiments, the chemically modified SMAD7 ODN is capable of activating TLR9, if the second level of IP-10 is decreased relative to the IP-10 baseline level. In some embodiments, the chemically modified SMAD7 ODN is capable of activating TLR9, if the second level of TNFα, TGFβ, IFNγ, IL-1β, IL10, PD-L1, IDO, or ICOS-L is increased relative to the baseline level.

In some embodiments, the chemically modified SMAD7 ODN is capable of activating TLR9, if the second level of IL-1β or IL-18 is increased relative to the baseline level. In some embodiments, the chemically modified SMAD7 ODN is capable of activating TLR9, if the second level of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR is increased relative to the baseline level.

In some embodiments, the chemically modified SMAD7 ODN is capable of activating TLR9, if the second level of CD123 (IL-3Rα) or CCR6 is decreased relative to the baseline level. In some embodiments, the chemically modified SMAD7 ODN is capable of activating TLR9, if the second level of bFGF, CCR6, CD123 (IL-3Rα), Eot, ICAM-1, IgG, IL-1α, IL-4, ILT77, ITAC, MCP-1, M-CSF, MIG, MIP-1α, PAI-1, uPA, or VCAM-1 is decreased relative to the baseline level.

In some embodiments, the chemically modified SMAD7 ODN is capable of modulating a TLR, if the level of the TLR pathway component increases by at least 1.5-fold, at least 2-fold, at least 3-fold, at least 4-fold, at least 5-fold, at least 6-fold, at least 7-fold, at least 8-fold, at least 9-fold, or at least 10-fold relative to the baseline level.

In some embodiments, the chemically modified SMAD7 ODN is capable of modulating a TLR, if the level of the TLR pathway component decreases by at least 10%, at least 20%, at least 30%, at least 40%, at least 50%, at least 60%, at least 70%, at least 80%, or at least 90% relative to the baseline level.

In some embodiments, the method comprises a) analyzing a baseline level of TNFα, TGFβ, IFNγ, IL-1β, IL10, PD-L1, IDO, ICOS-L, or IP-10 in a PBMC; b) contacting a chemically modified SMAD7 ODN with the PBMC in for a period of time, and c) analyzing a second level of TGFβ, IFNγ, IL-1β, IL10, PD-L1, IDO, or ICOS-L following the contacting, wherein, the chemically modified SMAD7 ODN is capable of modulating the TLR if the second level of IP-10 is decreased relative to the baseline level of IP-10, or if the second level of TNFα, TGFβ, IFNγ, IL-1β, IL10, PD-L1, IDO, or ICOS-L is increased relative to the baseline level of TNFα, TGFβ, IFNγ, IL-1β, IL10, PD-L1, IDO, or ICOS-L

In some embodiments, the method comprises a) analyzing a baseline level of bFGF, CCR6, CCR7, CD80, CD83, CD86, CD-69, CD123 (IL-3Rα), EGFR, Eot3, GARP, ICAM-1, IgG, IL-1α, IL1-β, IL-2, IL-4, IL-10Rα, IL-18, IL-23p19, ILT77, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1 in a PBMC; b) contacting a chemically modified SMAD7 ODN with the PBMC in for a period of time, and c) analyzing a second level of bFGF, CCR6, CCR7, CD80, CD83, CD86, CD69, CD123 (IL-3Rα), EGFR, Eot3, GARP, ICAM-1, IgG, IL-1α, IL1-β, IL-2, IL-4, IL-10Rα, IL-18, IL-23p19, ILT77, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1 following the contacting, wherein, the chemically modified SMAD7 ODN is capable of modulating the TLR if the second level of bFGF, CCR6, CD123 (IL-3Rα), Eot, ICAM-1, IgG, IL-1α, IL-4, ILT77, ITAC, MCP-1, M-CSF, MIG, MIP-1α, PAI-1, uPA, or VCAM-1 is decreased relative to the baseline level, or if the second level of CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR is increased relative to the baseline level

In another aspect, provided herein is a method of screening for a chemically modified SMAD7 ODN that is capable of modulating a TLR, comprising a) contacting a cell in a first cell culture with a TLR agonist to increase a TLR pathway component level in the cell in the absence of the modified SMAD7 ODN; b) contacting a cell in a second cell culture with a TLR agonist to increase the TLR pathway level in the cell in the presence of the chemically modified SMAD7 ODN; and c) analyzing and comparing the TLR pathway component level in the cell in the second cell culture in the presence and absence of the chemically modified SMAD7 ODN, wherein, the chemically modified SMAD7 ODN is a TLR antagonist, if the TLR pathway component level in the cell in the presence of the chemically modified SMAD7 ODN is lower than in the absence of the chemically modified SMAD7 ODN.

In some embodiments, the TLR agonist is a TLR3 agonist. In some embodiments, the TLR3 agonist is polyinosinic-polycytidylic acid (poly(I:C)). See, e.g., FIG. 8A.

In some embodiments, the TLR agonist is a TLR7 agonist. In some embodiments, the TLR7 agonist is imiquimod. See, e.g., FIG. 8B.

In some embodiments, the TLR agonist is a TLR9 agonist. In some embodiments, the TLR9 agonist is ODN2216. See, e.g., FIG. 8C

In some embodiments, a compound (e.g., a SMAD7 ODN or other test compound) is capable of modulating a TLR (e.g., capable of activating TLR9) if the compound can increase the expression (e.g., secretion) of IP10, TNFα and/or IL-6 protein by a plasmacytoid dendritic cell (pDC), when the compound is contacted with the pDC at a concentration of less than 1.0 μM (e.g., less than 0.1 μM, less than 0.2 μM, less than 0.3 μM, less than 0.4 μM, less than 0.5 μM, less than 0.6 μM, less than 0.7 μM, less than 0.8 μM, or less than 0.9 μM), relative to a pDC control not contacted with the compound, as determined in an immunoassay (e.g., an ELISA, FACS, or SPR assay).

In some embodiments, the compound capable of modulating a TLR can increase the expression (e.g., secretion) of IP10, TNFα and IL-6 proteins by the pDC.

In some embodiments, the compound capable of modulating the TLR can increase the expression (e.g., secretion) of IP10, TNFα and/or IL-6 protein by the pDC by at least 1.5-fold, at least 2.0-fold, at least 2.5-fold, at least 3-fold, at least 4-fold, at least 5-fold, at least 6-fold, at least 7-fold, at least 8-fold, at least 9-fold, or at least 10-fold.

In some embodiments, the compound capable of modulating a TLR increases the expression (e.g., secretion) of IP10 and IL6 proteins by a factor of 2-fold or more. See Example 4, Table 3.

In some embodiments, a compound (e.g., a SMAD7 ODN or other test compound) is capable of modulating a TLR (e.g., capable of activating TLR9) if the compound can increase the expression (e.g., secretion) of IL-1β or IL-18 protein by a PBMC, when the compound is contacted with the PBMC at a concentration of less than 10.0 μM (e.g., less than 1.0 μM, less than 0.1 μM, or less than 0.01 μM), relative to a PBMC control not contacted with the compound, as determined in an immunoassay (e.g., an ELISA, FACS, or SPR assay). See, e.g., FIG. 15.

In some embodiments, the compound capable of modulating the TLR can increase the expression (e.g., secretion) of IL-1β or IL-18 protein by the PBMC to a level of at least 1 pg/ml, at least 2 pg/ml, at least 3 pg/ml, at least 4 pg/ml, or at least 5 pg/ml.

In some embodiments, the compounds capable of modulating the TLR can increase the expression (e.g., secretion) of IL-1β or IL-18 protein by the PBMC to a level comparable to a CG ODN, such as ODN2006 (e.g., levels of IL-1β or IL-18 protein induced by the TLR modulator and CpG ODN differ by less than 50%, less than 40%, less than 30%, less than 20%, or less than 10%.).

In some embodiments, the compound capable of modulating the TLR can increase the expression (e.g., secretion) of IL-1β or IL-18 protein by the PBMC to a higher level than ODN150 or ODN7040 (e.g., at least 2-fold, 3-fold, 4-fold, 5-fold, 6-fold, 7-fold, 8-fold, 9-fold, or 10-fold higher).

In some embodiments, the compound capable of modulating a TLR increases the expression of IP10 and IL-6 proteins by a factor of 2-fold or more. See Example 4, Table 3.

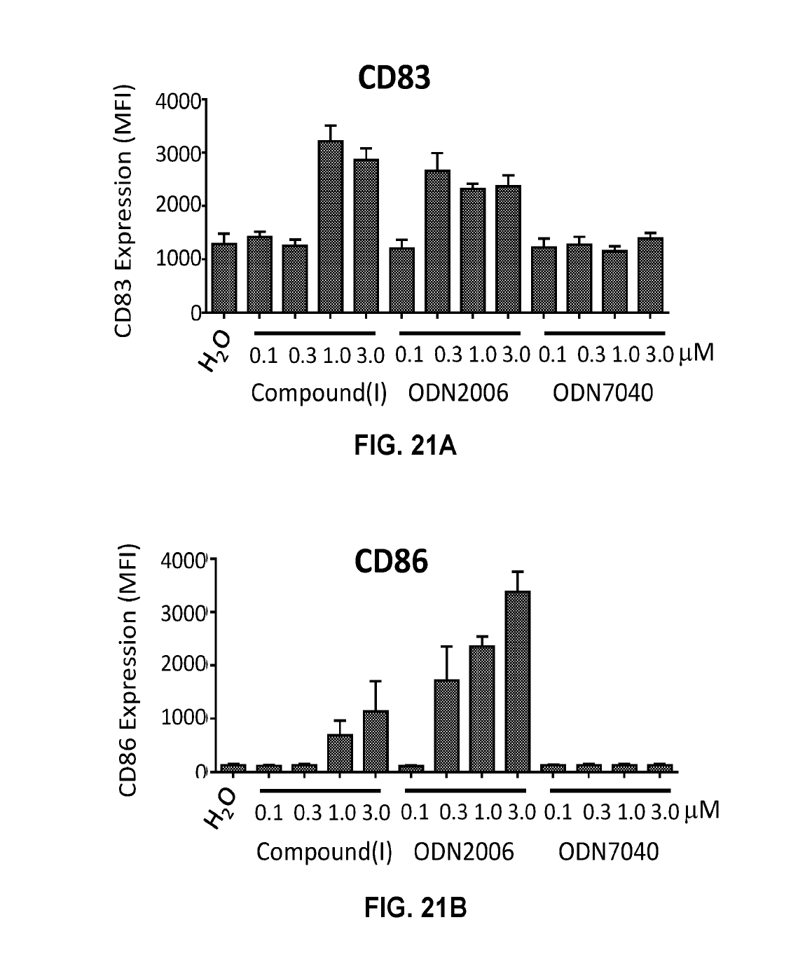

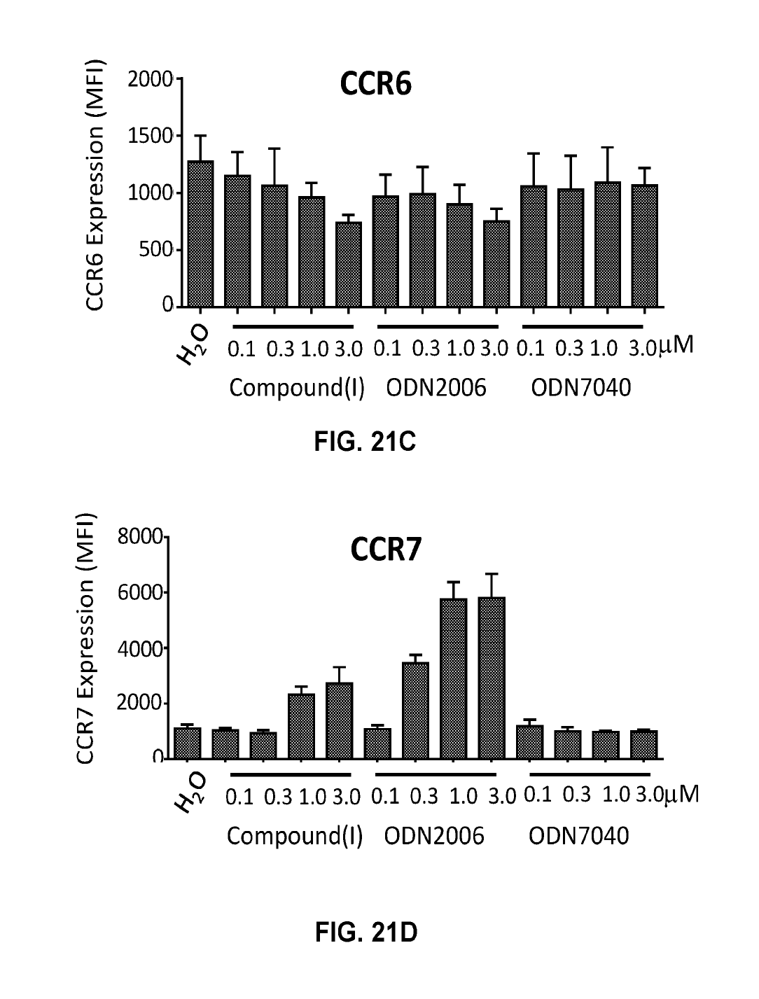

In some embodiments, a compound (e.g., a SMAD7 ODN or other test compound) is capable of modulating a TLR (e.g., capable of activating TLR9) if the compound can increase the expression of a pDC differentiation marker, such as CCR7, CD80, CD83, or CD86, on a pDC, when the compound is contacted with the pDC at a concentration of less than 10.0 μM (e.g., less than 3.0 μM, less than 1.0 μM, or less than 0.3 μM), relative to a pDC control not contacted with the compound, as determined in an immunoassay (e.g., an ELISA, FACS, or SPR assay). See, e.g., FIGS. 21A-D.

In some embodiments, the compound capable of modulating the TLR can increase the expression of the pDC differentiation marker, e.g., CCR7, CD80, CD83, or CD86, on the pDC to by at least 2-fold, 3-fold, 4-fold, 5-fold, 6-fold, 7-fold, 8-fold, 9-fold, or 10-fold relative to a control pDC not treated with the TLR modulator compound.

In some embodiments, the compound capable of modulating a TLR does not induce or only weakly induces B-cell proliferation, when the compound is contacted with the B-cell at a concentration of less than 10.0 μM (e.g., less than 3.0 μM, less than 1.0 μM, less than 0.3 μM, less than 0.1 μM, or less than 0.03 μM), e.g., as determined in a thymidine incorporation assay. In some embodiments, the compound capable of modulating the TLR induces less than 50%, less than 40%, less than 30%, less than 20%, less than 10%, less than 5%, less than 3%, or less than 1% of the level of B-cell proliferation induced by a CpG ODN, e.g., ODN2006, or by ODN150 or ODN7040. See, e.g., FIG. 17.

In some embodiments, the compound capable of modulating a TLR (e.g., 2.0 μM or more, 4.0 μM or more, 6.0 μM or more, 8.0 μM or more, 10.0 μM or more, 12.0 μM or more, 14.0 μM or more, 16.0 μM or more, 18 μM or more, or 20 μM or more) can induce pDC differentiation at similar levels as a CG ODN, e.g., ODN2006, and induce expression (e.g., secretion) of an inflammasome component, e.g., IL-1b or IL-18, at similar levels as a CG ODN, e.g., ODN2006 (e.g., similar levels differ by less than 50%, less than 40%, less than 30%, less than 20%, or less than 10%). In some embodiments, the compound capable of modulating the TLR can induce pDC differentiation to higher levels than ODN150 or ODN7040 (e.g., at least 2-fold, at least 3-fold, at least 4-fold, at least 5-fold, at least 6-fold, at least 7-fold, at least 8-fold, or at least 10-fold higher levels).

In some embodiments, the compound capable of modulating a TLR (e.g., at a concentration of about 1.0 μM or more (e.g., 2.0 μM or more, 4.0 μM or more, 6.0 μM or more, 8.0 μM or more, 10.0 μM or more, 12.0 μM or more, 14.0 μM or more, 16.0 μM or more, 18 μM or more, or 20 μM or more) can induce pDC differentiation at similar levels as a CG ODN, e.g., ODN2006, and induce expression (e.g., secretion) of an inflammasome component, e.g., IL-1β or IL-18, at similar levels as a CG ODN, e.g., ODN2006 (e.g., similar levels differ by less than 50%, less than 40%, less than 30%, less than 20%, or less than 10%), and induces no or only weak B-cell proliferation compared to a CG ODN, e.g., ODN2006 (e.g., less than 50%, less than 40%, less than 30%, less than 20%, less than 10%, or less than 5% of B-cell proliferation). In some embodiments, the compound capable of modulating the TLR can induce pDC differentiation to higher levels than ODN150 or ODN7040 (e.g., at least 2-fold, at least 3-fold, at least 4-fold, at least 5-fold, at least 6-fold, at least 7-fold, at least 8-fold, or at least 10-fold higher levels).

In some embodiments, the compound (e.g., a SMAD7 ODN or other compound) is capable of modulating a TLR (e.g., capable of activating TLR9), if the compound increases the expression of TNFα, IFNγ, TGFβ, IL-6, IL-10, PD-L1, IDO, or ICOS-L protein by a pDC and decreases the expression of IP10 protein by a pDC, when the compound is contacted with the pDC at a concentration of about 1.0 μM or more (e.g., 2.0 μM or more, 4.0 μM or more, 6.0 μM or more, 8.0 μM or more, 10.0 μM or more, 12.0 μM or more, 14.0 μM or more, 16.0 μM or more, 18 μM or more, or 20 μM or more), relative to a pDC control not contacted with the compound, as determined in an immunoassay (e.g., an ELISA, FACS, or SPR assay).

In some embodiments, the compound capable of modulating the TLR increases the expression of TNFα, IFNγ, TGFβ, IL-6, IL-10, PD-L1, IDO, and ICOS-L proteins by the pDC and decreases the expression of IP10 protein by the pDC.

In some embodiments, the compound capable of modulating the TLR decreases the expression of IP10 protein by the pDC by at least 10%, at least 20%, at least 30%, at least 40%, at least 50%, at least 60%, at least 70%, at least 80%, or at least 90%.

In some embodiments, the compound capable of modulating the TLR can decrease the expression of IP10 protein by the pDC by at least 2-fold, at least 3-fold, at least 4-fold, at least 5-fold, at least 6-fold, at least 7-fold, at least 8-fold, at least 9-fold, or at least 10-fold.

In some embodiments, the compound capable of modulating the TLR can increase the expression of TNFα, IFNγ, TGFβ, IL-6, IL-10, PD-L1, IDO, or ICOS-L protein by the pDC by at least 1.5-fold, at least 2.0-fold, at least 2.5-fold, at least 3-fold, at least 4-fold, at least 5-fold, at least 6-fold, at least 7-fold, at least 8-fold, at least 9-fold, or at least 10-fold.

In some embodiments, the compound capable of modulating a TLR decreases the expression of IP10 protein by a factor of 10-fold or more and the SMAD7 ODN increases the expression of each of TNFα, IL-6, and ICOS-L by a factor of 2-fold or more. See Example 4, Table 4.

In some embodiments, expression of a TLR pathway component (e.g., at the protein or mRNA level), such as TNFα, IFNγ, TGFβ, IL-6, IL-10, PD-L1, IDO, ICOS-L, and the like, is not detectable in the absence of a TLR modulator. In some embodiments, the TLR modulator can increase expression of an otherwise undetectable TLR pathway component at least 2-fold, at least 3-fold, at least 4-fold, at least 5-fold, at least 6-fold, at least 7-fold, at least 8-fold, at least 9-fold, or at least 10-fold over background (e.g., above the level of a “no TLR modulator” control).

In some embodiments, a compound (e.g., a SMAD7 ODN or other compound) is capable of modulating a TLR (e.g., capable of activating TLR9), if the compound can increase the expression of ICOS-L proteins by a pDC by a factor of 5-fold or more, when contacted with the pDC at a concentration of 1.0 μM or more, in the presence of a quinoline or quinine relative to a pDC control not contacted with the compound capable of modulating the TLR, as determined in an immunoassay, wherein the quinoline or quinine is present at a concentration below the threshold concentration at which the quinoline or quinine alone detectably increases ICOS-L expression.

In some embodiments, the quinoline is chloroquine (Aralen), hydroxylchloroquine (Plaquenil), amodiaquine (Camoquin), mefloquine (Lariam), 8-aminoquinoline or atovaquenone/proguanil (Malarone). In some embodiments, the quinoline is hydroxychloroquine. In some embodiments, the concentration of hydroxychloroquine is 10 μM or more. In some embodiments, the quinine is quinacrine (Mepacrine) or quinidine (Quinidex). See Example 3, FIGS. 6A-B.

In some embodiments, a compound (e.g., a SMAD7 ODN or other compound) is capable of modulating a TLR (e.g., capable of inhibiting TLR3) if the compound can reduce PolyI:C-induced IFN secretion of peripheral blood mononuclear cells (PBMCs), when the compound is contacted with the PBMCs at a concentration of 1.0 μM or less, relative to a PolyI:C-induced PBMC control not contacted with the compound. In some embodiments, the compound capable of modulating the TLR can reduce the PolyI:C-induced IFNα secretion of PBMCs by at least 10%, at least 20%, at least 30%, at least 40%, at least 50%, at least 60%, at least 70%, at least 80%, or at least 90%.

In some embodiments, the compound capable of modulating the TLR can reduce the PolyI:C-induced IFNα secretion of the PBMCs by 50% or more. See Example 6, FIG. 8A.

In some embodiments, a compound (e.g., a SMAD7 ODN or other compound) is capable of modulating a TLR (e.g., capable of inhibiting TLR7) if the compound can reduce the imiquimod-induced IFNα secretion of peripheral blood mononuclear cells (PBMCs), when the compound is contacted with the PBMCs at a concentration of 0.1 μM or less, relative to an imiquimod-induced PBMC control not contacted with the compound.

In some embodiments, the compound capable of modulating the TLR can reduce the imiquimod-induced IFNα secretion of PBMCs by at least 10%, at least 20%, at least 30%, at least 40%, at least 50%, at least 60%, at least 70%, at least 80%, or at least 90%.

In some embodiments, the compound capable of modulating the TLR can reduce the imiquimod-induced IFNα secretion of the PBMCs by 50% or more. See Example 6, FIG. 8B.

In some embodiments, a compound (e.g., a SMAD7 ODN or other compound) is capable of modulating a TLR (e.g., capable of inhibiting TLR9) if the compound can reduce the ODN2216-induced IFNα secretion of peripheral blood mononuclear cells (PBMCs), when the compound is contacted with the PBMCs at a concentration of 0.1 μM or less, relative to an imiquimod-induced PBMC control not contacted with the compound capable of modulating the TLR. In some embodiments, the compound capable of modulating the TLR can reduce the ODN2216-induced IFNα secretion of PBMCs by at least 10%, at least 20%, at least 30%, at least 40%, at least 50%, at least 60%, at least 70%, at least 80%, or at least 90%.

In some embodiments, the compound capable of modulating the TLR can reduce the ODN2216-induced IFNα secretion of the PBMCs by 50% or more. See Example 6, FIG. 8C.

In another aspect, provided herein is a method of screening for a compound capable of synergizing with an anti-SMAD7 therapeutic or with a chemically modified SMAD7 ODN described herein, comprising (a) contacting the anti-SMAD7 therapeutic or the chemically modified SMAD7 ODN with a cell of the immune system at a first concentration; (b) determining a first expression level of a TLR pathway component in the cell of the immune system; (c) contacting the cell of the immune system with the anti-SMAD7 therapeutic or the chemically modified SMAD7 ODN at the first concentration and a test compound at a second concentration, and (d) determining a second expression level of the TLR pathway component in the cell of the immune system, wherein the test compound is capable of synergizing with the anti-SMAD7 therapeutic or the chemically modified SMAD7 ODN, if the second expression level of the TLR pathway component in the cell of the immune system is higher that the first expression level of the TLR pathway component. See Example 3, FIGS. 6A-B.

In some embodiments, the anti-SMAD7 therapeutic or the chemically modified SMAD7 ODN alone at the first concentration is capable of increasing the TLR pathway component level in the immune cell less than 2-fold, compared to a control sample in which the anti-SMAD7 therapeutic or the chemically modified SMAD7 ODN is absent. See Example 3, FIGS. 6A-B.

In some embodiments, wherein the test compound alone at the second compound concentration does not detectably increase the expression level of the biomarker in the immune cell compared to a control sample in which the anti-SMAD7 therapeutic or the chemically modified SMAD7 ODN is absent. See Example 3, FIGS. 6A-B.

In some embodiments, the test compound is capable of synergizing with the anti-SMAD7 therapeutic or the chemically modified SMAD7 ODN, if the second expression level of the TLR pathway component in the cell of the immune system is at least 3-fold higher, at least 5-fold, at least 10-fold, at least 15-fold, or at least 20-fold higher that the first expression level of the TLR pathway component. See Example 3, FIGS. 6A-B.

In some embodiments, the second expression level of the TLR pathway component in the cell of the immune system is at least 4-fold, at least 5-fold, at least 6-fold, at least 7-fold, at least 8-fold, at least 9-fold, or at least 10-fold higher that the first expression level of the TLR pathway component.

In some embodiments, the cell of the immune system is a PBMC or a pDC.

In some embodiments, the TLR pathway component comprises TNFα, IFNγ, IL-1β, IL-10, TGFβ, PD-L1, ICOS-L or IP-10 (CXCL10).

In some embodiments, the TLR pathway component comprises bFGF, CCR6, CCR7, CD80, CD83, CD86, CD69, CD123 (IL-3Rα), EGFR, Eot3, GARP, ICAM-1, IgG, IL-1α, IL1-β, IL-2, IL-4, IL-10Rα, IL-18, IL-23p19, ILT77, IP-10, ITAC, MCP-1, M-CSF, MIG, MIP-1α, PAI-1, phospho-histone H3, phospho-p38 MAP kinase, phospho-ZAP70, RANKL, SLAMF7, tPA, uPA, uPAR, or VCAM-1.

7.10 Oligonucleotide Modifications

In some embodiments the SMAD7 ODNs (e.g., anti-SMAD7 ODNs) described herein are chemically modified SMAD7 ODNs. In certain specific embodiments, a SMAD7 ODN as described herein can have a sequence that is complementary to the nucleotide sequence of SMAD7 mRNA (i.e., the SMAD7 ODN can be an antisense oligonucleotide).

The chemically modified SMAD7 ODNs provided herein can include, e.g., non-naturally occurring nucleobases, modified internucleoside (backbone) linkages, sugar modifications or modified 5′- or 3′-ends. In some embodiments, the chemically modified SMAD7 ODNs comprise a non-naturally-occurring sequence tag. In some embodiments, the chemically modified SMAD7 ODNs comprise SMAD7 ODNs linked to other nucleic acid sequences (e.g., a TLR modulator) to yield non-naturally occurring nucleic acid sequences. Further oligonucleotide modifications can include labels, e.g., for the detection of ODNs, such as fluorescence labels or isotope labels (e.g., deuterium, tritium, C¹³, N¹⁵, O¹⁸).

The chemically modified SMAD7 ODNs described herein can include naturally occurring nucleobases, sugars, and covalent internucleoside (backbone) linkages, as well as non-naturally occurring portions. For example, the chemically modified SMAD7 ODNs can include a mixed-backbone, e.g., including one or more phosphorothioate linkages. In some embodiments, the chemically modified SMAD7 ODNs can have one or more cytosine residues replaced by 5-methylcytosine. In some embodiments the one or more cytosine residues form part of a CpG pair.

In some embodiments, the chemically modified SMAD7 ODNs include an artificial nucleoside, such as deoxycytidine and/or 5-methyl 2′-deoxycytidine, including, but not limited to, 5-methyl-2′-deoxycytidine 5′-monophosphate and 5-methyl-2′-deoxycytidine 5′-monophosphorothioate. In some embodiments, the chemically modified SMAD7 ODNs comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 artificial nucleosides.

In some embodiments, the SMAD7 ODN comprises a CG dinucleotide sequence. In some embodiments, the SMAD7 ODN comprises a GC dinucleotide sequence. In some embodiments, the CG or the GC dinucleotide sequence is a plurality of CG dinucleotide sequences and/or a plurality of GC dinucleotide sequences. In some embodiments, the plurality of CG or GC dinucleotide sequences is 2 or more, 3 or more, 4 or more, 5 or more, or 6 or more CG or GC dinucleotide sequences. In some embodiments, the plurality of CG or GC dinucleotide sequences comprises one or more CG dinucleotide sequences and one or more GC dinucleotide sequences. In some embodiments, the plurality of CG or GC dinucleotide sequences comprises only CG dinucleotide sequences or only GC dinucleotide sequences.

In some embodiments the SMAD7 ODN comprises at least one CG or GC dinucleotide sequence comprising a methylated base (e.g., 5-methyl-cytosine, 6-O-methyl-guanine; 7-methyl-guanine). In some embodiments, the cytosine in a CG or GC dinucleotide sequence is methylated (e.g., 5-methyl-cytosine). In some embodiments, the guanine in the CG or GC dinucleotide sequence is methylated (e.g., 6-O-methyl-guanine; 7-methyl-guanine). In some embodiments, the cytosine and the guanine in the CG or GC dinucleotide sequence is methylated. In some embodiments, the SMAD7 ODN comprises a plurality of CG or GC dinucleotide sequences comprising a methylated base (e.g., 5-methyl-cytosine, 6-O-methyl-guanine; 7-methyl-guanine). In some embodiments, the plurality of CG or GC dinucleotide sequences comprising a methylated base (e.g., 5-methyl-cytosine, 6-O-methyl-guanine; 7-methyl-guanine) is 2 or more, 3 or more, 4 or more, 5 or more, or 6 or more CG or GC dinucleotide sequences.

In some embodiments, the CG or GC dinucleotide sequence in the SMAD7 ODN is a CG or GC phosphate dinucleotide sequence. In some embodiments one or more CG or GC dinucleotide sequences in the SMAD7 ODN comprise a non-natural internucleoside linkage (e.g., a phosphorothioate linkage). In some embodiments, the CG or GC dinucleotide is a CG or GC phosphorothioate dinucleotide sequence. In some embodiments, a two or more CG or GC dinucleotide sequences in the SMAD7 ODN are phosphorothioate dinucleotide sequences. In some embodiments, all CG or GC dinucleotide sequences in the SMAD7 ODN are phosphorothioate dinucleotide sequences. In some embodiments, one or more of the CG or GC phosphorothioate dinucleotide sequences in the SMAD7 ODN comprise one or two methylated bases (e.g., 5-methyl-cytosine, 6-O-methyl-guanine; 7-methyl-guanine). In some embodiments, one or more CG or GC dinucleotide sequences in the SMAD7 ODN comprising a methylated base are phosphorothioate dinucleotide sequences. In some embodiments, all CG or GC dinucleotide sequences in the SMAD7 ODN comprising a methylated base are phosphorothioate dinucleotide sequences.

In some embodiments, the chemically modified SMAD7 ODNs include the nucleic acid sequence of SEQ ID NO: 4 (5′-GTXGCCCCTTCTCCCXGCAG-3)′, wherein X is 5-methyl 2′-deoxycytidine.

In some embodiments, the chemically modified SMAD7 ODNs include the nucleic acid sequence of SEQ ID NO: 5 (5′-GTXGCCCCTTCTCCCXGCAGC-3′), wherein X is 5-methyl 2′-deoxycytidine.

In some embodiments, the chemically modified SMAD7 ODNs include the nucleic acid sequence of SEQ ID NO: 6 (5′-GTXYCCCCTTCTCCCXYCAG-3′), whereby X is a nucleotide including a nitrogenous base selected from the group consisting of cytosine and 5-methylcytosine nucleoside or a 2′-O-methylcytosine nucleoside, and wherein Y is a nucleotide including a nitrogenous base selected from the group consisting of guanine and 5-methylguanine or a 2′-O-methylguanine nucleoside, optionally provided that at least one of the nucleotides X or Y comprises a methylated nitrogenous base.

In some embodiments, the chemically modified SMAD7 ODNs include the nucleic acid sequence of SEQ ID NO: 5: (5′-GTC* GCC CCT TCT CCC C*GC AGC-3′), whereby C* represents 5-methyl-2′-deoxycytidine. In some embodiments, at least one of the internucleoside linkages of the chemically modified SMAD7 ODN is an O,O-linked phosphorothioate. In some embodiments, all of the internucleotide linkages of the chemically modified SMAD7 ODNs can be O,O-linked phosphorothioates. In some embodiments, the chemically modified SMAD7 ODNs is a SMAD7 AON comprising a nucleotide sequence of SEQ ID NO: 5, wherein each of the 20 internucleotide linkages is an O,O-linked phosphorothioate linkage.

In some embodiments, the chemically modified SMAD7 ODNs include at least one internucleoside linkage, which is a phosphate linkage, e.g., a monophosphate linkage.

In some embodiments, the chemically modified SMAD7 ODNs include at least one internucleoside linkage, which is a phosphorothioate linkage. In some embodiments, the chemically modified SMAD7 ODNs include at least 2, 3, 4, 5, 6, 7, 8, 9, 10, 11, 12, 13, 14, 15, 16, 17, 18, 19, 20, 21, 22, 23, 24, 25, or more phosphorothioate linkages. In some embodiments, at least 5%, 10%, 20%, 25%, 30%, 35%, 40%, 45%, 50%, 55%, 60%, 65%, 70%, 75%, 80%, 85%, 90%, 95%, or 100% of internucleoside linkages in the chemically modified SMAD7 ODNs are phosphorothioate linkages. In some embodiments, all internucleoside linkages are phosphorothioate linkages.

In some embodiments, the chemically modified SMAD7 ODNs include at least one, unnatural nucleoside, e.g., 5-methyl-2′-deoxycytidine-5′-monophosphate and 5-methyl-2′-deoxycytidine-5′-monophosphorothioate. In some embodiments, the modified SMAD7 ODNs include 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidines. In some embodiments, at least 5%, 10%, 20%, 25%, 30%, 35%, 40%, 45%, 50%, 55%, 60%, 65%, 70%, 75%, 80%, 85%, 90%, 95%, or 100% of nucleotides in the chemically modified SMAD7 ODNs include deoxycytidine and/or 5-methyl-2′-deoxycytidine. In some embodiments, the chemically modified SMAD7 ODNs include at least 2, 3, 4, 5, 6, 7, 8, 9, 10, 11, 12, 13, 14, 15, 16, 17, 18, 19, 20, 21, 22, 23, 24, 25, or more deoxycytidine and/or 5-methyl 2′-deoxycytidine. In some embodiments, the chemically modified SMAD7 ODNs include one or more deoxycytidines and no 5-methyl 2′-deoxycytidine. In some embodiments, the chemically modified SMAD7 ODNs include one or more 5-methyl 2′-deoxycytidine and no deoxycytidine.

In some embodiments, the chemically modified SMAD7 ODNs include the nucleic acid sequence: 5′-GTXGCCCCTTCTCCCXGCAG-3′ (SEQ ID NO: 4), wherein X is 5-methyl-2′-deoxycytidine and wherein all internucleoside linkages are phosphorothioate linkages.

In some embodiments, the chemically modified SMAD7 ODNs include the nucleic acid sequence 5′-GTXGCCCCTTCTCCCXGCAGC-3′ (SEQ ID NO: 5), wherein X is 5-methyl-2′-deoxycytidine and wherein all internucleoside linkages are phosphorothioate linkages.

In some embodiments, the chemically modified SMAD7 ODNs include methylphosphonate linkages that are be placed at the 5′- and/or 3′-ends of the SMAD7 ODN.

In some embodiments, the chemically modified SMAD7 ODNs include pharmaceutically acceptable salts or solvates. In some embodiments, the solvates are hydrates. In some embodiments, the chemically modified SMAD7 ODNs are a sodium salt of the chemically modified SMAD7 ODNs including the nuclei acid sequence of SEQ ID NO: 5, that optionally can include 1 to 20 O,O-linked phosphorothioate internucleotide linkages. Contemplated salts of chemically modified SMAD7 ODNs include those that are fully neutralized, e.g., each phosphorothioate linkage is associated with an ion such as Na⁺. In some embodiments the salts of the chemically modified SMAD7 ODNs are only partially neutralized, e.g., less than all phosphorothioate linkages are associated with an ion (e.g., less than 99%, less than 95%, less than 90%, less than 85%, less than 80%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized).

The chemically modified SMAD7 ODNs described herein can include backbone modifications. For example, and without limitation, the chemically modified SMAD7 ODNs described herein can include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. The chemically modified SMAD7 ODNs described herein include various salts, mixed salts and free acid forms of the ODNs. Methods of preparing such chemically modified SMAD7 ODNs are known in the art.

In some embodiments, the chemically modified SMAD7 ODNs described herein can include backbone structures that, instead of a phosphorus atom, include short chain alkyl or cycloalkyl internucleoside linkages, mixed heteroatom and alkyl or cycloalkyl internucleoside linkages, or one or more short chain heteroatomic or heterocyclic internucleoside linkages. Such heteroatomic or heterocyclic internucleoside linkages can include, e.g., morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Methods of preparing modified SMAD7 ODNs with such backbone structures are well known in the art.

In some embodiments, the chemically modified SMAD7 ODNs described herein can have both the sugar and the internucleoside linkage, i.e., the backbone, of the nucleotide units replaced with non-natural groups. The base units are generally maintained for hybridization with a target SMAD7 nucleic acid. For example, the chemically modified SMAD7 ODNs can include peptide nucleic acids (PNAs). In PNAs, the sugar-backbone of an oligonucleotide is replaced with an amide containing backbone, in particular an aminoethylglycine backbone. The nucleobases of SMAD7 PNAs are retained and are bound directly or indirectly to aza nitrogen atoms of the amide portion of the backbone. Method of preparing such PNA compounds are well known in the art.

In some embodiments, the chemically modified SMAD7 ODNs described herein can include phosphorothioate backbones or oligonucleosides with heteroatom backbones, such as —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—].

In some embodiments, the chemically modified SMAD7 ODNs described herein can include one or more substituted sugar moieties. In some embodiments, the modified SMAD7 ODNs can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, the modified SMAD7 ODNs include are O[(CH₂)ₙO]ₘ CH₃, O(CH₂)ₙ OCH₃, O(CH₂)ₙ NH₂, O(CH₂)ₙ CH₃, O(CH₂)ₙ ONH₂, and O (CH₂)ₙ ON[(CH₂)ₙ CH₃)]₂, where n and m are from 1 to about 10. In some embodiments, the modified SMAD7 ODNs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modified SMAD7 ODNs include a 2′-methoxyethoxy (2′-O—CH₂ CH₂ OCH₃, also known as 2′-O-(2-methoxyethyl) or 2′-MOE), i.e., an alkoxyalkoxy group. In some embodiments, the modified SMAD7 ODNs include 2′-dimethylaminooxyethoxy, i.e., a O(CH₂)₂ ON(CH₃)₂ group, also known as 2′-DMAOE, and 2′-dimethylamino-ethoxyethoxy (also known in the art as 2′-O-dimethylaminoethoxyethyl or 2′-DMAEOE), i.e., 2′-O—CH₂—O—CH₂—N(CH₂)₂. Methods for making such ODN modifications are well known in the art.

In some embodiments, the chemically modified SMAD7 ODNs described herein include 2′-methoxy (2′-O—CH₃), 2′-aminopropoxy (2′-OCH₂ CH₂ CH₂ NH₂) and 2′-fluoro (2′-F) modifications. Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. The chemically modified SMAD7 ODNs can also have sugar mimetics such as cyclobutyl moieties in place of the pentofuranosyl sugar.

The chemically modified SMAD7 ODNs described herein can also include nucleobase modifications or substitutions. As used herein, “unmodified” or “natural” nucleobases include the purine bases adenine (A) and guanine (G), and the pyrimidine bases thymine (T), cytosine (C) and uracil (U). The chemically modified SMAD7 ODNs can include, e.g., synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. The chemically modified SMAD7 ODNs can further include nucleobases such as those disclosed in U.S. Pat. No. 3,687,808, those disclosed in The Concise Encyclopedia of Polymer Science And Engineering, pages 858-859, Kroschwitz, J. I., ed. John Wiley & Sons, 1990, those disclosed by Englisch et al., Angewandte Chemie, International Edition, 1991, 30, 613, or those disclosed by Sanghvi, Y. S., Chapter 15, Antisense Research and Applications, pages 289-302, Crooke, S. T. and Lebleu, B., ed., CRC Press, 1993. In some embodiments, the chemically modified SMAD7 ODNs include nucleobases that can increase the binding affinity of the chemically modified SMAD7 ODN. Such nucleobases can include, e.g., 5-substituted pyrimidines, 6-azapyrimidines and N-2, N-6 and 0-6 substituted purines, including 2-aminopropyladenine, 5-propynyluracil and 5-propynylcytosine 5-methylcytosine substitutions. In some embodiments, the chemically modified SMAD7 ODNs can include one or more of the above-mentioned modified nucleobases in combination with 2′-O-methoxyethyl sugar modifications. Methods for preparing such chemically modified ODNs are well known in the art.

In some embodiments, the chemically modified SMAD7 ODNs described herein can be covalently linked to one or more moieties or conjugates which enhance the activity, cellular distribution or cellular uptake of the chemically modified SMAD7 ODN. Such moieties include, without limitation, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Methods for conjugating ODNs to the exemplified moieties are known in the art.

In some embodiments, the chemically modified SMAD7 ODN s described herein are uniformly modified, e.g., all internucleoside linkages in the chemically modified SMAD7 ODN are phosphorothioate linkages. In some embodiments, the chemically modified SMAD7 ODNs are modified in one or more position, e.g., one or more internucleoside linkages in the chemically modified SMAD7 ODN are phosphorothioate linkages.

In some embodiments, the chemically modified SMAD7 ODNs described herein include pharmaceutically acceptable salts, esters, or salts of such esters.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The following examples are provided by way of illustration, not limitation.

### 8. EXAMPLES

### 8.1 Example 1: Effect of COMPOUND (I) on SMAD7 Expression

This Example demonstrates that COMPOUND (I) does not affect SMAD7 mRNA or protein expression in normal human PBMCs or normal human pDCs under experimental conditions where COMPOUND (I) is added to the cellular medium and not transfected into the PBMCs or pDCs. Under these conditions, without wishing to be bound by theory, COMPOUND (I) and certain other tested ODNs are believed to act as TLR9 agonists.

### Oligonucleotides

Table 2 lists the sequences and certain chemical properties of oligonucleotides (ODNs) used in the examples provided herein.

TABLE 2
Sequences and Chemistries of Exemplary Oligonucleotides

| | | Backbone Chemistry and | SEQ |
|---|---|---|---|
| ODN | Sequence | Nucleoside modifications | ID NO. |
| ODN150 | 5′-G*G*A*ACAGTTCGTCCAT*G*G*C-3′ | PO, | 145 |
| | | except where * = PS | |
| ODN150C | 5′-G*G*A*ACAGTTAGTCCATa*G*G*C-3′ | PO, | 146 |
| | | except where * = PS | |
| ODN7040 | 5′-CTGCCACGTTCTCCTGCA*C*C*-3′ | PO; * = 2′OMe | 147 |
| ODN7040C | 5′-CTGCCAAGTTCACCTGCA*C*C*-3′ | PO; * = 2′OMe | 148 |
| COMPOUND(I) | 5′-GTCGCCCCTTCTCCCCGCAGC-3′ | Fully PS, with two | 149 |
| | | 5′-methylC (C) in | |
| | | positions 3 and 16 | |
| ODN301C | 5′-GTAGCCCCATCACCCAGCAGC-3′ | Fully PS, | 150 |
| | | GC-containing | |
| ODN302 | 5′-GTCGCTCTTCTCCTCCGCCAG-3′ | Fully PS | 151 |
| ODN2006 | 5′-TCGTCGTTTTGTCGTTTTGTCGTT-3′ | Fully PS, | 152 |
| | | CG-containing | |
| ODN2006C | 5′-TGCTGCTTTTGTGCTTTTGTGCTT-3′ | Fully PS | 153 |
| ODN2216 | 5′-G*G*GGGACGATCGTC*G*G*G*G*G*G-3′ | PO, | 154 |
| | | except where * = PS | |
| ODN1826 | 5′-TCCATGACGTTCCTGACGTT-3′ | Fully PS | 155 |
| ODN1826C | 5′-TCCATGAGCTTCCTGAGCTT-3′ | Fully PS | 156 |
| ODN2137 | 5′-TGCTGCTTTTGTGCTTTTGTGCTT-3′ | Fully PS, | 157 |
| | | GC-containing | |
| ODN2216 | 5′-G*G*GGGACGATCGTCG*G*G*G*G*G*-3′ | PO, | 158 |
| | | except where * = PS | |

DIMS0150 (“Kappaproct®;” ODN150) and BL-7040 (“Monarsen™;” ODN7040) are oligonucleotides that were tested clinically as therapeutic agents for ulcerative colitis. In these clinical studies, ODN150 and ODN7040 showed some moderate beneficial effects in a subset of UC patients. Both ODN150 and ODN7040, without wishing to be bound by theory, are believed to act as TLR9 agonists. ODN150 is a 20-mer ODN having an antisense sequence targeting an mRNA of p65 of NF-kB (RelA). ODN7040 is a 20-mer ODN having an antisense sequence targeting an mRNA of acetylcholinesterase.

ODN2006 is a canonical CpG-B-type TLR9 agonist, which has been developed as an adjuvant in treating cancer.

ODN302 is a scrambled (non-SMAD7 AON) control sequence that contains two CG sequences.

### Cellular Assays

PBMC from healthy donors were isolated from Buffy Coat blood (Blood Center of NY) by density-gradient centrifugation over Ficoll-Hypaque (Pharmacia Biotech). The cells were washed with PBS and re-suspended in RPMI-1640 Media with 10% inactivated fetal bovine serum, penicillin (100 U/ml) and streptomycin (100 μg/ml) and 2 mM L-glutamine (complete medium, Life Technologies). PBMC were treated with ODNs for indicated time periods at indicated concentrations, generally between 0.0001 μM and 10 μM, with CD40L plus anti-HisTag antibody, or with a vehicle control (endotoxin-free sterile water control). No transfection reagent was added with the ODNs.

SMAD7 mRNA levels were determined by RT-PCR using standard protocols.

SMAD7 protein levels were determined using standard FACS protocols. PBMCs were incubated on ice in the presence of Fc-Block (BD Pharmingen) for 20 minutes, washed, and incubated with antibodies specific for surface molecules or isotype-matched control mAbs. PECY-7-labeled anti-human CCR6 mAb, and PerCP-Cy5.5 labeled anti-human CD123 mAb were purchased from BD Biosciences. SMAD7 antibody was purchased from Biorbyt, Cambridge, UK. The stained cells were then subjected to FACS (FACSCanto, BD Biosciences) using the FACSDiva software. FACS analysis was performed using FlowJo software. Single, viable cells in the CCR6+CD123+ pDC gates were used for analysis of, e.g., SMAD7 protein levels.

Effects of ODN Treatments on SMAD7 Expression in Human PBMCs and pDCs

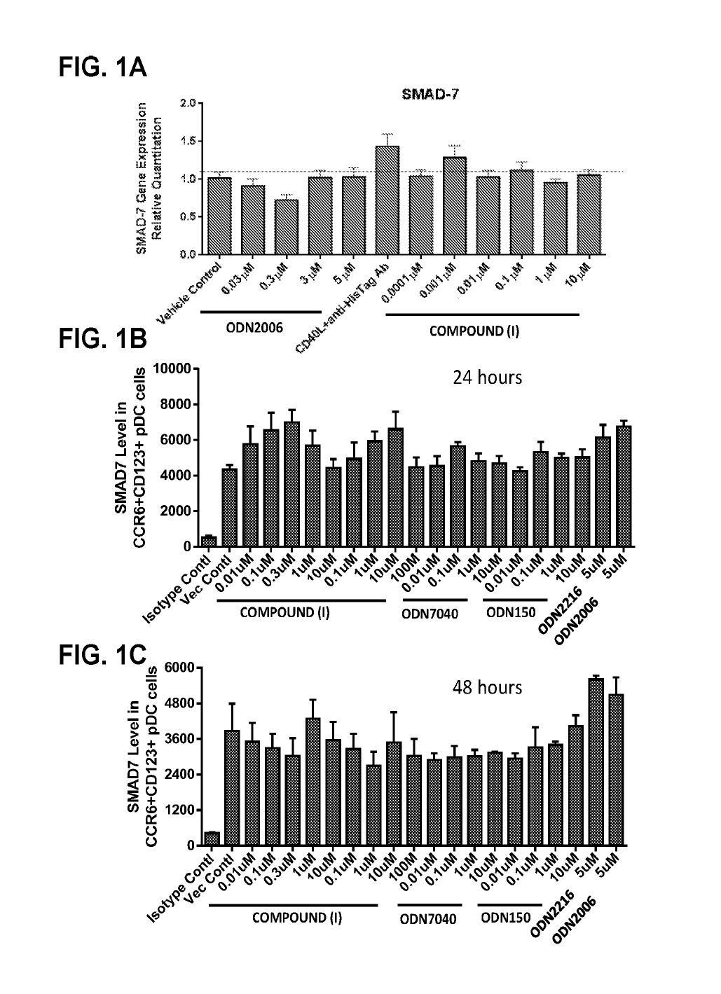

The results shown in FIG. 1A illustrate that COMPOUND (I) and ODN2006 do not affect SMAD7 mRNA levels in normal human PBMCs at any of the indicated concentrations.

The results shown in FIGS. 1A and 1B illustrate that COMPOUND (I), ODN7040, ODN150, ODN2216, and ODN2006 do not affect SMAD7 protein levels in human pDCs at any of the indicated concentrations of treatment periods. Similar results were observed in other normal human PBMCs (not shown), where SMAD7 protein levels are relatively low.

### 8.2 Example 2: COMPOUND (I) Induces TGFβ, PD-L1, IDO, ICOS-L, and IL-10 Expression in Human pDCs

This Example demonstrates COMPOUND (I) can induce TGFβ, PD-L1, IDO, ICOS-L, and IL-10 expression in human pDCs. Specifically, COMPOUND (I) was found to be a more rapid, more potent, and more efficacious activator of, e.g., IDO and ICOSL than other tested TLR9 agonistic ODNs, such as ODN7040 and ODN150.

### Cellular Assays

To evaluate intracellular IL-10, TGFβ 1 and IDO expression in human pDCs, the cells were first stained with PECY-7-labeled anti-human CCR6 mAb (BD Biosciences), PerCP-Cy5.5 labeled anti-human CD123 mAb (BD Biosciences). Stained cells were then fixed and permeabilized using a Cytofix/Cytoperm kit (BD Biosciences) according to the manufacturer's instructions and stained with PE-labeled anti-human IDO (Biolegend), APC-labeled anti-human IL-10 mAb and FITC-labeled anti-human TGF-β1 (BD Biosciences), or isotype-matched control mAbs. The stained cells were acquired by FACSCanto using the FACSDiva software. FACS analysis was performed using FlowJo software. Single, viable cells in the CCR6+CD123+ pDC gates were analyzed. The flow cytometric results are expressed as the percentage and MFI of IL-10-, TGF-β1- or IDO-expressing cells in each gate. Results are expressed as the mean±SEM of cells quantified.

To evaluate surface ICOS-L, PD-L1 expression in the CCR6+CD123+ pDC cell subsets, PBMCs were incubated on ice in the presence of Fc-Block (BD Pharmingen) for 20 minutes, washed, and incubated with antibodies specific for surface molecules or isotype-matched control mAbs. APC-labeled anti-human ICOS-L mAb were purchased from BioLegend, PECY-7-labeled anti-human CCR6 mAb, FITC-labeled anti-human PD-L1 mAb and PerCP-Cy5.5 labeled anti-human CD123 mAb were purchased from BD Biosciences. The stained cells were then subjected to FACS (FACSCanto, BD Biosciences) using FACSDiva software. FACS analysis was performed using FlowJo software. Single, viable cells in the CCR6+CD123+ pDC gates were analyzed. The flow cytometric results are expressed as the percentage and MFI of ICOSL-, or PD-L1-expressing cells in each gate.

IL10 was alternatively measured in cellular supernatants by ELISA using standard protocols.

FIG. 2A illustrates that COMPOUND (I), ODN301C, ODN150, ODN7040, ODN302, and ODN2006 can induce TGF-β1 protein in human pDCs.

FIG. 2B illustrates that COMPOUND (I), ODN301C, ODN150, ODN7040, ODN302, and ODN2006 can induce PD-L1 protein in human pDCs.

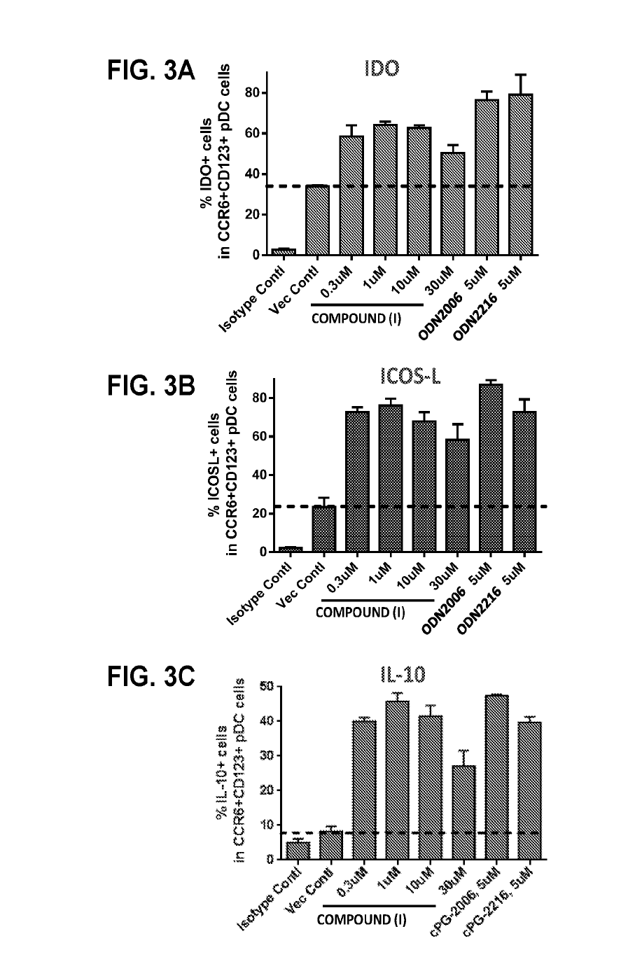

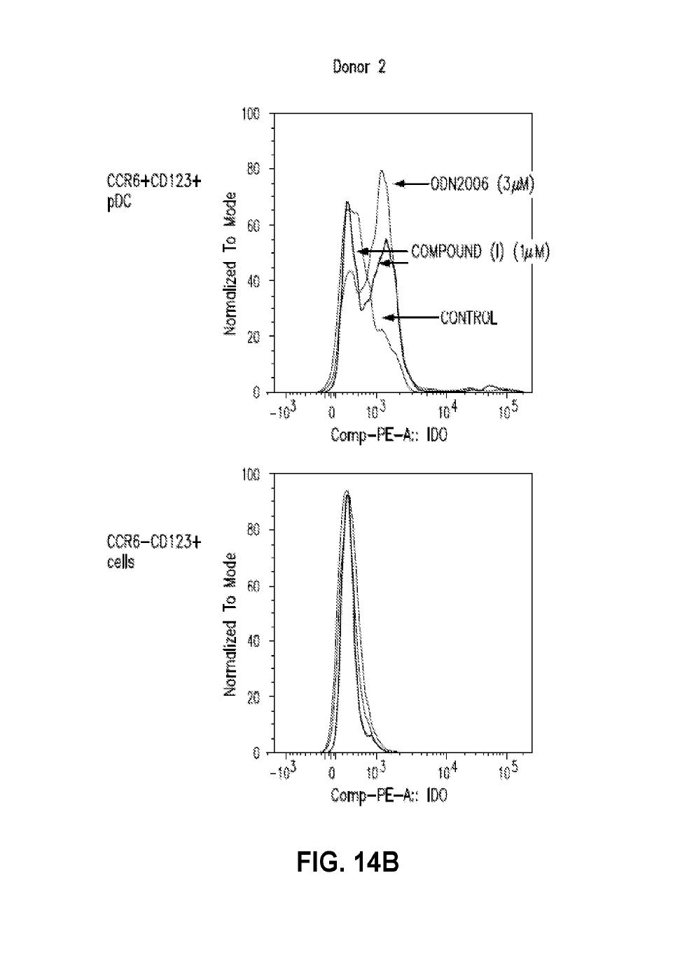

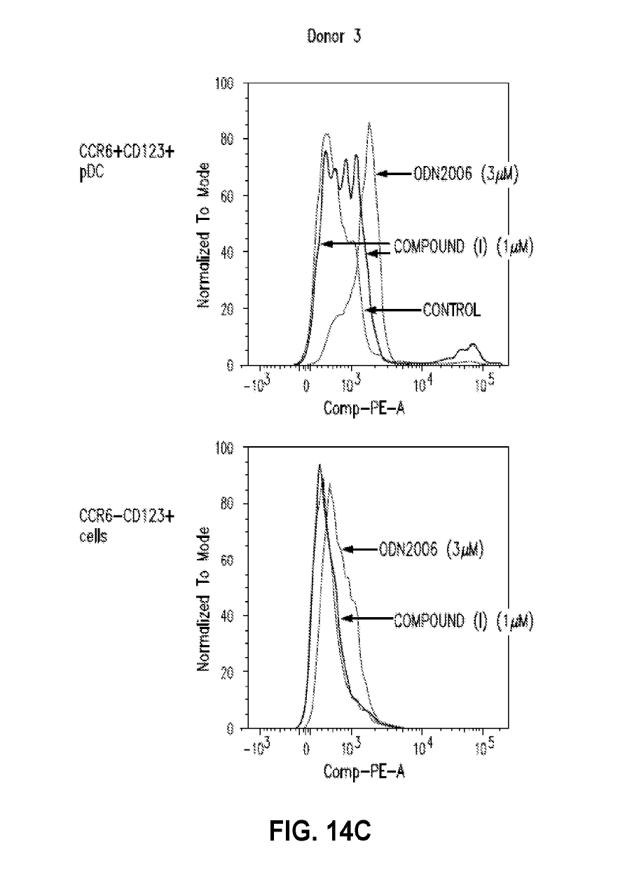

FIG. 3A illustrates that COMPOUND (I), ODN2006 and ODN2216 can induce IDO protein in human pDCs. See also, FIGS. 14A-C.

FIG. 3B illustrates that COMPOUND (I), ODN2006 and ODN2216 can induce ICOS-L protein in human pDCs.

FIG. 3C illustrates that COMPOUND (I), ODN2006 and ODN2216 can induce IL10 protein in human pDCs.

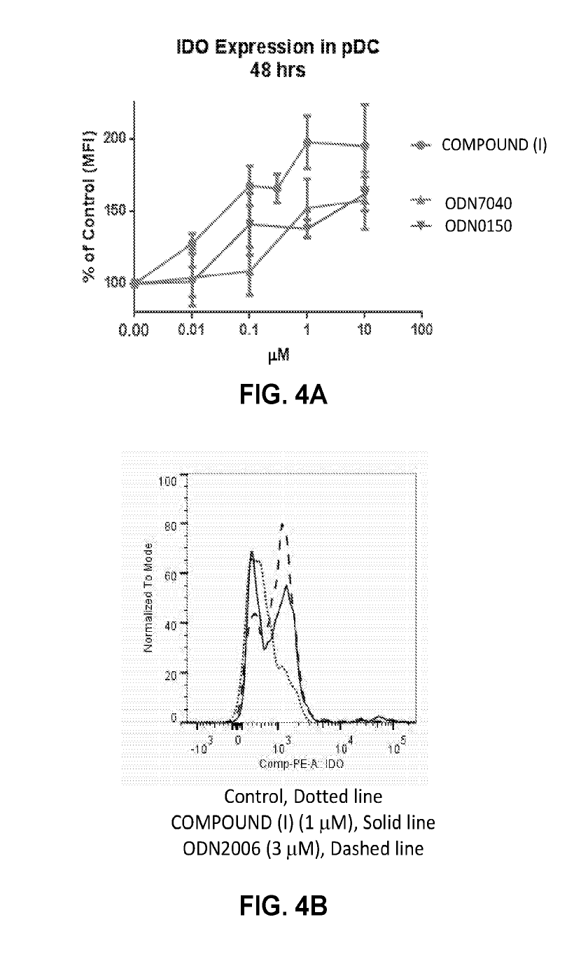

FIG. 4A illustrates that COMPOUND (I) can induce IDO protein expression in human pDCs to higher levels and with greater potency than ODN7040 or ODN150.

FIG. 4B illustrates that COMPOUND (I) can induce IDO protein expression in human pDCs to comparable levels as ODN2006. See also FIG. 14A-C.

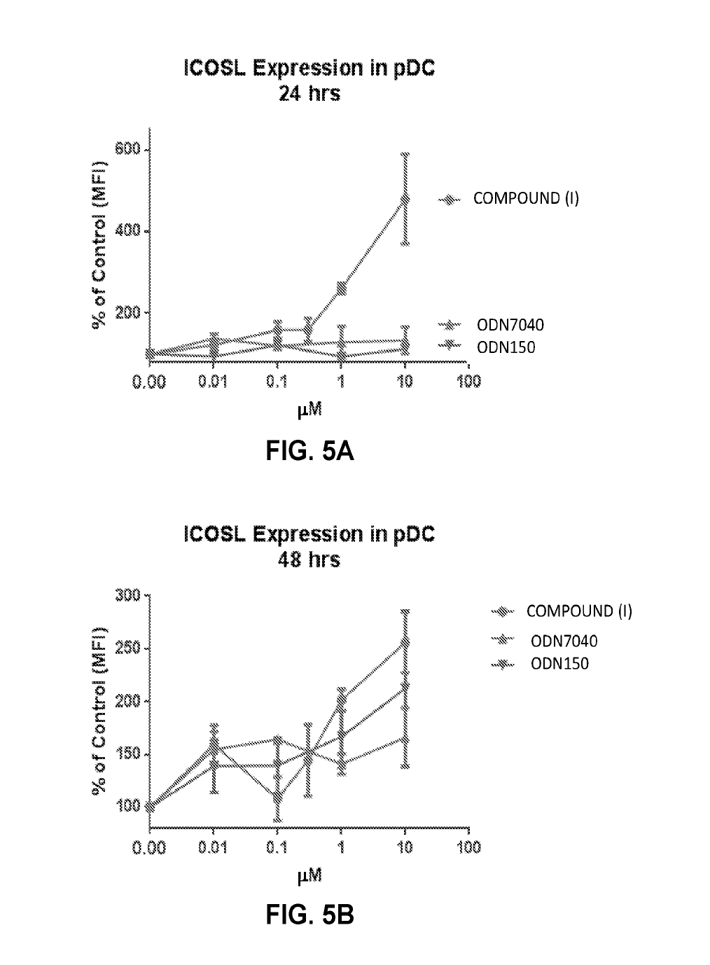

FIGS. 5A and 5B illustrate that COMPOUND (I) can induce ICOS-L protein expression in human pDCs with faster kinetics that ODN7040 and ODN150, e.g., already within 24h of incubation (FIG. 5A). Moreover COMPOUND (I) can induce ICOS-L protein to higher levels than ODN7040 or ODN150 (FIGS. 5A and 5B).

In conclusion, this Example demonstrates that COMPOUND (I) can induce an immunosuppressive pathway in human pDCs that promote differentiation into tolerogenic pDCs and induction of regulatory T-cells. As shown in this Example, COMPOUND (I) stimulated pDCs can produce IDO, IL-10, TGF-β and ICOS-L, which downstream can promote differentiation of regulatory T-cells (Tregs). Especially COMPOUND (I)-mediated TGF-β and IDO induction in human pDCs can be a key aspect for tolerogenic pDC and Treg induction.

### 8.3 Example 3: Hydroxychloroquine Acts Synergistically with COMPOUND (I) to Induce ICOS-L Protein Expression in Primary Human pDCs

This Example illustrates the surprising result that a TLR antagonist, such as the TLR7 and TLR9 antagonist hydroxylchloroquine (plaquenil; HCQ), can act synergistically with a SMAD7 AON, such as COMPOUND (I), to activate primary human pDCs. Specifically, hydroxychloroquine was found to boost the COMPOUND (I)-induced protein expression of ICOS Ligand (ICOS-L) on primary human pDCs. ICOS-L is generally considered a costimulatory factor that is involved in the induction of regulatory T-cells (Tregs) by tolerogenic pDCs.

pDCs were isolated and ICOS-L expression was analyzed as described in Example 2.

FIGS. 6A-B illustrate the surprising result that the efficacy of a SMAD7 AON, such as COMPOUND (I), can be increased by a concomitant treatment with hydroxylchloroquine (plaquenil). COMPOUND (I) was tested at concentrations of 1 μM and 10 μM, i.e., under conditions where COMPOUND (I) by itself induced moderate levels of ICOS-L expression in human pDCs. See, e.g., FIG. 6B (compare bars no. 6 and no. 7 from left relative to vehicle control). HCQ was applied at a concentration of 10 μM and under conditions where HCQ alone did not induce measurable levels of ICOS-L expression in pDCs. See, e.g., FIG. 6B (compare bar no. 8 from left with vehicle control). Under these conditions, HCQ increased COMPOUND (I) induced ICOS-L expression by 7.3-fold and 7.1-fold (at 1 μM and 10 μM, respectively) in one Experiment (FIG. 6A), and by 2.0-fold and 1.9-fold (at 1 μM and 10 μM, respectively) in another Experiment (FIG. 6B).

Without wishing to be bound by theory, induction of regulatory T-cells (Tregs) by tolerogenic pDCs is commonly believed to suppress immune responses resulting in amelioration of immune-mediated diseases.

### 8.4 Example 4: COMPOUND (I) Induces TGFβ, PD-L1, IDO, ICOS-L, and IL-10 Expression in Human PBMCs

This Example illustrates that COMPOUND (I) can act as a modest inducer of certain cytokines, including IP10, L6 and TNFα when administered ad low doses (0.1 μM) to normal human PBMCs. When administered at high doses (10 μM), COMPOUND (I) can inhibit the induction of certain cytokines, such as IP-10, and induce strong expression of other cytokines, including TNFα, IFNγ, and IL-1β.

Cytokines induced by at low-dose (0.1 μM) and high-dose (10 μM) ODNs were compared.

PBMC from 3 donors were isolated from Buffy Coat (Blood Center of NY). The cells were washed with PBS and re-suspended in RPMI-1640 Media with 5% Human AB Serum, 1% Pen/Strep and 2 mM L-glutamine. The cells were then plated in 96-well plates at 250,000 cells/well. PBMC's were treated with 0.1 μM or 10 μM of ODN2006, ODN2216, COMPOUND (I), ODN7040, ODN7040C, ODN150, ODN150C, ODN301C and ODN302 and then incubated for 24 hrs at 37° C. After 24 hrs, the supernatant was harvested and tested for cytokine production using MagPix Multi-Plex Technology. The following cytokines were tested: IL-6, IL-10, IL-12p40, IFN-α, IFN-γ, IP-10, IL-1β and TNF-α.

Table 3 shows results for cytokines induced by low-dose ODN treatments. Table 4 shows results for cytokines induced by high-dose ODN treatments.

TABLE 3
Cytokines induced by low-dose ODNs (0.1 μM)
(The values of IP10, TNF-α, IL-6, and IL-10 are fold change.
The values of IFN-γ, IL-12p40, and IL-1β are pg/ml)

| ODN | IP10 | TNF-α | IL-6 | IL-10 | IFN-γ | IL-12p40 | IL-1β |
|---|---|---|---|---|---|---|---|
| ODN2006 | 19 | 5.1 | 57 | 2.4 | 4 | 5.5 | 1.4 |
| ODN302 | 13.9 | 2.4 | 19 | 1.5 | 1.5 | 0.7 | 0.2 |
| COMPOUND | 3.5 | 1.8 | 2.5 | 1 | 0.6 | 0.1 | 1.1 |
| (I) | | | | | | | |
| ODN7040 | 4.3 | 1.3 | 1.3 | 1 | 0.2 | 0.4 | 0.4 |
| ODN150 | 1.5 | 1.2 | 1.2 | 0.9 | 0.2 | 0 | 0.2 |
| ODN2216 | 1.7 | 1.4 | 1.3 | 1 | 0.3 | 0.1 | 0.4 |
| ODN301C | 1.1 | 1.1 | 1.3 | 1 | 0 | 0 | 0.1 |
| ODN7040C | 1.1 | 1.1 | 1.2 | 1 | 0.2 | 0.1 | 0.6 |
| ODN150C | 1 | 1.2 | 1.2 | 1 | 0.1 | 0 | 0.2 |

Table 3 illustrates that among the tested ODNs ODN2006 and ODN302 showed strong upregulation of certain immunostimulatory cytokines in pDCs, especially of IP10 and IL6, as well as TNFα. By comparison, COMPOUND (I) was found to only moderately upregulate IP10, IL6 and TNFα production. ODN7040 was found to only induce IP10 production, and only at moderate levels.

TABLE 4
Cytokines induced by high-dose ODNs (10 μM)
(The values of IP10, TNF-α, IL-6, and IL-10 are fold change.
The values of IFN-γ, IL-12p40, and IL-1β are pg/ml)

| ODN | IP10 | TNF-α | IL-6 | IL-10 | IFN-γ | IL-12p40 | IL-1β |
|---|---|---|---|---|---|---|---|
| ODN2006 | 0.13 | 12.5 | 25 | 0.9 | 6 | 6.5 | 3.2 |
| ODN302 | 4.4 | 9.3 | 41 | 1.5 | 9.7 | 5.2 | 3.9 |
| COMPOUND | 0.06 | 5.2 | 5.4 | 1 | 6 | 1.1 | 2.3 |
| (I) | | | | | | | |
| ODN7040 | 3.2 | 1.2 | 3.3 | 1.2 | 0.5 | 0.3 | 0.6 |
| ODN150 | 8.1 | 2 | 11.8 | 1.6 | 0.2 | 0 | 0.6 |
| ODN2216 | 36 | 5.6 | 1.3 | 1 | 0.3 | 3 | 1 |
| ODN301C | 0.25 | 2.8 | 3.3 | 1 | 0 | 0.8 | 1.9 |
| ODN7040C | 1.4 | 1.4 | 2.1 | 1 | 0.2 | 0.1 | 0.5 |
| ODN150C | 1.5 | 1.4 | 1.4 | 1 | 0.1 | 0 | 0.3 |

Table 4 illustrates that at high doses ODN2006 and COMPOUND (I) can inhibit IP-10 protein expression in pDCs, whereas ODN7040 and ODN150 were found to induce IP-10 protein expression in PBMCs. Moreover, at high doses COMPOUND (I) was found to be a stronger inducer of TNFα, IFNγ, and IL-1β than ODN7040 or ODN150.

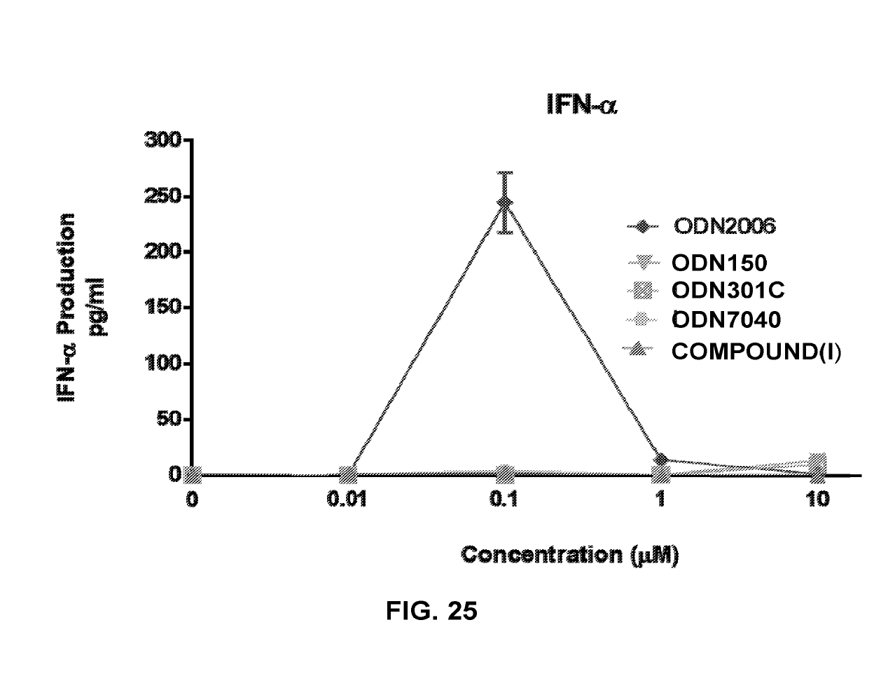

FIG. 25 shows that ODN2006 can induce IFNα secretion from PBMCs when incubated with the PBMCs for 24 hrs at a concentration of 0.1 μM. ODN2006 was not found to induce IFNα secretion from PBMCs at lower or higher concentrations, e.g., a 0.01 μM, 1.0 μM, or 10 μM. COMPOUND(I), ODN150, ODN301C and ODN7040 were not found to induce IFNα secretion from PBMCs at any tested concentrations between 0.01 μM and 10 μM.

### Example 5: COMPOUND (I) Increases B-Cell Activation in Human PBMCs

This Example illustrates that COMPOUND (I) can moderately induce B-cell activation in normal human PBMCs.

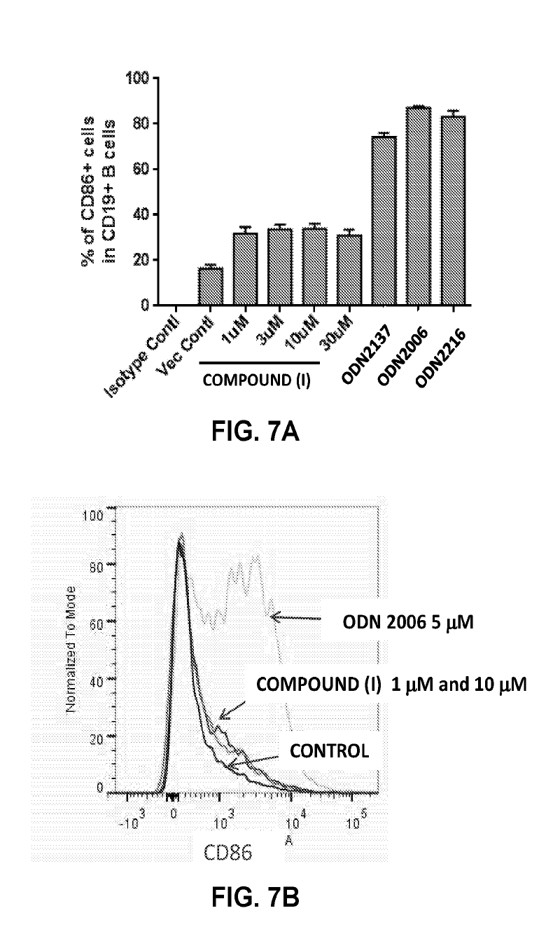

To investigate whether COMPOUND (I) can increase B-cell activation, CD86 expression levels were analyzed in CD19⁺ B-cell subsets. PBMC were incubated on ice in the presence of Fc-Block (BD Pharmingen) for 20 minutes, washed, and incubated with antibodies specific for surface molecules or isotype-matched control mAbs. APC-Cy7 labeled anti-human CD19 mAb and PE-labeled anti-human CD86 mAb were purchased from BioLegend. The stained cells were then subjected to FACS (FACSCanto, BD Biosciences) using the FACSDiva software. FACS analysis was performed using FlowJo software. Single, viable cells in the CD19+ B-cells gates were used for analysis. The flow cytometric results are expressed as the percentage of CD86-expressing cells (FIG. 7A) as the mean fluorescence intensity (MFI) of CD86-expressing cells in each gate (FIG. 7B). FIG. 7A shows a bar diagram illustrating the results of CD86⁺ cells in the CD19⁺ B-cell population. FIG. 7B shows exemplary FACS traces for selected experiments represented in FIG. 7A (COMPOUND (I) at 1 μM and 10 μM; ODN2006 at 5 μM).

FIG. 7 illustrates that COMPOUND (I) can increase B-cell activation in human PBMCs, as determined by CD86 expression. CD86 upregulation was found to be modest after 24 h (FIG. 7B) and was further increased at 36 h (not shown). Stronger B-cell activation was observed using ODN2137, ODN2006, or ODN2216 (FIG. 7A).

### 8.5 Example 6: COMPOUND (I) Inhibits IFNα Induced Through TLR3, TLR7 and TLR9 Pathways

This Example illustrates that COMPOUND (I) can inhibit activation of certain TLR pathways, including TLR3 TLR7 and TLR9 pathways.

The effect of COMPOUND (I) on TLR3, TLR7, and TLR9-mediated IFNα induction in PBMCs was analyzed. The cells were treated with COMPOUND (I) (0.0001 μM-10 μM) for 1 hour then stimulated with 10 μg/ml of polyl:C (TLR3), 5 μg/ml of Imiquimod (TLR7), or 6.3 μg/ml ODN2216 (TLR9) for 24 hours. After 24 hours, the supernatant was harvested and tested for IFNα using MagPix Multi-Plex technology.

FIG. 8 shows experimental results illustrating that COMPOUND (I) can inhibit the induction of IFNα through TLR3 (FIG. 8A), TLR7 (FIG. 8B) and TLR9 (FIG. 8C) pathways. COMPOUND (I) was found to be a more potent inhibitor of TLR3-mediated IFNα induction (IC₅₀˜0.1 μM; FIG. 8A) and TLR7-mediated IFNα induction (IC₅₀<0.1 μM; FIG. 8B) than TLR9-mediated IFNα induction (IC₅₀˜1.0 μM; FIG. 8C).

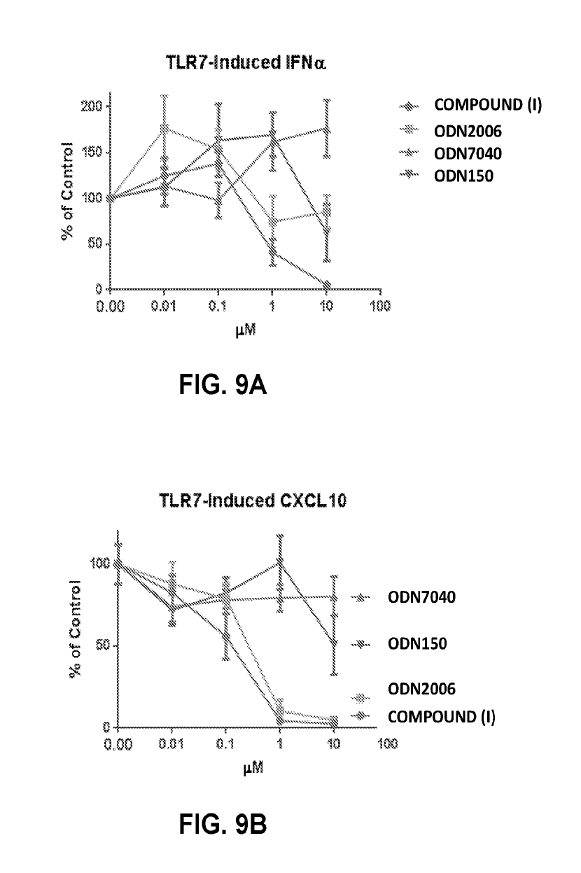

FIG. 9 shows experimental results illustrating that COMPOUND (I) is about as potent an inhibitor of TLR7-mediated IFNα induction as ODN2006 (FIG. 9A; IC₅₀<1 μM) and about as potent an inhibitor of TLR7-mediated IP10 (CXCL10) induction as ODN2006 (FIG. 9B; IC₅₀<1 μM). COMPOUND (I) and ODN2006 were found to be at least 10-fold more potent inhibitors of TLR7-mediated IFNα and IP10 induction than BL-7040 (IC₅₀˜10 μM) and ODN150 (no inhibitory activity up to 10 μM ODN150).

### 8.6 Example 7: COMPOUND (I) does not Activate the Canonical NF-kB Pathway

This Example demonstrates that COMPOUND (I) does not activate the canonical NF-KB pathway, suggesting that the effects of COMPOUND (I) on cytokine expression, e.g., of IDO, ICOS-L, TGFβ, in human PBMCs, described, e.g., in Examples 2-4 may be mediated through the non-canonical NF-κB pathway.

Mouse RAW264.7 macrophage cells containing a reporter construct driven by a canonical NF-κB promoter were plated in 96-well plates at 50,000 cells/well. The cells were treated with 0.1 μM, 1 μM, 10 μM, 30 μM, or 100 μM of ODN7040, COMPOUND (I), ODN150, and ODN302, and then incubated for 24 hrs. The supernatant was harvested and tested for optical density.

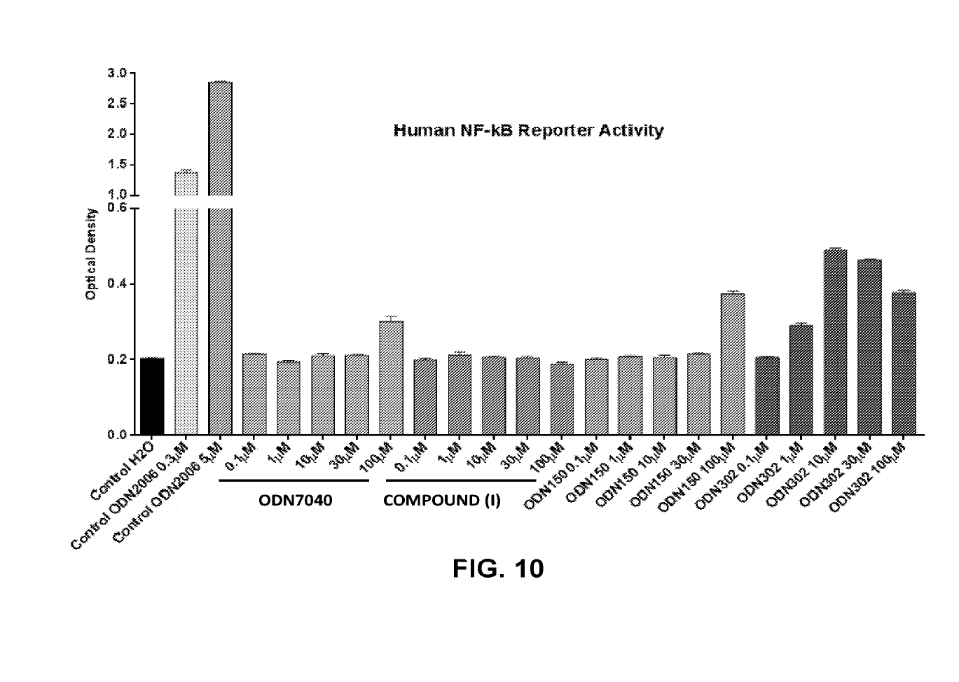

FIG. 10 shows results illustrating that COMPOUND (I) did not activate the NF-κB construct in mouse RAW264.7 macrophage cells at concentration of up to 100 μM (FIG. 10). Moderate activation of the NF-κB construct was observed for ODN302 starting at a ODN concentration of about 1 μM and for ODN7040 and ODN150 at much higher concentrations (100 μM).

In addition, the effect of COMPOUND (I) on canonical NF-κB promoter activity in mouse macrophage Raw264 cells was tested. Mouse Raw Blue Reporter Cells (Invivogen), containing a secreted embryonic alkaline phosphatase (SEAP) reporter gene driven by canonical NF-κB promoter, were plated in 96-well plates at 50,000 cells/well in DMEM Media supplemented with 10% FBS, 2 mM L-glutamine, 1% Pen/Strep, 100 mg/ml Normacin. The cells were treated with 0.01 μM to 10 μM or 0.1 μM to 100 μM of ODN1826, ODN1826C, COMPOUND (I), ODN7040, ODN7040C, ODN150, ODN150C, ODN302, and ODN301C, and then incubated for 24 hrs. The supernatant was harvested and tested for SEAP production by measuring OD at 630 nM.

FIG. 11 shows results illustrating that COMPOUND (I) does not activate a canonical NF-κB reporter in mouse macrophages at concentrations of up to 10 μM. By contrast, ODN1826 was found to be a potent activator of the NF-κB reporter in mouse macrophages, even at concentrations as low as 0.01 μM. ODN7040 and ODN150 were found to activate the NF-κB reporter in mouse macrophages starting at concentration of 1 μM (ODN7040) or higher 10 μM (ODN150). The NF-κB activating effects of ODN7040 and ODN150 were found to be dependent on the presence of CG-dinucleotide sequences. ODN7040C and ODN150C control oligonucleotides, which lack CG-dinucleotide sequences, did not activate the canonical NF-κB reporter in mouse macrophages.

The effect of COMPOUND (I) on induction of TNFα or IL-10 was further explored in mouse macrophages. Mouse Raw Blue Reporter Cells (Invivogen) were plated in 96-well plates at 50,000 cells/well in DMEM Media supplemented with 10% FBS, 2 mM L-glutamine, 1% Pen/Strep, 100 mg/ml Normacin. The cells were treated with 0.01 μM to 10 μM or 0.1 μM to 100 μM of ODN1826, ODN1826C, COMPOUND (I), ODN7040, ODN7040C, ODN150, ODN150C, ODN302, and ODN301C, and then incubated for 24 hrs. The supernatant was harvested and tested for TNFα and IL-10 production using MagPix Multi-Plex technology.

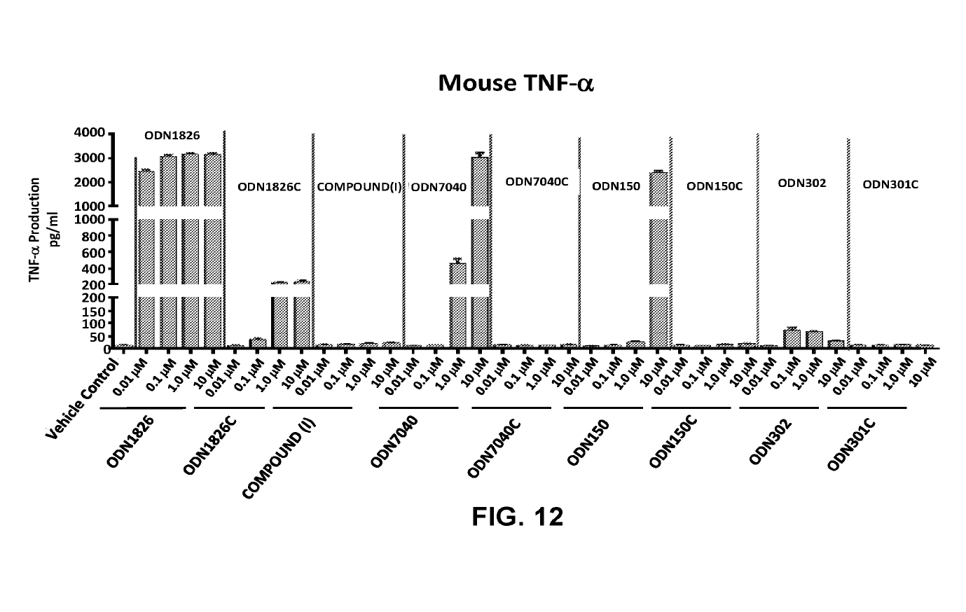

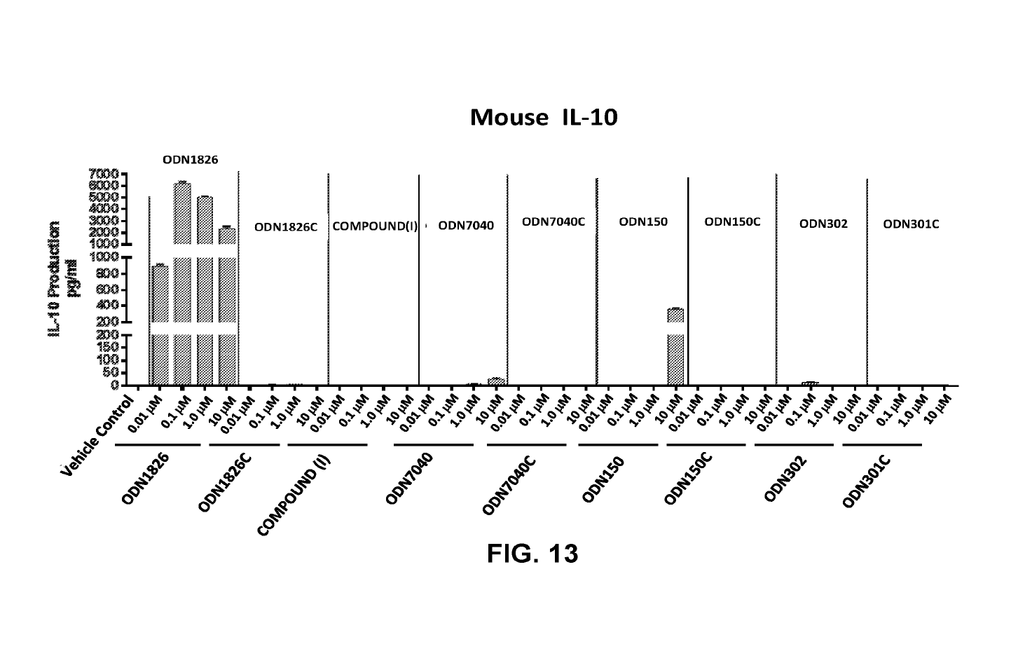

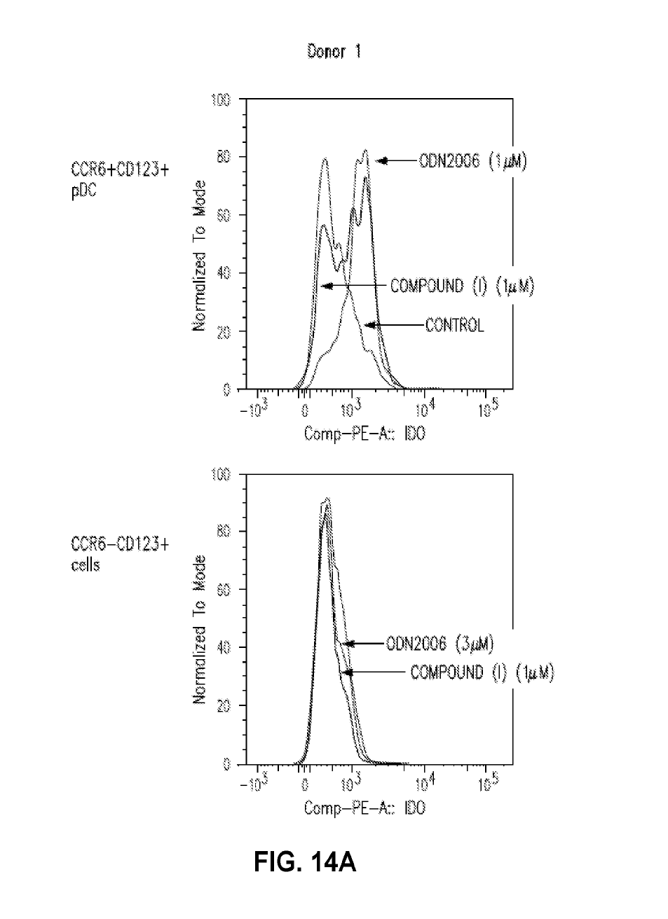

FIGS. 12 and 13 show results illustrating that COMPOUND (I) does not induce TNFα or IL-10 protein expression in mouse macrophages up to concentrations of 10 μM. These results are consistent with the lack of canonical NF-κB activation by COMPOUND (I) in mouse macrophages, which drives, e.g., TNFα expression. Consistent with their observed activity on canonical NF-κB activation, ODN1826, ODN7040 and ODN150 were found to induce TNFα and IL-10 protein expression in mouse macrophages.

In conclusion, without wishing to be bound by theory, the Examples described herein, illustrate the following:
COMPOUND (I) can induce IDO, a key mediator of immunosuppressive activity, e.g., in pDCs. COMPOUND (I) can induce ICOS-L, a key mediator of regulatory T-cell induction. COMPOUND (I) can induce IL-10 in cells of the immune systems, such as PBMCs or pDCs. These activities of COMPOUND (I) demonstrate that COMPOUND (I) can activate TLR9 and exert immunosuppressive activity.
COMPOUND (I) does not generally stimulate canonical NF-kB signaling in mouse or human reporter cell lines. This observation is consistent with COMPOUND (I)'s ability to induce IDO and to exert immunosuppressive activity, which are mediated through noncanonical NF-kB signaling downstream of, e.g., TLR9.
COMPOUND (I) can act as an inhibitor of TLR7 signaling.
COMPOUND (I), a SMAD7 AON, can have a dual mechanism of action, including the downregulation of SMAD7 mRNA expression through an antisense mechanism, and COMPOUND (I)'s activity as a TLR modulator, e.g., as a TLR9 and/or TLR7 modulator.

### 8.7 Example 8: COMPOUND (I) Cellular Activity Profile is Differentiated from Cellular Activity Profiles of CpG-A and CpG-B Oligonucleotides

This Example demonstrates that COMPOUND (I) can activate TLR9 signalling in immune cells from peripheral blood. This Example further demonstrates that the cellular activity profile of COMPOUND (I) is highly differentiated from the cellular activity profile of other TLR9 agonists of the CG-oligonucleotide (CG ODN) class, including ODN2216 (Class A ODN) and ODN2006 (Class B ODN). COMPOUND (I)'s activities with respect to TLR9 and inflammasome activation in immune cells can be beneficial for the treatment of diseases such as IBD, e.g., through the promotion of intestinal tissue repair.

Generally, CG ODNs are considered to be a class of short synthetic single-stranded DNA molecules containing unmethylated CG dinucleotides (CG motifs). CG ODNs commonly have patially or completely phosphorothioated (PS) backbones. CG ODNs are often classified into three categories based on their structural characteristics and activity on human PBMCs, in particular B-cells and pDCs:
Class A ODNs (“CpG-A”) are characterized by a PO central CG-containing palindromic motif and a PS-modified 3′ poly-G string. CpG-A ODNs generally induce high IFN-α production from pDCs and are generally weak stimulators of TLR9-dependent NF-κB signaling or pro-inflammatory cytokine (e.g., IL-6) production.
CpG-B ODNs contain a full PS backbone with one or more CG dinucleotides. CpG-B ODNs generally strongly induce B-cell proliferation, pDC activation and differentiation and TLR9-dependent NF-κB signaling, while only weakly stimulating IFN-α secretion.
CpG-C ODNs combine features of both classes of A and B. CG-ODNs contain a complete PS backbone and a CG-containing palindromic motif. C-class CG ODNs generally induce strong IFN-α production from pDC and strong B-cell stimulation.

Table 5 compares the relative cellular activities observed for COMPOUND (I) with typical activities for CpG-A ODN (e.g., ODN2216) and CpG-B ODN (e.g., ODN2006) reference modulators of TLR9. Cellular activities of CpG-A, CpG-B (and CpG-C) ODNs are described in the art. See, e.g., Vollmer et al., Characterization of three CpG oligonucleotide classes with distinct immunostimulatory activities. EUR. J. IMMUNOL. Vol. 34:251-262 (2004). Specifically, COMPOUND (I) was found to share a number of immune-cell related activities with ODN2006, including the ability to induce the so-called inflammasome protein complex (IL-1β/IL-18 secretion) in human PBMCs, and the ability to induce human pDC differentiation. However, the activity profile of COMPOUND (I) was distinguishable from the ODN2006 profile, e.g., based on COMPOUND (I)'s much lower activity in inducing B-cell proliferation (no detectable activity) or in activating the NF-κB pathway (low activity). The activity profile of COMPOUND (I) was distinguishable from a typical CpG-A (e.g., ODN2216) profile, e.g., based on COMPOUND (I)'s comparatively lower activity in inducing IFN-α, IL-6, or L-12 secretion from human pDCs or PBMCs, and COMPOUND (I)'s comparatively greater activity in inducing pDC differentiation. The observed activity profile of COMPOUND (I) is distinguishable from a typical activity profile of CpG-C ODNs, e.g., based on COMPOUND (I)'s relatively lower activity with respect to IFN-α induction, e.g., in PBMCs.

TABLE 5
Comparison of Cellular Activities of COMPOUND
(I) and CpG-A and CpG-B Oligonucleotides and

| Cellular | | | | COMPOUND |
|---|---|---|---|---|
| Activity | Cell Type | CpG-A | CpG-B | (I) |
| TLR pathway | Reporter Cell | ↑ ↑ | ↑ ↑ ↑ ↑ | ↑ |
| activation | | | | |
| (NF-κB) | | | | |
| B-cell | B-Cell | — | ↑ ↑ ↑ ↑ | — |
| proliferation | | | | |
| IFNα | pDC | ↑ ↑ ↑ ↑ | ↑ ↑ | — |
| IL-6, IL-12 | PBMC | ↑ ↑ ↑ | ↑ ↑ ↑ ↑ | ↑ |
| TNFα, IFNγ | PBMC | ↑ ↑ ↑ | ↑ ↑ ↑ ↑ | ↑ ↑ |
| IL-1β | PBMC | ↑ ↑ | ↑ ↑ ↑ | ↑ ↑ ↑ |
| pDC | pDC | ↑ | ↑ ↑ ↑ ↑ | ↑ ↑ ↑ |
| differentiation | | | | |

Relative strengths of cellular activities of CpG-A, CpG-B, and COMPOUND (I) in indicated assays are illustrated by one or more arrows, with increasing numbers of arrows indicating increased relative activity. For example, (↑) indicates a weak but detectable activity, (↑↑) and (↑↑↑) indicate different levels of moderate activities, (↑↑↑↑) indicates a strong activity, and (−) indicates no detectable activity. Reported activities represent activities in cell-based assay formats described throughout the examples provided herein, e.g., ELISA, FACS, reporter gene or thymidine incorporation assays. Exemplary data is described herein, e.g., in Examples 4, 7, 9, and 12.

In conclusion, this Example demonstrates that COMPOUND (I) has a cellular activity profile that overlaps with the profile of ODN2006, a canonical CpG-B-type TLR9 activator. However, the cellular activity profile of COMPOUND (I) can be differentiated from the ODN2006 profile, e.g., based on COMPOUND (I)'s low or absent activity on B-cell proliferation.

### 8.8 Example 9: COMPOUND (I) Cellular Activity Profile is Differentiated from Cellular Activity Profiles of ODN150 and ODN7040 Oligonucleotides

This Example demonstrates that the cellular activity profile of COMPOUND (I) is differentiated from the cellular activity profiles of ODN150 (Kappaproct®) and ODN7040 (Monarsen™).

Tables 6 and 7 show summaries of relative cellular activities of COMPOUND (I), ODN150, and ODN7040. The activity profile of COMPOUND (I) was distinguishable from the ODN150 and ODN7040 profiles, e.g., based on COMPOUND (I)'s ability to induce strong inflammasome activity in PBMCs and to strongly induce pDC differentiation, as compared to ODN150 and ODN7040. See Table 6. In addition, COMPOUND (I) was observed to have much weaker, if any, activity with respect to IFN-α induction in pDCs, as compared to ODN150 and ODN7040. See Table 6.

With respect to TLR modulation COMPOUND (I) was found to share some TLR9 agonistic properties with ODN150 and ODN7040. In addition COMPOUND (I) was found to share TLR3 and TLR7 antagonistic activity with ODN150. The TLR modulation profile of COMPOUND (I) was distinguishable from the ODN150 profile, e.g., based on the much stronger TLR8 agonistic activity of ODN150 compared to COMPOUND (I). The TLR modulation profile of COMPOUND (I) was distinguishable from the ODN7040 profile, e.g., based on the stronger TLR3 and TLR7 antagonistic activity of COMPOUND (I).

TABLE 6
Comparison of Cellular Activities of COMPOUND (I), ODN150 and ODN7040

| Cellular | | ODN150 | ODN7040 | COMPOUND |
|---|---|---|---|---|
| Activity | Cell Type | (Kappaproct ®) | (Monarsen ™) | (I) |
| TLR pathway | Reporter Cell | ↑ ↑ | ↑ | ↑ |
| activation | | | | |
| (NF-κB) | | | | |
| B-cell | B-Cell | — | — | — |
| proliferation | | | | |
| IFN-α | pDC | ↑ ↑ | ↑ ↑ | — |
| IL-6, IL-12 | PBMC | ↑ | —/↑ | ↑ |
| TNFα, IFNγ | PBMC | —/↑ | —/↑ | ↑ ↑ |
| IL-1β | PBMC | — | — | ↑ ↑ ↑ |
| pDC | pDC | ↑ | ↑ | ↑ ↑ ↑ |
| differentiation | | | | |

Relative strengths of cellular activities of ODN150, ODN7040, and COMPOUND (I) in indicated assays are illustrated by one or more arrows, with increasing numbers of arrows indicating increased relative activity. For example, (↑) indicates a weak but detectable activity, (↑↑) and (↑↑↑) indicate different levels of moderate activities, (↑↑↑↑) indicates a strong activity, and (−) indicates no detectable activity. (−/↑) indicates a low activity close to the detection limit. Reported activities represent activities in cell-based assay formats described throughout the examples provided herein, e.g., ELISA, FACS, reporter gene or thymidine incorporation assays. Exemplary data is described herein, e.g., in Examples 4, 7, 9, and 12.

TABLE 7
Comparison of COMPOUND (I), ODN150, and ODN2006
Activities in the Modulation of TLR/NOD-Induced
NF-κB in TLR-Expressing Cell Lines

| | COMPOUND (I) | | ODN150 | | ODN7040 | |
|---|---|---|---|---|---|---|
| | | Antag- | | Antag- | | Antag- |
| | Agonism | onism | Agonism | onism | Agonism | onism |
| TLR9 | + | −− | ++ | −− | ++ | −− |
| TLR7 | −− | ++ | −− | ++ | −− | −− |
| TLR8 | −− | NA | ++ | NA | −− | NA |
| TLR3 | −− | ++ | −− | ++ | −− | + |

Relative strengths of TLR modulating activities of COMPOUND (I), ODN150 and ODN2006 in recombinant TLR-expressing HEK293 reporter cell lines are indicated by one or more plus symbols (+).
Minus symbols (−−) indicate no activity.
NA means that the experimental condition was not analyzed.
Exemplary data is described herein, e.g., in Example 6.

FIG. 15 shows results of an experiment in which human PBMCs were incubated for 24 hours with the indicated concentrations of COMPOUND (I) or oligonucleotides ODN150, ODN2006, or ODN7040, and IL-1β secretion was measured by ELISA. The results show that COMPOUND (I) potently induced IL-1β in human PBMCs. COMPOUND (I) induced IL-1β with a potency similar to that of the canonical CpG-B oligonucleotide ODN2006. ODN301C was also found to induce IL-1β, demonstrating that neither a SMAD7 nor a CG-containing ODN sequence is required for IL-1β induction in this assay. Other TLR9 modulators, such as ODN150 and ODN7040, did not induce IL-1β.

It is generally believed that IL-1β secretion, e.g., from PBMCs, requires activation of the so-called inflammasome, a protein complex implicated in IBD. Human and mouse genetics of IBD appear to be consistent with the notion that an impaired inflammasome-mediated immune response represents an underlying defect in IBD. Other inflammasome components or mediators that have been identified as containing possible risk alleles in IBD include, e.g., NACHT, LRR and PYD domains-containing protein 3 (NLRP3), IL-18, IL-18RAP, and nucleotide-binding oligomerization domain-containing protein 2 (NOD2), also known as inflammatory bowel disease protein 1 (IBD1). The activity of COMPOUND (I) with respect to inflammasome activation can be beneficial in the treatment of IBD through the promotion of inflammasome mediated tissue repair.

FIGS. 16A-B show results of experiments in which recombinant TLR-expressing HEK293 cells carrying an NF-κB reporter gene were incubated with COMPOUND(I), ODN150, ODN2006, or ODN7040 for 48 hours at the indicated concentrations. The results show that COMPOUND (I) activated the NF-κB reporter gene in a TLR9-dependent manner. See FIG. 16A. COMPOUND (I) activity is inhibited by hydroxychloroquine. However, COMPOUND (I) activated NF-κB to much lower levels of activation than ODN150, ODN2006, or ODN7040. See FIG. 16B.

FIG. 17 shows results of an experiment in which purified human B-cells were incubated for 96 hours at the indicated concentrations with COMPOUND (I), ODN150, ODN302, ODN2006, or ODN2008C. B-cells were isolated from 3 donors of buffy coat (Blood Center of NY) using the RosetteSep™ Human B Cell Enrichment Kit (Stem Cell, Cat#15024) and following the manufacturers' procedures. The B Cells were then cultured in RPMI-1640 medium supplemented with 10% FBS, 2 mM L-glutamine, 1 mM Sodium Pyruvate and 1% Pen/Strep. Purified B cells were plated at 100,000 cells/well in 180 μl using 96-well flat bottom plates. The cells were immediately treated with 20 μl 10× COMPOUND (I), ODN301C, DIMS0150, ODN2006, and ODN7040. The final concentrations were 0, 0.0001, 0.001, 0.01, 0.1, 1, 10 μM. The plates were incubated for 96 hrs at 37° C., 5% CO₂. After 72 hrs, tritiated thymidine (Perkin Elmer, stock concentration of 1 mCi/ml) was added at 1 μCi/well (1:20 dilution) in Complete RPMI-1640 media and incubated at 37° C. for 24 hrs. After 24 hrs, the plates were harvested on the cell harvester, air dried overnight and analyzed on the Top Count Reader (added 20 μl scintillation fluid prior to analysis). The results show that COMPOUND (I) did not detectably induce B-cell proliferation.

FIG. 18 shows results of an experiment in which human purified pDCs were incubated with COMPOUND (I), ODN150, ODN2006, or ODN7040, in the presence or absence of IL-3, for 48 hours, at indicated concentrations, and IFN-α secretion was measured by ELISA. The results show that COMPOUND (I) did not detectably induce IFNα secretion from purified pDCs (no IL-3) or from pDCs matured by IL-3 treatment. By contrast, ODN2006 induced IFN-α secretion from purified and matured pDCs, and ODN150 and ODN7040 induced IFN-α secretion from matured pDCs.

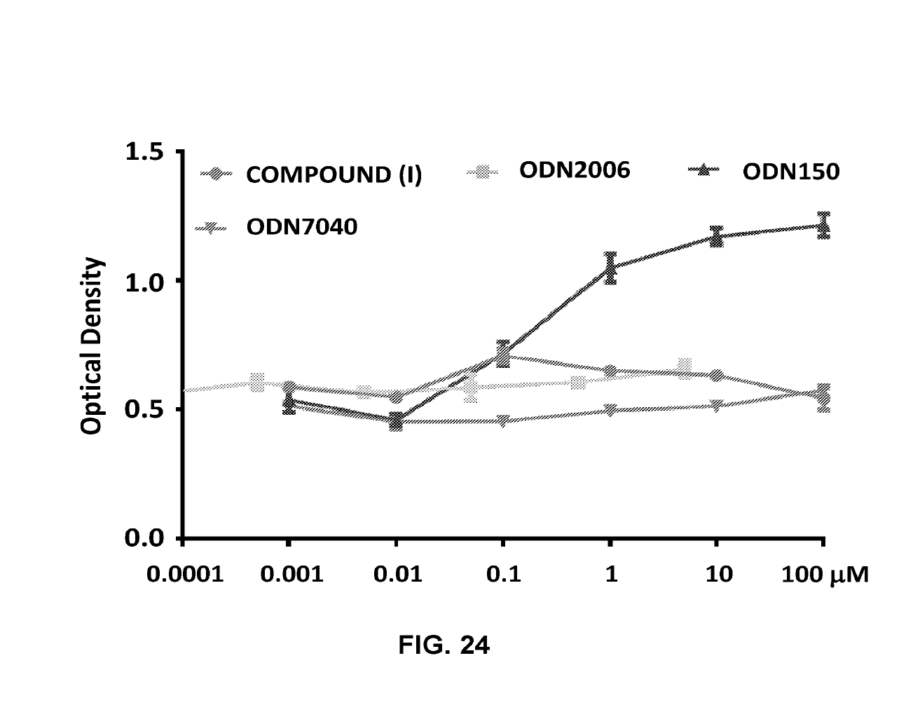

FIG. 24 shows results of an experiment in which HEK Blue TLR8 reporter cell lines (InvivoGen, San Diego, Calif.) were transfected with COMPOUND (I), ODN150, ODN2006, or ODN7040 at indicated concentrations and TLR8 activation was measured. Briefly, the reporter cells were engineered HEK293 cells that express a TLR8 gene and are specially designed for monitoring activity of NF-kβ inducible SEAP (secreted embryonic alkaline phosphatase). Reporter cells were seeded at a density of around 60000/well in 96 wells plates for 72 hrs. Growth medium was removed and Lipofectamine™ (Life Technologies, Foster City, Calif.) 2000/oligonucleotide complex and agonist was added and incubated for 6 hrs. The complex was removed and Detection Medium was added for 16 hrs of incubation. Optical density of SEAP was measured by spectrophotometer at 640 nM. The results show that of the tested ODNs, only ODN150 substantially induced TLR8 activation. See also, Table 7.

In conclusion, this Example demonstrates that COMPOUND (I) has a distinguishable cellular activity profile from ODN150 or ODN7040 oligonucleotides, e.g., in terms of TLR modulation or in its interaction with cells of the immune system, such as human PBMCs or pDCs. In particular, the ability of COMPOUND (I) to induce inflammasome activity, e.g., in human PBMCs, and the ability of COMPOUND (I) to induce human pDC differentiation differentiates COMPOUND (I) from ODN150 and ODN7040. In addition, this Example demonstrates the possible utility of COMPOUND (I) for the treatment of diseases involving inflammasome activation, such as tissue repair of IBD-associated cancer.

### 8.9 Example 10: COMPOUND (I) Acts Synergistically with NOD2 Ligand L18-MDP to Activate Inflammasome Complex in Human PBMCs

This Example demonstrates that COMPOUND (I) can act synergistically with a NOD2 receptor ligand, such as MDP or L18-MDP, to activate the inflammasome complex in human PBMCs.

Muramyl dipeptide (MDP) is commonly considered a minimal bioactive peptidoglycan motif common to all bacteria, and commonly used for its adjuvant activity in vaccines. MDP has been shown to be recognized by NOD2, but not TLR2, nor TLR2/1 or TLR2/6 associations. Numerous derivatives of MDP are known in the art and commercially available (e.g., Invivogen, San Diego, Calif.). Among them, L18-MDP (e.g., Invivogen cat. no. tlrl-lmdp), a 6-O-acyl derivative with a stearoyl fatty acid, is known for its very high activity. For example, in HEK-Blue™ NOD2 cells, L18-MDP is known to be a 10-fold more efficient NF-κB activator than MDP.

PBMCs were isolated from 3 donors of buffy coat (Blood Center of NY) using density gradient centrifugation. The PBMCs were resuspended in RPMI-1640 media supplemented with 10% FBS, 2 mM L-glutamine and 100 units/ml Penicillin and 100 μg/ml Streptomycin. The cells were plated in 96-well flat bottom plates at 250,000 cells/well. The cells were pre-treated for 1 hr with vehicle control (endotoxin-free water), 0.0001, 0.001, 0.01, 0.1, 1 and 10 μM COMPOUND (I) and ODN2006. After 1 hr, the cells were stimulated with and without L-18 MDP at 100 ng/ml (Invivogen, San Diego, Calif.) or 1 ng/ml LPS (Sigma, St. Louis, Mo.). The cells were then incubated for 24 hrs at 37° C., 5% CO₂. All compounds or ODNs were resuspended in endotoxin-free water. After 24 hrs, the supernatant was collected and analyzed for cytokine production. The supernatants were analyzed in duplicate for cytokine production in a magnetic multi-plex bead format using the MagPix instrument (Millipore, Billerica, Mass.). IL-1β was tested using a bead-based human cytokine/chemokine kit (Millipore, Cat# HCYTOMAG-60K-09). The manufacturer's procedures were followed accordingly. Data analysis was performed using Milliplex Analyst Software (Millipore).

FIG. 19 shows results of an experiment in which human PBMCs were stimulated about 51-fold with L-18 MDP (100 ng/ml) and further incubated with COMPOUND (I), ODN301C, ODN302, ODN2006, or ODN2006C at indicated concentrations, and IL-1β secretion was measured by ELISA. The results show that COMPOUND (I) can synergize with a NOD2 ligand, such as L-18 MDP, to further induce IL-1β secretion, and therefore inflammasome activation, in human PBMCs.

### 8.10 Example 11: COMPOUND (I) Acts Synergistically with LPS to Activate Inflammasome Complex in Human PBMCs

This Example demonstrates that COMPOUND (I) can act synergistically with a lipopolysaccharide (LPS) to activate the inflammasome complex in human PBMCs.

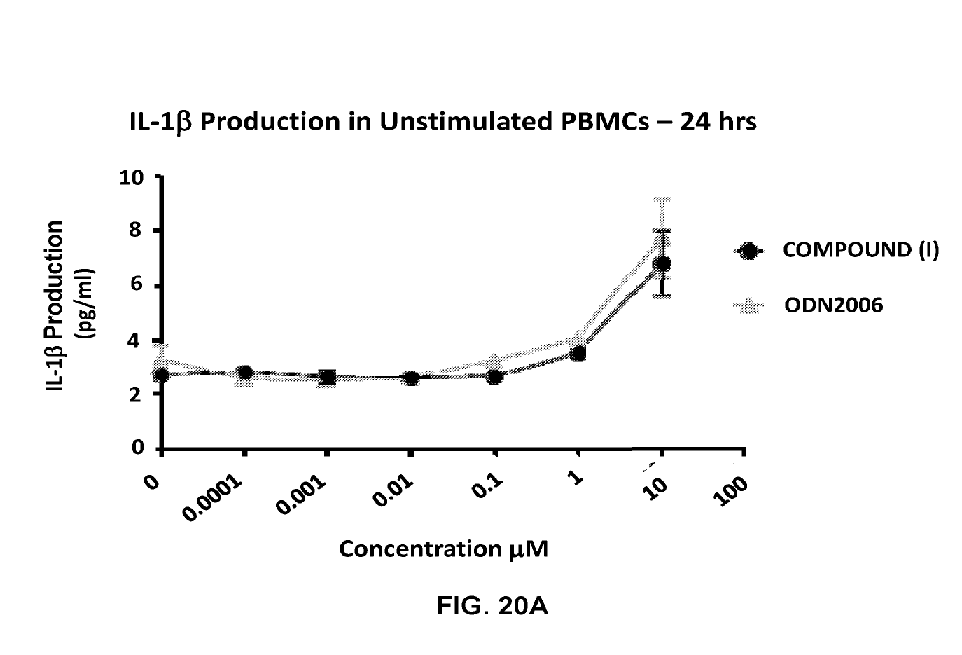

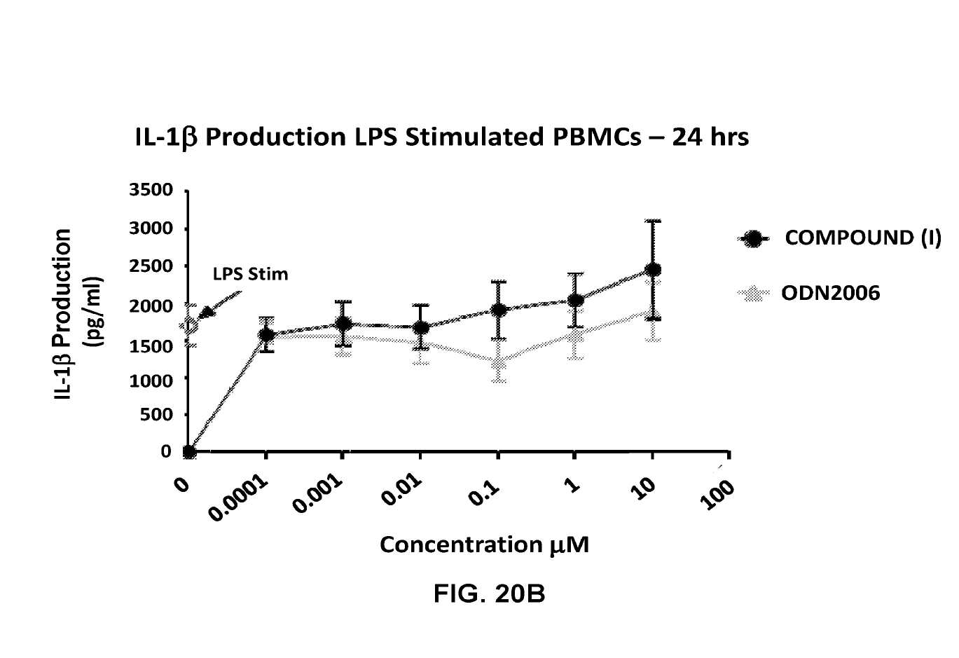

FIGS. 20A-B show results of experiments in which human PBMCs were incubated for 24 hours with COMPOUND (I) or ODN2006 at indicated concentrations, in the absence (FIG. 20A) or presence (FIG. 20B) of LPS and IL-1β secretion was measured by ELISA. The results show that LPS stimulates ˜1,000 higher levels of IL-1β secretion than COMPOUND (I) or ODN2006. Coincubation with COMPOUND (I) can further increase LPS-stimulated IL-1β secretion at COMPOUND (I) concentrations between 0.1 μM and 10 μM.

### 8.11 Example 12: COMPOUND (I) Induces pDC Differentiation

This Example demonstrates that COMPOUND (I) can induce differentiation of human pDCs.

FIG. 21 shows results of an experiment in which purified human pDCs were cultured in the presence of COMPOUND (I), ODN2006, or ODN7040 at indicated concentrations and differentiation markers CD86, CD83, CCR6, and CCR7 were analyzed by flow cytometry. The results show that COMPOUND (I) and ODN2006 induced differentiation of the purified human pDCs, as indicated by upregulation of CD83, CD86, and CCR7. By contrast, ODN7040 did not induce pDC differentiation, as determined using differentiation markers CD86, CD83, CCR6, and CCR7.

### 8.12 Example 13: Primary Cell Activity Profile of COMPOUND (I)

This Example summarizes results obtained from the profiling of COMPOUND (I) against a panel of twelve primary cell assays, using the BioMap® platform (DiscoveRx Corp., Fremont, Calif.).

COMPOUND(I) was characterized in thirteen primary human cell-based tissue and disease models as set forth in Table 8, below. These systems cover a broad range of vascular, epithelial (skin and respiratory), stromal, immune and inflammation biology.

TABLE 6
Cell-based Tissue and Disease Models

| | Primary Human Cell | | Disease/Tissue | Readout |
|---|---|---|---|---|
| System | Types | Stimuli | Relevance | Parameters |
| 3C | Venular endothelial | IL1β + TNFα + | Cardiovascular | MCP-1, VCAM-1, |
| | cells | IFNγ | disease, chronic | TM, TF, ICAM-1, |
| | | | inflammation | E-selectin, uPAR, |
| | | | | IL-6, MIG, HLA- |
| | | | | DR, Proliferation, |
| | | | | SRB |
| 4H | Venular endothelial | IL4 + histamine | Asthma, allergy, | MCP-1, Eotaxin-3, |
| | cells | | autoimmunity | VCAM-1, P- |
| | | | | selectin, uPAR, |
| | | | | SRB, VEGFRII |
| LPS | Peripheral blood | TLR4 agonist | Cardiovascular | MCP-1, VCAM-1, |
| | mononuclear cells + | | disease, chronic | TM, TF, CD40, E- |
| | venular endothelial cells | | inflammation | selectin, CD69, IL- |
| | | | | 8, IL1α, M-CSF, |
| | | | | sPGE2, SRB, |
| | | | | sTNF-α |
| SAg | Peripheral blood | TCR agonist | Autoimmune | MCP-1, CD38, |
| | mononuclear cells + | | disease, chronic | CD40, E-selectin, |
| | venular endothelial cells | | inflammation | CD69, IL-8, MIG, |
| | | | | PBMC, Cytotoxicity, |
| | | | | Proliferation, SRB |
| BT | B cells + peripheral | Anti-IgM + TCR | Asthma, allergy, | B cell proliferation, |
| | blood mononuclear | | oncology, | PBMC, |
| | cells | | autoimmunity | cytotoxicity, |
| | | | | secreted IgG, |
| | | | | sIL17A, sIL17GF, |
| | | | | sIL-2, sIL-6, |
| | | | | sTNFα |
| BF4T | Bronchial epithelial | TNFα + IL4 | Asthma, allergy, | MCP-1, eotaxin-3. |
| | cells + | | fibrosis, lung | VCAM-1, ICAM-1, |
| | dermal fibroblasts | | inflammation | CD90, IL-8, IL1α, |
| | | | | keratin 8/18, MMP- |
| | | | | 1, MMP-3, MMP-9, |
| | | | | PAI-1, SRB, tPA, |
| | | | | uPA |
| BE3C | Bronchial epithelial cells | IL1β + TNFα + | Lung inflammation, | ICAM-1, uPAR, IP- |
| | | IFNγ | COPD | 10, I-TAC, IL-8, |
| | | | | MIG, EGFR, HLA- |
| | | | | DR, IL1α, Keratin |
| | | | | 8/18, MMP-1, |
| | | | | MMP-9, PAI-1, |
| | | | | SRB, tPA, uPA |
| CASM3C | Coronary artery smooth | IL1β + TNFα + | Cardiovascular | MCP-1, VCAM-1, |
| | muscle cells | IFNγ | inflammation, | TM, TF, uPAR, IL- |
| | | | restenosis | 8, MIG, HLA-DR, |
| | | | | IL1-6, LDLR, M- |
| | | | | CSF, PAI-1, |
| | | | | proliferation, SAA, |
| | | | | SRB |
| HDF3CGF | Dermal fibroblasts | IL1β + TNFα + | Fibrosis, chronic | MCP-1, VCAM-1, |
| | | IFNγ + EGF + | inflammation | ICAM-1, collagen |
| | | bFGF + PDGF-BB | | I, collagen III, IP- |
| | | | | 10, i-TAC, IL-8, |
| | | | | MIG, EGFR, M- |
| | | | | CSF, MMP-1, PAI- |
| | | | | 1, proliferation 72 |
| | | | | hr, SRB, TIMP-1, |
| | | | | TIMP-2 |
| KF3CT | Keratinocytes + | IL1β + TNFα + | Psoriasis, | MCP-1, ICAM-1, |
| | dermal fibroblasts | IFNγ + TGFβ | dermatitis, skin | IP-10, IL-8, MIG, |
| | | | biology | Il1α, MMP-9, PAI- |
| | | | | 1, SRB, TIMP-2, uPA |
| MyoF | Lung fibroblasts | TNFα + TGFβ | Fibrosis, chronic | α-SM actin, bFGF, |
| | | | inflammation, | VCAM-1, collagen |
| | | | wound healing, | I, collagen III, |
| | | | matrix remodeling | collagen IV, IL-8, |
| | | | | decorin, MMP-1, |
| | | | | PAI-1, SRB, TIMP-1 |
| Mphg | Venular endothelial | TLR2 agonist | Cardiovascular | MCP-1, MIP-1α, |
| | cells + macrophages | | inflammation, | VCAM-1 , CD40, E- |
| | | | restenosis, chronic | selectin, CD69, IL- |
| | | | inflammation | 8, IL1α, M-CSF, |
| | | | | sIL10, SRB, SRB- |
| | | | | Mphg |
| HDSFAg | Dermal fibroblasts + | TCR agonist | Autoimmune | MCP-1, VCAM-1, |
| | peripheral blood | | disease, chronic | collagen I, IP-10, |
| | mononuclear cells | | inflammation, | MMP-1, sIL-10, |
| | | | rheumatoid | sIL17A, sIL17F, |
| | | | arthritis | sIL-2, sIL-6, SRB, |
| | | | | sTGFb, sTNFα, |
| | | | | sVEGF, IL-8, MIG, |
| | | | | MCSF |

Primary Human Cells. All studies followed the guidelines for human subjects research under United States HHS human subjects regulations (45 CFR Part 46). Preparation and culture of primary human cell types and methods for the systems were substantially as previously described (Kunkel E J et al, “Rapid structure-activity and selectivity analysis of kinase inhibitors by BioMAP analysis in complex human primary cell-based models,” Assay Drug Dev Technol., 2, 431-41 (2004); Berg E L et al. “Chemical target and pathway toxicity mechanisms defined in primary human cell systems,” Journal of Pharmacological and Toxicological Methods, 61, 3-15 (2010); Bergamini G et al., “A selective inhibitor reveals PI3Kγ dependence of T(H)17 cell differentiation,” Nature Chemical Biology, 8, 576-82 (2012); Xu D et al., “RN486 [6-Cyclopropyl-8-fluoro-2-(2-hydroxymethyl-3-{1-methyl-5-[5-(4-methylpiperazin-1-yl)-pyridin-2-ylamino]-6-oxo-1,6-dihydro-pyridin-3-yl}-phenyl)-2Hisoquinolin-1-one], a selective Bruton's tyrosine kinase (Btk) inhibitor, abrogates immune hypersensitivity responses and arthritis in rodents,” Journal of Pharmacology and Experimental Therapeutics, 3, (2012)). Human neonatal foreskin fibroblasts (HDFn) from 3 donors were pooled and cultured according to the supplier's (Lonza, Inc., Allendale, N.J.) recommendation, and plated in low serum conditions for 24 hr before assay initiation. Primary human bronchial epithelial cells (Cell Applications, Inc., San Diego, Calif.), arterial smooth muscle cells, adult lung fibroblasts (Lonza, Inc., Allendale, N.J.), and keratinocytes (Cambrex, Inc., East Rutherford, N.J.) were cultured according to methods recommended by the commercial suppliers. Peripheral blood mononuclear cells (PBMC) were prepared from buffy coats from normal human donors (Kunkel, E J et al., “An integrative biology approach for analysis of drug action in models of human vascular inflammation,” The FASEB Journal, 2004, 18, 1279-81). CD19+ B cells and CD14+ monocytes were obtained from AllCells, Inc. (Emeryville, Calif.). Macrophages were prepared by culturing CD14+ monocytes for 7 days. Concentrations/amounts of agents added to confluent microtiter plates to build each system were as follows: cytokines (IL-1β, 1 ng/ml; TNF-α, 5 ng/ml; IFN-γ, 20 ng/ml; IL-4, 5 ng/ml), activators (histamine, 10 microM; superantigens (TCR ligands), 20 ng/ml; or LPS, 2 ng/ml), growth factors (TGF-β, 5 ng/ml; EGF, bFGF, and PDGF-BB, 10 ng/ml; Zymosan 10 μg/ml; Anti-IgM, 500 ng/ml), B cells (2.5×104), PBMC (7.5×104 cells/well for LPS, Sag or HDFSAg systems or 2.5×104 cells/well for BT system) or macrophages (7500 cells/well). All primary human cells utilized were used at early passage (≤P4) to minimize adaptation to cell culture and preserve physiological signaling responses.

BioMAP Systems. Cell types and stimuli used in each system (Table 6, above) are as follows: 3C system (umbilical vein endothelial cells (HuVEC)/IL-1β, TNFα and IFNγ), 4H system (HuVEC/IL-4 and histamine), LPS system (PBMC and HuVEC/LPS), SAg system (PBMC and HuVEC/TCR ligands), BT system (CD19+B cells and PBMC/anti-IgM+TCR ligands), BE3C system (bronchial epithelial cells/IL-1β, TNFα and IFNγ), BF4T system (bronchial epithelial cells and human dermal fibroblasts/TNFα and IL-4), HDF3CGF system (human dermal fibroblasts/IL-1β, TNFα and IFNγ, EGF, bFGF and PDGF-BB), KF3CT system (keratinocytes and dermal fibroblasts/IL-1β, TNFα and IFNγ), CASM3C system (coronary artery smooth muscle cells/IL-1β, TNFα and IFNγ), MyoF system (differentiated lung myofibroblasts/TNF and TGFβ), /Mphg system (HuVEC and M1 macrophages/TLR2 ligands) and HDFSAg (PBMC and HDF/TCR ligands). For each assay, adherent cell types were first cultured until confluent at 37° C. Then PBMC (SAg, LPS and BT systems) and CD19+B cells (BT system) were added followed by test agents for 1 hr followed by addition of appropriate stimuli. Assay plates were then incubated for 24 hr except for the MyoF system (48 hr) and BT system (either 72 hr for soluble readouts or 6 d for secreted IgG). For proliferation assays, individual cell types were cultured at subconfluence and read at 48 hr, 72 hr (HDF3CGF), or 96 hr (BT system).

Test agents. COMPOUND(I) was prepared in water and added at indicated concentrations. COMPOUND(I) was added 1 hr before stimulation of the cells, and was present during the subsequent 24-96 hr period. Final DMSO concentration was <0.1%. Positive control samples including colchicine, 1.1 μM, and non-stimulated samples were included as controls on every plate. DMSO 0.1% was tested at 6 or more replicates per plate.

Endpoint measurements. The levels of readout parameters were measured by ELISA as described (Berg, 2010; Bergamini, 2012; Melton, 2013; Xu, 2012). Briefly, microtiter plates were treated, blocked, and then incubated with primary antibodies or isotype control antibodies (0.01-0.5 pg/ml) for 1 hr. After washing, plates were incubated with a peroxidase-conjugated anti-mouse IgG secondary antibody or a biotin-conjugated anti-mouse IgG antibody for 1 hr followed by streptavidin-HRP for 30 min. Plates were washed and developed with TMB substrate and the absorbance (OD) was read at 450 nm (subtracting the background absorbance at 650 nm). Quantitation of soluble readouts was done using commercially available kits according to the manufacturer's directions. Proliferation of PBMC (T cells) is quantified by Alamar blue reduction and proliferation of adherent cell types was quantified by SRB staining (Berg, 2010). SRB is performed by staining cells with 0.1% sulforhodamine B after fixation with 10% TCA, and reading wells at 560 nm (Ahmed, S A et al., “A new rapid and simple nonradioactive assay to monitor and determine the proliferation of lymphocytes: an alternative to [³H]thymidine incorporation assay,” J. Immunol. Methods, 1994, 170(2):211-24). PBMC viability is assessed by adding Alamar blue to PBMC that had been cultured for 24 hours in the presence of activators and compounds and measuring its reduction after 8 hr.

Data analysis. Measurement values for each parameter in a treated sample were divided by the mean value from at least six buffer control samples (from the same plate) to generate a ratio. All ratios were then log 10 transformed. Significance prediction envelopes were calculated from historical negative control samples tested (e.g., 95%). Given the control distribution for each system-readout combination, the significance of an individual readout ratio was computed from the empirical distribution by taking the 95^{th} percentile, or 99th, or 99.9th, e.g., as compared to the control ratios. Overtly cytotoxic compounds were identified as those that reduce the levels of total protein (sulforhodamine blue, SRB or Alamar blue, PBMC cytotoxicity) below 50%. For analysis of profile similarities, overtly cytotoxic compound profiles were removed and comparisons are assessed by mathematical correlation. The correlation metric is a combination of similarity metrics in addition to Pearson's correlation (Berg, 2010). Similar profiles were identified as those having Pearson correlations above a selected threshold>0.7 (or as otherwise indicated). Clustering analysis (function similarity map) uses the results of pairwise correlation analysis to project the “proximity” of compound profiles from multi-dimensional space to two dimensions. The two dimensional projection coordinates were generated by applying a modified nonlinear mapping technique as described (Berg, 2010). A gradient descent minimization method was used to minimize the modified stress function, starting from a set of initial positions (e.g., from principal components analysis).

Assay acceptance criteria. The BioMAP platform generated multi-parameter data sets for each compound tested. Assays were plate-based and performance was assessed by positive and negative controls for each assay. Negative controls include buffer (e.g., DMSO). For stimulated systems, positive controls include the non-stimulated condition (non-stim) and a positive control test agent (colchicine). Data acceptance criteria were based on plate performance (% CV of negative control wells), and the performance of positive controls across assays with a comparison to historical controls. The performance of each BioMAP system in a given assay was evaluated using the Pearson statistic for the positive control, calculated individually for each assay compared to the positive control reference dataset. This test, the QA/QC Pearson Test, was performed by first establishing the 1% false negative Pearson cutoff from the positive reference dataset. The process was iterated through each profile in the positive control reference dataset, calculating Pearson values between this profile and the mean of the rest of the profiles in the dataset, so the number of Pearson values calculated was the number of profiles in the reference dataset. The Pearson at the one percentile of all Pearson values calculated was the 1% false negative Pearson cutoff. If the Pearson between a new positive control profile and the mean of positive control reference profiles exceeded this 1% false negative Pearson cutoff, then these plates passed the test. Assays were accepted when the positive control passes the Pearson test and 95% of plates have % CV<20%.

Identified activities of COMPOUND (I) include:
inflammation-related activities:
   decreased expression of eotaxin-3 (Eot-3, CCL20), interleukine (IL-1α), soluble TNF-α, interferon-inducible T-cell alpha chemoattractant (I-TAC), vascular cell adhesion molecule-1 (VCAM-1), IFN-γ inducible protein 10 (IP-10), monocyte chemoattractant protein-1 (MCP-1), intercellular adhesion molecule 1 (ICAM-1), macrophage inflammatory protein 1 alpha (MIP-1α), and modulated monokine induced by gamma interferon (modulated MIG);
immunomodulatory activities:
   decreased expression of macrophage colony-stimulating factor, soluble IgG, soluble IL-10,
   increased expression of cluster of differentiation 69 (CD69), soluble IL-6;
tissue remodeling activities:
   decreased expression of urinary-type plasminogen activator (uPA), basic fibroblast growth factor (bFGF), plasminogen activator inhibitor-1 (PAI-I);
   increased expression of urokinase receptor (uPAR), tissue plasminogen activator (tPA), epithelial growth factor receptor (EGFR)

Both immune stimulatory and immune suppressive activities of COMPOUND (I) were detected in the BioMap® screen. However, the profile, on balance, was immunosuppressive, as evidenced by profile matches with known small molecule inhibitors of Src-family kinases. A search against the BioSeek® database (DiscoveRx Corp., Fremont, Calif.) for compounds having comparable cellular activity profiles to COMPOUND (I) returned the Src Family inhibitor SU6656, the thymidylate synthase inhibitor Raltitrexed and the dihydrofolate reductase inhibitor methotrexate as the closest matches.

In conclusion, this example suggests that COMPOUND (I) is a compound with on balance immunosuppressive activity. Combinations of COMPOUND (I) with a Src-kinase inhibitor (e.g., SU6656), thymidylate synthase inhibitor (e.g., Raltitrexed), or dihydrofolate reductase inhibitor (methotrexate) can be useful combination therapies to treat inflammatory or other diseases, including cancer.

### 8.13 Example 14: Primity Profile of COMPOUND (I)

This Example summarizes results obtained from the profiling of COMPOUND (I) against a panel of cellular pathway assays, using the PrimityBio Inc. surface profiling platform (Primity Bio, Inc., Fremont, Calif.).

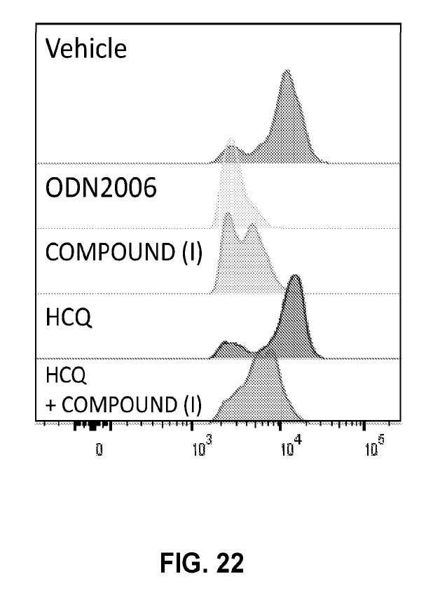

Over 400 cell surface markers and intracellular cytokines and signaling mediators were screened by flow cytometry following 30 min treatments with COMPOUND(I), ODN2006, or a no ODN/antibody control:
COMPOUND (I) and ODN2006 were found to induce similar phosphoprotein profiles in human pDCs, including phosphorylated versions of histone H3, p38 mitogen-activated protein (MAP) kinase (COMPOUND (I) showed weaker induction than ODN2006), and zeta-chain-associated protein kinase 70 (Zap 70);
COMPOUND (I) induced a subset (MCP-1, receptor activator of nuclear factor kappa-B ligand (RANKL), IL-2) of cytokines at a level comparable to ODN2006. Both equally inhibited IL-4;
COMPOUND (I) induced glycoprotein A repetitions predominant (GARP, a TGF-β regulator) and IL-23p19, but to a lesser extent than ODN2006;
ODN2006 strongly induced IL-27, while COMPOUND (I) did not;
COMPOUND (I) strongly induced IL-1β, while ODN2006 inhibited it;
COMPOUND (I) reduced CXCL10 (IP-10), while ODN2006 did not;
COMPOUND (I) and ODN2006 decrease CD123 (IL3R) in human PBMCs; COMPOUND (I) reduced CXCL10 (IP-10), whereas ODN2006 did not, see also FIG. 22;
COMPOUND (I) was shown to decrease the expression of immunoglobulin-like transcript 7 (ILT7) in pDCs, and increase expression of IL10Rα and SLAM family member 7 (Slamf7), which are immunomodulatory receptors believed to be involved in Treg induction and immune suppression;
COMPOUND (I) reduced CD123 and CCR6 expression in a concentration dependent manner, see also FIG. 23.

In conclusion, this example suggests that COMPOUND (I) is a compound with on balance immunosuppressive activity. A number of proteins were identified, which can be useful as biomarkers, e.g., for the monitoring of COMPOUND (I) efficacy and general pharmacology.

In summary, the Examples provided herein demonstrate that COMPOUND (I) has a subset of activities that are associated broadly with multiple classes of oligonucleotides as well as other activities that are more selective. For example, COMPOUND (I) was shown to induce tolerogenic pDCs and thereby induce Tregs, which can result in the desirable suppression of pathogenic T effector cells, e.g., in an inflammatory disease condition. Induction of tolerogenic pDCs can be achieved using a number of oligonucleotides, including, e.g., ODN150 or ODN7040. On the other hand, COMPOUND (I) was shown to induce pDC differentiation (see, e.g., COMPOUND (I) mediated downregulation of CD123 and CCR6 and upregulation of CCR7, SLAMF7, CD80, CD83, and CD86) and to induce the inflammasome (e.g., IL-1β and IL-18 upregulation). COMPOUND (I) shares these latter activities with CpG-B-type TLR9 agonists, such as ODN2006, but not, e.g., with ODN150 or ODN7040. Homeostatic inflammasome activation can be useful, e.g., in the treatment of IBD through the promotion of epithelial restitution (mucosal healing), or in the treatment of other diseases involving wound repair. Chronic inflammasome activation can promote inflammatory effects, e.g., through upregulation of chemokines such as IL-1β and IL-18, and other proinflammatory mediators.

The present disclosure is not to be limited in scope by the specific embodiments described herein. Indeed, various modifications of the subject matter provided herein, in addition to those described, will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

TABLE 5
Sequence Listing

| ID | SEQUENCE | Comment; Reference |
|---|---|---|
| SEQ ID NO: 1 | atg ttcaggacca aacgatctgc gctcgtccgg | Coding |
| | cgtctctgga ggagccgtgc gcccggcggc gaggacgagg | Sequence: CDS |
| | aggagggcgc agggggaggt ggaggaggag gcga [symbol] | (288-1568) of |
| | [symbol] gaca gccgagcgca tggggccggt | NM_005904.3; |
| | ggcggcggcc cgggcagggc tggatgctgc ctgggcaagg | Homo sapiens |
| | cggtgcgagg tgccaaaggt caccaccatc cccacccgcc | SMAD family |
| | agccgcgggc gccggcgcgg ccgggggcgc cgaggcggat | member 7 |
| | ctgaaggcgc tcacgcactc ggtgctcaag aaactgaagg | (SMAD7), |
| | agcggcagct ggagctgctg ctccaggccg tggagtcccg | transcript |
| | cggcgggacg cgcaccgcgt gcctcctgct gcccggccgc | variant 1, mRNA |
| | ctggactgca ggctgggccc gggggcgccc gccggcgcgc | (region 108-128 |
| | agcctgcgca gccgccctcg tcctactcgc tccccctcct | underlined) |
| | gctgtgcaaa gtgttcaggt ggccggatct caggcattcc | |
| | tcggaagtca agaggctgtg ttgctgtgaa tcttacggga | |
| | agatcaaccc cgagctggtg tgctgcaacc cccatcacct | |
| | tagccgactc tgcgaactag agtctccccc ccctccttac | |
| | tccagatacc cgatggattt tctcaaacca actgcagact | |
| | gtccagatgc tgtgccttcc tccgctgaaa cagggggaac | |
| | gaattatctg gcccctgggg ggctttcaga ttcccaactt | |
| | cttctggagc ctggggatcg gtcacactgg tgcgtggtgg | |
| | catactggga ggagaagacg agagtgggga ggctctactg | |
| | tgtccaggag ccctctctgg atatcttcta tgatctacct | |
| | caggggaatg gcttttgcct cggacagctc aattcggaca | |
| | acaagagtca gctggtgcag aaggtgcgga gcaaaatcgg | |
| | ctgcggcatc cagctgacgc gggaggtgga tggtgtgtgg | |
| | gtgtacaacc gcagcagtta ccccatcttc atcaagtccg | |
| | ccacactgga caacccggac tccaggacgc tgttggtaca | |
| | caaggtgttc cccggtttct ccatcaaggc tttcgactac | |
| | gagaaggcgt acagcctgca gcggcccaat gaccacgagt | |
| | ttatgcagca gccgtggacg ggctttaccg tgcagatcag | |
| | ctttgtgaag ggctggggcc agtgctacac ccgccagttc | |
| | atcagcagct gcccgtgctg gctagaggtc atcttcaaca | |
| | gccggtag | |
| SEQ ID NO: 2 | 5′-gtcgccccttctccccgcag-3′ | |
| SEQ ID NO: 3 | 5′-gtcgccccttctccccgcagc-3′ | |
| SEQ ID NO: 4 | 5′-gtxgccccttctcccxgcag-3′, wherein X is 5- | |
| | methyl 2′-deoxycytidine | |
| SEQ ID NO: 5 | 5′-gtxgccccttctcccxgcagc-3′, wherein X is 5- | |
| | methyl 2′-deoxycytidine | |
| SEQ ID NO: 6 | 5′-gtxyccccttctcccxycag-3′, whereby X is a | |
| | nucleoside comprising a nitrogenous base | |
| | selected from the group consisting of cytosine, | |
| | 5-methylcytosine and 2-O-methylcytosine, and | |
| | wherein Y is a nucleoside comprising a | |
| | nitrogenous base selected from the group | |
| | consisting of guanine, 5-methylguanine and 2-O- | |
| | methylguanine, optionally provided that at | |
| | least one of the nucleosides X or Y comprises a | |
| | methylated nitrogenous base. | |
| | In some embodiments, at least one of the | |
| | internucleoside linkages of the SMAD7 AON is a | |
| | phosphorothioate linkage. | |
| | In some embodiments, all of the internucleoside | |
| | linkages of the SMAD7 AON are phosphorothioate | |
| | linkages. | |
| SEQ ID NO: 7 | 5′-gtxgccccttctcccxgcagc-3′, whereby X is a | |
| | nucleoside comprising 5-methyl-2′- | |
| | deoxycytidine. | |
| | In some embodiments, at least one of the | |
| | internucleoside linkages of the SMAD7 AON is a | |
| | phosphorothioate linkage. | |
| | In some embodiments, all of the internucleoside | |
| | linkages of the SMAD7 AON are phosphorothioate | |
| | linkages. | |
| SEQ ID NO: 8 | ggcacgagcg gagagccgcg cagggcgcgg gccgcgcggg | Homo sapiens |
| | gtggggcagc cggagcgcag gcccccgatc cccggcgggc | SMAD7 mRNA, |
| | gcccccgggc ccccgcgcgc gccccggcct ccgggagact | GenBank AF0010193 |
| | ggcgcatgcc acggagcgcc cctcgggccg ccgccgctcc | |
| | tgcccgggcc cctgctgctg ctgctgtcgc ctgcgcctgc | |
| | tgccccaact cggcgcccga cttcttcatg gtgtgcggag | |
| | gtcatgttcg ctccttagca ggcaaacgac ttttctcctc | |
| | gcctcctcgc cccgcatgtt caggaccaaa cgatctgcgc | |
| | tcgtccggcg tctctggagg agccgtgcgc ccggcggcga | |
| | ggacgaggag gagggcgcag ggggaggtgg aggaggaggc | |
| | gagctgcggg gagaaggggc gacggacagc cgagcgcatg | |
| | gggccggtgg cggcggcccg ggcagggctg gatgctgcct | |
| | gggcaaggcg gtgcgaggtg ccaaaggtca ccaccatccc | |
| | cacccgccag ccgcgggcgc cggcgcggcc gggggcgccg | |
| | aggcggatct gaaggcgctc acgcactcgg tgctcaagaa | |
| | actgaaggag cggcagctgg agctgctgct ccaggccgtg | |
| | gagtcccgcg gcgggacgcg caccgcgtgc ctcctgctgc | |
| | ccggccgcct ggactgcagg ctgggcccgg gggcgcccgc | |
| | cggcgcgcag cctgcgcagc cgccctcgtc ctactcgctc | |
| | cccctcctgc tgtgcaaagt gttcaggtgg ccggatctca | |
| | ggcattcctc ggaagtcaag aggctgtgtt gctgtgaatc | |
| | ttacgggaag atcaaccccg agctggtgtg ctgcaacccc | |
| | catcacctta gccgactctg cgaactagag tctccccccc | |
| | ctccttactc cagatacccg atggattttc tcaaaccaac | |
| | tgcagactgt ccagatgctg tgccttcctc cgctgaaaca | |
| | gggggaacga attatctggc ccctgggggg ctttcagatt | |
| | cccaacttct tctggagcct ggggatcggt cacactggtg | |
| | cgtggtggca tactgggagg agaagacgag agtggggagg | |
| | ctctactgtg tccaggagcc ctctctggat atcttctatg | |
| | atctacctca ggggaatggc ttttgcctcg gacagctcaa | |
| | ttcggacaac aagagtcagc tggtgcagaa ggtgcggagc | |
| | aaaatcggct gcggcatcca gctgacgcgg gaggtggatg | |
| | gtgtgtgggt gtacaaccgc agcagttacc ccatcttcat | |
| | caagtccgcc acactggaca acccggactc caggacgctg | |
| | ttggtacaca aggtgttccc cggtttctcc atcaaggctt | |
| | tcgactacga gaaggcgtac agcctgcagc ggcccaatga | |
| | ccacgagttt atgcagcagc cgtggacggg ctttaccgtg | |
| | cagatcagct ttgtgaaggg ctggggtcag tgctacaccc | |
| | gccagttcat cagcagctgc ccgtgctggc tagaggtcat | |
| | cttcaacagc cggtagccgc gtgcggaggg gacagagcgt | |
| | gagctgagca ggccacactt caaactactt tgctgctaat | |
| | attttcctcc tgagtgcttg cttttcatgc aaactctttg | |
| | gtcgtttttt ttttgtttgt tggttggttt tcttcttctc | |
| | gtcctcgttt gtgttctgtt ttgtttcgct ctttgagaaa | |
| | tagcttatga aaagaattgt tgggggtttt tttggaagaa | |
| | ggggcaggta tgatcggcag gacaccctga taggaagagg | |
| | ggaagcagaa atccaagcac caccaaacac agtgtatgaa | |
| | ggggggcggt catcatttca cttgtcagga gtgtgtgtga | |
| | gtgtgagtgt gcggctgtgt gtgcacgcgt gtgcaggagc | |
| | ggcagatggg gagacaacgt gctctttgtt ttgtgtctct | |
| | tatggatgtc cccagcagag aggtttgcag tcccaagcgg | |
| | tgtctctcct gccccttgga cacgctcagt ggggcagagg | |
| | cagtacctgg gcaagctggc ggctggggtc ccagcagctg | |
| | ccaggagcac ggctctgtcc ccagcctggg aaagcccctg | |
| | cccctcctct ccctcatcaa ggacacgggc ctgtccacag | |
| | gcttctgagc agcgagcctg ctagtggccg aaccagaacc | |
| | aattattttc atccttgtct tattcccttc ctgccagccc | |
| | ctgccattgt agcgtctttc ttttttggcc atctgctcct | |
| | ggatctccct gagatgggct tcccaagggc tgccggggca | |
| | gccccctcac agtattgctc acccagtgcc ctctcccctc | |
| | agcctctccc ctgcctgccc tggtgacatc aggtttttcc | |
| | cggacttaga aaaccagctc agcactgcct gctcccatcc | |
| | tgtgtgttaa gctctgctat taggccagca agcggggatg | |
| | tccctgggag ggacatgctt agcagtcccc ttccctccaa | |
| | gaaggatttg gtccgtcata acccaaggta ccatcctagg | |
| | ctgacaccta actcttcttt catttcttct acaactcata | |
| | cactcgtatg atacttcgac actgttctta gctcaatgag | |
| | catgtttaga ctttaacata agctattttt ctaactacaa | |
| | aggtttaaat gaacaagaga agcattctca ttggaaattt | |
| | agcattgtag tgctttgaga gagaaaggac tcctgaaaaa | |
| | aaacctgaga tttattaaag aaaaaaatgt attttatgtt | |
| | atatataaat atattattac ttgtaaatat aaagacgttt | |
| | tataagcatc attatttatg tattgtgcaa tgtgtataaa | |
| | caagaaaaat aaagaaaaga tgcactttgc tttaatataa | |
| | atgcaaataa caaatgccaa attaaaaaag ataaacacaa | |
| | gattggtgtt ttttcctatg ggtgttatca cctagctgaa | |
| | tgtttttcta aaggagttta tgttccatta aacgattttt | |
| | aaaatgtaca cttgaaaaaa aaaaaaaaaa a | |
| SEQ ID NO: 9 | cgagtgcggc gcggcgagcc cccagcggcg gcagaaggac | Mus musculus |
| | tcgagcgcca ggagagggcg gacgggggac gaggaggctc | SMAD7 mRNA, |
| | cggggcgcga cgaagagagt ctccgaggaa gaggctgcga | GenBank NM_008543 |
| | gaggacaccc gggcctcctg ccgccactgt cgggtcgggg | |
| | ccagcagctc atgagagcag ccccggcggc cacccgcggc | |
| | caggagaagg agcaccggag gcccccacac tagcctgtgc | |
| | cctcgggggc gagagcttgc gacccgccgg agcccgccgc | |
| | cgcgccgccc tcccccgcgc tgacagcccc ccggggcgca | |
| | gccgccgccg cagcatcttc tgtccctgct tccccagcgc | |
| | ggaggaagtc cccgccgagg acctgagccc ccgggaacgc | |
| | aggaggaaag accagagact ctaaaacacc cagatacgca | |
| | agattgaagc agcctagcca gacctttctg tggattaaaa | |
| | gaaatacgat tttttttttt tttttttggc agaagaaaag | |
| | gaaaggaaga ccggctgggt tcagcaagga aaaaaagggg | |
| | gatgtaactc gtggatacgg tttttttccc ccacccttcc | |
| | aacatcttgt tttattttgt aaacattttc tcttttaaac | |
| | ccgggctcca tccggtgccc tccagacctc cgaggtgcga | |
| | ggaggtggtg tgttttttca ttgggggctt tgcatatttt | |
| | ggttttgggg gttttgagag accctccaga catctcacga | |
| | ggggtgaagt ctactcggtc ccctcccgca agtcttcgcg | |
| | tgcacagaat tcgaggagat ccggttacta aggatataga | |
| | agaaaaaaaa taaatcgtgc ctgccttttt tttttaattg | |
| | cctgcttctc cccaccccca aattaagttg cttagcaagg | |
| | gggaaagagg ctttttcctc cctttagtag ctcagcctaa | |
| | cgtctttcgt tttttttttt tttttttttt ttttgccccc | |
| | gaggatcttc catgtaggaa gccgaggctg gcgagcccga | |
| | cactcgggag ccactgtagg ggggcctttt ttgggggagg | |
| | cgtctaccgg ggttgcctcg gccgccccca gggaagcggc | |
| | ggccgcgttc ctccagggca cgccggggcc cgaaagccgc | |
| | gcagggcgcg ggccgcgccg ggtggggcag ccgaagcgca | |
| | gccccccgat ccccggcagg cgcccctggg cccccgcgcg | |
| | cgccccggcc tctgggagac tggcgcatgc cacggagcgc | |
| | ccctcgggcc gccgccgctt ctgcccgggc ccctgctgtt | |
| | gctgctgtcg cctgcgcctg ctgccccaac tcggcgcccg | |
| | acttcttcat ggtgtgcgga ggtcatgttc gctccttagc | |
| | cggcaaacga cttttctcct cgcctcctcg ccccgcatgt | |
| | tcaggaccaa acgatctgcg ctcgtccggc gtctctggag | |
| | gagccgtgcg cccggcggcg aggacgagga ggagggcgtg | |
| | gggggtggcg gcggaggagg cgagctgcgg ggagaagggg | |
| | cgacggacgg ccgggcttat ggggctggtg gcggcggtgc | |
| | gggcagggct ggctgctgcc tgggcaaggc agtccgaggt | |
| | gccaaaggtc accaccatcc ccatccccca acctcgggtg | |
| | ccggggcggc cgggggcgcc gaggcggatc tgaaggcgct | |
| | cacgcactcg gtgctcaaga aactcaagga gcggcagctg | |
| | gagctgctgc ttcaggccgt ggagtcccgc ggcggtacgc | |
| | gcaccgcgtg cctcctgctg cccggccgcc tggactgcag | |
| | gctgggcccg ggggcgcccg ccagcgcgca gcccgcgcag | |
| | ccgccctcgt cctactcgct ccccctcctg ctgtgcaaag | |
| | tgttcaggtg gccggatctc aggcattcct cggaagtcaa | |
| | gaggctgtgt tgctgtgaat cttacgggaa gatcaacccc | |
| | gagctggtgt gctgcaaccc ccatcacctt agtcgactct | |
| | gtgaactaga gtctccccct cctccttact ccagataccc | |
| | aatggatttt ctcaaaccaa ctgcaggctg tccagatgct | |
| | gtaccttcct ccgcggaaac cgggggaacg aattatctgg | |
| | cccctggggg gctttcagat tcccaacttc ttctggagcc | |
| | tggggatcgg tcacactggt gcgtggtggc atactgggag | |
| | gagaagactc gcgtggggag gctctactgt gtccaagagc | |
| | cctccctgga tatcttctat gatctacctc aggggaatgg | |
| | cttttgcctc ggacagctca attcggacaa caagagtcag | |
| | ctggtacaga aagtgcggag caagatcggc tgtggcatcc | |
| | agctgacgcg ggaagtggat ggcgtgtggg tttacaaccg | |
| | cagcagttac cccatcttca tcaagtccgc cacactggac | |
| | aacccggact ccaggacgct gttggtgcac aaagtgttcc | |
| | ctggtttctc catcaaggct tttgactatg agaaagccta | |
| | cagcctgcag cggcccaatg accacgagtt catgcagcaa | |
| | ccatggacgg gtttcaccgt gcagatcagc tttgtgaagg | |
| | gctggggcca gtgctacacc cgccagttca tcagcagctg | |
| | cccgtgctgg ctggaggtca tcttcaacag ccggtagtcg | |
| | gtcgtgtggt ggggagaaga ggacagggcg gatcgtgagc | |
| | cgagcaggcc accgttcaaa ctacttgctg ctaatctttc | |
| | ccgagtgatt gcttttcatg caaactcttt ggttggtgtt | |
| | gttattgcca ttcattgttg gttttgtttt gttctgttct | |
| | ggtttgtttt tttttttttt cctccccaag ggctgccggg | |
| | acagccccag tcacagtatt gctaccccag taccctctca | |
| | ggcccttcca ccgggtccca gccgtggtgg ttttttcatc | |
| | aggtttctcc cagatgtgga aagtcagctc agcatcccat | |
| | cccccatcct gtgtgctgag ctctgtagac cagcgagggg | |
| | catcagggag ggacctgcgc agtgcccccc cttcctgctg | |
| | agaagggtgt agccccgtca caacaaaggt accatccctt | |
| | ggctggctcc cagcccttct ctcagctcat acgctcgctc | |
| | gtatgatact ttgacactgt tcttagctca atgagcatgt | |
| | ttagaattta acataagcta tttttctaac tacaaaggtt | |
| | taaatgaaca agagaagcat tctcattgga aatttagcat | |
| | tgtagtgctt tgagagagga aaggactcct taaaagaaaa | |
| | aaaaagctga gatttattaa agaaaaatgt attttatgtt | |
| | atatataaat atattattac ttgtaaatat aaagacgttt | |
| | tataagcatc attatttatg tattgtgcaa tgtgtataaa | |
| | cgagaagaat aaagaaaaga tgcactttgc tttaatataa | |
| | atgtaaataa catgccaaat taaaaaaaaa aagataaaca | |
| | caagattggt gtttttttct atgggtgtta tcacctagct | |
| | gaatgttttt ctaaaggagt ttatgttcca ttaaacaatt | |
| | tttaaaatgt taaaaaaaaa aaaaaaaaaa aaaaaaaaaa a | |
| SEQ ID NO: 10 | tgagtgcggc gcggcgagcc cccagcggcg gcagaaggac | Rattus norvegicus |
| | tcgagcgcca ggagagggcg gacgggggac gaggaggctc | SMAD7 mRNA, |
| | ccgggcgcga cgaagagagt ctcggaggaa gaggctgcga | GenBank NM_030858 |
| | gaggacaccc gggcctcctg ccgccactgt cgggtcgggg | |
| | ccagcagctt atgcgagcag ccccagcgac caccctcggc | |
| | caggagaagg ggcaccggca gcccccacgc tagctagcct | |
| | gccgcctgtg ccctcggggg cgagagcttg cgacccgccg | |
| | gagcccgccg ccgcgccgcc ctcccccgcg ctgacagccc | |
| | cccggggcgc agccgccgcc gcagcatctt ctgtccctgc | |
| | ttccccagcg cggaggaagt ccccgccgag gacctgggcc | |
| | cccgggagcg caggaggaaa gaccagagac tctaaaacac | |
| | ccagatacgc aagattgaag cagcctaacc agacctttct | |
| | gtggattaaa agaaatacga tttttttttt gacagaagaa | |
| | aaggaaagga agaccggcgg ggttcagcaa ggaaaaaaag | |
| | gggatgtaac tcgtggatac ggtttttccc cccacccttc | |
| | caacatcttg ttctactttg taaacatttt ctctttttaa | |
| | accccggctc catccggtgc cctccagacc tccgaggtgc | |
| | gagaaggtgg tgtgtttttt cactgggggc tttgcatatt | |
| | tggttttggg gtttttgaga gaccctccag acatctcacg | |
| | aggggtgaag tctactcggc cccctccctc aagtcttcgc | |
| | gtgcacagaa ttcgaggaga tccggttact aaggatatag | |
| | aagaaaaaaa taaatcgtgt gcctgccttt ttttttttta | |
| | attgcctgct tctccccacc cccaaattaa gttgcttagc | |
| | aagggggaaa gaggcttttt cctcccttca gtagctcagc | |
| | ctaacgtctt tcgttttttg cccctgagga tcttccatgt | |
| | aggaagccga ggctggcgag cccgacactc gggagccact | |
| | gtaggggggc ctttttgggg agaggcgtcg accggggctg | |
| | cctcggccgc ccccagggaa gcggcggccg cgttcctcag | |
| | gggcacgccg gggcccgaga gccgcgcagg gcgcgggccg | |
| | cgccgggtgg ggcagccgaa gcgcaggccc ccgatccccg | |
| | gcgggcgccc ctgggccccc gcgcgcgccc cggcctccgg | |
| | gagactggcg catgccacgg agcgcccctc gggccgccgc | |
| | cgcttctgcc cgggcccctg ctgttgttgc tgtcgcctgc | |
| | gcctgctgcc ccaactcggc gcccgacttc ttcatggtgt | |
| | gcggaggtca tgttcgctcc ttagccggca aacgactttt | |
| | ctcctcgcct cctcgccccg catgttcagg accaaacgat | |
| | ctgcgctcgt ccggcgtctc tggaggagcc gtgcgcccgg | |
| | cggcgaggac gaggaggagg gcgtgggggg tggcggcggc | |
| | ggaggcgacc tgcggggaga aggggcgacg gacggccggg | |
| | cttatggggc tggtggcggc ggtgcgggca gggctggctg | |
| | ctgcctgggt aaggcagtcc gaggtgccaa aggtcaccac | |
| | catccccatc ccccatcctc gggtgccggg gcggccgggg | |
| | gcgccgaggc ggatctgaag gcgctcacgc actcggtgct | |
| | caagaaactc aaggagcggc agctggagct gctgcttcag | |
| | gccgtggagt cccgcggcgg tacgcgcacc gcgtgcctcc | |
| | tgctgcccgg ccgcctggac tgcaggctgg gcccgggggc | |
| | gcccgccagc gcgcagcccg cgcagccgcc ctcgtcctac | |
| | tcgctccccc tcctgctgtg caaagtgttc aggtggccgg | |
| | atctcaggca ttcctcggaa gtcaagaggc tgtgttgctg | |
| | tgaatcttac gggaagatca accccgagct ggtgtgctgc | |
| | aacccccatc accttagtcg actctgtgaa ctagagtctc | |
| | cccctcctcc ttactccaga tacccgatgg attttctcaa | |
| | accaactgca gactgtccag acgctgtacc ttcctccgat | |
| | gaaaccgggg gaacgaatta tctggcccct ggggggcttt | |
| | cagattccca acttcttctg gagcctgggg atcggtcaca | |
| | ctggtgcgtg gtggcatact gggaggagaa gactcgagtg | |
| | gggaggctct actgtgtcca agagccctcc ctggatatct | |
| | tctatgatct acctcagggg aatggctttt gcctcggaca | |
| | gctcaattcg gacaacaaga gtcagctggt acagaaagtg | |
| | aggagcaaga tcggctgtgg catccagctg acaagggaag | |
| | tggatggcgt gtgggtttac aaccgcagca gttaccccat | |
| | cttcatcaag tccgccacac tggacaaccc ggactccagg | |
| | acgctgttgg tgcacaaagt gttccctggt ttctccatca | |
| | aggcttttga ctatgaaaag gcctacagcc tgcagcggcc | |
| | caatgaccac gagttcatgc agcagccatg gacgggcttc | |
| | accgtgcaga ttagcttcgt gaagggctgg ggccagtgct | |
| | acacccgcca gttcatcagc agttgcccgt gctggctgga | |
| | ggtcatcttc aacagccggt agtctcccgg tgtggggaga | |
| | agaggacagg acggaggggt gagccgagca ggccaccgtt | |
| | caaactactt gctgctaatc tttcatgcaa aactctttcg | |
| | gtcggttttg ttgtttgcca ttcattgttg gttctgtttt | |
| | gttttgtttt cctttttttt ttttcttcct tcttcttttt | |
| | cctcctttct tgtcactctt gtgtcctgtg tgtctcgttc | |
| | tttgagaaaa tatgatgcgg atttttggtt gtgtgttttt | |
| | ttttttcgtt tgtttgtttg ttgttgttgt ttgtgttttg | |
| | aggtggtggt gggtgcggtt ggcaggacac cccgatacaa | |
| | aaacgggaag caagagtcag cactgccaag cgtggtgtgc | |
| | gaaagtgggt accaccttcc cctttggatc agcatttcag | |
| | ttgtcagtgt gtgtgtgtga ggggggtgta cgtgaatgac | |
| | agatggggga atggcgtgct ttttttgtgt tctttatgga | |
| | tgtccccagc tgagaggctt gcagttccaa gctgtgtgtc | |
| | tctcactgtg tgtctctctc atgagccttt cggacatgct | |
| | cggtggggca gaggctgtac ctgggcagac tggcagcagg | |
| | tgtcccagca ggtgccgagc tctgctccgc tgaagctccc | |
| | ccgcccccgc ccccttcccc acaggacacg ggcctatcca | |
| | caggcttctg agaagccagc ctgctagaag gctgaaccag | |
| | aaccaattgt tttcatccct gtcttactgc ctcctgtcac | |
| | ccgctgccat tgtcgaaggc tgtctttttt ggccatctgc | |
| | tcctggatct ctcttgagat gggcttccca agggctgccg | |
| | ggacagcccc agtcacagta ttgctacccc agtaccttct | |
| | caggcccttc caccggtccc agccgtggtt ttttcatcag | |
| | gtttctccca gatctggaaa gtcagctcag caccccatcc | |
| | cccagcctgt gtgctgagct ctgtagacca gcgaggggca | |
| | tcagggaggg acctgctcag tgcccaccca cccccccttc | |
| | ccgctgagaa gggtgtagcc ccgtcataac aaaggtacca | |
| | tcgtaggctg gctcccagcc cttctctcgg ctcatacact | |
| | cgtatgatac tctgacactg ttcttggctc aatgagcatg | |
| | ctcacacttt aatataagct atttttctaa ctacaaaggt | |
| | ttaaatgaac aagagaggcg ttctcatcgg aaatttagca | |
| | tcgtagtgct ttgagagagg aaaggactcc ttaaaagaga | |
| | aaaaaaaaag ctgagattta ttaaagaaaa aaatgtattt | |
| | tatgttatat ataaatatat tattacttgt aaatataaag | |
| | acgttttata agcatcatta tttatgtatt gtgcaatgtg | |
| | tataaacgag aataaagaaa agatgcactt tgctttaata | |
| | taaacgcaaa taacatgcca aattaaaaaa aaaaaagata | |
| | aacacaagat tggtgttttt ttctatgggt gttatcacct | |
| | agctgaatgt ttttctaaag gagtttatgt tccattaaac | |
| | aatttttaaa atgtataaaa aaaaaaaaaa a | |
| SEQ ID NO: 11 | cttcggctgc cccacccg | |
| SEQ ID NO: 12 | atcgtttggt cctgaacat | |
| SEQ ID NO: 13 | ccctcctcct cgtcctcg | |
| SEQ ID NO: 14 | gtcgcccctt ctccccgcag | |
| SEQ ID NO: 15 | gccgtccgtc gccccttc | |
| SEQ ID NO: 16 | agcaccgagt gcgtgagc | |
| SEQ ID NO: 17 | agttcacaga gtcgacta | |
| SEQ ID NO: 18 | ggcaaaagcc attcccct | |
| SEQ ID NO: 19 | gccgatcttg ctccgcac | |
| SEQ ID NO: 20 | ctccggctgc cccacccc | |
| SEQ ID NO: 21 | cgaacatgac ctccgcac | |
| SEQ ID NO: 22 | atcgtttggt cctgaacat | |
| SEQ ID NO: 23 | ccctcctcct cgtcctcg | |
| SEQ ID NO: 24 | gctgtccgtc gccccttc | |
| SEQ ID NO: 25 | agcaccgagt gcgtgagc | |
| SEQ ID NO: 26 | agttcgcaga gtcggcta | |
| SEQ ID NO: 27 | ggcaaaagcc attcccct | |
| SEQ ID NO: 28 | gccgattttg ctccgcac | |
| SEQ ID NO: 29 | ctgccccttc ttccaaaa | |
| SEQ ID NO: 30 | actcacacac actcctga | |
| SEQ ID NO: 31 | tgcccaggta ctgcctct | |
| SEQ ID NO: 32 | gagatccagg agcagatg | |
| SEQ ID NO: 33 | cttcggctgc cccacccg | |
| SEQ ID NO: 34 | atcgtttggt cctgaacat | |
| SEQ ID NO: 35 | ccctcctcct cgtcctcg | |
| SEQ ID NO: 36 | gtcgcccctt ctccccgcag | |
| SEQ ID NO: 37 | gccgtccgtc gccccttc | |
| SEQ ID NO: 38 | agcaccgagt gcgtgagc | |
| SEQ ID NO: 39 | agttcacaga gtcgacta | |
| SEQ ID NO: 40 | ggcaaaagcc attcccct | |
| SEQ ID NO: 41 | gccgatcttg ctcctcac | |
| SEQ ID NO: 42 | gtcgcccctt ctcccccgca g | |
| SEQ ID NO: 43 | gtcgcaccgt ctcacagcag | |
| SEQ ID NO: 44 | atggacaata tgtct | |
| SEQ ID NO: 45 | ctgcggggag aaggggcgac | |
| SEQ ID NO: 46 | guucaggacc aaacgaucug c | |
| SEQ ID NO: 47 | gcagaucguu ugguccugaa cau | |
| SEQ ID NO: 48 | cucacgcacu cggugcucaa g | |
| SEQ ID NO: 49 | cuugagcacc gagugcguga gcg | |
| SEQ ID NO: 50 | cucggcgccc gacuucuucu u | |
| SEQ ID NO: 51 | gaagaagucg ggcgccgagu u | |
| SEQ ID NO: 52 | acgacuuuuc uccucgccuu u | |
| SEQ ID NO: 53 | aggcgaggag aaaagucguu u | |
| SEQ ID NO: 54 | acgaucugcg cucguccggu u | |
| SEQ ID NO: 55 | ccggacgagc gcagaucguu u | |
| SEQ ID NO: 56 | ggcgcucacg cacucggugu u | |
| SEQ ID NO: 57 | caccgagugc gugagcgccu u | |
| SEQ ID NO: 58 | ggagcggcag cuggagcugu u | |
| SEQ ID NO: 59 | cagcuccagc ugccgcuccu u | |
| SEQ ID NO: 60 | aguguucagg uggccggauu u | |
| SEQ ID NO: 61 | auccggccac cugaacacuu u | |
| SEQ ID NO: 62 | gucaagaggc uguguugcuu u | |
| SEQ ID NO: 63 | agcaacacag ccucuugacu u | |
| SEQ ID NO: 64 | gaggcugugu ugcugugaau u | |
| SEQ ID NO: 65 | uucacagaca cacagccucu u | |
| SEQ ID NO: 66 | ucuuacggga agaucaaccu u | |
| SEQ ID NO: 67 | gguugaucuu cccguaagau u | |
| SEQ ID NO: 68 | gaucaacccc gagcuggugu u | |
| SEQ ID NO: 69 | caccagcucg ggguugaucu u | |
| SEQ ID NO: 70 | ccccgagcug gugugcugcu u | |
| SEQ ID NO: 71 | gcagcacacc agcucggggu u | |
| SEQ ID NO: 72 | cgaauuaucu ggccccuggu u | |
| SEQ ID NO: 73 | ccaggggcca gauaauucgu u | |
| SEQ ID NO: 74 | cuucuucugg agccuggggu u | |
| SEQ ID NO: 75 | ccccaggcuc cagaagaagu u | |
| SEQ ID NO: 76 | uggcuuuugc cucggacagu u | |
| SEQ ID NO: 77 | cuguccgagg caaaagccau u | |
| SEQ ID NO: 78 | uucggacaac aagagucagu u | |
| SEQ ID NO: 79 | cugacucuug uuguccgaau u | |
| SEQ ID NO: 80 | ccgcagcagu uaccccaucu u | |
| SEQ ID NO: 81 | gaugggguaa cugcugcggu u | |
| SEQ ID NO: 82 | guccgccaca cuggacaacu u | |
| SEQ ID NO: 83 | guuguccagu guggcggacu u | |
| SEQ ID NO: 84 | cccggacucc aggacgcugu u | |
| SEQ ID NO: 85 | cagcguccug gaguccgggu u | |
| SEQ ID NO: 86 | atgttcagga ccaaacgatc tgcg | |
| SEQ ID NO: 87 | agctgccgct ccttcagttt ctt | |
| SEQ ID NO: 88 | atgttcagga ccaaacgatc tgcgctcgtc cggcgtctct | Homo sapiens |
| | ggaggagccg tgcgcccggc ggcgaggacg aggaggaggg | SMAD7 mRNA, |
| | cgcaggggga ggtggaggag gaggcgagct gcggggagaa | GenBank XM_033746 |
| | ggggcgacgg acagccgagc gcatggggcc ggtggcggcg | |
| | gcccgggcag ggctggatgc tgcctgggca aggcggtgcg | |
| | aggtgccaaa ggtcaccacc atccccaccc gccagccgcg | |
| | ggcgccggcg cggccggggg cgccgaggcg gatctgaagg | |
| | cgctcacgca ctcggtgctc aagaaactga aggagcggca | |
| | gctggagctg ctgctccagg ccgtggagtc ccgcggcggg | |
| | acgcgcaccg cgtgcctcct gctgcccggc cgcctggact | |
| | gcaggctggg cccgggggcg cccgccggcg cgcagcctgc | |
| | gcagccgccc tcgtcctact cgctccccct cctgctgtgc | |
| | aaagtgttca ggtggccgga tctcaggcat tcctcggaag | |
| | tcaagaggct gtgttgctgt gaatcttacg ggaagatcaa | |
| | ccccgagctg gtgtgctgca acccccatca ccttagccga | |
| | ctctgcgaac tagagtctcc cccccctcct tactccagat | |
| | acccgatgga ttttctcaaa ccaactgcag actgtccaga | |
| | tgctgtgcct tcctccgctg aaacaggggg aacgaattat | |
| | ctggcccctg gggggctttc aggattccca acttcttctg | |
| | gagcctgggg atcggtcaca ctggtgcgtg gtggcatact | |
| | gggaggagaa gacgagagtg gggaggctct actgtgtcca | |
| | ggagccctct ctggatatct tctatgatct acctcagggg | |
| | aatggctttt gcctcggaca gctcaattcg gacaacaaga | |
| | gtcagctggt gcagaaggtg cggagcaaaa tcggctgcgg | |
| | catccagctg acgcgggagg tggatggtgt gtgggtgtac | |
| | aaccgcagca gttaccccat cttcatcaag tccgccacac | |
| | tggacaaccc ggactccagg acgctgttgg tacacaaggt | |
| | gttccccggt ttctccatca aggctttcga ctacgagaag | |
| | gcgtacagcc tgcagcggcc caatgaccac gagtttatgc | |
| | agcagccgtg gacgggcttt accgtgcaga tcagctttgt | |
| | gaagggctgg ggccagtgct acacccgcca gttcatcagc | |
| | agctgcccgt gctggctaga ggtcatcttc aacagccggt ag | |
| SEQ ID NO: 89 | cggagagccg cgcagggcgc gggccgcgcg gggtggggca | Homo sapiens |
| | gccggagcgc aggcccccga tccccggcgg gcgcccccgg | SMAD7 mRNA, |
| | gcccccgcgc gcgccccggc ctccgggaga ctggcgcatg | GenBank XM_008803 |
| | ccacggagcg cccctcgggc cgccgccgct cctgcccggg | |
| | cccctgctgc tgctgctgtc gcctgcgcct gctgccccaa | |
| | ctcggcgccc gacttcttca tggtgtgcgg aggtcatgtt | |
| | cgctccttag caggcaaacg acttttctcc tcgcctcctc | |
| | gccccgcatg ttcaggacca aacgatctgc gctcgtccgg | |
| | cgtctctgga ggagccgtgc gcccggcggc gaggacgagg | |
| | aggagggcgc agggggaggt ggaggaggag gcgagctgcg | |
| | gggagaaggg gcgacggaca gccgagcgca tggggccggt | |
| | ggcggcggcc cgggcagggc tggatgctgc ctgggcaagg | |
| | cggtgcgagg tgccaaaggt caccaccatc cccacccgcc | |
| | agccgcgggc gccggcgcgg ccgggggcgc cgaggcggat | |
| | ctgaaggcgc tcacgcactc ggtgctcaag aaactgaagg | |
| | agcggcagct ggagctgctg ctccaggccg tggagtcccg | |
| | cggcgggacg cgcaccgcgt gcctcctgct gcccggccgc | |
| | ctggactgca ggctgggccc gggggcgccc gccggcgcgc | |
| | agcctgcgca gccgccctcg tcctactcgc tccccctcct | |
| | gctgtgcaaa gtgttcaggt ggccggatct caggcattcc | |
| | tcggaagtca agaggctgtg ttgctgtgaa tcttacggga | |
| | agatcaaccc cgagctggtg tgctgcaacc cccatcacct | |
| | tagccgactc tgcgaactag agtctccccc ccctccttac | |
| | tccagatacc cgatggattt tctcaaacca actgcagact | |
| | gtccagatgc tgtgccttcc tccgctgaaa cagggggaac | |
| | gaattatctg gcccctgggg ggctttcagg attcccaact | |
| | tcttctggag cctggggatc ggtcacactg gtgcgtggtg | |
| | gcatactggg aggagaagac gagagtgggg aggctctact | |
| | gtgtccagga gccctctctg gatatcttct atgatctacc | |
| | tcaggggaat ggcttttgcc tcggacagct caattcggac | |
| | aacaagagtc agctggtgca gaaggtgcgg agcaaaatcg | |
| | gctgcggcat ccagctgacg cgggaggtgg atggtgtgtg | |
| | ggtgtacaac cgcagcagtt accccatctt catcaagtcc | |
| | gccacactgg acaacccgga ctccaggacg ctgttggtac | |
| | acaaggtgtt ccccggtttc tccatcaagg ctttcgacta | |
| | cgagaaggcg tacagcctgc agcggcccaa tgaccacgag | |
| | tttatgcagc agccgtggac gggctttacc gtgcagatca | |
| | gctttgtgaa gggctggggc cagtgctaca cccgccagtt | |
| | catcagcagc tgcccgtgct ggctagaggt catcttcaac | |
| | agccggtagc cgcgtgcgga ggggacagag cgtgagctga | |
| | gcaggccaca cttcaaacta ctttgctgct aatattttcc | |
| | tcctgagtgc ttgcttttca tgcaaactct ttggtcgttt | |
| | tttttttgtt tgttggttgg ttttcttctt ctcgtcctcg | |
| | tttgtgttct gttttgtttc gctctttgag aaatagctta | |
| | tgaaaagaat tgttgggggt ttttttggaa gaaggggcag | |
| | gtatgatcgg caggacaccc tgataggaag aggggaagca | |
| | gaaatccaag caccaccaaa cacagtgtat gaaggggggc | |
| | ggtcatcatt tcacttgtca ggagtgtgtg tgagtgtgag | |
| | tgtgcggctg tgtgtgcacg cgtgtgcagg agcggcagat | |
| | ggggagacaa cgtgctcttt gttttgtgtc tcttatggat | |
| | gtccccagca gagaggtttg cagtcccaag cggtgtctct | |
| | cctgcccctt ggacacgctc agtggggcag aggcagtacc | |
| | tgggcaagct ggcggctggg gtcccagcag ctgccaggag | |
| | cacggctctg tccccagcct gggaaagccc ctgcccctcc | |
| | tctccctcat caaggacacg ggcctgtcca caggcttctg | |
| | agcagcgagc ctgctagtgg ccgaaccaga accaattatt | |
| | ttcatccttg tcttattccc ttcctgccag cccctgccat | |
| | tgtagcgtct ttcttttttg gccatctgct cctggatctc | |
| | cctgagatgg gcttcccaag ggctgccggg gcagccccct | |
| | cacagtattg ctcacccagt gccctctccc ctcagcctct | |
| | cccctgcctg ccctggtgac atcaggtttt tcccggactt | |
| | agaaaaccag ctcagcactg cctgctccca gcctgtgtgt | |
| | taagctctgc tattaggcca gcaagcgggg atgtccctgg | |
| | gagggacatg cttagcagtc cccttccctc caagaaggat | |
| | ttggtccgtc ataacccaag gtaccatcct aggctgacac | |
| | ctaactcttc tttcatttct tctacaactc atacactcgt | |
| | atgatacttc gacactgttc ttagctcaat gagcatgttt | |
| | agactttaac ataagctatt tttctaacta caaaggttta | |
| | aatgaacaag agaagcattc tcattggaaa tttagcattg | |
| | tagtgctttg agagagaaag gactcctgaa aaaaaacctg | |
| | agatttatta aagaaaaaaa tgtattttat gttatatata | |
| | aatatattat tacttgtaaa tataaagacg ttttataagc | |
| | atcattattt atgtattgtg caatgtgtat aaacaagaaa | |
| | aataaagaaa agatgcactt tgctttaata taaatgcaaa | |
| | taacaaatgc caaattaaaa aagataaaca caagattggt | |
| | gtttttttct atgggtgtta tcacctagct gaatgttttt | |
| | ctaaaggagt ttatgttcca ttaaacgatt tttaaaatgt | |
| | acacttg | |
| SEQ ID NO: 90 | atgttcagga ccaaacgatc tgcgctcgtc cggcgtctct | Homo sapiens |
| | ggaggagccg tgcgcccggc ggcgaggacg aggaggaggg | SMAD7 mRNA, |
| | cgcaggggga ggtggaggag gaggcgagct gcggggagaa | GenBank AF015261 |
| | ggggcgacgg acagccgagc gcatggggcc ggtggcggcg | |
| | gcccgggcag ggctggatgc tgcctgggca aggcggtgcg | |
| | aggtgccaaa tgtcaccacc atccccaccc gccagccgcg | |
| | ggcgccggcg cggccggggg cgccgaggcg gatctgaagg | |
| | cgctcacgca ctcggtgctc aagaaactga aggagcggca | |
| | gctggagctg ctgctccagg ccgtggagtc ccgcggcggg | |
| | acgcgcaccg cgtgcctcct gctgcccggc cgcctggact | |
| | gcaggctggg cccgggggcg cccgccggcg cgcagcctgc | |
| | gcagccgccc tcgtcctact cgctccccct cctgctgtgc | |
| | aaagtgttca ggtggccgga tctcaggcat tcctcggaag | |
| | tcaagaggct gtgttgctgt gaatcttacg ggaagatcaa | |
| | ccccgagctg gtgtgctgca acccccatca ccttagccga | |
| | ctctgcgaac tagagtctcc cccccctcct tactccagat | |
| | acccgatgga ttttctcaaa ccaactgcag actgtccaga | |
| | tgctgtgcct tcctccgctg aaacaggggg aacgaattat | |
| | ctggcccctg gggggctttc agattcccaa cttcttctgg | |
| | agcctgggga tcggtcacac tggtgcgtgg tggcatactg | |
| | ggaggagaag acgagagtgg ggaggctcta ctgtgtccag | |
| | gagccctctc tggatatctt ctatgatcta cctcagggga | |
| | atggcttttg cctcggacag ctcaattcgg acaacaagag | |
| | tcagctggtg cagaaggtgc ggagcaaaat cggctgcggc | |
| | atccagctga cgcgggaggt ggatggtgtg tgggtgtaca | |
| | accgcagcag ttaccccatc ttcatcaagt ccgccacact | |
| | ggacaacccg gactccagga cgctgttggt acacaaggtg | |
| | ttccccggtt tctccatcaa ggctttcgac tacgagaagg | |
| | cgtacagcct gcagcggccc aatgaccacg agtttatgca | |
| | gcagccgtgg acgggcttta ccgtgcagat cagctttgtg | |
| | aagggctggg gccagtgcta cacccgccag ttcatcagca | |
| | gctgcccgtg ctggctagag gtcatcttca acagccggta g | |
| SEQ ID NO: 91 | gtnncccctt ctcccnncag | |
| | modified_base | |
| | (3) cytosine, 5-methylcytosine, or 2′-O- | |
| | methylcytosine | |
| | (4) guanine, 5-methylguanine, or 2′-O- | |
| | methylguanine | |
| | (16) cytosine, 5-methylcytosine, or 2′-O- | |
| | methylcytosine | |
| | (17) guanine, 5-methylguanine, or 2′-O- | |
| | methylguanine | |
| SEQ ID NO: 92 | gtngcccctt ctcccngcag | |
| | modified_base | |
| | (3) 5-methyl 2′-deoxycytidine 5′-monophosphate | |
| | (16) 5-methyl 2′-deoxycytidine 5′-monophosphate | |
| SEQ ID NO: 93 | ntngcccctt ctcccngcan | |
| | modified_base | |
| | (1) 2′-deoxyguanosine methylphosphonate | |
| | (3) 5-methyl 2′-deoxycitidine 5′-monophosphate | |
| | 5-methyl 2′-deoxycitidine 5′-monophosphate | |
| | (20) 2′-deoxyguanosine methylphosphonate | |
| SEQ ID NO: 94 | gtttggtcct gaacatgc | |
| SEQ ID NO: 95 | gtttggtcct gaacat | |
| SEQ ID NO: 96 | gtttggtcct gaacatg | |
| SEQ ID NO: 97 | agcaccgagt gcgtgagc | |
| SEQ ID NO: 98 | ngcacngagt gngtgagn | |
| | modified_base | |
| | (1) 2′-deoxyadenosine methylphosphonate | |
| | (6) 5-methyl 2′ deoxycitidine 5′ monophosphate | |
| | (12) 5-methyl 2′ deoxycitidine 5′ monophosphate | |
| | (18) 2′-deoxycytosine methylphosphonate | |
| SEQ ID NO: 99 | cgaacatgac ctccgcac | |
| SEQ ID NO: 100 | ngaacatgac ctcngcac | |
| | modified_base | |
| | (1) 5-methyl 2′ deoxycitidine 5′ monophosphate | |
| | (14) 5-methyl 2′ deoxycitidine 5′ monophosphate | |
| SEQ ID NO: 101 | gtngcccctt ctcccngcag | |
| | modified_base | |
| | (3) 5-methyl 2′ deoxycitidine 5′ monophosphate | |
| | (16) 5-methyl 2′ deoxycitidine 5′ monophosphate | |
| SEQ ID NO: 102 | gatcgtttgg tcctgaa | |
| SEQ ID NO: 103 | atcgtttggt cctgaac | |
| SEQ ID NO: 104 | ntngcccctt ctcccngcan c | |
| | modified_base | |
| | (1) 2′-deoxyguanosine methylphosphonate | |
| | (3) 5-methyl 2′-deoxycytidine 5′-monophosphate | |
| | (16) 5-methyl 2′-deoxycytidine 5′-monophosphate | |
| | (20) 2′-deoxyguanosine methylphosphonate | |
| SEQ ID NO: 105 | gtngcccctt ctcccngcag c | |
| | modified_base | |
| | (3) 5-methyl 2′-deoxycitidine 5′ monophosphate | |
| | (16) 5-methyl 2′-deoxycytidine 5′-monophosphate | |
| SEQ ID NO: 106 | gtngcccctt ctcccngcag | |
| | modified_base | |
| | (3) chemically modified nucleoside | |
| | (16) chemically modified nucleoside | |
| SEQ ID NO: 107 | tccgtcatcg ctcctcaggg | |
| SEQ ID NO: 108 | atgcattctg cccccaagga | |
| SEQ ID NO: 109 | tgtgaatctt acgggaagat caac | |
| SEQ ID NO: 110 | agttcgcaga gtcggctaag g | |
| SEQ ID NO: 111 | ccgagctggt gtgctgcaac cc | |
| SEQ ID NO: 112 | gaaggtgaag gtcggagtc | |
| SEQ ID NO: 113 | gaagatggtg atgggatttc | |
| SEQ ID NO: 114 | caagcttccc gttctcagcc | |
| SEQ ID NO: 115 | cgacagcagc agcagcag | |
| SEQ ID NO: 116 | aggaggcgag gagaaaag | |
| SEQ ID NO: 117 | gctgtccgtc gccccttc | |
| SEQ ID NO: 118 | aggcggccgg gcagcagg | |
| SEQ ID NO: 119 | agcctcttga cttccgag | |
| SEQ ID NO: 120 | agtctgcagt tggtttga | |
| SEQ ID NO: 121 | agaagttggg aatctgaa | |
| SEQ ID NO: 122 | gtatgccacc acgcacca | |
| SEQ ID NO: 123 | tggacacagt agagcctc | |
| SEQ ID NO: 124 | gccgattttg ctccgcac | |
| SEQ ID NO: 125 | ggacttgatg aagatggg | |
| SEQ ID NO: 126 | accttgtgta ccaacagc | |
| SEQ ID NO: 127 | ctgatctgca cggtaaag | |
| SEQ ID NO: 128 | ggcagctgct gatgaact | |
| SEQ ID NO: 129 | gctcagctca cgctctgt | |
| SEQ ID NO: 130 | tgcatgaaaa gcaagcac | |
| SEQ ID NO: 131 | aaaacagaac acaaacga | |
| SEQ ID NO: 132 | tgcttggatt tctgcttc | |
| SEQ ID NO: 133 | gttgtctccc catctgcc | |
| SEQ ID NO: 134 | ttgggactgc aaacctct | |
| SEQ ID NO: 135 | aattggttct ggttcggc | |
| SEQ ID NO: 136 | aaaacctgat gtcaccag | |
| SEQ ID NO: 137 | acacacagga tgggagca | |
| SEQ ID NO: 138 | ggacatcccc gcttgctg | |
| SEQ ID NO: 139 | gatggtacct tgggttat | |
| SEQ ID NO: 140 | gtagaagaaa tgaaagaa | |
| SEQ ID NO: 141 | aacctttgta gttagaaa | |
| SEQ ID NO: 142 | atttccaatg agaatgct | |
| SEQ ID NO: 143 | caatcttgtg tttatctt | |
| SEQ ID NO: 144 | attcagctag gtgataac | |

What is claimed is:

1. A method of treating a disease in a patient in need thereof, comprising administering to the patient effective amounts of a SMAD7 antisense oligonucleotide (AON) comprising a nucleotide sequence complementary to region 108-128 of the human SMAD7 nucleotide sequence of SEQ ID NO: 1, and a compound capable of modulating a TLR.

2. The method of claim 1, wherein the SMAD7 AON targets nucleotides 403, 233, 294, 295, 296, 298, 299, or 533 of the nucleic acid sequence of SEQ ID NO: 1.

3. The method of claim 1 or 2, wherein the SMAD7 AON comprises COMPOUND (I).

4. The method of any one of claims 1 to 3, wherein the TLR is TLR3, TLR7, TLR8, or TLR9.

5. The method of claim 5, wherein the TLR is TLR9.

6. The method of any one of claims 1 to 5, wherein a compound is capable of modulating the TLR if the compound can increase the expression of IP10, TNFα or IL-6 protein by a peripheral dendritic cell (pDC), when the compound is contacted with the pDC at a concentration of less than 1.0 μM, relative to a pDC control not contacted with the compound, as determined in an immunoassay.

7. The method of any one of claims 1 to 5, wherein a compound is capable of modulating the TLR if the compound can increase the expression of TNFα, IFNγ, TGFβ, IL-6, IL-10, PD-L1, IDO, or ICOS-L protein and decrease the expression of IP10 protein by a pDC, when the compound is contacted with the pDC at a concentration of about 1.0 μM or more, relative to a pDC control not contacted with the compound, as determined in an immunoassay.

8. The method of any one of claims 1 to 5, wherein the compound is capable of modulating the TLR if the compound increases the expression of ICOS-L proteins by a pDC by a factor of 5-fold or more, when contacted with the pDC at a concentration of 1.0 μM or more, in the presence of a quinoline or quinine relative to a pDC control not contacted with the compound, as determined in an immunoassay, wherein the quinoline or quinine is present at a concentration below the threshold concentration at which the quinoline or quinine alone can detectably increase ICOS-L expression.

9. The method of claim 8, wherein the quinoline is hydroxychloroquine.

10. The method of any one of claims 1 to 5, wherein the compound is capable of modulating the TLR if the compound can reduce the PolyI:C-induced IFNα secretion of peripheral blood mononuclear cells (PBMCs), when the compound is contacted with the PBMCs at a concentration of 1.0 μM or less, relative to a PolyI:C-induced PBMC control not contacted with the compound.

11. The method of any one of claims 1 to 5, wherein the compound is capable of modulating the TLR if the compound can reduce the imiquimod-induced IFNα secretion of peripheral blood mononuclear cells (PBMCs), when the compound is contacted with the PBMCs at a concentration of 0.1 μM or less, relative to an imiquimod-induced PBMC control not contacted with the compound.

12. The method of any one of claims 1 to 5, wherein the compound capable of modulating TLR is BL-7040, CYT003, CYT003-QbG10, AZD1419, DIMS0150, E6446, CpG ODN2088, IMO-8400, IMO-3100, CL075, VTX-2337, ODN2006, or naltrexone.

13. The method of any one of claims 1 to 5, wherein the compound capable of modulating the TLR is an antimalarial therapeutic selected from the group consisting of a quinine, a chloroquine, an amodiaquine, a mefloquine, a primaquine, or a derivative thereof.

14. The method of claim 13, wherein the antimalarial therapeutic is hydroxylchloroquine (Plaquenil).

15. The method of any one of claims 1 to 5, wherein the compound capable of modulating the TLR induces IL-1β secretion from a PBMC and induces pDC differentiation, when contacted with the pDC or PBMC at a concentration of 1.0 μM or more.

16. The method of any one of claims 1 to 5, wherein the compound capable of modulating the TLR does not induce B-cell proliferation, when contacted with a B-cell at a concentration of 10.0 μM or less.

17. The method of any one of claims 1 to 5, wherein the TLR pathway component is CCR7, CD80, CD83, CD86, CD69, EGFR, GARP, IL1-β, IL-2, IL-10Rα, IL-18, IL-23p19, MIP-1α, phospho-histone H3, phospho-p38 MAP kinase, phospho-ZAP70, RANKL, SLAMF7, tPA, or uPAR.

18. The method of any one of claims 1 to 17, wherein the disease is selected from the group consisting of an inflammatory disease, an autoimmune disorder, an airway disease, an allergic disorder, a metabolic disorder, cancer, central nervous system (CNS) disorder, and a skin disease.

19. The method of any one of claims 1 to 17, wherein the disease is selected from the group consisting of inflammatory bowel disease (IBD), Crohn's disease (CD), ulcerative colitis (UC), Sjogren's Syndrome, systemic lupus erythematosus (SLE), dry eye, autoimmune encephalitis, rheumatoid arthritis, multiple sclerosis, systemic sclerosis, psoriasis, colitis, uveitis, asthma, chronic pulmonary disease (COPD), allergic rhinitis, atopic dermatitis, Malaria, Hashimoto's encephalopathy, amoebiasis, diabetes, hyperlipidemia, non-alcoholic fatty liver disease, lung cancer, pancreas cancer, leukemic cancer, lymphoid cancer, pancreas cancer, breast cancer, prostate cancer, ovarian cancer, testicular cancer, melanoma, myeloma, glioblastoma, neuroblastoma, colorectal cancer, stomach cancer, multiple sclerosis, basal cell carcinoma, and actinic keratosis.

20. A method of treating a disease in a patient in need thereof, comprising (a) administering to the patient an effective amount of a SMAD7 AON comprising a nucleotide sequence complementary to region 108-128 of the human SMAD7 nucleotide sequence of SEQ ID NO: 1; (b) determining the patient's response to the ODN, and (c) if the patient does not respond to the SMAD7 AON, then, administering to the patient an effective amount of the SMAD7 AON and an effective amount of a compound capable of modulating a TLR.

21. The method of claim 20, wherein determining the patient's response to the SMAD7 AON comprises (a) analyzing a first level of a biomarker before administering the SMAD7 AON to the patient, and (b) analyzing a second level of the biomarker after administering the SMAD7 AON to the patient, wherein the patient responds to the SMAD7 AON if the second biomarker level is lower than the first biomarker level.

22. The method of claim 20 or 21, wherein the SMAD7 AON comprises COMPOUND (I).

23. The method of any one of claims 20 to 22, wherein the disease is IBD.

24. The method of any one of claims 20 to 23, wherein the disease is Crohn's Disease or Ulcerative Colitis.

25. The method of any one of claims 20 to 24, wherein the SMAD7 AON is COMPOUND (I), and wherein the compound capable of modulating a TLR is hydroxychloroquine.

26. A pharmaceutical composition comprising a SMAD7 AON comprising a nucleotide sequence complementary to region 108-128 of the human SMAD7 nucleotide sequence of SEQ ID NO: 1, a compound capable of modulating a TLR, and an excipient.

27. The pharmaceutical composition of claim 26, wherein the SMAD7 AON is COMPOUND (I).

28. The pharmaceutical composition of claims 26 or 27, wherein the TLR is TLR3, TLR7, TLR8, or TLR9.

29. The pharmaceutical composition of claim 38, wherein the TLR is TLR9.

30. The pharmaceutical composition of any one of claims 26 to 29, wherein the compound capable of modulating the TLR is hydroxychloroquine.

31. The pharmaceutical composition of any one of claims 26 to 30, wherein the ODN and the compound capable of modulating TLR are covalently linked.


SEQUENCE LISTING (158 sequences)

SEQ ID NO: 1 - DNA, 1281 bp, Homo sapiens
  human SMAD family member 7 (SMAD7) variant 1 mRNA
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 2 - DNA, 20 bp, Artificial Sequence
  SMAD7 ODN
gtcgccccttctccccgcag

SEQ ID NO: 3 - DNA, 21 bp, Artificial Sequence
  SMAD7 ODN
gtcgccccttctccccgcagc

SEQ ID NO: 4 - DNA, 20 bp, Artificial Sequence
  modified SMAD7 ODN
  misc_feature 3, 16: n = 5-methyl 2′-deoxycytidine
gtngccccttctcccngcag

SEQ ID NO: 5 - DNA, 21 bp, Artificial Sequence
  modified SMAD7 ODN
  misc_feature 3, 16: n = 5-methyl 2′-deoxycytidine
gtngccccttctcccngcagc

SEQ ID NO: 6 - DNA, 20 bp, Artificial Sequence
  modified SMAD7 ODN
  misc_feature 3, 16: n = nucleoside comprising a nitrogenous base selected from the group consisting of cytosine, 5-methylcytosine and 2-O-methylcytosine
  misc_feature 4, 17: n = nucleoside comprising a nitrogenous base selected from the group consisting of guanine, 5-methylguanine and 2-O-methylguanine
gtnnccccttctcccnncag

SEQ ID NO: 7 - DNA, 21 bp, Artificial Sequence
  modified SMAD7 ODN
  misc_feature 3, 16: n = nucleoside comprising 5-methyl 2′-deoxycytidine
gtngccccttctcccngcagc

SEQ ID NO: 8 - DNA, 3111 bp, Homo sapiens
  human SMAD 7 sequence (GenBank AF0010193)
ggcacgagcggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgttttttcctatgggtgttatcacctagctgaatgtttttctaaaggagtttatgttccattaaacgatttttaaaatgtacacttgaaaaaaaaaaaaaaaaa

SEQ ID NO: 9 - DNA, 3681 bp, Mus musculus
  mouse SMAD7 mRNA (GenBank NM_008543)
cgagtgcggcgcggcgagcccccagcggcggcagaaggactcgagcgccaggagagggcggacgggggacgaggaggctccggggcgcgacgaagagagtctccgaggaagaggctgcgagaggacacccgggcctcctgccgccactgtcgggtcggggccagcagctcatgagagcagccccggcggccacccgcggccaggagaaggagcaccggaggcccccacactagcctgtgccctcgggggcgagagcttgcgacccgccggagcccgccgccgcgccgccctcccccgcgctgacagccccccggggcgcagccgccgccgcagcatcttctgtccctgcttccccagcgcggaggaagtccccgccgaggacctgagcccccgggaacgcaggaggaaagaccagagactctaaaacacccagatacgcaagattgaagcagcctagccagacctttctgtggattaaaagaaatacgattttttttttttttttttggcagaagaaaaggaaaggaagaccggctgggttcagcaaggaaaaaaagggggatgtaactcgtggatacggtttttttcccccacccttccaacatcttgttttattttgtaaacattttctcttttaaacccgggctccatccggtgccctccagacctccgaggtgcgaggaggtggtgtgttttttcattgggggctttgcatattttggttttgggggttttgagagaccctccagacatctcacgaggggtgaagtctactcggtcccctcccgcaagtcttcgcgtgcacagaattcgaggagatccggttactaaggatatagaagaaaaaaaataaatcgtgcctgccttttttttttaattgcctgcttctccccacccccaaattaagttgcttagcaagggggaaagaggctttttcctccctttagtagctcagcctaacgtctttcgtttttttttttttttttttttttttgcccccgaggatcttccatgtaggaagccgaggctggcgagcccgacactcgggagccactgtaggggggccttttttgggggaggcgtctaccggggttgcctcggccgcccccagggaagcggcggccgcgttcctccagggcacgccggggcccgaaagccgcgcagggcgcgggccgcgccgggtggggcagccgaagcgcagccccccgatccccggcaggcgcccctgggcccccgcgcgcgccccggcctctgggagactggcgcatgccacggagcgcccctcgggccgccgccgcttctgcccgggcccctgctgttgctgctgtcgcctgcgcctgctgccccaactcggcgcccgacttcttcatggtgtgcggaggtcatgttcgctccttagccggcaaacgacttttctcctcgcctcctcgccccgcatgttcaggaccaaacgatctgcgctcgtccggcgtctctggaggagccgtgcgcccggcggcgaggacgaggaggagggcgtggggggtggcggcggaggaggcgagctgcggggagaaggggcgacggacggccgggcttatggggctggtggcggcggtgcgggcagggctggctgctgcctgggcaaggcagtccgaggtgccaaaggtcaccaccatccccatcccccaacctcgggtgccggggcggccgggggcgccgaggcggatctgaaggcgctcacgcactcggtgctcaagaaactcaaggagcggcagctggagctgctgcttcaggccgtggagtcccgcggcggtacgcgcaccgcgtgcctcctgctgcccggccgcctggactgcaggctgggcccgggggcgcccgccagcgcgcagcccgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagtcgactctgtgaactagagtctccccctcctccttactccagatacccaatggattttctcaaaccaactgcaggctgtccagatgctgtaccttcctccgcggaaaccgggggaacgaattatctggcccctggggggctttcagattcccaacttcttctggagcctggggatcggtcacactggtgcgtggtggcatactgggaggagaagactcgcgtggggaggctctactgtgtccaagagccctccctggatatcttctatgatctacctcaggggaatggcttttgcctcggacagctcaattcggacaacaagagtcagctggtacagaaagtgcggagcaagatcggctgtggcatccagctgacgcgggaagtggatggcgtgtgggtttacaaccgcagcagttaccccatcttcatcaagtccgccacactggacaacccggactccaggacgctgttggtgcacaaagtgttccctggtttctccatcaaggcttttgactatgagaaagcctacagcctgcagcggcccaatgaccacgagttcatgcagcaaccatggacgggtttcaccgtgcagatcagctttgtgaagggctggggccagtgctacacccgccagttcatcagcagctgcccgtgctggctggaggtcatcttcaacagccggtagtcggtcgtgtggtggggagaagaggacagggcggatcgtgagccgagcaggccaccgttcaaactacttgctgctaatctttcccgagtgattgcttttcatgcaaactctttggttggtgttgttattgccattcattgttggttttgttttgttctgttctggtttgttttttttttttttcctccccaagggctgccgggacagccccagtcacagtattgctaccccagtaccctctcaggcccttccaccgggtcccagccgtggtggttttttcatcaggtttctcccagatgtggaaagtcagctcagcatcccatcccccatcctgtgtgctgagctctgtagaccagcgaggggcatcagggagggacctgcgcagtgcccccccttcctgctgagaagggtgtagccccgtcacaacaaaggtaccatcccttggctggctcccagcccttctctcagctcatacgctcgctcgtatgatactttgacactgttcttagctcaatgagcatgtttagaatttaacataagctatttttctaactacaaaggtttaaatgaacaagagaagcattctcattggaaatttagcattgtagtgctttgagagaggaaaggactccttaaaagaaaaaaaaagctgagatttattaaagaaaaatgtattttatgttatatataaatatattattacttgtaaatataaagacgttttataagcatcattatttatgtattgtgcaatgtgtataaacgagaagaataaagaaaagatgcactttgctttaatataaatgtaaataacatgccaaattaaaaaaaaaaagataaacacaagattggtgtttttttctatgggtgttatcacctagctgaatgtttttctaaaggagtttatgttccattaaacaatttttaaaatgttaaaaaaaaaaaaaaaaaaaaaaaaaaaaaa

SEQ ID NO: 10 - DNA, 4311 bp, Rattus norvegicus
  rat SMAD7 mRNA (GenBank NM_030858)
tgagtgcggcgcggcgagcccccagcggcggcagaaggactcgagcgccaggagagggcggacgggggacgaggaggctcccgggcgcgacgaagagagtctcggaggaagaggctgcgagaggacacccgggcctcctgccgccactgtcgggtcggggccagcagcttatgcgagcagccccagcgaccaccctcggccaggagaaggggcaccggcagcccccacgctagctagcctgccgcctgtgccctcgggggcgagagcttgcgacccgccggagcccgccgccgcgccgccctcccccgcgctgacagccccccggggcgcagccgccgccgcagcatcttctgtccctgcttccccagcgcggaggaagtccccgccgaggacctgggcccccgggagcgcaggaggaaagaccagagactctaaaacacccagatacgcaagattgaagcagcctaaccagacctttctgtggattaaaagaaatacgattttttttttgacagaagaaaaggaaaggaagaccggcggggttcagcaaggaaaaaaaggggatgtaactcgtggatacggtttttccccccacccttccaacatcttgttctactttgtaaacattttctctttttaaaccccggctccatccggtgccctccagacctccgaggtgcgagaaggtggtgtgttttttcactgggggctttgcatatttggttttggggtttttgagagaccctccagacatctcacgaggggtgaagtctactcggccccctccctcaagtcttcgcgtgcacagaattcgaggagatccggttactaaggatatagaagaaaaaaataaatcgtgtgcctgccttttttttttttaattgcctgcttctccccacccccaaattaagttgcttagcaagggggaaagaggctttttcctcccttcagtagctcagcctaacgtctttcgttttttgcccctgaggatcttccatgtaggaagccgaggctggcgagcccgacactcgggagccactgtaggggggcctttttggggagaggcgtcgaccggggctgcctcggccgcccccagggaagcggcggccgcgttcctcaggggcacgccggggcccgagagccgcgcagggcgcgggccgcgccgggtggggcagccgaagcgcaggcccccgatccccggcgggcgcccctgggcccccgcgcgcgccccggcctccgggagactggcgcatgccacggagcgcccctcgggccgccgccgcttctgcccgggcccctgctgttgttgctgtcgcctgcgcctgctgccccaactcggcgcccgacttcttcatggtgtgcggaggtcatgttcgctccttagccggcaaacgacttttctcctcgcctcctcgccccgcatgttcaggaccaaacgatctgcgctcgtccggcgtctctggaggagccgtgcgcccggcggcgaggacgaggaggagggcgtggggggtggcggcggcggaggcgacctgcggggagaaggggcgacggacggccgggcttatggggctggtggcggcggtgcgggcagggctggctgctgcctgggtaaggcagtccgaggtgccaaaggtcaccaccatccccatcccccatcctcgggtgccggggcggccgggggcgccgaggcggatctgaaggcgctcacgcactcggtgctcaagaaactcaaggagcggcagctggagctgctgcttcaggccgtggagtcccgcggcggtacgcgcaccgcgtgcctcctgctgcccggccgcctggactgcaggctgggcccgggggcgcccgccagcgcgcagcccgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagtcgactctgtgaactagagtctccccctcctccttactccagatacccgatggattttctcaaaccaactgcagactgtccagacgctgtaccttcctccgatgaaaccgggggaacgaattatctggcccctggggggctttcagattcccaacttcttctggagcctggggatcggtcacactggtgcgtggtggcatactgggaggagaagactcgagtggggaggctctactgtgtccaagagccctccctggatatcttctatgatctacctcaggggaatggcttttgcctcggacagctcaattcggacaacaagagtcagctggtacagaaagtgaggagcaagatcggctgtggcatccagctgacaagggaagtggatggcgtgtgggtttacaaccgcagcagttaccccatcttcatcaagtccgccacactggacaacccggactccaggacgctgttggtgcacaaagtgttccctggtttctccatcaaggcttttgactatgaaaaggcctacagcctgcagcggcccaatgaccacgagttcatgcagcagccatggacgggcttcaccgtgcagattagcttcgtgaagggctggggccagtgctacacccgccagttcatcagcagttgcccgtgctggctggaggtcatcttcaacagccggtagtctcccggtgtggggagaagaggacaggacggaggggtgagccgagcaggccaccgttcaaactacttgctgctaatctttcatgcaaaactctttcggtcggttttgttgtttgccattcattgttggttctgttttgttttgttttccttttttttttttcttccttcttctttttcctcctttcttgtcactcttgtgtcctgtgtgtctcgttctttgagaaaatatgatgcggatttttggttgtgtgtttttttttttcgtttgtttgtttgttgttgttgtttgtgttttgaggtggtggtgggtgcggttggcaggacaccccgatacaaaaacgggaagcaagagtcagcactgccaagcgtggtgtgcgaaagtgggtaccaccttcccctttggatcagcatttcagttgtcagtgtgtgtgtgtgaggggggtgtacgtgaatgacagatgggggaatggcgtgctttttttgtgttctttatggatgtccccagctgagaggcttgcagttccaagctgtgtgtctctcactgtgtgtctctctcatgagcctttcggacatgctcggtggggcagaggctgtacctgggcagactggcagcaggtgtcccagcaggtgccgagctctgctccgctgaagctcccccgcccccgcccccttccccacaggacacgggcctatccacaggcttctgagaagccagcctgctagaaggctgaaccagaaccaattgttttcatccctgtcttactgcctcctgtcacccgctgccattgtcgaaggctgtcttttttggccatctgctcctggatctctcttgagatgggcttcccaagggctgccgggacagccccagtcacagtattgctaccccagtaccttctcaggcccttccaccggtcccagccgtggttttttcatcaggtttctcccagatctggaaagtcagctcagcaccccatcccccagcctgtgtgctgagctctgtagaccagcgaggggcatcagggagggacctgctcagtgcccacccacccccccttcccgctgagaagggtgtagccccgtcataacaaaggtaccatcgtaggctggctcccagcccttctctcggctcatacactcgtatgatactctgacactgttcttggctcaatgagcatgctcacactttaatataagctatttttctaactacaaaggtttaaatgaacaagagaggcgttctcatcggaaatttagcatcgtagtgctttgagagaggaaaggactccttaaaagagaaaaaaaaaagctgagatttattaaagaaaaaaatgtattttatgttatatataaatatattattacttgtaaatataaagacgttttataagcatcattatttatgtattgtgcaatgtgtataaacgagaataaagaaaagatgcactttgctttaatataaacgcaaataacatgccaaattaaaaaaaaaaaagataaacacaagattggtgtttttttctatgggtgttatcacctagctgaatgtttttctaaaggagtttatgttccattaaacaatttttaaaatgtataaaaaaaaaaaaaaa

SEQ ID NO: 11 - DNA, 18 bp, Artificial Sequence
  SMAD7 ODN
cttcggctgccccacccg

SEQ ID NO: 12 - DNA, 19 bp, Artificial Sequence
  SMAD7 ODN
atcgtttggtcctgaacat

SEQ ID NO: 13 - DNA, 18 bp, Artificial Sequence
  SMAD7 ODN
ccctcctcctcgtcctcg

SEQ ID NO: 14 - DNA, 20 bp, Artificial Sequence
  SMAD7 ODN
gtcgccccttctccccgcag

SEQ ID NO: 15 - DNA, 18 bp, Artificial Sequence
  SMAD7 ODN
gccgtccgtcgccccttc

SEQ ID NO: 16 - DNA, 18 bp, Artificial Sequence
  SMAD7 ODN
agcaccgagtgcgtgagc

SEQ ID NO: 17 - DNA, 18 bp, Artificial Sequence
  SMAD7 ODN
agttcacagagtcgacta

SEQ ID NO: 18 - DNA, 18 bp, Artificial Sequence
  SMAD7 ODN
ggcaaaagccattcccct

SEQ ID NO: 19 - DNA, 18 bp, Artificial Sequence
  SMAD7 ODN
gccgatcttgctccgcac

SEQ ID NO: 20 - DNA, 18 bp, Artificial Sequence
  SMAD7 ODN
ctccggctgccccacccc

SEQ ID NO: 21 - DNA, 18 bp, Artificial Sequence
  SMAD7 ODN
cgaacatgacctccgcac

SEQ ID NO: 22 - DNA, 19 bp, Artificial Sequence
  SMAD7 ODN
atcgtttggtcctgaacat

SEQ ID NO: 23 - DNA, 18 bp, Artificial Sequence
  SMAD7 ODN
ccctcctcctcgtcctcg

SEQ ID NO: 24 - DNA, 18 bp, Artificial Sequence
  SMAD7 ODN
gctgtccgtcgccccttc

SEQ ID NO: 25 - DNA, 18 bp, Artificial Sequence
  SMAD7 ODN
agcaccgagtgcgtgagc

SEQ ID NO: 26 - DNA, 18 bp, Artificial Sequence
  SMAD7 ODN
agttcgcagagtcggcta

SEQ ID NO: 27 - DNA, 18 bp, Artificial Sequence
  SMAD7 ODN
ggcaaaagccattcccct

SEQ ID NO: 28 - DNA, 18 bp, Artificial Sequence
  SMAD7 ODN
gccgattttgctccgcac

SEQ ID NO: 29 - DNA, 18 bp, Artificial Sequence
  SMAD7 ODN
ctgccccttcttccaaaa

SEQ ID NO: 30 - DNA, 18 bp, Artificial Sequence
  SMAD7 ODN
actcacacacactcctga

SEQ ID NO: 31 - DNA, 18 bp, Artificial Sequence
  SMAD7 ODN
tgcccaggtactgcctct

SEQ ID NO: 32 - DNA, 18 bp, Artificial Sequence
  SMAD7 ODN
gagatccaggagcagatg

SEQ ID NO: 33 - DNA, 18 bp, Artificial Sequence
  SMAD7 ODN
cttcggctgccccacccg

SEQ ID NO: 34 - DNA, 19 bp, Artificial Sequence
  SMAD7 ODN
atcgtttggtcctgaacat

SEQ ID NO: 35 - DNA, 18 bp, Artificial Sequence
  SMAD7 ODN
ccctcctcctcgtcctcg

SEQ ID NO: 36 - DNA, 20 bp, Artificial Sequence
  SMAD7 ODN
gtcgccccttctccccgcag

SEQ ID NO: 37 - DNA, 18 bp, Artificial Sequence
  SMAD7 ODN
gccgtccgtcgccccttc

SEQ ID NO: 38 - DNA, 18 bp, Artificial Sequence
  SMAD7 ODN
agcaccgagtgcgtgagc

SEQ ID NO: 39 - DNA, 18 bp, Artificial Sequence
  SMAD7 ODN
agttcacagagtcgacta

SEQ ID NO: 40 - DNA, 18 bp, Artificial Sequence
  SMAD7 ODN
ggcaaaagccattcccct

SEQ ID NO: 41 - DNA, 18 bp, Artificial Sequence
  SMAD7 ODN
gccgatcttgctcctcac

SEQ ID NO: 42 - DNA, 21 bp, Artificial Sequence
  SMAD7 ODN
gtcgccccttctcccccgcag

SEQ ID NO: 43 - DNA, 20 bp, Artificial Sequence
  SMAD7 ODN
gtcgcaccgtctcacagcag

SEQ ID NO: 44 - DNA, 15 bp, Artificial Sequence
  SMAD7 ODN
atggacaatatgtct

SEQ ID NO: 45 - DNA, 20 bp, Artificial Sequence
  SMAD7 ODN
ctgcggggagaaggggcgac

SEQ ID NO: 46 - RNA, 21 nt, Artificial Sequence
  SMAD7 ODN
guucaggaccaaacgaucugc

SEQ ID NO: 47 - RNA, 23 nt, Artificial Sequence
  SMAD7 ODN
gcagaucguuugguccugaacau

SEQ ID NO: 48 - RNA, 21 nt, Artificial Sequence
  SMAD7 ODN
cucacgcacucggugcucaag

SEQ ID NO: 49 - RNA, 23 nt, Artificial Sequence
  SMAD7 ODN
cuugagcaccgagugcgugagcg

SEQ ID NO: 50 - RNA, 21 nt, Artificial Sequence
  SMAD7 ODN
cucggcgcccgacuucuucuu

SEQ ID NO: 51 - RNA, 21 nt, Artificial Sequence
  SMAD7 ODN
gaagaagucgggcgccgaguu

SEQ ID NO: 52 - RNA, 21 nt, Artificial Sequence
  SMAD7 ODN
acgacuuuucuccucgccuuu

SEQ ID NO: 53 - RNA, 21 nt, Artificial Sequence
  SMAD7 ODN
aggcgaggagaaaagucguuu

SEQ ID NO: 54 - RNA, 21 nt, Artificial Sequence
  SMAD7 ODN
acgaucugcgcucguccgguu

SEQ ID NO: 55 - RNA, 21 nt, Artificial Sequence
  SMAD7 ODN
ccggacgagcgcagaucguuu

SEQ ID NO: 56 - RNA, 21 nt, Artificial Sequence
  SMAD7 ODN
ggcgcucacgcacucgguguu

SEQ ID NO: 57 - RNA, 21 nt, Artificial Sequence
  SMAD7 ODN
caccgagugcgugagcgccuu

SEQ ID NO: 58 - RNA, 21 nt, Artificial Sequence
  SMAD7 ODN
ggagcggcagcuggagcuguu

SEQ ID NO: 59 - RNA, 21 nt, Artificial Sequence
  SMAD7 ODN
cagcuccagcugccgcuccuu

SEQ ID NO: 60 - RNA, 21 nt, Artificial Sequence
  SMAD7 ODN
aguguucagguggccggauuu

SEQ ID NO: 61 - RNA, 21 nt, Artificial Sequence
  SMAD7 ODN
auccggccaccugaacacuuu

SEQ ID NO: 62 - RNA, 21 nt, Artificial Sequence
  SMAD7 ODN
gucaagaggcuguguugcuuu

SEQ ID NO: 63 - RNA, 21 nt, Artificial Sequence
  SMAD7 ODN
agcaacacagccucuugacuu

SEQ ID NO: 64 - RNA, 21 nt, Artificial Sequence
  SMAD7 ODN
gaggcuguguugcugugaauu

SEQ ID NO: 65 - RNA, 21 nt, Artificial Sequence
  SMAD7 ODN
uucacagacacacagccucuu

SEQ ID NO: 66 - RNA, 21 nt, Artificial Sequence
  SMAD7 ODN
ucuuacgggaagaucaaccuu

SEQ ID NO: 67 - RNA, 21 nt, Artificial Sequence
  SMAD7 ODN
gguugaucuucccguaagauu

SEQ ID NO: 68 - RNA, 21 nt, Artificial Sequence
  SMAD7 ODN
gaucaaccccgagcugguguu

SEQ ID NO: 69 - RNA, 21 nt, Artificial Sequence
  SMAD7 ODN
caccagcucgggguugaucuu

SEQ ID NO: 70 - RNA, 21 nt, Artificial Sequence
  SMAD7 ODN
ccccgagcuggugugcugcuu

SEQ ID NO: 71 - RNA, 21 nt, Artificial Sequence
  SMAD7 ODN
gcagcacaccagcucgggguu

SEQ ID NO: 72 - RNA, 21 nt, Artificial Sequence
  SMAD7 ODN
cgaauuaucuggccccugguu

SEQ ID NO: 73 - RNA, 21 nt, Artificial Sequence
  SMAD7 ODN
ccaggggccagauaauucguu

SEQ ID NO: 74 - RNA, 21 nt, Artificial Sequence
  SMAD7 ODN
cuucuucuggagccugggguu

SEQ ID NO: 75 - RNA, 21 nt, Artificial Sequence
  SMAD7 ODN
ccccaggcuccagaagaaguu

SEQ ID NO: 76 - RNA, 21 nt, Artificial Sequence
  SMAD7 ODN
uggcuuuugccucggacaguu

SEQ ID NO: 77 - RNA, 21 nt, Artificial Sequence
  SMAD7 ODN
cuguccgaggcaaaagccauu

SEQ ID NO: 78 - RNA, 21 nt, Artificial Sequence
  SMAD7 ODN
uucggacaacaagagucaguu

SEQ ID NO: 79 - RNA, 21 nt, Artificial Sequence
  SMAD7 ODN
cugacucuuguuguccgaauu

SEQ ID NO: 80 - RNA, 21 nt, Artificial Sequence
  SMAD7 ODN
ccgcagcaguuaccccaucuu

SEQ ID NO: 81 - RNA, 21 nt, Artificial Sequence
  SMAD7 ODN
gaugggguaacugcugcgguu

SEQ ID NO: 82 - RNA, 21 nt, Artificial Sequence
  SMAD7 ODN
guccgccacacuggacaacuu

SEQ ID NO: 83 - RNA, 21 nt, Artificial Sequence
  SMAD7 ODN
guuguccaguguggcggacuu

SEQ ID NO: 84 - RNA, 21 nt, Artificial Sequence
  SMAD7 ODN
cccggacuccaggacgcuguu

SEQ ID NO: 85 - RNA, 21 nt, Artificial Sequence
  SMAD7 ODN
cagcguccuggaguccggguu

SEQ ID NO: 86 - DNA, 24 bp, Artificial Sequence
  SMAD7 ODN
atgttcaggaccaaacgatctgcg

SEQ ID NO: 87 - DNA, 23 bp, Artificial Sequence
  SMAD7 ODN
agctgccgctccttcagtttctt

SEQ ID NO: 88 - DNA, 1282 bp, Homo sapiens
  human SMAD7 mRNA (GenBank XM_033746)
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcaggattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 89 - DNA, 3087 bp, Homo sapiens
  human SMAD7 mRNA (GenBank XM_008803)
cggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcaggattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccagcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgtttttttctatgggtgttatcacctagctgaatgtttttctaaaggagtttatgttccattaaacgatttttaaaatgtacacttg

SEQ ID NO: 90 - DNA, 1281 bp, Homo sapiens
  human SMAD7 mRNA (GenBank AF015261)
atgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaatgtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtag

SEQ ID NO: 91 - DNA, 20 bp, Artificial Sequence
  modified SMAD7 ODN
  misc_feature 3, 16: n = cytosine, 5-methylcytosine, or 2-O-methylcytosine
  misc_feature 4, 17: n = guanine, 5-methylguanine, or 2-O-methylguanine
gtnnccccttctcccnncag

SEQ ID NO: 92 - DNA, 20 bp, Artificial Sequence
  modified SMAD7 ODN
  misc_feature 3, 16: n = 5-methyl 2′-deoxycytidine 5′-monophosphate
gtngccccttctcccngcag

SEQ ID NO: 93 - DNA, 20 bp, Artificial Sequence
  modified SMAD7 ODN
  misc_feature 1, 20: n = 2′-deoxyguanosine methylphosphonate
  misc_feature 3, 16: n = 5-methyl-2′-deoxycytidine-5′-monophosphate
ntngccccttctcccngcan

SEQ ID NO: 94 - DNA, 18 bp, Artificial Sequence
  SMAD7 ODN
gtttggtcctgaacatgc

SEQ ID NO: 95 - DNA, 16 bp, Artificial Sequence
  SMAD7 ODN
gtttggtcctgaacat

SEQ ID NO: 96 - DNA, 17 bp, Artificial Sequence
  SMAD7 ODN
gtttggtcctgaacatg

SEQ ID NO: 97 - DNA, 18 bp, Artificial Sequence
  SMAD7 ODN
agcaccgagtgcgtgagc

SEQ ID NO: 98 - DNA, 18 bp, Artificial Sequence
  modified SMAD7 ODN
  misc_feature 1, 18: n = 2′-deoxyadenosine methylphosphonate
  misc_feature 6, 12: n = 5-methyl-2′-deoxycytidine-5′-monophosphate
ngcacngagtgngtgagn

SEQ ID NO: 99 - DNA, 18 bp, Artificial Sequence
  SMAD7 ODN
cgaacatgacctccgcac

SEQ ID NO: 100 - DNA, 18 bp, Artificial Sequence
  modified SMAD7 ODN
  misc_feature 1, 14: n = 5-methyl-2′-deoxycytidine-5′-monophosphate
ngaacatgacctcngcac

SEQ ID NO: 101 - DNA, 20 bp, Artificial Sequence
  modified SMAD7 ODN
  misc_feature 3, 16: n = 5-methyl-2′-deoxycytidine-5′-monophosphate
gtngccccttctcccngcag

SEQ ID NO: 102 - DNA, 17 bp, Artificial Sequence
  SMAD7 ODN
gatcgtttggtcctgaa

SEQ ID NO: 103 - DNA, 17 bp, Artificial Sequence
  SMAD7 ODN
atcgtttggtcctgaac

SEQ ID NO: 104 - DNA, 21 bp, Artificial Sequence
  modified SMAD7 ODN
  misc_feature 1, 20: n = 2′-deoxyadenosine methylphosphonate
  misc_feature 3, 16: n = 5-methyl-2′-deoxycytidine-5′-monophosphate
ntngccccttctcccngcanc

SEQ ID NO: 105 - DNA, 21 bp, Artificial Sequence
  modified SMAD7 ODN
  misc_feature 3, 16: n = 5-methyl-2′-deoxycytidine-5′-monophosphate
gtngccccttctcccngcagc

SEQ ID NO: 106 - DNA, 20 bp, Artificial Sequence
  modified SMAD7 ODN
  misc_feature 3, 16: n = chemically modified nucleoside
gtngccccttctcccngcag

SEQ ID NO: 107 - DNA, 20 bp, Artificial Sequence
  SMAD7 ODN
tccgtcatcgctcctcaggg

SEQ ID NO: 108 - DNA, 20 bp, Artificial Sequence
  SMAD7 ODN
atgcattctgcccccaagga

SEQ ID NO: 109 - DNA, 24 bp, Artificial Sequence
  SMAD7 ODN
tgtgaatcttacgggaagatcaac

SEQ ID NO: 110 - DNA, 21 bp, Artificial Sequence
  SMAD7 ODN
agttcgcagagtcggctaagg

SEQ ID NO: 111 - DNA, 22 bp, Artificial Sequence
  SMAD7 ODN
ccgagctggtgtgctgcaaccc

SEQ ID NO: 112 - DNA, 19 bp, Artificial Sequence
  SMAD7 ODN
gaaggtgaaggtcggagtc

SEQ ID NO: 113 - DNA, 20 bp, Artificial Sequence
  SMAD7 ODN
gaagatggtgatgggatttc

SEQ ID NO: 114 - DNA, 20 bp, Artificial Sequence
  SMAD7 ODN
caagcttcccgttctcagcc

SEQ ID NO: 115 - DNA, 18 bp, Artificial Sequence
  SMAD7 ODN
cgacagcagcagcagcag

SEQ ID NO: 116 - DNA, 18 bp, Artificial Sequence
  SMAD7 ODN
aggaggcgaggagaaaag

SEQ ID NO: 117 - DNA, 18 bp, Artificial Sequence
  SMAD7 ODN
gctgtccgtcgccccttc

SEQ ID NO: 118 - DNA, 18 bp, Artificial Sequence
  SMAD7 ODN
aggcggccgggcagcagg

SEQ ID NO: 119 - DNA, 18 bp, Artificial Sequence
  SMAD7 ODN
agcctcttgacttccgag

SEQ ID NO: 120 - DNA, 18 bp, Artificial Sequence
  SMAD7 ODN
agtctgcagttggtttga

SEQ ID NO: 121 - DNA, 18 bp, Artificial Sequence
  SMAD7 ODN
agaagttgggaatctgaa

SEQ ID NO: 122 - DNA, 18 bp, Artificial Sequence
  SMAD7 ODN
gtatgccaccacgcacca

SEQ ID NO: 123 - DNA, 18 bp, Artificial Sequence
  SMAD7 ODN
tggacacagtagagcctc

SEQ ID NO: 124 - DNA, 18 bp, Artificial Sequence
  SMAD7 ODN
gccgattttgctccgcac

SEQ ID NO: 125 - DNA, 18 bp, Artificial Sequence
  SMAD7 ODN
ggacttgatgaagatggg

SEQ ID NO: 126 - DNA, 18 bp, Artificial Sequence
  SMAD7 ODN
accttgtgtaccaacagc

SEQ ID NO: 127 - DNA, 18 bp, Artificial Sequence
  SMAD7 ODN
ctgatctgcacggtaaag

SEQ ID NO: 128 - DNA, 18 bp, Artificial Sequence
  SMAD7 ODN
ggcagctgctgatgaact

SEQ ID NO: 129 - DNA, 18 bp, Artificial Sequence
  SMAD7 ODN
gctcagctcacgctctgt

SEQ ID NO: 130 - DNA, 18 bp, Artificial Sequence
  SMAD7 ODN
tgcatgaaaagcaagcac

SEQ ID NO: 131 - DNA, 18 bp, Artificial Sequence
  SMAD7 ODN
aaaacagaacacaaacga

SEQ ID NO: 132 - DNA, 18 bp, Artificial Sequence
  SMAD7 ODN
tgcttggatttctgcttc

SEQ ID NO: 133 - DNA, 18 bp, Artificial Sequence
  SMAD7 ODN
gttgtctccccatctgcc

SEQ ID NO: 134 - DNA, 18 bp, Artificial Sequence
  SMAD7 ODN
ttgggactgcaaacctct

SEQ ID NO: 135 - DNA, 18 bp, Artificial Sequence
  SMAD7 ODN
aattggttctggttcggc

SEQ ID NO: 136 - DNA, 18 bp, Artificial Sequence
  SMAD7 ODN
aaaacctgatgtcaccag

SEQ ID NO: 137 - DNA, 18 bp, Artificial Sequence
  SMAD7 ODN
acacacaggatgggagca

SEQ ID NO: 138 - DNA, 18 bp, Artificial Sequence
  SMAD7 ODN
ggacatccccgcttgctg

SEQ ID NO: 139 - DNA, 18 bp, Artificial Sequence
  SMAD7 ODN
gatggtaccttgggttat

SEQ ID NO: 140 - DNA, 18 bp, Artificial Sequence
  SMAD7 ODN
gtagaagaaatgaaagaa

SEQ ID NO: 141 - DNA, 18 bp, Artificial Sequence
  SMAD7 ODN
aacctttgtagttagaaa

SEQ ID NO: 142 - DNA, 18 bp, Artificial Sequence
  SMAD7 ODN
atttccaatgagaatgct

SEQ ID NO: 143 - DNA, 18 bp, Artificial Sequence
  SMAD7 ODN
caatcttgtgtttatctt

SEQ ID NO: 144 - DNA, 18 bp, Artificial Sequence
  SMAD7 ODN
attcagctaggtgataac

SEQ ID NO: 145 - DNA, 19 bp, Artificial Sequence
  SMAD7 ODN
ggaacagttcgtccatggc

SEQ ID NO: 146 - DNA, 19 bp, Artificial Sequence
  SMAD7 ODN
ggaacagttagtccatggc

SEQ ID NO: 147 - DNA, 20 bp, Artificial Sequence
  SMAD7 ODN
ctgccacgttctcctgcacc

SEQ ID NO: 148 - DNA, 20 bp, Artificial Sequence
  SMAD7 ODN
ctgccaagttcacctgcacc

SEQ ID NO: 149 - DNA, 21 bp, Artificial Sequence
  SMAD7 ODN
gtcgccccttctccccgcagc

SEQ ID NO: 150 - DNA, 21 bp, Artificial Sequence
  SMAD7 ODN
gtagccccatcacccagcagc

SEQ ID NO: 151 - DNA, 21 bp, Artificial Sequence
  SMAD7 ODN
gtcgctcttctcctccgccag

SEQ ID NO: 152 - DNA, 24 bp, Artificial Sequence
  SMAD7 ODN
tcgtcgttttgtcgttttgtcgtt

SEQ ID NO: 153 - DNA, 24 bp, Artificial Sequence
  SMAD7 ODN
tgctgcttttgtgcttttgtgctt

SEQ ID NO: 154 - DNA, 20 bp, Artificial Sequence
  SMAD7 ODN
gggggacgatcgtcgggggg

SEQ ID NO: 155 - DNA, 20 bp, Artificial Sequence
  SMAD7 ODN
tccatgacgttcctgacgtt

SEQ ID NO: 156 - DNA, 20 bp, Artificial Sequence
  SMAD7 ODN
tccatgagcttcctgagctt

SEQ ID NO: 157 - DNA, 24 bp, Artificial Sequence
  SMAD7 ODN
tgctgcttttgtgcttttgtgctt

SEQ ID NO: 158 - DNA, 20 bp, Artificial Sequence
  SMAD7 ODN
gggggacgatcgtcgggggg